(19) United States
(12) Patent Application Publication
(10) Pub. No.: US 2025/0002886 A1
(43) Pub. Date: Jan. 2, 2025

(54) ENGINEERED AND CHIMERIC NUCLEASES

(71) Applicant: Metagenomi, Inc., Emeryville, CA (US)

(72) Inventors: Brian C. Thomas, Berkeley, CA (US); Alan Brooks, Clayton, CA (US); Christopher Brown, Albany, CA (US); Cristina Noel Butterfield, Oakland, CA (US); Daniela S.A. Goltsman, Oakland, CA (US); Jyun-Liang Lin, Richmond, CA (US); Isabel Nocedal, Oakland, CA (US); Morayma Temoche-Diaz, Emeryville, CA (US)

(21) Appl. No.: 18/885,463

(22) Filed: Sep. 13, 2024

Related U.S. Application Data
(63) Continuation of application No. PCT/US2023/064351, filed on Mar. 14, 2023.
(60) Provisional application No. 63/482,294, filed on Jan. 30, 2023.
(60) Provisional application No. 63/392,814, filed on Jul. 27, 2022.
(60) Provisional application No. 63/335,542, filed on Apr. 27, 2022.
(60) Provisional application No. 63/319,725, filed on Mar. 14, 2022.

(51) Int. Cl. C12N 9/22; C12N 15/11

(52) U.S. Cl. CPC C12N 9/22; C12N 15/111; C12N 2310/20

(57) ABSTRACT

Disclosed herein are engineered nucleases and nuclease systems, including chimeric nucleases and chimeric nuclease systems. Engineered and chimeric nucleases disclosed herein include nucleic acid guided nuclease. Additionally disclosed herein are methods of generating engineered nucleases and methods of using the same.

### CROSS-REFERENCE

This application is a continuation of International Application No. PCT/US2023/064351, filed Mar. 14, 2023, which claims the benefit of and priority to U.S. Provisional Patent Application No. 63/319,725, filed Mar. 14, 2022, U.S. Provisional Patent Application No. 63/335,542, filed Apr. 27, 2022, U.S. Provisional Patent Application No. 63/392,814, filed Jul. 27, 2022, and U.S. Provisional Patent Application No. 63/482,294, filed Jan. 30, 2023, each of which is incorporated by reference in its entirety herein.

### SEQUENCE LISTING

The contents of the electronic sequence listing (MTG-010WO_SL.xml; Size: 5,041,672 bytes; and Date of Creation: Jul. 14, 2023) is herein incorporated by reference in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self-nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Mar. 14, 2023, is named MTG-010WO_SL.xml and is 4,575,581 bytes in size.

### SUMMARY

In some aspects, the present disclosure provides for a method of disrupting a VCP locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the VCP locus, wherein the engineered guide RNA is configured to hybridize to or comprises a targeting sequence having at least 80% identity to SEQ ID NOs: 739-754 or 763-770; or wherein the engineered guide RNA comprises a sequence having at least 80% identity to any one of SEQ ID NOs: 723-738 or 755-762. In some embodiments, the class 2, type II Cas endonuclease comprises any of the engineered nucleases described herein. In some embodiments, the class 2, type II Cas endonuclease comprises a fusion endonuclease having at least 55% identity to SEQ ID NO: 10 or a variant thereof. In some embodiments, the engineered guide RNA comprises a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863. In some embodiments, the engineered guide RNA is complementary to or comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 743-745, 749-752, 754, or 769. In some embodiments, the engineered guide RNA comprises a nucleotide sequence of any one of the guide RNAs from Table 22B, comprising the chemical modifications recited in Table 22B.

In some cases, the present disclosure provides for a fusion endonuclease comprising: (a) an N-terminal sequence comprising at least part of a RuvC-I domain, a REC domain, a RuvC-II domain, an HNH domain, or a RuvC-III domain of an endonuclease having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696 or a variant thereof; and (b) a C-terminal sequence comprising WED or PI domains of an endonuclease having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to MG3-1, MG3-2, MG3-3, MG3-4, MG3-5, MG3-6, MG3-7, MG3-8, MG3-18, MG3-22, MG3-24, MG3-30, MG3-42, MG3-78, MG3-89, MG3-90, MG3-91, MG3-92, MG3-93, MG3-94, MG3-95, MG3-96, MG3-101, MG3-103, MG3-104, MG150-1, MG150-2, MG150-3, MG150-4, MG150-5, MG150-6, MG150-7, MG150-8, MG150-9, MG150-10, MG15-1, MG15-54, MG15-66, MG15-94, MG15-115, MG15-135, MG15-146, MG15-164, MG15-166, MG15-171, MG15-172, MG15-174, MG15-177, MG15-184, MG15-187, MG15-191, MG15-193, MG15-195, MG15-217, MG15-218, or MG15-219, or variants thereof, wherein the N-terminal sequence and the C-terminal sequence do not naturally occur together in a same reading frame. In some embodiments, the N-terminal sequence and the C-terminal sequence are derived from different organisms. In some embodiments, the fusion endonuclease comprises a sequence having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 771-862, or a variant thereof. In some embodiments, the fusion endonuclease is configured to be selective for a PAM that is not nnRGGnT. In some embodiments, the fusion endonuclease is configured to be selective for a PAM that comprises any one of SEQ ID NOs: 865-919. In some embodiments, the fusion endonuclease comprises a WED domain from an endonuclease with at least 55% sequence identity to MG3-8, and a PI domain from an endonuclease with at least 55% sequence identity to at least one of MG3-1, MG3-2, MG3-3, MG3-4, MG3-5, MG3-6, MG3-7, MG3-8, MG3-18, MG3-22, MG3-24, MG3-30, MG3-42, MG3-78, MG3-89, MG3-90, MG3-91, MG3-92, MG3-93, MG3-94, MG3-95, MG3-96, MG3-101, MG3-103, MG3-104, MG150-1, MG150-2, MG150-3, MG150-4, MG150-5, MG150-6, MG150-7, MG150-8, MG150-9, MG150-10, MG15-1, MG15-54, MG15-66, MG15-94, MG15-115, MG15-135, MG15-146, MG15-164, MG15-166, MG15-171, MG15-172, MG15-174, MG15-177, MG15-184, MG15-187, MG15-191, MG15-193, MG15-195, MG15-217, MG15-218, or MG15-219, or variants thereof.

In some aspects, the present disclosure provides for an endonuclease comprising an engineered amino acid sequence having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 771-862, or a variant thereof. In some embodiments, the endonuclease comprises an engineered amino acid sequence having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 809-823, 827, 829-830, 832, 834, or 838-839.

In some aspects, the present disclosure provides for an engineered nuclease system, comprising: (a) any of the endonucleases described herein; and (b) an engineered guide ribonucleic structure configured to form a complex with the endonuclease comprising: a guide ribonucleic acid configured to hybridize to a target deoxyribonucleic acid sequence; wherein the guide ribonucleic acid sequence is configured to bind to the endonuclease. In some embodiments, the guide ribonucleic acid further comprises a tracer ribonucleic acid sequence configured to bind the endonuclease. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the endonuclease comprises a sequence having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 771-862, or a variant thereof. In some embodiments, the guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting a TRAC locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the TRAC locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 925-927, 992-1011, 1184-1279, or 1321-1361; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 922-924, 972-991, 1088-1183, or 1280-1320. In some embodiments, the class 2, type II Cas endonuclease comprises the fusion endonuclease described herein or comprises a sequence having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 771. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting an AAVS1 locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the AAVS1 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 950-971 or 1050-1087; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 928-949 or 1012-1049. In some embodiments, the class 2, type II Cas endonuclease comprises the fusion endonuclease described herein or comprises a sequence having at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 771. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting an PCSK9 locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the PCSK9 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 1377-1391; or wherein the engineered guide RNA comprises a sequence having at least 80% identity to any one of SEQ ID NOs: 1362-1376. In some embodiments, the class 2, type II Cas endonuclease comprises the fusion endonuclease described herein. In some embodiments, the class 2, type II Cas endonuclease comprises a fusion endonuclease having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting an ANGPTL3 locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the ANGPTL3 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 1490-1587; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1392-1489. In some embodiments, the class 2, type II Cas endonuclease comprises the fusion endonuclease described herein. In some embodiments, the class 2, type II Cas endonuclease comprises a fusion endonuclease having at least 55%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting a GPR146 locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the GPR146 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 1657-1725; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1588-1656. In some embodiments, the class 2, type II Cas endonuclease comprises the fusion endonuclease described herein. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a method of disrupting an APOA1 locus in a cell, comprising introducing to the cell: (a) a class 2, type II Cas endonuclease; and (b) an engineered guide RNA, wherein the engineered guide RNA is configured to form a complex with the endonuclease and the engineered guide RNA comprises a targeting sequence configured to hybridize to a region of the APOA1 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 1745-1763 or 1775-1785; or wherein the engineered guide RNA comprises a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1726-1744 or 1764-1774. In some embodiments, the class 2, type II Cas endonuclease comprises the fusion endonuclease described herein. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722 or SEQ ID NO: 863.

In some aspects, the present disclosure provides for a lipid nanoparticle comprising: (a) any of the endonucleases described herein; (b) any of the engineered guide RNAs described herein: (c) a cationic lipid; (d) a sterol; (e) a neutral lipid; and (f) a PEG-modified lipid. In some embodiments, the cationic lipid comprises C12-200, the sterol comprises cholesterol, the neutral lipid comprises DOPE, or the PEG-modified lipid comprises DMG-PEG2000. In some embodiments, the cationic lipid comprises any of the cationic lipids depicted in FIG. 45.

In some aspects, the present disclosure provides for a fusion endonuclease comprising: (a) an N-terminal sequence comprising at least part of a RuvC domain, a REC domain, or an HNH domain of an endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696 or a variant thereof; and (b) a C-terminal sequence comprising WED, TOPO, or CTD domains of an endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721 or variants thereof, wherein the N-terminal sequence and the C-terminal sequence do not naturally occur together in a same reading frame. In some embodiments, the endonuclease is a Class 2, type II Cas endonuclease. In some embodiments, the endonuclease is a Class 2, type V Cas endonuclease. In some embodiments, the N-terminal sequence and the C-terminal sequence are derived from different organisms. In some embodiments, the N-terminal sequence further comprises RuvC-I, BH, or RuvC-II domains. In some embodiments, the C-terminal sequence further comprises a PAM-interacting domain. In some embodiments, the fusion endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or 108. In some embodiments, the fusion endonuclease is configured to bind to a PAM that is not nnRGGnT.

In some aspects, the present disclosure provides for an endonuclease comprising an engineered amino acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or 108, or a variant thereof.

In some aspects, the present disclosure provides for an endonuclease comprising an engineered amino acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 109-110, or a variant thereof.

In some aspects, the present disclosure provides for a nucleic acid comprising a sequence encoding any of the endonucleases, fusion endonucleases, or Cas enzymes described herein. In some aspects, the sequence is codon-optimized for expression in a host cell. In some embodiments, the host cell is prokaryotic, eukaryotic, mammal, or human.

In some aspects, the present disclosure provides for a vector comprising any of the nucleic acid sequences described herein.

In some aspects, the present disclosure provides for a host cell comprising any of the vectors, systems, or nucleic acids described herein. In some embodiments, the host cell is prokaryotic, eukaryotic, mammal, or human.

In some aspects, the present disclosure provides for an engineered nuclease system, comprising: (a) any of the nucleases, Cas enzymes, or fusion endonucleases described herein; and (b) an engineered guide ribonucleic structure configured to form a complex with the endonuclease comprising: a guide ribonucleic acid configured to hybridize to a target deoxyribonucleic acid sequence; wherein the guide ribonucleic acid sequence is configured to bind to the endonuclease. In some embodiments, the guide ribonucleic acid further comprises a tracr ribonucleic acid sequence configured to bind the endonuclease. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the system further comprises a source of Mg²⁺. In some embodiments, the endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 8-12, 26-27, or 108, or a variant thereof. In some embodiments, the guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to non-degenerate nucleotides of any one of SEQ ID NOs: 33, 34, 44, 45, 78, 84, or 87.

In some aspects, the present disclosure provides for an engineered nuclease comprising: (a) a class 2, type II Cas enzyme RuvC or HNH domain having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a RuvC or HNH domain of any one of SEQ ID NOs: 1-27, 108, or 109-110, or variants thereof; and (b) a class 2, type II Cas enzyme PAM-interacting (PI) domain having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a PAM-interacting (PI) domain any one of SEQ ID NOs: 1-27, 108, or 109-110, or variants thereof. In some embodiments, (a) and (b) do not naturally occur together. In some embodiments, the class 2, type II Cas enzyme is derived from an uncultivated microorganism. In some embodiments, the endonuclease has less than 86% identity to a SpyCas9 endonuclease. In some embodiments, the engineered nuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or a variant thereof.

In some aspects, the present disclosure provides for an engineered nuclease system, comprising: (a) any of the endonucleases described herein; and (b) an engineered guide ribonucleic structure configured to form a complex with the endonuclease comprising: a guide ribonucleic acid sequence configured to hybridize to a target deoxyribonucleic acid sequence and configured to bind to the endonuclease. In some embodiments, the guide ribonucleic acid further comprises a tracr ribonucleic acid sequence configured to bind the endonuclease. In some embodiments, the guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of any one of SEQ ID NOs: 28-32 or 33-44, or a variant thereof. In some embodiments, the system further comprises a PAM sequence compatible with the nuclease adjacent to the target nucleic acid site. In some embodiments, the PAM sequence is located 3′ of the target deoxyribonucleic acid sequence. In some embodiments, the PAM sequence is located 5′ of the target deoxyribonucleic acid sequence.

In some aspects, the present disclosure provides for a method of targeting the albumin gene, comprising introducing any of the systems described herein to a cell, wherein the guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity any one of SEQ ID NOs: 67-86. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of targeting the HAO1 gene or locus, comprising introducing any of the systems described herein to a cell, wherein the guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 611-633. In some embodiments, the guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 615, 618, 620, 624, or 626. In some embodiments, the guide ribonucleic acid comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 605-610 or 1789-1865. In some embodiments, the guide ribonucleic acid comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 1812-1824, 1835, 1849, 1858, or 1861, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 1812-1824, 1835, 1849, 1858, or 1861. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some embodiments, the present disclosure provides for a method of disrupting an HAO-1 locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the HAO-1 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 611-633. In some embodiments, the endonuclease is a class 2, type II Cas endonuclease. In some embodiments, the class 2, type II Cas endonuclease comprises any of the fusion or engineered endonucleases described herein.

In some embodiments the endonuclease comprises any of the fusion or engineered endonucleases described herein. In some embodiments, the class 2, type II Cas endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 618, 620, 624, or 626, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a targeting sequence of any one of SEQ ID NOs: 618, 620, 624, or 626. In some embodiments, the engineered guide RNA comprises the nucleotide sequence of any one of the guide RNAs from Tables 9A-9E, Table 12, Table 18, Table 20, or Table 23. In some embodiments, the engineered guide RNA comprises a nucleotide sequence of any one of the guide RNAs from Tables 9A-9E, comprising the chemical modifications recited in Tables 9A-9E. In some embodiments, the engineered guide RNA comprises a nucleotide sequence of any one of the guide RNAs from Table 12, comprising the chemical modifications recited in Table 12. In some embodiments, the engineered guide RNA comprises a nucleotide sequence of any one of the guide RNAs from Table 18, comprising the chemical modifications recited in Table 18.

In some embodiments, the engineered guide RNA comprises a nucleotide sequence of any one of the guide RNAs from Table 20, comprising the chemical modifications recited in Table 20. In some embodiments, the engineered guide RNA comprises a nucleotide sequence of any one of the guide RNAs from Table 23, comprising the chemical modifications recited in Table 23. In some embodiments, the cell is a mammalian cell. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of disrupting a TRAC locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the TRAC locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 139-158; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 119-138. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease comprises any of the fusion endonucleases described herein. In some embodiments, the class 2, type II Cas endonuclease comprises the fusion endonuclease having at least 55% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 121, 132, 136, 130, 134, 135, or 137, or a sequence having at least 80% identity to a targeting sequence of any one of SEQ ID NOs: 121, 132, 136, 130, 134, 135, or 137. In some embodiments, the engineered guide RNA comprises a nucleotide sequence of any one of the guide RNAs from Table 9A. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some embodiments, the present disclosure provides for a method of disrupting a B2M locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the B2M locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 185-210; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 159-184. In some embodiments, the endonuclease is a class 2, type II Cas endonuclease. In some embodiments, the class 2, type II Cas endonuclease comprises any of the fusion or engineered endonucleases described herein.

In some embodiments the endonuclease comprises any of the fusion or engineered endonucleases described herein. In some embodiments, the class 2, type II Cas endonuclease comprises any of the fusion endonucleases described herein. In some embodiments, the class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 159, 165, 168, 174, or 184, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 159, 165, 168, 174, or 184. In some embodiments, the engineered guide RNA comprises a nucleotide sequence of any one of the guide RNAs from Table 9B. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of disrupting a TRBC1 locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the TRBC1 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 252-292; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 211-251. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease comprises any of the fusion endonucleases described herein. In some embodiments, the class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, the engineered guide RNA is comprises a sequence having at least 80% identity to any one of SEQ ID NOs: 211, 212, 215, 241, or 242, or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 211, 212, 215, 241, or 242. In some embodiments, the engineered guide RNA comprises a nucleotide sequence of any one of the guide RNAs from Table 9C. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of disrupting a TRBC2 locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the TRBC2 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 338-382; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 293-337. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease any of the fusion endonucleases described herein. In some embodiments, the class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 296, 306, or 332, or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID Nos: 296, 306, or 332. In some embodiments, the engineered guide RNA comprises a nucleotide sequence of any one of the guide RNAs from Table 9C. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of disrupting an ANGPTL3 locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the ANGPTL3 locus, wherein the engineered guide RNA is configured to hybridize to or comprises a targeting sequence having at least 80% identity to SEQ ID NOs: 478-572; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 383-477. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease comprises any of the fusion endonucleases described herein. In some embodiments, the class 2, type II Cas endonuclease comprises a fusion endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a non-degenerate nucleotides of SEQ ID NO: 722. In some embodiments, the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 419, 425, 431, 439, 447, 453, 461, 467, 471, or 473, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 419, 425, 431, 439, 447, 453, 461, 467, 471, or 473. In some embodiments, the engineered guide RNA comprises a nucleotide sequence of any one of the guide RNAs from Table 9D. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for a method of disrupting a PCSK9 locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the PCSK9 locus, wherein the engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 588-602; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 573-587. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease comprises any of the fusion endonucleases described herein. In some embodiments, the class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, the engineered guide comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 574, 578, 581, or 585. In some embodiments, the engineered guide RNA comprises a nucleotide sequence of any one of the guide RNAs from Table 9E. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some embodiments, the present disclosure provides for a method of disrupting an albumin locus in a cell, comprising introducing to the cell: (a) any of the endonucleases described herein; and (b) an engineered guide RNA, wherein the engineered guide RNA is configured to form a complex with the endonuclease and the engineered guide RNA comprises a targeting sequence configured to hybridize to a region of the albumin locus, wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 67-86 or 646-695, or wherein the engineered guide RNA comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 67-86 or 646-695. In some embodiments, the endonuclease is a class 2, type II Cas endonuclease. In some embodiments, the class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease comprises any of the type II Cas endonucleases described herein. In some embodiments, the class 2, type II Cas endonuclease comprises a fusion endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, the engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, the engineered guide RNA is complementary to or comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 67, 68, 70, 71, 72, 76, 79, 80, 647, 648, 649, 653, 654, 655, 656, 673, 680, 681, or 682. In some embodiments, the engineered guide RNA comprises a nucleotide sequence of any one of the guide RNAs from Table 6. In some embodiments, introducing to the cell further comprises contacting the cell with a nucleic acid or vector encoding the fusion protein or the guide polynucleotide. or comprises contacting the cell with a lipid nanoparticle (LNP) comprising the vector or nucleic acid. In some embodiments, introducing to the cell further comprises contacting the cell with a ribonucleoprotein complex (RNP) comprising the fusion protein or the guide polynucleotide or comprises contacting the cell with a lipid nanoparticle (LNP) comprising the RNP.

In some aspects, the present disclosure provides for an endonuclease comprising an engineered amino acid sequence having at least 55% sequence identity to any one of SEQ ID NOs: 1-27, 108, or 109-110.

In some aspects, the present disclosure provides an engineered nuclease system, comprising the endonuclease described herein, and an engineered guide ribonucleic structure configured to form a complex with the endonuclease comprising: a guide ribonucleic acid sequence configured to hybridize to a target deoxyribonucleic acid sequence; and a tracr ribonucleic acid sequence configured to bind to the endonuclease. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the system further comprises a source of MG²⁺.

In some aspects, the present disclosure provides for an engineered nuclease comprising: (a) a class 2, type II Cas enzyme RuvC and HNH domain having at least 55% sequence identity to a RuvC and HNH domain of any one of SEQ ID NOs: 1-27, 108, or 109-110; and (b) a class 2, type II Cas enzyme PAM-interacting (PI) domain having at least 55% sequence identity to a PAM-interacting (PI) domain any one of SEQ ID NOs: 1-27, 108, or 109-110. In some embodiments, (a) and (b) do not naturally occur together. In some embodiments, the class 2, type II Cas enzyme is derived from an uncultivated microorganism. In some embodiments, the endonuclease has less than 86% identity to a SpyCas9 endonuclease. In some embodiments, the engineered nuclease comprises a sequence having at least 55% sequence identity to any one of SEQ ID NOs: 1-27.

In some aspects, the present disclosure provides for an engineered nuclease system, comprising: an endonuclease according to any of the aspects or embodiments described herein; and an engineered guide ribonucleic structure configured to form a complex with the endonuclease comprising: a guide ribonucleic acid sequence configured to hybridize to a target deoxyribonucleic acid sequence; and a tracr ribonucleic acid sequence configured to bind to the endonuclease. In some embodiments, the guide ribonucleic acid sequence comprises a sequence having at least 80% sequence identity to non-degenerate nucleotides of any one of SEQ ID NOs: 28-32 or 33-44, or a variant thereof. In some embodiments, the system further comprises a PAM sequence compatible with the nuclease adjacent to the target nucleic acid site. In some embodiments, the PAM sequence is located 3′ of the target deoxyribonucleic acid sequence.

In some embodiments, the present disclosure provides for an engineered single-molecule heterologous guide polynucleotide compatible with a class 2, type II enzyme according to any of the aspects or embodiments described herein, wherein the heterologous guide polynucleotide comprises chemical modifications according to any one of SEQ ID NOs: 605-610 or 1789-1865.

In some aspects, the present disclosure provides for a method of targeting the albumin gene, comprising introducing a system according to any one of the aspects or embodiments described herein to a cell, wherein the guide ribonucleic acid sequence is configured to hybridize to a sequence comprising any one of SEQ ID NOs: 67-86.

In some aspects, the present disclosure provides for a method of targeting the HAO1 gene, comprising introducing a system according to any one of the aspects or embodiments described herein to a cell, wherein the guide ribonucleic acid sequence is configured to hybridize to any one of SEQ ID NOs: 611-633. In some embodiments, the guide ribonucleic acid sequence is configured to hybridize to any one of SEQ ID NOs: 615, 618, 620, 624, or 626. In some embodiments, the guide ribonucleic acid comprises a sequence according to any one of SEQ ID NOs: 605-610 or 1789-1865. In some embodiments, the guide ribonucleic acid comprises a sequence according to any one of SEQ ID NOs: 1812-1824, 1835, 1849, 1858, or 1861.

In some aspects, the present disclosure provides cells comprising the endonucleases described herein. In some aspects, the present disclosure provides cells comprising any nucleic acid molecule described herein. In some aspects, the present disclosure provides cells comprising any engineered nuclease system described herein.

Described herein, in certain embodiments, are engineered endonucleases, comprising: a) an N-terminal portion comprising a sequence having at least 80% sequence identity to SEQ ID NO: 696; and b) a C-terminal portion comprising a sequence having at least 80% sequence identity to any one of SEQ ID NOs: 697-721. In some embodiments, the N-terminal portion and the C-terminal portion are fused directly to each other. In some embodiments, the N-terminal portion and the C-terminal portion are joined by a linker. In some embodiments, the linker is a glycine and/or serine-rich linker, a large protein domain, a long helix structure, or a short helix structure. In some embodiments, the linker is (GGGGS)n, and wherein n is an integer from 1 to 20. (SEQ ID NO: 2950) In some embodiments, the linker is GGGGS (SEQ ID NO: 2864). In some embodiments, the N-terminal portion comprises a sequence having at least 90% sequence identity to SEQ ID NO: 696. In some embodiments, the N-terminal portion comprises a sequence having 100% sequence identity to SEQ ID NO: 696. In some embodiments, the C-terminal portion comprises a sequence having at least 90% sequence identity to any one of SEQ ID NOs: 697-721. In some embodiments, the C-terminal portion comprises a sequence having 100% sequence identity to any one of SEQ ID NOs: 697-721. In some embodiments, the engineered endonuclease is configured to bind to a PAM that comprises any one of SEQ ID NOs: 60-66, 117, 865-919, and 2855-2863.

Described herein, in certain embodiments, are engineered endonucleases comprising a sequence having at least 80% sequence identity to any one of SEQ ID NOs: 1-27 and 771-862. In some embodiments, the engineered endonuclease comprises a sequence having at least 90% sequence identity to any one of SEQ ID NOs: 1-27 and 771-862. In some embodiments, the engineered endonuclease comprises a sequence having 100% sequence identity to any one of SEQ ID NOs: 1-27 and 771-862.

Described herein, in certain embodiments, are engineered endonucleases comprising a sequence having at least 80% sequence identity to SEQ ID NO: 10. In some embodiments, the engineered endonuclease comprises a sequence having at least 90% sequence identity to SEQ ID NO: 10. In some embodiments, the engineered endonuclease comprises a sequence having 100% sequence identity to SEQ ID NO: 10.

Described herein, in certain embodiments, are engineered endonucleases comprising a sequence having at least 80% sequence identity to any one of SEQ ID NOs: 109-110 and 2842-2854. In some embodiments, the engineered endonuclease comprises a sequence having at least 90% sequence identity to any one of SEQ ID NOs: 109-110 and 2842-2854. In some embodiments, the engineered endonuclease comprises a sequence having 100% sequence identity to any one of SEQ ID NOs: 109-110 and 2842-2854.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and to hybridize to a target nucleic acid sequence. In some embodiments, the engineered endonuclease comprises a sequence having at least 90% sequence identity to any one of SEQ ID NOs: 1-27 and 771-862. In some embodiments, the engineered endonuclease comprises a sequence having 100% sequence identity to any one of SEQ ID NOs: 1-27 and 771-862. In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the engineered endonuclease binds non-covalently to the engineered guide polynucleotide. In some embodiments, the endonuclease is covalently linked to the engineered guide polynucleotide. In some embodiments, the endonuclease is fused to the engineered guide polynucleotide. In some embodiments, the engineered guide polynucleotide comprises a sequence having at least 90% sequence identity to any one of SEQ ID NOs: 28-45, 605-610, 646-695, 863, and 1789-1826. In some embodiments, the engineered guide polynucleotide comprises a sequence having 100% sequence identity to any one of SEQ ID NOs: 28-45, 605-610, 646-695, 863, and 1789-1826. In some embodiments, the engineered endonuclease is configured to bind to a PAM that comprises any one of SEQ ID NOs: 60-66, 865-919, and 2863.

Described herein, in certain embodiments, are engineered nuclease systems, comprising: a) an engineered endonuclease comprising a sequence having at least 80% sequence identity to any one of SEQ ID NOs: 109-110 and 2842-2854; and b) an engineered guide polynucleotide configured to form a complex with the endonuclease and to hybridize to a target nucleic acid sequence. In some embodiments, the engineered endonuclease comprises a sequence having at least 90% sequence identity to any one of SEQ ID NOs: 109-110 and 2842-2854. In some embodiments, the engineered endonuclease comprises a sequence having 100% sequence identity to any one of SEQ ID NOs: 109-110 and 2842-2854. In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the engineered endonuclease binds non-covalently to the engineered guide polynucleotide. In some embodiments, the endonuclease is covalently linked to the engineered guide polynucleotide. In some embodiments, the endonuclease is fused to the engineered guide polynucleotide. In some embodiments, the engineered guide polynucleotide comprises a sequence having at least 90% sequence identity to any one of SEQ ID NOs: 111-113. In some embodiments, the engineered guide polynucleotide comprises a sequence having 100% sequence identity to any one of SEQ ID NOs: 111-113. In some embodiments, the engineered endonuclease is configured to bind to a PAM that comprises any one of SEQ ID NOs: 117 and 2855-2862.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least 90% sequence identity to any one of SEQ ID NOs: 67-86.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least 90% sequence identity to any one of SEQ ID NOs: 119-138, 922-924, 972-991, 1088-1183, 1280-1320, 2390-2485, and 2582-2617. In some embodiments, the target nucleic acid sequence comprises a sequence having at least 90% sequence identity to any one of SEQ ID NOs: 139-158, 925-927, 992-1011, 1184-1279, 1321-1361, 2486-2581, and 2618-2653.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least 90% sequence identity to any one of SEQ ID NOs: 159-184. In some embodiments, the target nucleic acid sequence comprises a sequence having at least 90% sequence identity to any one of SEQ ID NOs: 185-210.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least 90% sequence identity to any one of SEQ ID NOs: 211-251. In some embodiments, the target nucleic acid sequence comprises a sequence having at least 90% sequence identity to any one of SEQ ID NOs: 252-292.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least 90% sequence identity to any one of SEQ ID NOs: 293-337. In some embodiments, the target nucleic acid sequence comprises a sequence having at least 90% sequence identity to any one of SEQ ID NOs: 338-382.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least 90% sequence identity to any one of SEQ ID NOs: 383-477, 1392-1489, 2120-2215, and 2312-2350. In some embodiments, the target nucleic acid sequence comprises a sequence having at least 90% sequence identity to any one of SEQ ID NOs: 478-572, 1490-1587, 2216-2311, and 2351-2389.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least 90% sequence identity to any one of SEQ ID NOs: 573-587 and 1362-1376. In some embodiments, the target nucleic acid sequence comprises a sequence having at least 90% sequence identity to any one of SEQ ID NOs: 588-602 and 1377-1391.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least 90% sequence identity to any one of SEQ ID NOs: 723-738 and 755-762. In some embodiments, the target nucleic acid sequence comprises a sequence having at least 90% sequence identity to any one of SEQ ID NOs: 739-754 and 763-770.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least 90% sequence identity to any one of SEQ ID NOs: 928-949 and 1012-1049. In some embodiments, the target nucleic acid sequence comprises a sequence having at least 90% sequence identity to any one of SEQ ID NOs: 950-971 and 1050-1087.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least 90% sequence identity to any one of SEQ ID NOs: 1588-1656. In some embodiments, the target nucleic acid sequence comprises a sequence having at least 90% sequence identity to any one of SEQ ID NOs: 1657-1725.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least 90% sequence identity to any one of SEQ ID NOs: 1726-1744, 1764-1774, 1866-1961 and 2058-2088. In some embodiments, the target nucleic acid sequence comprises a sequence having at least 90% sequence identity to any one of SEQ ID NOs: 1745-1763, 1775-1785, 1962-2057, and 2089-2119.

Described herein, in certain embodiments, are engineered nuclease systems, comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least 90% sequence identity to any one of SEQ ID NOs: 611-633, 1789-1826, and 1827-1865.

Described herein, in certain embodiments, are methods for modifying a target nucleic acid sequence comprising contacting the target nucleic acid sequence using the endonuclease described herein or the engineered nuclease system described herein. In some embodiments, modifying the target nucleic acid sequence comprises binding, nicking, or cleaving, the target nucleic acid sequence. In some embodiments, the target nucleic acid sequence comprises genomic DNA, viral DNA, viral RNA, or bacterial DNA. In some embodiments, the modification is in vitro. In some embodiments, the modification is in vivo. In some embodiments, the modification is ex vivo. In some embodiments, the gRNA is encoded by a sequence having any one of SEQ ID NOs: 251-260, 271-274, and 279-290. In some embodiments, the target nucleic acid sequence comprises a sequence having any one of SEQ ID NOs: 261-270, 275-278, and 291-302.

Described herein, in certain embodiments, are methods of modifying a target nucleic acid sequence in a mammalian cell comprising contacting the mammalian cell using the endonuclease described herein or the engineered nuclease system described herein. In some embodiments, the method further comprises selecting cells comprising the modification.

Described herein, in certain embodiments, are methods of modifying an albumin gene comprising contacting the albumin gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the albumin gene or within an intron of an endogenous gene, the engineered guide polynucleotide comprising a sequence having at least 90% sequence identity to any one of SEQ ID NOs: 67-86.

Described herein, in certain embodiments, are methods of modifying a TRAC gene comprising contacting the TRAC gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the TRAC gene or within an intron of an endogenous gene, the engineered guide polynucleotide comprising a sequence having at least 90% sequence identity to any one of SEQ ID NOs: 119-138, 922-924, 972-991, 1088-1183, 1280-1320, 2390-2485, and 2582-2617. In some embodiments, the target nucleic acid sequence comprises a sequence having at least 90% sequence identity to any one of SEQ ID NOs: 139-158, 925-927, 992-1011, 1184-1279, 1321-1361, 2486-2581, and 2618-2653.

Described herein, in certain embodiments, are methods of modifying a B2M gene comprising contacting the B2M gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the B2M gene or within an intron of an endogenous gene, the engineered guide polynucleotide comprising a sequence having at least 90% sequence identity to any one of SEQ ID NOs: 159-184. In some embodiments, the target nucleic acid sequence comprises a sequence having at least 90% sequence identity to any one of SEQ ID NOs: 185-210.

Described herein, in certain embodiments, are methods of modifying a TRBC1 gene comprising contacting the TRBC1 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the TRBC1 gene or within an intron of an endogenous gene, the engineered guide polynucleotide comprising a sequence having at least 90% sequence identity to any one of SEQ ID NOs: 211-251. In some embodiments, the target nucleic acid sequence comprises a sequence having at least 90% sequence identity to any one of SEQ ID NOs: 252-292.

Described herein, in certain embodiments, are methods of modifying a TRBC2 gene comprising contacting the TRBC2 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the TRBC2 gene or within an intron of an endogenous gene, the engineered guide polynucleotide comprising a sequence having at least 90% sequence identity to any one of SEQ ID NOs: 293-337. In some embodiments, the target nucleic acid sequence comprises a sequence having at least 90% sequence identity to any one of SEQ ID NOs: 338-382.

Described herein, in certain embodiments, are methods of modifying an ANGPTL3 gene comprising contacting the ANGPTL3 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the ANGPTL3 gene or within an intron of an endogenous gene, the engineered guide polynucleotide comprising a sequence having at least 90% sequence identity to any one of SEQ ID NOs: 383-477, 1392-1489, 2120-2215, and 2312-2350. In some embodiments, the target nucleic acid sequence comprises a sequence having at least 90% sequence identity to any one of SEQ ID NOs: 478-572, 1490-1587, 2216-2311, and 2351-2389.

Described herein, in certain embodiments, are methods of modifying a PCSK9 gene comprising contacting the PCSK9 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the PCSK9 gene or within an intron of an endogenous gene, the engineered guide polynucleotide comprising a sequence having at least 90% sequence identity to any one of SEQ ID NOs: 573-587 and 1362-1376. In some embodiments, the target nucleic acid sequence comprises a sequence having at least 90% sequence identity to any one of SEQ ID NOs: 588-602 and 1377-1391.

Described herein, in certain embodiments, are methods of modifying a VCP gene comprising contacting the VCP gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the VCP gene or within an intron of an endogenous gene, the engineered guide polynucleotide comprising a sequence having at least 90% sequence identity to any one of SEQ ID NOs: 723-738 and 755-762. In some embodiments, the target nucleic acid sequence comprises a sequence having at least 90% sequence identity to any one of SEQ ID NOs: 739-754 and 763-770.

Described herein, in certain embodiments, are methods of modifying an AAVS1 locus comprising contacting the AAVS1 locus using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the AAVS1 locus or within an intron of an endogenous gene, the engineered guide polynucleotide comprising a sequence having at least 90% sequence identity to any one of SEQ ID NOs: 928-949 and 1012-1049. In some embodiments, the target nucleic acid sequence comprises a sequence having at least 90% sequence identity to any one of SEQ ID NOs: 950-971 and 1050-1087.

Described herein, in certain embodiments, are methods of modifying a GPR146 gene comprising contacting the GPR146 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the GPR146 gene or within an intron of an endogenous gene, the engineered guide polynucleotide comprising a sequence having at least 90% sequence identity to any one of SEQ ID NOs: 1588-1656. In some embodiments, the target nucleic acid sequence comprises a sequence having at least 90% sequence identity to any one of SEQ ID NOs: 1657-1725.

Described herein, in certain embodiments, are methods of modifying an APOA1 gene comprising contacting the APOA1 gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within the APOA1 gene or within an intron of an endogenous gene, the engineered guide polynucleotide comprising a sequence having at least 90% sequence identity to any one of SEQ ID NOs: 1726-1744, 1764-1774, 1866-1961 and 2058-2088. In some embodiments, the target nucleic acid sequence comprises a sequence having at least 90% sequence identity to any one of SEQ ID NOs: 1745-1763, 1775-1785, 1962-2057, and 2089-2119.

Described herein, in certain embodiments, are methods of modifying a TRAC gene comprising contacting the TRAC gene using an engineered nuclease system comprising: a) an engineered endonuclease comprising a sequence having at least 80% sequence identity to any one of SEQ ID NOs: 1-27 and 771-862; and b) an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least 90% sequence identity to any one of SEQ ID NOs: 611-633, 1789-1826, and 1827-1865.

Described herein, in certain embodiments, are cells comprising the endonuclease described herein or the engineered nuclease system described herein. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-1B depict the natural PAM specificities of various effectors described herein. FIG. 1A shows a phylogenetic tree of the various effectors described herein. FIG. 1B is a table of the PAM specificities of natural RNA guided CRISPR-associated endonucleases.

FIG. 2 demonstrates the concept of domain swapping between RNA guided CRISPR-associated nucleases.

FIGS. 3A and 3B depict the alignment of multiple sequences to guide the determination of an optimal breakpoint. FIG. 3A shows SaCas9 and SpCas9 aligned to several proteins described herein and the terminal conserved residue (an alanine residue) of these sequences are identified as the proposed C-terminus of the swapped section. FIG. 3B depicts the C-terminal domain of a SaCas9 protein to be swapped spans of the RuvC-III, WED, TOPO, and CTD domains. The PAM Interaction domain is composed of the TOPO domain and the CTD domain. Active site residues (D10, E477, and H701 of RuvC domain and D556, D557, and N580 of the NHN domain) are not included in the swapped C-terminal domain.

FIG. 4 depicts the screening of chimeras with an in vitro PAM enrichment assay when recombining MG3-6 with various C-terminal domains from closely and distantly related nucleases. sgRNAs from N-terminal parental domains were used for RNA guided nuclease activities.

FIGS. 5A-5B depict PAM sequences (FIG. 5A) and Seq Logo depictions of PAM sequences (FIG. 5B) of functional chimeras described herein. Given the breakpoint swapping of predicted C-terminal domains of RuvC-III, WED, TOPO and CTD, chimeras were functional if recombined with closely related nucleases. The engineered chimeras tended to preserve PAM specificities from the natural protein's PAM interacting domains, even if the natural protein was not functional in the same experiment.

FIG. 6 shows the screening of chimeras with an in vitro PAM enrichment assay with chimeras recombining MG3-6 with various c-terminal domains from closely and distantly related nucleases. sgRNAs from C-terminal parental domains were used for RNA guided nuclease activities. Numbers in parentheses indicate sgRNA species. Using sgRNAs from C-terminal parental domains did not rescue activities.

FIG. 7 shows predicted structures of MG3-6 and MG15-1. The WED and PI domains of MG3-6 were swapped with those of MG15-1 counterparts to generate chimera 1 (C1). Alternatively, the PI domain of MG3-6 was swapped with MG15-1's counterpart to generate chimera 2 (C2).

FIGS. 8A-8B depict an in vitro PAM enrichment assay and Sanger sequencing results for PAM specificities. C1: MG3-6+MG15-1(WP) and C2: MG3-6+MG15-1(P). The engineered chimeras tend to preserve PAM specificities from the natural proteins' PAM interacting domains. PAM enrichment assay was performed in triplicate. (FIG. 8A) shows an agarose gel depiction of the assay indicating that sequences were cleaved in the presence of the active enzymes and (FIG. 8B) shows SeqLogo depictions of PAM sequences determined by the assay.

FIGS. 9A-9B depict the activity of a chimera described herein in mammalian cells. mRNA codifying for the chimera was co-transfected with 20 different sgRNAs (see e.g., SEQ ID Nos: 67-86) into Hepa 1-6 cells. Editing was assessed by Sanger sequencing and Inference of CRISPR edits (ICE). FIG. 9A shows the editing efficiency of the tested guides. Two biological replicates are shown. FIG. 9B shows the indel profiles created by representative guides.

FIG. 10 depicts the results of a guide screen in Hepa1-6 cells; guides were delivered as mRNA and gRNA using lipofectamine Messenger Max.

FIG. 11A depicts the structural portion of the MG3-6/3-4 guide (SEQ ID NO: 2925).

FIG. 11B depicts the structural portion of the MG3-6 guide (SEQ ID NO: 2925).

FIG. 12 depicts the activity of chemically modified MG3-6/3-4 guides in Hepa1-6 cells when delivered as mRNA and gRNA using lipofectamine Messenger Max.

FIG. 13 depicts the stability of chemically modified MG3-6/3-4 guides over 9 hours at 37° C.

FIG. 14 depicts the stability of chemically modified MG3-6/3-4 guides over 21 hours at 37° C.

FIGS. 15A-15B depict the in vitro screening of Type V-A chimeras. FIG. 15A depicts the agarose gel of amplified cleavage products for each cleavage reaction. Positive enrichment is observed with the MG29-1+MG29-5 chimera, domain swap from the same family (numbers in parentheses indicate sgRNA species). FIG. 15B depicts Seqlogo depictions of PAMs for parent enzymes and the chimeras derived therefrom.

FIG. 16 depicts the gene-editing outcomes at the DNA level for TRAC in HEK293T cells.

FIG. 17 depicts the gene-editing outcomes at the DNA level for B2M in HEK293T cells.

FIG. 18 depicts the gene-editing outcomes at the DNA and phenotypic levels for TRAC in T cells.

FIG. 19 depicts the gene-editing outcomes at the DNA level for B2M in T cells.

FIG. 20 depicts the gene-editing outcomes at the phenotypic level for TRBC1 and TRBC2 in T cells.

FIG. 21 depicts the gene-editing outcomes at the DNA level for ANGPTL3 in Hep3B cells.

FIG. 22 depicts the gene-editing outcomes at the DNA level for PCSK9 in Hep3B cells.

FIG. 23 depicts genome editing at the HAO-1 locus by MG3-6/3-4 in wild type mice analyzed by next generation sequencing.

FIG. 24 depicts glycolate oxidase protein levels in the liver of mice treated with MG3-6/3-4 mRNA and guide RNA targeting the HAO-1 gene.

FIG. 25 depicts genome editing at the HAO-1 locus in wild type mice treated with MG3-6/3-4 mRNA and guide RNA 7 (G7) targeting HAO-1 with 4 different chemical modifications.

FIG. 26 depicts Western blot analysis of glycolate oxidase (GO)/HAO-1 protein levels in the liver of mice at 11 days after treatment with LNP encapsulating MG3-6/3-4 mRNA and sgRNA 7 (G7) with 4 different chemical modifications, as described in Example 19. Individual mice are denoted by numbers.

FIG. 27 depicts the activity of chemically modified guides in Hep3B cells when delivered as mRNA and gRNA using lipid transfection to target HAO-1 as in Example 20.

FIG. 28 depicts the activity of chemically modified guides in Primary Human Hepatocytes when delivered as mRNA and gRNA using lipid transfection to target HAO-1 as described in Example 21.

FIG. 29 depicts demonstration of mammalian cell editing and determination of PAM sequences for two MG3-6 chimeric enzymes as assessed in Example 22.

FIG. 30 depicts the evaluation of MG3-6/3-4 guide chemistries on editing activity in the liver of mice after in vivo delivery in LNP as in Example 23.

FIG. 31 depicts the gene-editing outcomes at the DNA level for VCP in K562 cells as in Example 24.

FIG. 32 depicts gene-editing outcomes at the DNA level in K562 cells. Guides were designed to target TRAC and AAVS1. MG3-6_3-3RWP is a chimera swapping partial RuvC-III, WED, and PID of MG3-6 with MG3-3.

FIG. 33 depicts gene-editing outcomes at the DNA level in K562 cells. Guides were designed to target TRAC and AAVS1. MG3-6_3-7RWP is a chimera swapping partial RuvC-III, WED, and PID of MG3-6 with MG3-7.

FIG. 34 depicts gene-editing outcomes at the DNA level in K562 cells. Guides were designed to target TRAC. MG3-6_3-8RWP is a chimera swapping partial RuvC-III, WED, and PID of MG3-6 with MG3-8.

FIG. 35 depicts gene-editing outcomes at the DNA level in K562 cells. Guides were designed to target TRAC. MG3-6_3-8RWP is a chimera swapping partial RuvC-III, WED, and PID of MG3-6 with MG3-8.

FIG. 36 depicts gene-editing outcomes at the DNA level in human Hep3B cells. Guides were designed to target PCSK9. MG3-6_3-4RWP is a chimera swapping partial RuvC-III, WED, and PID of MG3-6 with MG3-4.

FIG. 37 depicts gene-editing outcomes at the DNA level in human Hep3B cells. Guides were designed to target ANGPTL3. MG3-6_3-4RWP is a chimera swapping partial RuvC-III, WED, and PID of MG3-6 with MG3-4.

FIG. 38 depicts gene-editing outcomes at the DNA level human Hep3B cells. Guides were designed to target APOA1. MG3-6_3-8RWP is a chimera swapping partial RuvC-III, WED, and PID of MG3-6 with MG3-8.

FIG. 39 depicts gene-editing outcomes at the DNA level in mouse Hepa1-6 cells. Guides were designed to target GPR146. MG3-6_3-7RWP is a chimera swapping partial RuvC-III, WED, and PID of MG3-6 with MG3-7RWP.

FIG. 40 depicts gene-editing outcomes at the DNA level in mouse Hepa1-6 cells. Guides were designed to target APOA1. MG3-6_3-4RWP is a chimera swapping partial RuvC-III, WED, and PID of MG3-6 with MG3-4.

FIG. 41 depicts the design strategy of MG3-6 chimeras. Panel (a) of FIG. 41 (top left) depicts predicted domain boundaries of MG3-6 and the breakpoint for recombination with MG3 and MG150 members. Panel (b) of FIG. 41 (bottom left) depicts the predicted 3-D structure of MG3-6 and the breakpoint for protein recombination. The structure is predicted by Novafold. Panel (c) of FIG. 41 (right) depicts a multiple sequence alignment (MSA) of MG3 and MG150 members. The breakpoint is indicated with an arrow.

FIGS. 42A-42B depict screening of MG3-6 chimeras and characterization of PAM specificities. FIG. 42A (left) depicts a DNA agarose gel electrophoresis analysis assessing enzyme activity. FIG. 42B (right) depicts a phylogenetic tree and Seqlogos for functional chimeras.

FIG. 43 depicts design strategy of MG3-6_3-8 chimeras. Panel (a) of FIG. 43 (top left) depicts predicted domain boundaries of MG3-6_3-8 and the breakpoint for recombination with MG3 and MG150 members. Panel (b) of FIG. 43 (bottom left) depicts predicted 3-D structure of MG3-6 and the breakpoint for protein recombination. The structure is predicted by Novafold. Panel (c) of FIG. 43 (middle) depicts a multiple sequence alignment (MSA) of MG3 and MG150 members. Panel (d) of FIG. 43 (right) depicts a multiple sequence alignment (MSA) of MG15 members. The breakpoints are indicated with arrows.

FIGS. 44A-44B depict screening of MG3-6_3-8 chimeras and characterization of PAM specificities. FIG. 44A (top) depicts a DNA agarose gel electrophoresis analysis assessing enzyme activity. FIG. 44B (bottom) depicts Seqlogos consensus depictions of PAM sequences for functional chimeras.

FIG. 45 depicts structures of example cationic lipids that can be used in lipid nanoparticles described herein.

FIG. 46 depicts the evaluation of MG3-6/3-4 guide chemistries on HAO-1 gene editing and mRNA knockdown activity in the liver of mice after in vivo delivery in LNP as described in Example 28.

FIG. 47 depicts gene-editing outcomes at the DNA level for mApoa1 in Hepa1-6 cells.

FIG. 48 depicts gene-editing outcomes at the DNA level for mAngptl3 in Hepa1-6 cells.

FIG. 49 depicts gene-editing outcomes at the DNA level for mTrac in Hepa1-6 cells.

FIG. 50 depicts genome editing at the APOA1 and ANGPTL3 loci by MG3-6/3-8 in wild-type mice analyzed by next generation sequencing.

FIG. 51A depicts a phylogenetic tree of various MG29 effectors described herein.

FIG. 51B depicts the three-dimensional structure of the MG29-1 effector predicted using Alphafold2. The nucleic acids forming the R-loop are modelled through a structural alignment with Cpf1 (PDB ID: 5XH7). REC, RuvC, and NUC domains are colored in gray, cyan, and pink, respectively. The PAM-interacting domains, i.e. the WED-II, WED-III, and PID domains are colored in yellow and orange, respectively.

FIG. 52A depicts phylogenetic analysis of Type V-A nucleases. The phylogenetic tree was inferred with RAxML from MAFFT global (g-ins-i) multiple sequence alignments. Ancestral WedII, PI, and WedIII domains were generated for three ancestral nodes (nodes highlighted with a closed circle: MG29-229, MG29-230, and MG29-231). FIG. 52B depicts 3D structure prediction of ancestral domains from MG29-28 vs. the predicted structure of MG29-1. High similarity of these domains is observed by the overlap between the structures (top) as well as conservation of key residues in the sequence alignment (bottom). Important residues involved in PAM binding are indicated by dark boxes above the alignment as well as by stick cartoon visualization in the 3D alignment.

FIGS. 53A, 53B, and 53C depict multiple sequence alignment of MG29-1 homologs to determine an optimal breakpoint for chimeragenesis. FIG. 53A depicts MG29-1 and FnCas12a aligned to several proteins described herein. The WED-II, PID, and WED-III domains are annotated to highlight the domains being swapped. The dashed lines depict the cut sites to swap these enzyme domains. FIG. 53B: The C-terminal end of MG29-1 protein and its homologs consists of a breakpoint at the edge of REC domain and WED-II domain (SEQ ID NOS: 2926-2937, respectively, in order of appearance). FIG. 53C: The N-terminal end of the MG29-1 protein and its homologs consists of a breakpoint at the edge of WED-III and RuvC domain. Overall, the swapped region spans the WED-II, PID, and WED-III domains (SEQ ID NOS: 2938-2949, respectively, in order of appearance).

FIG. 54 depicts in vitro screening of Type V-A chimeras. The agarose gel depicts the amplified cleavage products for each cleavage reaction. Positive enrichment was observed for chimeras with MG29-1 and several of its homologs, namely, MG29-13, MG29-14, MG29-16, MG29-20, and MG29-18.

FIG. 55 depicts in vitro screening of Type V-A ancestral sequences and their corresponding chimeras with MG29-1. The agarose gel depicts the amplified cleavage products for each cleavage reaction. Positive enrichment was observed only for ancestrally reconstructed MG29-230 and MG29-231. However, all the corresponding chimeras were found to be active.

FIG. 56 depicts PAMs associated with the chimeras derived from MG29-1 and its chimeras.

### BRIEF DESCRIPTION OF THE SEQUENCE LISTING

The Sequence Listing filed herewith provides example polynucleotide and polypeptide sequences for use in methods, compositions, and systems according to the disclosure. Below are example descriptions of sequences therein.

### MG3-6 Chimeras

SEQ ID NOs: 1-27 and 771-862 show the full-length peptide sequences of MG3-6 chimeric nucleases.

SEQ ID NO: 108 shows the nucleotide sequence of an MG3-6/3-4 nuclease containing 5′ UTR, NLS, CDS, NLS, 3′ UTR, and polyA tail.

SEQ ID NO: 722 shows the nucleotide sequence of a MG3-6/3-4 guide sgRNA scaffold.

SEQ ID NOs: 28-45, 605-610, 646-695, 863, and 1789-1826 show the nucleotide sequences of sgRNAs engineered to function with an MG3-6 chimeric nuclease.

SEQ ID NO: 603 shows the DNA coding sequence for MG3-6/3-4.

SEQ ID NO: 604 shows the protein sequence of the MG3-6/3-4 cassette coding sequence.

### MG29-1 Chimeras

SEQ ID NOs: 109-110 and 2842-2854 show the full-length peptide sequences of MG29-1 chimeric nucleases.

SEQ ID NOs: 111-113 show the nucleotide sequences of sgRNAs engineered to function with an MG29-1 chimeric nuclease.

### MG Chimeras

SEQ ID NO: 696 shows a N-terminal peptide sequence (1-742) of MG3-6.

SEQ ID NOs: 697-721 show C-terminal peptide sequences.

### Albumin Targeting

SEQ ID NOs: 67-86 show the nucleotide sequences of sgRNAs engineered to function with an MG3-6/3-4 nuclease in order to target albumin.

### TRAC Targeting

SEQ ID NOs: 119-138 show the nucleotide sequences of sgRNAs engineered to function with an MG3-6/3-4 nuclease in order to target TRAC.

SEQ ID NOs: 139-158 show the DNA sequences of TRAC target sites.

SEQ ID NOs: 922-924 show the nucleotide sequences of sgRNAs engineered to function with an MG3-6/3-3 nuclease in order to target TRAC.

SEQ ID NOs: 925-927 show the DNA sequences of TRAC target sites.

SEQ ID NOs: 972-991 show the nucleotide sequences of sgRNAs engineered to function with an MG3-6/3-7 nuclease in order to target TRAC.

SEQ ID NOs: 992-1011 show the DNA sequences of TRAC target sites.

SEQ ID NOs: 1088-1183, 1280-1320, 2390-2485, and 2582-2617 show the nucleotide sequences of sgRNAs engineered to function with an MG3-6/3-8 nuclease in order to target TRAC.

SEQ ID NOs: 1184-1279, 1321-1361, 2486-2581, and 2618-2653 show the DNA sequences of TRAC target sites.

### B2M Targeting

SEQ ID NOs: 159-184 show the nucleotide sequences of sgRNAs engineered to function with an MG3-6/3-4 nuclease in order to target B2M.

SEQ ID NOs: 185-210 show the DNA sequences of B2M target sites.

### TRBC1 Targeting

SEQ ID NOs: 211-251 show the nucleotide sequences of sgRNAs engineered to function with an MG3-6/3-4 nuclease in order to target TRBC1.

SEQ ID NOs: 252-292 show the DNA sequences of TRBC1 target sites.

### TRBC2 Targeting

SEQ ID NOs: 293-337 show the nucleotide sequences of sgRNAs engineered to function with an MG3-6/3-4 nuclease in order to target TRBC2.

SEQ ID NOs: 338-382 show the DNA sequences of TRBC2 target sites.

### ANGPTL3 Targeting

SEQ ID NOs: 383-477 and 1392-1489 show the nucleotide sequences of sgRNAs engineered to function with an MG3-6/3-4 nuclease in order to target ANGPTL3.

SEQ ID NOs: 478-572 and 1490-1587 show the DNA sequences of ANGPTL3 target sites.

SEQ ID NOs: 2120-2215 and 2312-2350 show the nucleotide sequences of sgRNAs engineered to function with an MG3-6/3-8 nuclease in order to target ANGPTL3.

SEQ ID NOs: 2216-2311 and 2351-2389 show the DNA sequences of ANGPTL3 target sites.

### PCSK9 Targeting

SEQ ID NOs: 573-587 and 1362-1376 show the nucleotide sequences of sgRNAs engineered to function with an MG3-6/3-4 nuclease in order to target PCSK9.

SEQ ID NOs: 588-602 and 1377-1391 show the DNA sequences of PCSK9 target sites.

### VCP R155 Targeting

SEQ ID NOs: 723-738 show the nucleotide sequences of sgRNAs engineered to function with an MG3-6/3-8 nuclease in order to target VCP R155.

SEQ ID NOs: 739-754 show the DNA sequences of VCPR155 target sites.

SEQ ID NOs: 755-762 show the nucleotide sequences of sgRNAs engineered to function with an MG3-6/3-4 nuclease in order to target VCP R155.

SEQ ID NOs: 763-770 show the DNA sequences of VCPR155 target sites.

### AAVS1 Targeting

SEQ ID NOs: 928-949 show the nucleotide sequences of sgRNAs engineered to function with an MG3-6/3-3 nuclease in order to target AAVS1.

SEQ ID NOs: 950-971 show the DNA sequences of AAVS1 target sites.

SEQ ID NOs: 1012-1049 show the nucleotide sequences of sgRNAs engineered to function with an MG3-6/3-7 nuclease in order to target AAVS1.

SEQ ID NOs: 1050-1087 show the DNA sequences of AAVS1 target sites.

### GPR146 Targeting

SEQ ID NOs: 1588-1656 show the nucleotide sequences of sgRNAs engineered to function with an MG3-6/3-7 nuclease in order to target M. musculus GPR146.

SEQ ID NOs: 1657-1725 show the DNA sequences of M. musculus GPR146 target sites.

### APOA1 Targeting

SEQ ID NOs: 1726-1744 show the nucleotide sequences of sgRNAs engineered to function with an MG3-6/3-4 nuclease in order to target M. musculus APOA1.

SEQ ID NOs: 1745-1763 show the DNA sequences of M. musculus APOA1 target sites.

SEQ ID NOs: 1764-1774 show the nucleotide sequences of sgRNAs engineered to function with an MG3-6/3-4 nuclease in order to target H. sapiens APOA1.

SEQ ID NOs: 1775-1785 show the DNA sequences of H. sapiens APOA1 target sites.

SEQ ID NOs: 1866-1961 and 2058-2088 show the nucleotide sequences of sgRNAs engineered to function with an MG3-6/3-8 nuclease in order to target mouse APOA1.

SEQ ID NOs: 1962-2057 and 2089-2119 show the DNA sequences of mouse APOA1 target sites.

### HAO1 Targeting

SEQ ID NOs: 611-633 and 1789-1826 show the nucleotide sequences of sgRNAs engineered to function with an MG3-6/3-4 nuclease in order to target M. musculus HAO1.

SEQ ID NOs: 1827-1865 show the nucleotide sequences of sgRNAs engineered to function with an MG3-6/3-4 nuclease in order to target human HAO1.

### Other Sequences

SEQ ID NOs: 87-102, 118, 634-645, and 1786-1787 show primer sequences

SEQ ID NOs: 103-107, 920-921, and 1788 show plasmid sequences.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” refers to a biological cell. A cell may be the basic structural, functional, or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, Cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots.

Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [RI10]dCTP, [R6G]dCTP, [TAMRA]dCTP, [JOE]ddATP, [R6G]ddATP, [FAM]ddCTP, [RI10]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. In a polynucleotide when referring to a T, a T means U (Uracil) in RNA and T (Thymine) in DNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions, or deletions. A non-native sequence may exhibit or encode for an activity (e.g., enzymatic activity, methyltransferase activity, acetyltransferase activity, kinase activity, ubiquitinating activity, etc.) that may also be exhibited by the nucleic acid or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A “basal promoter”, also referred to as a “core promoter”, may refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, refers to the process by which a nucleic acid sequence or a polynucleotide is transcribed from a DNA template (such as into mRNA or other RNA transcript) or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

The terms “engineered,” “synthetic,” and “artificial” are used interchangeably herein to refer to an object that has been modified by human intervention. For example, the terms may refer to a polynucleotide or polypeptide that is non-naturally occurring. An engineered peptide may have, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. For example, VPR and VP64 domains are synthetic transactivation domains. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

The term “tracrRNA” or “tracr sequence” means trans-activating CRISPR RNA. tracrRNA interacts with the CRISPR (cr) RNA to form guide (g) RNA in type II and subtype V-B CRISPR-Cas systems. If the tracrRNA is engineered, it may have about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes, S. aureus). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes, S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” or “guide polynucleotide” refers to a nucleic acid that may hybridize to a target nucleic acid and thereby directs an associated nuclease to the target nucleic acid. A guide nucleic acid may be RNA (guide RNA or gRNA). A guide nucleic acid may be DNA. A guide nucleic acid may be a mixture of RNA and DNA. A guide nucleic acid may comprise a crRNA or a tracrRNA or a combination of both. A guide nucleic acid may be engineered. The guide nucleic acid may be programmed to specifically bind to the target nucleic acid. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence,” or a “spacer.” A nucleic acid-targeting segment may comprise a sub-segment that may be referred to as a “protein binding segment” or “protein binding sequence” or “Cas protein binding segment.”

As used herein, the terms “gene editing” and “genome editing” can be used interchangeably. Gene editing or genome editing means to change the nucleic acid sequence of a gene or a genome. Genome editing can include, for example, insertions, deletions, and mutations.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “Wedge” (WED) domain refers to a domain (e.g., present in a Cas protein) interacting primarily with repeat:anti-repeat duplex of the sgRNA and PAM duplex. A WED domain can generally be identified by alignment to documented domain sequences, structural alignment to proteins with annotated domains, or by comparison to Hidden Markov Models (HMMs) built based on documented domain sequences.

As used herein, the term “PAM interacting domain” or “PI domain” refers to a domain interacting with the protospacer-adjacent motif (PAM) external to the seed sequence in a region targeted by a Cas protein. Examples of PAM-interacting domains include, but are not limited to, Topoisomerase-homology (TOPO) domains and C-terminal domains (CTD) present in Cas proteins. A PAM interacting domain or segments thereof can generally be identified by alignment to documented domain sequences, structural alignment to proteins with annotated domains, or by comparison to Hidden Markov Models (HMMs) built based on documented domain sequences.

As used herein, the term “REC domain” refers to a domain (e.g., present in a Cas protein) comprising at least one of two segments (REC1 or REC2) that are alpha helical domains thought to contact the guide RNA. A REC domain or segments thereof can generally be identified by alignment to documented domain sequences, structural alignment to proteins with annotated domains, or by comparison to Hidden Markov Models (HMMs) built based on documented domain sequences (e.g., Pfam PF19501 for domain REC1).

As used herein, the term “BH domain” refers to a domain (e.g., present in a Cas protein) that is a bridge helix between NUC and REC lobes of a Type II Cas enzyme. A BH domain or segments thereof can generally be identified by alignment to documented domain sequences, structural alignment to proteins with annotated domains, or by comparison to Hidden Markov Models (HMMs) built based on documented domain sequences (e.g., Pfam PF16593 for domain BH).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g., non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein. In some embodiments, such conservatively substituted variants are functional variants. Such functional variants can encompass sequences with substitutions such that the activity of critical active site residues of the endonuclease is not disrupted. In some embodiments, a functional variant of any of the systems described herein lack substitution of at least one of the conserved or functional residues described herein. In some embodiments, a functional variant of any of the systems described herein lacks substitution of all of the conserved or functional residues described herein.

Conservative substitution tables providing functionally similar amino acids are available from a variety of references (see, for example, Creighton, Proteins: Structures and Molecular Properties (W H Freeman & Co.; 2nd Edition (December 1993))). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence may involve both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class 1 CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class 2 CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Cas II nucleases are documented as DNA nucleases. Type 2 effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again documented as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g., Cas13) comprises two HEPN ribonuclease domains. Differing from both Type II and V systems, Type VI systems also appear to, in some embodiments, not require a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class 2 CRISPR-Cas have been most widely adopted for engineering and development as designer nuclease/genome editing applications.

One of the early adaptations of such a system for in vitro use involved (i) recombinantly-expressed, purified full-length Cas9 (e.g., a Class 2, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. A later improved, engineered system involved the crRNA of (ii) joined to the 5′ end of (iii) by a linker (e.g., GAAA) to form a single fused synthetic guide RNA (sgRNA) capable of directing Cas9 to a target by itself.

Such engineered systems can be adapted for use in mammalian cells by providing DNA vectors encoding (i) an ORF encoding codon-optimized Cas9 (e.g., a Class 2, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

### Engineered Endonucleases

Described herein, in certain embodiments, are engineered endonucleases. In some embodiments, the engineered endonuclease is a chimera of two or more endonucleases. In some embodiments, the engineered endonuclease is a fusion of two or more endonucleases.

In some embodiments, the engineered endonuclease comprises one or more fragments or domains of a nuclease, such as nucleic acid-guided nuclease. In some embodiments, the engineered endonuclease comprises one or more fragments or domains of a nuclease from orthologs of organisms, genus, species, or other phylogenetic groups described herein. In some embodiments, the engineered endonuclease comprises one or more fragments or domains from nuclease orthologs of different species.

In some embodiments, the engineered endonuclease comprises one or more fragments or domains from at least two different nucleases. In some embodiments, the engineered endonuclease comprises one or more fragments or domains from at least 2, 3, 4, 5, 6, 7, 8, 9, 10, or more different nucleases. In some embodiments, the engineered endonuclease comprises one or more fragments or domains from at least 2, 3, 4, 5, 6, 7, 8, 9, 10, or more nucleases from different species. In some embodiments, the engineered endonuclease comprises 2 fragments or domains, each from a different nuclease. In some embodiments, the engineered endonuclease comprises 3 fragments or domains, each from a different nuclease. In some embodiments, the engineered endonuclease comprises 4 fragments or domains, each from a different nuclease. In some embodiments, the engineered endonuclease comprises 5 fragments or domains, each from a different nuclease. In some embodiments, the engineered endonuclease comprises 3 fragments or domains, wherein at least one fragment or domain is from a different nuclease. In some embodiments, the engineered endonuclease comprises 4 fragments or domains, wherein at least one fragment or domain is from a different nuclease. In some embodiments, the engineered endonuclease comprises 5 fragments or domains, wherein at least one fragment or domain is from a different nuclease.

In some embodiments, the engineered endonucleases are functional in prokaryotic or eukaryotic cells for in vitro, in vivo, or ex vivo applications.

In some embodiments, the endonuclease is derived from an uncultivated microorganism. In some embodiments, the engineered endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the engineered endonuclease has less than 86% identity to a SpyCas9 endonuclease.

In some embodiments, junctions between fragments or domains from different nucleases or species occur in stretches of unstructured regions. Unstructured regions in polynucleotides include, for example, regions that have no predicted secondary structure elements such as alpha helices or beta strands. Unstructured regions may include for example, regions which are exposed within a protein structure, loop regions, or regions that are not conserved within various protein orthologs as predicted by sequence or structural alignments.

Described herein, in certain embodiments, are engineered endonucleases (e.g., chimeric endonucleases or fusion endonucleases) comprising an N-terminal portion comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696; and a C-terminal portion comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721. In some embodiments, the engineered endonuclease comprises an N-terminal portion comprising a sequence having at least about 70% identity to SEQ ID NO: 696 and a C-terminal portion comprising a sequence having at least about 70% identity to any one of SEQ ID NOs: 697-721. In some embodiments, the engineered endonuclease comprises an N-terminal portion comprising a sequence having at least about 75% identity to SEQ ID NO: 696 and a C-terminal portion comprising a sequence having at least about 75% identity to any one of SEQ ID NOs: 697-721. In some embodiments, the engineered endonuclease comprises an N-terminal portion comprising a sequence having at least about 80% identity to SEQ ID NO: 696 and a C-terminal portion comprising a sequence having at least about 80% identity to any one of SEQ ID NOs: 697-721. In some embodiments, the engineered endonuclease comprises an N-terminal portion comprising a sequence having at least about 85% identity to SEQ ID NO: 696 and a C-terminal portion comprising a sequence having at least about 85% identity to any one of SEQ ID NOs: 697-721. In some embodiments, the engineered endonuclease comprises an N-terminal portion comprising a sequence having at least about 90% identity to SEQ ID NO: 696 and a C-terminal portion comprising a sequence having at least about 90% identity to any one of SEQ ID NOs: 697-721. In some embodiments, the engineered endonuclease comprises an N-terminal portion comprising a sequence having at least about 95% identity to SEQ ID NO: 696 and a C-terminal portion comprising a sequence having at least about 95% identity to any one of SEQ ID NOs: 697-721. In some embodiments, the engineered endonuclease comprises an N-terminal portion comprising a sequence having at least about 96% identity to SEQ ID NO: 696 and a C-terminal portion comprising a sequence having at least about 96% identity to any one of SEQ ID NOs: 697-721. In some embodiments, the engineered endonuclease comprises an N-terminal portion comprising a sequence having at least about 97% identity to SEQ ID NO: 696 and a C-terminal portion comprising a sequence having at least about 97% identity to any one of SEQ ID NOs: 697-721. In some embodiments, the engineered endonuclease comprises an N-terminal portion comprising a sequence having at least about 98% identity to SEQ ID NO: 696 and a C-terminal portion comprising a sequence having at least about 98% identity to any one of SEQ ID NOs: 697-721. In some embodiments, the engineered endonuclease comprises an N-terminal portion comprising a sequence having at least about 99% identity to SEQ ID NO: 696 and a C-terminal portion comprising a sequence having at least about 99% identity to any one of SEQ ID NOs: 697-721. In some embodiments, the engineered endonuclease comprises an N-terminal portion comprising a sequence having 100% identity to SEQ ID NO: 696 and a C-terminal portion comprising a sequence having 100% identity to any one of SEQ ID NOs: 697-721.

Described herein, in certain embodiments, are engineered endonucleases (e.g., chimeric endonucleases or fusion endonucleases) comprising an N-terminal portion comprising RuvC, REC, or HNH domains of a Cas endonuclease and having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696; and a C-terminal portion comprising WED, TOPO, or CTD domains of a Cas endonuclease and having at least 55% at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721.

In some embodiments, the N-terminal portion of the engineered endonuclease comprises a RuvC domain, a REC domain, an HNH domain, a BH domain, or combinations thereof. In some embodiments, the C-terminal portion of the engineered endonuclease comprises a WED domain, TOPO domain, CTD domain, a PAM-interacting domain, or combinations thereof. In some embodiments, the N-terminal portion of the engineered endonuclease comprises RuvC, REC, and HNH domains. In some embodiments, the N-terminal portion of the engineered endonuclease comprises RuvC and HNH domains. In some embodiments, the N-terminal portion of the engineered endonuclease further comprises RuvC, REC, HNH, RuvC-I, BH, and RuvC-II domains. In some embodiments, the C-terminal portion of the engineered endonuclease comprises WED, TOPO, and CTD domains.

In some embodiments, the C-terminal portion of the engineered endonuclease comprises a PAM-interacting domain.

In some embodiments, the N-terminal portion of the engineered endonuclease and the C-terminal portion of the engineered endonuclease do not naturally occur together in a same reading frame. In some embodiments, the N-terminal portion of the engineered endonuclease and the C-terminal portion of the engineered endonuclease are derived from different organisms.

In some embodiments, the N-terminal portion of the engineered endonuclease and the C-terminal portion of the engineered endonuclease are fused directly. In some embodiments, the N-terminal portion of the engineered endonuclease and the C-terminal portion of the engineered endonuclease are joined by a linker. In some embodiments, the linker is a glycine and/or serine-rich linker, a large protein domain, a long helix structure, or a short helix structure. In some embodiments, the linker is (G4S)n, and wherein n is an integer from 1 to 20 (SEQ ID NO: 2950). In some embodiments, the linker is GGGGS (SEQ ID NO: 2864). In some embodiments, the linker comprises a sequence selected from the group consisting of (GS)n (SEQ ID NO: 2951), (G2S)n (SEQ ID NO: 2952), (G3S)n (SEQ ID NO: 2953), (G4S)n (SEQ IDS NO: 2950), and (G)n (SEQ ID NO: 2954), and wherein n is an integer from 1 to 20. In some embodiments, the one or more linkers comprises a sequence selected from the group consisting of (GGSGGD)n (SEQ ID NO: 2955) or (GGSGGE)n (SEQ ID NO: 2956), and wherein n is an integer from 1 to 6. In some embodiments, the one or more linkers comprises a sequence selected from the group consisting of (GGGSGGG)n (SEQ ID NO: 2957), (GGGSGSGGGGS)n (SEQ ID NO: 2958) and (GGGGGPGGGGP)n (SEQ ID NO: 2959), and wherein n is an integer from 1 to 3. In some embodiments, the one or more linkers comprises a sequence selected from the group consisting of (GX)n, (GGX)n, (GGGX)n, (GGGGX)n, and (GzX)n, wherein z is between 1 and 20, and wherein n is at least 8. In some embodiments, X is serine, aspartic acid, glutamic acid, threonine, or proline.

In some embodiments, the engineered endonuclease (e.g., chimeric endonuclease or fusion endonuclease) comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity sequence identity to any one of SEQ ID NOs: 1-27 and 771-862. In some embodiments, the engineered endonuclease comprises a sequence having at least about 70% identity to any one of SEQ ID NOs: 1-27 and 771-862. In some embodiments, the engineered endonuclease comprises a sequence having at least about 75% identity to any one of SEQ ID NOs: 1-27 and 771-862. In some embodiments, the engineered endonuclease comprises a sequence having at least about 80% identity to any one of SEQ ID NOs: 1-27 and 771-862. In some embodiments, the engineered endonuclease comprises a sequence having at least about 85% identity to any one of SEQ ID NOs: 1-27 and 771-862. In some embodiments, the engineered endonuclease comprises a sequence having at least about 90% identity to any one of SEQ ID NOs: 1-27 and 771-862. In some embodiments, the engineered endonuclease comprises a sequence having at least about 95% identity to any one of SEQ ID NOs: 1-27 and 771-862. In some embodiments, the engineered endonuclease comprises a sequence having at least about 96% identity to any one of SEQ ID NOs: 1-27 and 771-862. In some embodiments, the engineered endonuclease comprises a sequence having at least about 97% identity to any one of SEQ ID NOs: 1-27 and 771-862. In some embodiments, the engineered endonuclease comprises a sequence having at least about 98% identity to any one of SEQ ID NOs: 1-27 and 771-862. In some embodiments, the engineered endonuclease comprises a sequence having at least about 99% identity to any one of SEQ ID NOs: 1-27 and 771-862. In some embodiments, the engineered endonuclease comprises a sequence having 100% identity to any one of SEQ ID NOs: 1-27 and 771-862.

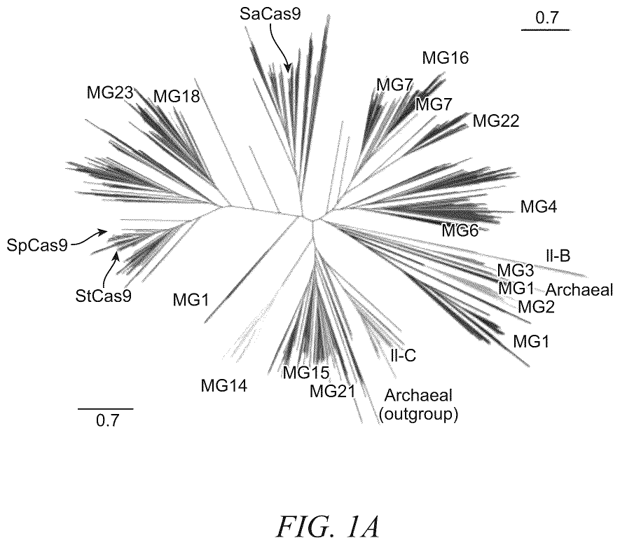

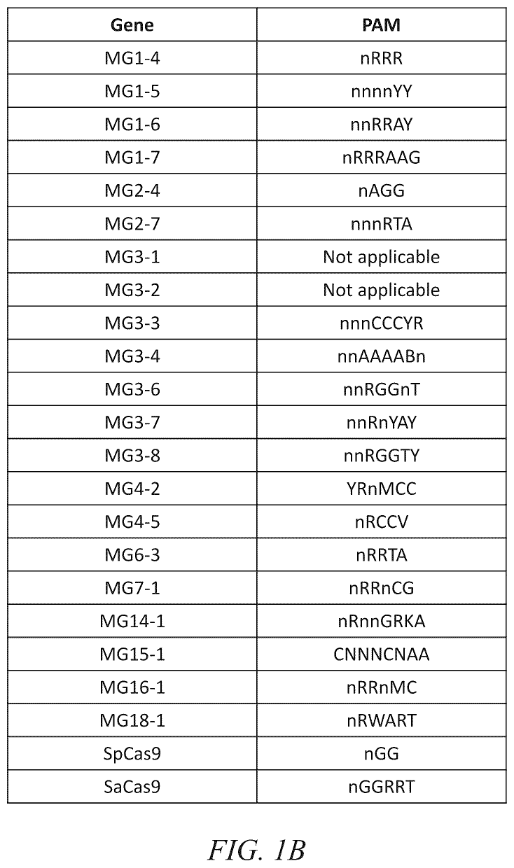

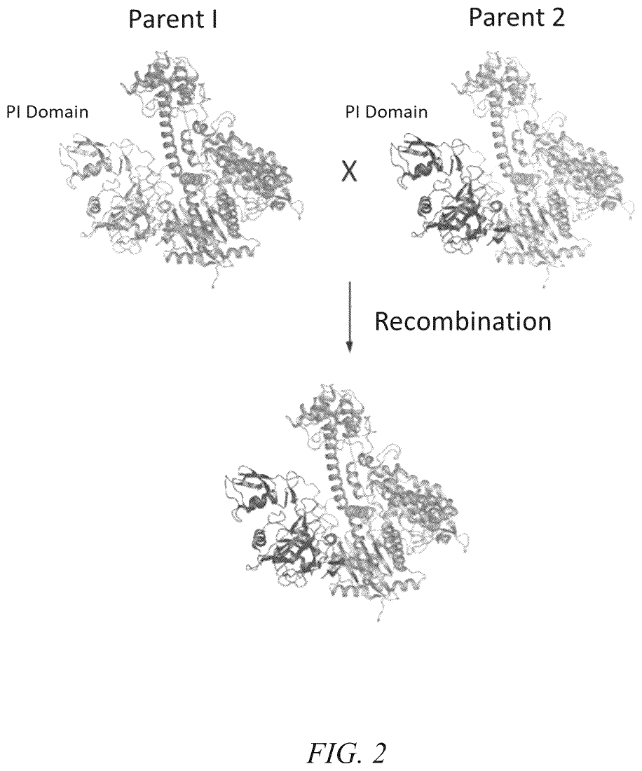

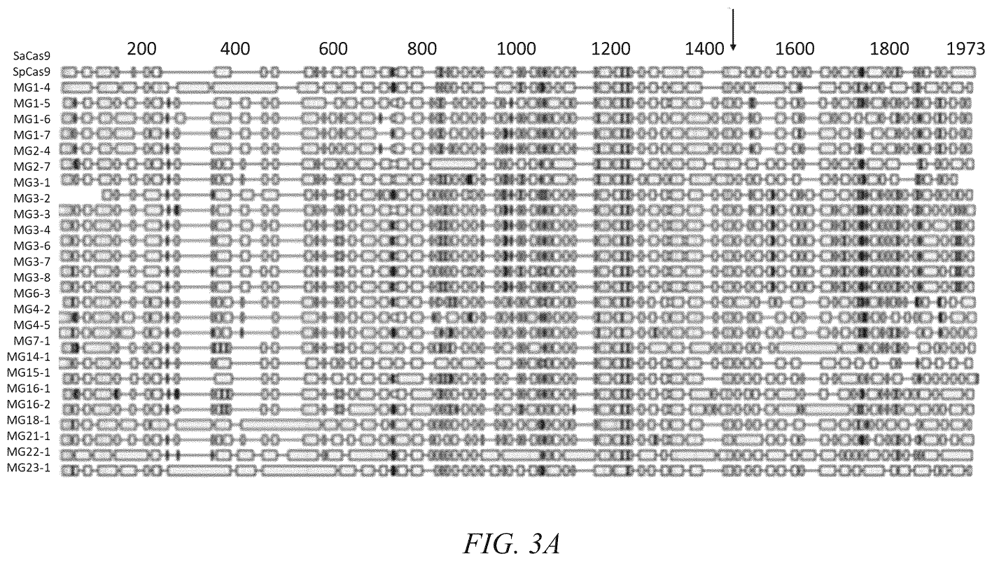

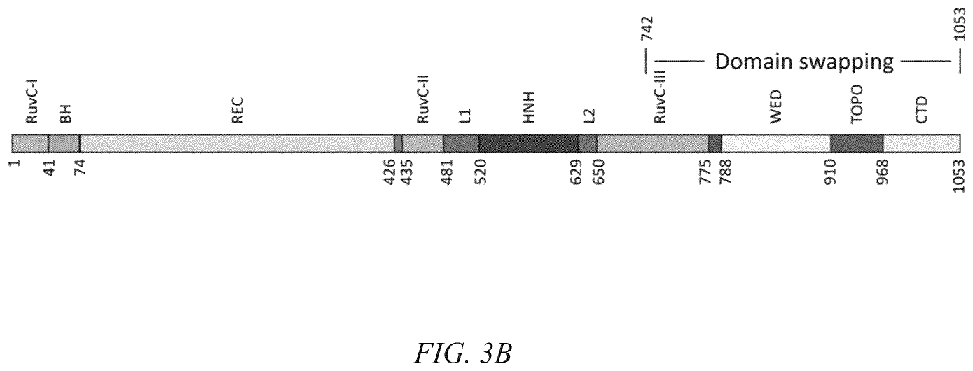

In some embodiments, the engineered endonuclease described herein have natural PAM specificities (see FIG. 1B). In some embodiments, the present disclosure provides for the enablement of PAM specificity by protein engineering. In some embodiments, the enablement of PAM specificity is achieved by the domain swapping of RNA guided CRISPR-associated nucleases (see FIG. 2). In some embodiments, there is an optimal breakpoint in the process of domain swapping and recombination. In some embodiments, the optimal breakpoint is guided by the alignment of multiple sequences described herein (see FIG. 3A).

In some embodiments, the engineered endonuclease is configured to bind to a PAM that is not nnRGGnT. In some embodiments, the engineered endonuclease is configured to bind to a PAM in Table 1A. In some embodiments, the engineered endonuclease is configured to bind to a PAM comprising a sequence of any one of nnRGGnT, nnnmtY, nnnCCCy, nnraww, nnRnYAY, nnnctC, nnRGGTY, nnRGGTY, nnnctC, nnRnYAY, nnnMWTY, nnrRTwy, nnRRTWY, nyrnwYY, nnrCyY, nnncMMc, nnrnYCC, nnncMMc, nnrRnCm, nnnYCMw, nnnmCmC, nnrnynnn, nnnmCm, nnRnYCYr, nnRAYAC, nnnmCm, nnnRMYT, nnTYCm, nnTMCy, nnrMTCr, nnnmCCY, nnrmww, nnrRkyY, nnRnYhy, nnnmwTY, nnnmyY, nnRnYhY, nnnmhTY, nnRMAC, nnrRtwy, nnrwwYY, nnrmyY, nnrwYCC, nnRGnCr, nnnMhYy, nnyCCMww, nnnMCMCw, nnRwYhWw, nnRrYCYr, nnrwyhh, ntrMCm, nnrwyhh, nnRRTWY, ntTYCM, nnTCCC, nnrnyhh, nnnCCCYR, nnRnYAYn, nnRRnnYn, and nnRRnnYn. In some embodiments, the engineered endonuclease is configured to bind to a PAM by recognizing the PAM sequence. In some embodiments, the engineered endonuclease recognizes and binds a PAM sequence.

TABLE 1A
PAM Sequences

| Category | Description | Type | Organism | Sequence |
|---|---|---|---|---|
| MG3-6 | MG3-6 | nucleotide | artificial | nnRGGnT |
| | PAM | | sequence | |
| MG3-6 chimera | MG3-6_3-2 | nucleotide | artificial | nnnmtY |
| PAM | PAM | | sequence | |
| MG3-6 chimera | MG3-6_3-3 | nucleotide | artificial | nnnCCCy |
| PAM | PAM | | sequence | |
| MG3-6 chimera | MG3-6_3-3 | nucleotide | artificial | nnnCCCYR |
| PAM | PAM | | sequence | |
| MG3-6 chimera | MG3-6_3-4 | nucleotide | artificial | nnraww |
| PAM | PAM | | sequence | |
| MG3-6 chimera | MG3-6_3-7 | nucleotide | artificial | nnRnYAY |
| PAM | PAM | | sequence | |
| MG3-6 chimera | MG3-6_3-7 | nucleotide | artificial | nnRnYAYn |
| PAM | PAM | | sequence | |
| MG3-6 chimera | MG3-6_3-7 | nucleotide | artificial | nnnctC |
| PAM | PAM | | sequence | |
| MG3-6 chimera | MG3-6_3-8 | nucleotide | artificial | nnRGGTY |
| PAM | PAM | | sequence | |
| MG3-6 chimera | MG3-6_3-8 | nucleotide | artificial | nnRRnnYn |
| PAM | PAM | | sequence | |
| MG3-6 chimera | MG3-6_3- | nucleotide | artificial | nnRGGTY |
| PAM | 18 PAM | | sequence | |
| MG3-6 chimera | MG3-6_3- | nucleotide | artificial | nnnctC |
| PAM | 22 PAM | | sequence | |
| MG3-6 chimera | MG3-6_3- | nucleotide | artificial | nnRnYAY |
| PAM | 24 PAM | | sequence | |
| MG3-6 chimera | MG3-6_3- | nucleotide | artificial | nnnMWTY |
| PAM | 38 PAM | | sequence | |
| MG3-6 chimera | MG3-6_3- | nucleotide | artificial | nnrRTwy |
| PAM | 42 PAM | | sequence | |
| MG3-6 chimera | MG3-6_3- | nucleotide | artificial | nnRRTWY |
| PAM | 78 PAM | | sequence | |
| MG3-6 chimera | MG3-6_3- | nucleotide | artificial | nyrnwYY |
| PAM | 89 PAM | | sequence | |
| MG3-6 chimera | MG3-6_3- | nucleotide | artificial | nnrCyY |
| PAM | 90 PAM | | sequence | |
| MG3-6 chimera | MG3-6_3- | nucleotide | artificial | nnncMMc |
| PAM | 91 PAM | | sequence | |
| MG3-6 chimera | MG3-6_3- | nucleotide | artificial | nnrnYCC |
| PAM | 91 PAM | | sequence | |
| MG3-6 chimera | MG3-6_3- | nucleotide | artificial | nnncMMc |
| PAM | 93 PAM | | sequence | |
| MG3-6 chimera | MG3-6_3- | nucleotide | artificial | nnrRnCm |
| PAM | 94 PAM | | sequence | |
| MG3-6 chimera | MG3-6_3- | nucleotide | artificial | nnnYCMw |
| PAM | 94 PAM | | sequence | |
| MG3-6 chimera | MG3-6_3- | nucleotide | artificial | nnnmCmC |
| PAM | 95 PAM | | sequence | |
| MG3-6 chimera | MG3-6_3- | nucleotide | artificial | nnrnynnn |
| PAM | 96 PAM | | sequence | |
| MG3-6 chimera | MG3-6_3- | nucleotide | artificial | nnnmCm |
| PAM | 101 PAM | | sequence | |
| MG3-6 chimera | MG3-6_3- | nucleotide | artificial | nnRnYCYr |
| PAM | 103 PAM | | sequence | |
| MG3-6 chimera | MG3-6_3- | nucleotide | artificial | nnRAYAC |
| PAM | 104 PAM | | sequence | |
| MG3-6 chimera | MG3-6_3- | nucleotide | artificial | nnnmCm |
| PAM | 150-1 PAM | | sequence | |
| MG3-6 chimera | MG3-6_3- | nucleotide | artificial | nnnRMYT |
| PAM | 150-6 PAM | | sequence | |
| MG3-6 chimera | MG3-6_3- | nucleotide | artificial | nnTYCm |
| PAM | 150-7 PAM | | sequence | |
| MG3-6 chimera | MG3-6_3- | nucleotide | artificial | nnTMCy |
| PAM | 150-8 PAM | | sequence | |
| MG3-6_3-8 | MG3-6_3- | nucleotide | artificial | nnrMTCr |
| chimera PAM | 8_3-2 PAM | | sequence | |
| MG3-6_3-8 | MG3-6_3- | nucleotide | artificial | nnnmCCY |
| chimera PAM | 8_3-3 PAM | | sequence | |
| MG3-6_3-8 | MG3-6_3- | nucleotide | artificial | nnrmww |
| chimera PAM | 8_3-4 PAM | | sequence | |
| MG3-6_3-8 | MG3-6_3- | nucleotide | artificial | nnrRkyY |
| chimera PAM | 8_3-6 PAM | | sequence | |
| MG3-6_3-8 | MG3-6_3- | nucleotide | artificial | nnRnYhy |
| chimera PAM | 8_3-7 PAM | | sequence | |
| MG3-6_3-8 | MG3-6_3- | nucleotide | artificial | nnnmwTY |
| chimera PAM | 8_3-18 | | sequence | |
| | PAM | | | |
| MG3-6_3-8 | MG3-6_3- | nucleotide | artificial | nnnmy Y |
| chimera PAM | 8_3-22 | | sequence | |
| | PAM | | | |
| MG3-6_3-8 | MG3-6_3- | nucleotide | artificial | nnRnYhY |
| chimera PAM | 8_3-24 | | sequence | |
| | PAM | | | |
| MG3-6_3-8 | MG3-6_3- | nucleotide | artificial | nnnmhTY |
| chimera PAM | 8 3-38 | | sequence | |
| | PAM | | | |
| MG3-6_3-8 | MG3-6_3- | nucleotide | artificial | nnRMAC |
| chimera PAM | 8_3-42 | | sequence | |
| | PAM | | | |
| MG3-6_3-8 | MG3-6_3- | nucleotide | artificial | nnrRtwy |
| chimera PAM | 8_3-78 | | sequence | |
| | PAM | | | |
| MG3-6_3-8 | MG3-6_3- | nucleotide | artificial | nnrwwYY |
| chimera PAM | 8_3-89 | | sequence | |
| | PAM | | | |
| MG3-6_3-8 | MG3-6_3- | nucleotide | artificial | nnrmyY |
| chimera PAM | 8_3-90 | | sequence | |
| | PAM | | | |
| MG3-6_3-8 | MG3-6_3- | nucleotide | artificial | nnrwYCC |
| chimera PAM | 8_3-91 | | sequence | |
| | PAM | | | |
| MG3-6_3-8 | MG3-6_3- | nucleotide | artificial | nnRGnCr |
| chimera PAM | 8_3-92 | | sequence | |
| | PAM | | | |
| MG3-6_3-8 | MG3-6_3- | nucleotide | artificial | nnnMhYy |
| chimera PAM | 8_3-93 | | sequence | |
| | PAM | | | |
| MG3-6_3-8 | MG3-6_3- | nucleotide | artificial | nnyCCMww |
| chimera PAM | 8_3-94 | | sequence | |
| | PAM | | | |
| MG3-6_3-8 | MG3-6_3- | nucleotide | artificial | nnnMCMCw |
| chimera PAM | 8_3-95 | | sequence | |
| | PAM | | | |
| MG3-6_3-8 | MG3-6_3- | nucleotide | artificial | nnRwYhWw |
| chimera PAM | 8_3-96 | | sequence | |
| | PAM | | | |
| MG3-6_3-8 | MG3-6_3- | nucleotide | artificial | nnRrYCYr |
| chimera PAM | 8_3-103 | | sequence | |
| | PAM | | | |
| MG3-6_3-8 | MG3-6_3- | nucleotide | artificial | nnrwyhh |
| chimera PAM | 8_3-104 | | sequence | |
| | PAM | | | |
| MG3-6_3-8 | MG3-6_3- | nucleotide | artificial | ntrMCm |
| chimera PAM | 8_3-150-1 | | sequence | |
| | PAM | | | |
| MG3-6_3-8 | MG3-6_3- | nucleotide | artificial | nnrwyhh |
| chimera PAM | 8_3-150-2 | | sequence | |
| | PAM | | | |
| MG3-6_3-8 | MG3-6_3- | nucleotide | artificial | nnRRTWY |
| chimera PAM | 8_3-150-3 | | sequence | |
| | PAM | | | |
| MG3-6_3-8 | MG3-6_3- | nucleotide | artificial | ntTYCM |
| chimera PAM | 8_3-150-7 | | sequence | |
| | PAM | | | |
| MG3-6_3-8 | MG3-6_3- | nucleotide | artificial | nnTCCC |
| chimera PAM | 8_3-150-8 | | sequence | |
| | PAM | | | |
| MG3-6_3-8 | MG3-6_3- | nucleotide | artificial | nnrnyhh |
| chimera PAM | 8_3-150-9 | | sequence | |
| | PAM | | | |
| MG29-1 | MG29- | nucleic acid | artificial | TTTn |
| chimera PAM | 1_29-13 | | sequence | |
| sequence | PAM | | | |
| MG29-1 | MG29- | nucleic acid | artificial | TYYn |
| chimera PAM | 1_29-14 | | sequence | |
| sequence | PAM | | | |
| MG29-1 | MG29- | nucleic acid | artificial | TTTn |
| chimera PAM | 1_29-16 | | sequence | |
| sequence | PAM | | | |
| MG29-1 | MG29- | nucleic acid | artificial | nYTn |
| chimera PAM | 1_29-18 | | sequence | |
| sequence | PAM | | | |
| MG29-1 | MG29- | nucleic acid | artificial | TTYn |
| chimera PAM | 1_29-20 | | sequence | |
| sequence | PAM | | | |
| MG29-1 | MG29- | nucleic acid | artificial | nTTn |
| chimera PAM | 1_29-229 | | sequence | |
| sequence | PAM | | | |
| MG29-1 | MG29- | nucleic acid | artificial | nYYn |
| chimera PAM | 1_29-230 | | sequence | |
| sequence | PAM | | | |
| MG29-1 | MG29- | nucleic acid | artificial | nYYn |
| chimera PAM | 1 29-231 | | sequence | |
| sequence | PAM | | | |
| MG3-6 chimera | MG3-6_3-8 | nucleic acid | artificial | nnRRnnYn |
| PAM sequence | PAM | | sequence | |

In some embodiments, the engineered endonuclease (e.g., chimeric endonuclease or fusion endonuclease) comprises sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 109-110 and 2842-2854. In some embodiments, the engineered endonuclease comprises a sequence having at least about 70% identity to any one of SEQ ID NOs: 109-110 and 2842-2854. In some embodiments, the engineered endonuclease comprises a sequence having at least about 75% identity to any one of SEQ ID NOs: 109-110 and 2842-2854. In some embodiments, the engineered endonuclease comprises a sequence having at least about 80% identity to any one of SEQ ID NOs: 109-110 and 2842-2854. In some embodiments, the engineered endonuclease comprises a sequence having at least about 85% identity to any one of SEQ ID NOs: 109-110 and 2842-2854. In some embodiments, the engineered endonuclease comprises a sequence having at least about 90% identity to any one of SEQ ID NOs: 109-110 and 2842-2854. In some embodiments, the engineered endonuclease comprises a sequence having at least about 95% identity to any one of SEQ ID NOs: 109-110 and 2842-2854. In some embodiments, the engineered endonuclease comprises a sequence having at least about 96% identity to any one of SEQ ID NOs: 109-110 and 2842-2854. In some embodiments, the engineered endonuclease comprises a sequence having at least about 97% identity to any one of SEQ ID NOs: 109-110 and 2842-2854. In some embodiments, the engineered endonuclease comprises a sequence having at least about 98% identity to any one of SEQ ID NOs: 109-110 and 2842-2854. In some embodiments, the engineered endonuclease comprises a sequence having at least about 99% identity to any one of SEQ ID NOs: 109-110 and 2842-2854. In some embodiments, the engineered endonuclease comprises a sequence having 100% identity to any one of SEQ ID NOs: 109-110 and 2842-2854.

In some embodiments, the engineered endonuclease is configured to bind to a PAM that is not nnRGGnT. In some embodiments, the engineered endonuclease is configured to bind to a PAM in Table 1A. In some embodiments, the engineered endonuclease is configured to bind to a PAM that comprises any one of TTTn, TYYn, TTTn, nYTn, TTYn, nTTn, nYYn, and nYYn. In some embodiments, the engineered endonuclease is configured to bind to a PAM by recognizing the PAM sequence. In some embodiments, the engineered endonuclease recognizes and binds a PAM sequence.

### Guide Polynucleotides

Disclosed herein, in certain embodiments, are endonuclease systems comprising (a) an engineered nuclease disclosed herein, and (b) a guide polynucleotide e.g., a guide ribonucleic acid (gRNA), a single gRNA, or a dual guide RNA. In a polynucleotide when referring to a T, a T means U (Uracil) in RNA and T (Thymine) in DNA.

In some embodiments, the engineered guide polynucleotide is configured to form a complex with the engineered endonuclease. In some cases, the engineered guide polynucleotide comprises a spacer sequence. In some cases, the spacer sequence is configured to hybridize to a target nucleic acid sequence. In some cases, the endonuclease is configured to bind to a protospacer adjacent motif (PAM) sequence.

In some embodiments, the guide polynucleotide (e.g., gRNA) targets a gene or locus in a cell. In some embodiments, the guide polynucleotide targets a gene or locus in a mammalian cell. In some embodiments, the mammalian cell is a pig, a cow, a goat, a sheep, a rodent, a rat, a mouse, a non-human primate, or a human cell. In some embodiments, the target gene or target locus is albumin, TRAC, B2M, TRBC1, TRBC2, ANGPTL3, PCSK9, VCP R155, AAVS1, GPR146, APOA1, or HAO1.

In some embodiments, the target gene is albumin. In some embodiments, the guide polynucleotide is encoded by any one of SEQ ID NOs: 67-86 or a sequence having at least 90%, 95%, 97%, 98%, or 99% sequence identity to any one of SEQ ID NOs: 67-86. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 67-86. In some embodiments, the guide polynucleotide is encoded by a sequence having at least about 80% identity to any one of SEQ ID NOs: 67-86. In some embodiments, the guide polynucleotide is encoded by a sequence having at least about 85% identity to any one of SEQ ID NOs: 67-86. In some embodiments, the guide polynucleotide is encoded by a sequence having at least about 90% identity to any one of SEQ ID NOs: 67-86. In some embodiments, the guide polynucleotide is encoded by a sequence having at least about 95% identity to any one of SEQ ID NOs: 67-86. In some embodiments, the guide polynucleotide is encoded by a sequence having at least about 96% identity to any one of SEQ ID NOs: 67-86.

In some embodiments, the guide polynucleotide is encoded by a sequence having at least about 97% identity to any one of SEQ ID NOs: 67-86. In some embodiments, the guide polynucleotide is encoded by a sequence having at least about 98% identity to any one of SEQ ID NOs: 67-86. In some embodiments, the guide polynucleotide is encoded by a sequence having at least about 99% identity to any one of SEQ ID NOs: 67-86. In some embodiments, the guide polynucleotide is encoded by a sequence having 100% identity to any one of SEQ ID NOs: 67-86.

In some embodiments, the guide polynucleotide hybridizes or targets a sequence complementary to a target nucleic acid sequence within the albumin gene or within an intron of an endogenous gene. In some embodiments, the guide polynucleotide hybridizes or targets a sequence complementary to any one of SEQ ID NOs: 67-86 or a sequence having at least 90%, 95%, 97%, 98%, or 99% sequence identity to any one of SEQ ID NOs: 67-86. In some embodiments, the guide polynucleotide hybridizes or targets a sequence complementary to a sequence having at least about 80% identity to any one of SEQ ID NOs: 67-86. In some embodiments, the guide polynucleotide hybridizes or targets a sequence complementary to a sequence having at least about 85% identity to any one of SEQ ID NOs: 67-86. In some embodiments, the guide polynucleotide hybridizes or targets a sequence complementary to a sequence having at least about 90% identity to any one of SEQ ID NOs: 67-86. In some embodiments, the guide polynucleotide hybridizes or targets a sequence complementary to a sequence having at least about 95% identity to any one of SEQ ID NOs: 67-86. In some embodiments, the guide polynucleotide hybridizes or targets a sequence complementary to a sequence having at least about 96% identity to any one of SEQ ID NOs: 67-86. In some embodiments, the guide polynucleotide hybridizes or targets a sequence complementary to a sequence having at least about 97% identity to any one of SEQ ID NOs: 67-86. In some embodiments, the guide polynucleotide hybridizes or targets a sequence complementary to a sequence having at least about 98% identity to any one of SEQ ID NOs: 67-86. In some embodiments, the guide polynucleotide hybridizes or targets a sequence complementary to a sequence having at least about 99% identity to any one of SEQ ID NOs: 67-86. In some embodiments, the guide polynucleotide hybridizes or targets a sequence complementary to a sequence having 100% identity to any one of SEQ ID NOs: 67-86.

In some embodiments, the target gene is TRAC. In some embodiments, the guide polynucleotide is encoded by any one of SEQ ID NOs: 119-138, 922-924, 972-991, 1088-1183, 1280-1320, 2390-2485, and 2582-2617 or a sequence having at least 90%, 95%, 97%, 98%, or 99% sequence identity to any one of SEQ ID NOs: 119-138, 922-924, 972-991, 1088-1183, 1280-1320, 2390-2485, and 2582-2617. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 80% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 85% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 90% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 95% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 96% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 97% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 98% identity to any one of SEQ ID NOs: 119-138, 922-924, 972-991, 1088-1183, 1280-1320, 2390-2485, and 2582-2617. In some embodiments, the guide polynucleotide is encoded by a sequence having at least about 99% identity to any one of SEQ ID NOs: 119-138, 922-924, 972-991, 1088-1183, 1280-1320, 2390-2485, and 2582-2617. In some embodiments, the guide polynucleotide is encoded by a sequence having 100% identity to any one of SEQ ID NOs: 119-138, 922-924, 972-991, 1088-1183, 1280-1320, 2390-2485, and 2582-2617.

In some embodiments, the guide polynucleotide hybridizes or targets a sequence complementary to a target nucleic acid sequence within the TRAC gene or within an intron of an endogenous gene. In some embodiments, the guide polynucleotide hybridizes or targets a sequence complementary to any one of SEQ ID NOs: 119-138, 922-924, 972-991, 1088-1183, 1280-1320, 2390-2485, and 2582-2617 or a sequence having at least 90%, 95%, 97%, 98%, or 99% sequence identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 80% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 85% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 90% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 95% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 96% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 97% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 98% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having at least about 99% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to a sequence having 100% identity to any one of SEQ ID NOs: 119-138, 922-924, 972-991, 1088-1183, 1280-1320, 2390-2485, and 2582-2617.

In some embodiments, the guide polynucleotide hybridizes or targets a sequence within the TRAC gene or within an intron of an endogenous gene. In some embodiments, the guide polynucleotide hybridizes or targets a sequence according to any one of SEQ ID NOs: 139-158,925-927,992-1011, 1184-1279, 1321-1361, 2486-2581, and 2618-2653 or a sequence having at least 90%, 95%, 97%, 98%, or 99% sequence identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 80% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 85% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 90% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 95% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 96% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 97% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 98% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having at least about 99% identity to any one of SEQ ID NOs: 139-158, 925-927, 992-1011, 1184-1279, 1321-1361, 2486-2581, and 2618-2653. In some embodiments, the guide polynucleotide hybridizes or targets a sequence having 100% identity to any one of SEQ ID NOs: 139-158, 925-927, 992-1011, 1184-1279, 1321-1361, 2486-2581, and 2618-2653.

In some embodiments, the target gene is B2M. In some embodiments, the guide polynucleotide is encoded by any one of SEQ ID NOs: 159-184 or a sequence having at least 90%, 95%, 97%, 98%, or 99% sequence identity to any one of SEQ ID NOs: 159-184. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 159-184. In some embodiments, the guide polynucleotide is encoded by a sequence having at least about 80% identity to any one of SEQ ID NOs: 159-184. In some embodiments, the guide polynucleotide is encoded by a sequence having at least about 85% identity to any one of SEQ ID NOs: 159-184. In some embodiments, the guide polynucleotide is encoded by a sequence having at least about 90% identity to any one of SEQ ID NOs: 159-184. In some embodiments, the guide polynucleotide is encoded by a sequence having at least about 95% identity to any one of SEQ ID NOs: 159-184. In some embodiments, the guide polynucleotide is encoded by a sequence having at least about 96% identity to any one of SEQ ID NOs: 159-184. In some embodiments, the guide polynucleotide is encoded by a sequence having at least about 97% identity to any one of SEQ ID NOs: 159-184. In some embodiments, the guide polynucleotide is encoded by a sequence having at least about 98% identity to any one of SEQ ID NOs: 159-184. In some embodiments, the guide polynucleotide is encoded by a sequence having at least about 99% identity to any one of SEQ ID NOs: 159-184. In some embodiments, the guide polynucleotide is encoded by a sequence having 100% identity to any one of SEQ ID NOs: 159-184.

In some embodiments, the guide polynucleotide hybridizes or targets a sequence complementary to a target nucleic acid sequence within the B2M gene or within an intron of an endogenous gene. In some embodiments, the guide polynucleotide hybridizes or targets a sequence complementary to any one of SEQ ID NOs: 159-184 or a sequence having at least 90%, 95%, 97%, 98%, or 99% sequence identity to any one of SEQ ID NOs: 159-184. In some embodiments, the guide polynucleotide hybridizes or targets a sequence complementary to a sequence having at least about 80% identity to any one of SEQ ID NOs: 159-184. In some embodiments, the guide polynucleotide hybridizes or targets a sequence complementary to a sequence having at least about 85% identity to any one of SEQ ID NOs: 159-184. In some embodiments, the guide polynucleotide hybridizes or targets a sequence complementary to a sequence having at least about 90% identity to any one of SEQ ID NOs: 159-184. In some embodiments, the guide polynucleotide hybridizes or targets a sequence complementary to a sequence having at least about 95% identity to any one of SEQ ID NOs: 159-184. In some embodiments, the guide polynucleotide hybridizes or targets a sequence complementary to a sequence having at least about 96% identity to any one of SEQ ID NOs: 159-184. In some embodiments, the guide polynucleotide hybridizes or targets a sequence complementary to a sequence having at least about 97% identity to any one of SEQ ID NOs: 159-184. In some embodiments, the guide polynucleotide hybridizes or targets a sequence complementary to a sequence having at least about 98% identity to any one of SEQ ID NOs: 159-184. In some embodiments, the guide polynucleotide hybridizes or targets a sequence complementary to a sequence having at least about 99% identity to any one of SEQ ID NOs: 159-184. In some embodiments, the guide polynucleotide hybridizes or targets a sequence complementary to a sequence having 100% identity to any one of SEQ ID NOs: 159-184.

In some embodiments, the guide polynucleotide hybridizes or targets a sequence within the B2M gene or within an intron of an endogenous gene. In some embodiments, the guide polynucleotide hybridizes or targets a sequence according to any one of SEQ ID NOs: 185-210 or a sequence having at least 90%, 95%, 97%, 98%, or 99% sequence identity to any one of SEQ ID NOs: 185-210. In some embodiments, the guide polynucleotide hybridizes or targets a sequence having at least about 80% identity to any one of SEQ ID NOs: 185-210. In some embodiments, the guide polynucleotide hybridizes or targets a sequence having at least about 85% identity to any one of SEQ ID NOs: 185-210. In some embodiments, the guide polynucleotide hybridizes or targets a sequence having at least about 90% identity to any one of SEQ ID NOs: 185-210. In some embodiments, the guide polynucleotide hybridizes or targets a sequence having at least about 95% identity to any one of SEQ ID NOs: 185-210. In some embodiments, the guide polynucleotide hybridizes or targets a sequence having at least about 96% identity to any one of SEQ ID NOs: 185-210. In some embodiments, the guide polynucleotide hybridizes or targets a sequence having at least about 97% identity to any one of SEQ ID NOs: 185-210. In some embodiments, the guide polynucleotide hybridizes or targets a sequence having at least about 98% identity to any one of SEQ ID NOs: 185-210. In some embodiments, the guide polynucleotide hybridizes or targets a sequence having at least about 99% identity to any one of SEQ ID NOs: 185-210. In some embodiments, the guide polynucleotide hybridizes or targets a sequence having 100% identity to any one of SEQ ID NOs: 185-210.

In some embodiments, the target gene is TRBC1. In some embodiments, the guide polynucleotide is encoded by any one of SEQ ID NOs: 211-251 or a sequence having at least 90%, 95%, 97%, 98%, or 99% sequence identity to any one of SEQ ID NOs: 211-251. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 211-251. In some embodiments, the guide polynucleotide is encoded by a sequence having at least about 80% identity to any one of SEQ ID NOs: 211-251. In some embodiments, the guide polynucleotide is encoded by a sequence having at least about 85% identity to any one of SEQ ID NOs: 211-251. In some embodiments, the guide polynucleotide is encoded by a sequence having at least about 90% identity to any one of SEQ ID NOs: 211-251. In some embodiments, the guide polynucleotide is encoded by a sequence having at least about 95% identity to any one of SEQ ID NOs: 211-251. In some embodiments, the guide polynucleotide is encoded by a sequence having at least about 96% identity to any one of SEQ ID NOs: 211-251. In some embodiments, the guide polynucleotide is encoded by a sequence having at least about 97% identity to any one of SEQ ID NOs: 211-251. In some embodiments, the guide polynucleotide is encoded by a sequence having at least about 98% identity to any one of SEQ ID NOs: 211-251. In some embodiments, the guide polynucleotide is encoded by a sequence having at least about 99% identity to any one of SEQ ID NOs: 211-251. In some embodiments, the guide polynucleotide is encoded by a sequence having 100% identity to any one of SEQ ID NOs: 211-251.

In some embodiments, the guide polynucleotide hybridizes or targets a sequence complementary to a target nucleic acid sequence within the TRBC1 gene or within an intron of an endogenous gene. In some embodiments, the guide polynucleotide hybridizes or targets a sequence complementary to any one of SEQ ID NOs: 211-251 or a sequence having at least 90%, 95%, 97%, 98%, or 99% sequence identity to any one of SEQ ID NOs: 211-251. In some embodiments, the guide polynucleotide hybridizes or targets a sequence complementary to a sequence having at least about 80% identity to any one of SEQ ID NOs: 211-251. In some embodiments, the guide polynucleotide hybridizes or targets a sequence complementary to a sequence having at least about 85% identity to any one of SEQ ID NOs: 211-251. In some embodiments, the guide polynucleotide hybridizes or targets a sequence complementary to a sequence having at least about 90% identity to any one of SEQ ID NOs: 211-251. In some embodiments, the guide polynucleotide hybridizes or targets a sequence complementary to a sequence having at least about 95% identity to any one of SEQ ID NOs: 211-251. In some embodiments, the guide polynucleotide hybridizes or targets a sequence complementary to a sequence having at least about 96% identity to any one of SEQ ID NOs: 211-251. In some embodiments, the guide polynucleotide hybridizes or targets a sequence complementary to a sequence having at least about 97% identity to any one of SEQ ID NOs: 211-251. In some embodiments, the guide polynucleotide hybridizes or targets a sequence complementary to a sequence having at least about 98% identity to any one of SEQ ID NOs: 211-251. In some embodiments, the guide polynucleotide hybridizes or targets a sequence complementary to a sequence having at least about 99% identity to any one of SEQ ID NOs: 211-251. In some embodiments, the guide polynucleotide hybridizes or targets a sequence complementary to a sequence having 100% identity to any one of SEQ ID NOs: 211-251.

In some embodiments, the guide polynucleotide hybridizes or targets a sequence within the TRBC1 gene or within an intron of an endogenous gene. In some embodiments, the guide polynucleotide hybridizes or targets a sequence according to any one of SEQ ID NOs: 252-292 or a sequence having at least 90%, 95%, 97%, 98%, or 99% sequence identity to any one of SEQ ID NOs: 252-292. In some embodiments, the guide polynucleotide hybridizes or targets a sequence having at least about 80% identity to any one of SEQ ID NOs: 252-292. In some embodiments, the guide polynucleotide hybridizes or targets a sequence having at least about 85% identity to any one of SEQ ID NOs: 252-292. In some embodiments, the guide polynucleotide hybridizes or targets a sequence having at least about 90% identity to any one of SEQ ID NOs: 252-292. In some embodiments, the guide polynucleotide hybridizes or targets a sequence having at least about 95% identity to any one of SEQ ID NOs: 252-292. In some embodiments, the guide polynucleotide hybridizes or targets a sequence having at least about 96% identity to any one of SEQ ID NOs: 252-292. In some embodiments, the guide polynucleotide hybridizes or targets a sequence having at least about 97% identity to any one of SEQ ID NOs: 252-292. In some embodiments, the guide polynucleotide hybridizes or targets a sequence having at least about 98% identity to any one of SEQ ID NOs: 252-292. In some embodiments, the guide polynucleotide hybridizes or targets a sequence having at least about 99% identity to any one of SEQ ID NOs: 252-292. In some embodiments, the guide polynucleotide hybridizes or targets a sequence having 100% identity to any one of SEQ ID NOs: 252-292.

In some embodiments, the target gene is TRBC2. In some embodiments, the guide polynucleotide is encoded by any one of SEQ ID NOs: 293-337 or a sequence having at least 90%, 95%, 97%, 98%, or 99% sequence identity to any one of SEQ ID NOs: 293-337. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 293-337. In some embodiments, the guide polynucleotide is encoded by a sequence having at least about 80% identity to any one of SEQ ID NOs: 293-337. In some embodiments, the guide polynucleotide is encoded by a sequence having at least about 85% identity to any one of SEQ ID NOs: 293-337. In some embodiments, the guide polynucleotide is encoded by a sequence having at least about 90% identity to any one of SEQ ID NOs: 293-337. In some embodiments, the guide polynucleotide is encoded by a sequence having at least about 95% identity to any one of SEQ ID NOs: 293-337. In some embodiments, the guide polynucleotide is encoded by a sequence having at least about 96% identity to any one of SEQ ID NOs: 293-337. In some embodiments, the guide polynucleotide is encoded by a sequence having at least about 97% identity to any one of SEQ ID NOs: 293-337. In some embodiments, the guide polynucleotide is encoded by a sequence having at least about 98% identity to any one of SEQ ID NOs: 293-337. In some embodiments, the guide polynucleotide is encoded by a sequence having at least about 99% identity to any one of SEQ ID NOs: 293-337. In some embodiments, the guide polynucleotide is encoded by a sequence having 100% identity to any one of SEQ ID NOs: 293-337.

In some embodiments, the guide polynucleotide hybridizes or targets a sequence complementary to a target nucleic acid sequence within the TRBC2 gene or within an intron of an endogenous gene. In some embodiments, the guide polynucleotide hybridizes or targets a sequence complementary to any one of SEQ ID NOs: 293-337 or a sequence having at least 90%, 95%, 97%, 98%, or 99% sequence identity to any one of SEQ ID NOs: 293-337. In some embodiments, the guide polynucleotide hybridizes or targets a sequence complementary to a sequence having at least about 80% identity to any one of SEQ ID NOs: 293-337. In some embodiments, the guide polynucleotide hybridizes or targets a sequence complementary to a sequence having at least about 85% identity to any one of SEQ ID NOs: 293-337. In some embodiments, the guide polynucleotide hybridizes or targets a sequence complementary to a sequence having at least about 90% identity to any one of SEQ ID NOs: 293-337. In some embodiments, the guide polynucleotide hybridizes or targets a sequence complementary to a sequence having at least about 95% identity to any one of SEQ ID NOs: 293-337. In some embodiments, the guide polynucleotide hybridizes or targets a sequence complementary to a sequence having at least about 96% identity to any one of SEQ ID NOs: 293-337. In some embodiments, the guide polynucleotide hybridizes or targets a sequence complementary to a sequence having at least about 97% identity to any one of SEQ ID NOs: 293-337. In some embodiments, the guide polynucleotide hybridizes or targets a sequence complementary to a sequence having at least about 98% identity to any one of SEQ ID NOs: 293-337. In some embodiments, the guide polynucleotide hybridizes or targets a sequence complementary to a sequence having at least about 99% identity to any one of SEQ ID NOs: 293-337. In some embodiments, the guide polynucleotide hybridizes or targets a sequence complementary to a sequence having 100% identity to any one of SEQ ID NOs: 293-337.

In some embodiments, the guide polynucleotide hybridizes or targets a sequence within the TRBC2 gene or within an intron of an endogenous gene. In some embodiments, the guide polynucleotide hybridizes or targets a sequence according to any one of SEQ ID NOs: 338-382 or a sequence having at least 90%, 95%, 97%, 98%, or 99% sequence identity to any one of SEQ ID NOs: 338-382. In some embodiments, the guide polynucleotide hybridizes or targets a sequence having at least about 80% identity to any one of SEQ ID NOs: 338-382. In some embodiments, the guide polynucleotide hybridizes or targets a sequence having at least about 85% identity to any one of SEQ ID NOs: 338-382. In some embodiments, the guide polynucleotide hybridizes or targets a sequence having at least about 90% identity to any one of SEQ ID NOs: 338-382. In some embodiments, the guide polynucleotide hybridizes or targets a sequence having at least about 95% identity to any one of SEQ ID NOs: 338-382. In some embodiments, the guide polynucleotide hybridizes or targets a sequence having at least about 96% identity to any one of SEQ ID NOs: 338-382. In some embodiments, the guide polynucleotide hybridizes or targets a sequence having at least about 97% identity to any one of SEQ ID NOs: 338-382. In some embodiments, the guide polynucleotide hybridizes or targets a sequence having at least about 98% identity to any one of SEQ ID NOs: 338-382. In some embodiments, the guide polynucleotide hybridizes or targets a sequence having at least about 99% identity to any one of SEQ ID NOs: 338-382. In some embodiments, the guide polynucleotide hybridizes or targets a sequence having 100% identity to any one of SEQ ID NOs: 338-382.

In some embodiments, the target gene is ANGPTL3. In some embodiments, the guide polynucleotide is encoded by any one of SEQ ID NOs: 383-477, 1392-1489, 2120-2215, and 2312-2350 or a sequence having at least 90%, 95%, 97%, 98%, or 99% sequence identity to any one of SEQ ID NOs: 383-477, 1392-1489, 2120-2215, and 2312-2350. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 383-477, 1392-1489, 2120-2215, and 2312-2350. In some embodiments, the guide polynucleotide is encoded by a sequence having at least about 80% identity to any one of SEQ ID NOs: 383-477, 1392-1489, 2120-2215, and 2312-2350. In some embodiments, the guide polynucleotide is encoded by a sequence having at least about 85% identity to any one of SEQ ID NOs: 383-477, 1392-1489, 2120-2215, and 2312-2350. In some embodiments, the guide polynucleotide is encoded by a sequence having at least about 90% identity to any one of SEQ ID NOs: 383-477, 1392-1489, 2120-2215, and 2312-2350. In some embodiments, the guide polynucleotide is encoded by a sequence having at least about 95% identity to any one of SEQ ID NOs: 383-477, 1392-1489, 2120-2215, and 2312-2350. In some embodiments, the guide polynucleotide is encoded by a sequence having at least about 96% identity to any one of SEQ ID NOs: 383-477, 1392-1489, 2120-2215, and 2312-2350. In some embodiments, the guide polynucleotide is encoded by a sequence having at least about 97% identity to any one of SEQ ID NOs: 383-477, 1392-1489, 2120-2215, and 2312-2350. In some embodiments, the guide polynucleotide is encoded by a sequence having at least about 98% identity to any one of SEQ ID NOs: 383-477, 1392-1489, 2120-2215, and 2312-2350. In some embodiments, the guide polynucleotide is encoded by a sequence having at least about 99% identity to any one of SEQ ID NOs: 383-477, 1392-1489, 2120-2215, and 2312-2350. In some embodiments, the guide polynucleotide is encoded by a sequence having 100% identity to any one of SEQ ID NOs: 383-477, 1392-1489, 2120-2215, and 2312-2350.

In some embodiments, the guide polynucleotide hybridizes or targets a sequence complementary to a target nucleic acid sequence within the ANGPTL3 gene or within an intron of an endogenous gene. In some embodiments, the guide polynucleotide hybridizes or targets a sequence complementary to any one of SEQ ID NOs: 383-477, 1392-1489, 2120-2215, and 2312-2350 or a sequence having at least 90%, 95%, 97%, 98%, or 99% sequence identity to any one of SEQ ID NOs: 383-477, 1392-1489, 2120-2215, and 2312-2350. In some embodiments, the guide polynucleotide hybridizes or targets a sequence complementary to a sequence having at least about 80% identity to any one of SEQ ID NOs: 383-477, 1392-1489, 2120-2215, and 2312-2350. In some embodiments, the guide polynucleotide hybridizes or targets a sequence complementary to a sequence having at least about 85% identity to any one of SEQ ID NOs: 383-477, 1392-1489, 2120-2215, and 2312-2350. In some embodiments, the guide polynucleotide hybridizes or targets a sequence complementary to a sequence having at least about 90% identity to any one of SEQ ID NOs: 383-477, 1392-1489, 2120-2215, and 2312-2350. In some embodiments, the guide polynucleotide hybridizes or targets a sequence complementary to a sequence having at least about 95% identity to any one of SEQ ID NOs: 383-477, 1392-1489, 2120-2215, and 2312-2350. In some embodiments, the guide polynucleotide hybridizes or targets a sequence complementary to a sequence having at least about 96% identity to any one of SEQ ID NOs: 383-477, 1392-1489, 2120-2215, and 2312-2350. In some embodiments, the guide polynucleotide hybridizes or targets a sequence complementary to a sequence having at least about 97% identity to any one of SEQ ID NOs: 383-477, 1392-1489, 2120-2215, and 2312-2350. In some embodiments, the guide polynucleotide hybridizes or targets a sequence complementary to a sequence having at least about 98% identity to any one of SEQ ID NOs: 383-477, 1392-1489, 2120-2215, and 2312-2350. In some embodiments, the guide polynucleotide hybridizes or targets a sequence complementary to a sequence having at least about 99% identity to any one of SEQ ID NOs: 383-477, 1392-1489, 2120-2215, and 2312-2350. In some embodiments, the guide polynucleotide hybridizes or targets a sequence complementary to a sequence having 100% identity to any one of SEQ ID NOs: 383-477, 1392-1489, 2120-2215, and 2312-2350.

In some embodiments, the guide polynucleotide hybridizes or targets a sequence within the ANGPTL3 gene or within an intron of an endogenous gene. In some embodiments, the guide polynucleotide hybridizes or targets a sequence according to any one of SEQ ID NOs: 478-572, 1490-1587, 2216-2311, and 2351-2389 or a sequence having at least 90%, 95%, 97%, 98%, or 99% sequence identity to any one of SEQ ID NOs: 478-572, 1490-1587, 2216-2311, and 2351-2389. In some embodiments, the guide polynucleotide hybridizes or targets a sequence having at least about 80% identity to any one of SEQ ID NOs: 478-572, 1490-1587, 2216-2311, and 2351-2389. In some embodiments, the guide polynucleotide hybridizes or targets a sequence having at least about 85% identity to any one of SEQ ID NOs: 478-572, 1490-1587, 2216-2311, and 2351-2389. In some embodiments, the guide polynucleotide hybridizes or targets a sequence having at least about 90% identity to any one of SEQ ID NOs: 478-572, 1490-1587, 2216-2311, and 2351-2389. In some embodiments, the guide polynucleotide hybridizes or targets a sequence having at least about 95% identity to any one of SEQ ID NOs: 478-572, 1490-1587, 2216-2311, and 2351-2389. In some embodiments, the guide polynucleotide hybridizes or targets a sequence having at least about 96% identity to any one of SEQ ID NOs: 478-572, 1490-1587, 2216-2311, and 2351-2389. In some embodiments, the guide polynucleotide hybridizes or targets a sequence having at least about 97% identity to any one of SEQ ID NOs: 478-572, 1490-1587, 2216-2311, and 2351-2389. In some embodiments, the guide polynucleotide hybridizes or targets a sequence having at least about 98% identity to any one of SEQ ID NOs: 478-572, 1490-1587, 2216-2311, and 2351-2389. In some embodiments, the guide polynucleotide hybridizes or targets a sequence having at least about 99% identity to any one of SEQ ID NOs: 478-572, 1490-1587, 2216-2311, and 2351-2389. In some embodiments, the guide polynucleotide hybridizes or targets a sequence having 100% identity to any one of SEQ ID NOs: 478-572, 1490-1587, 2216-2311, and 2351-2389.

In some embodiments, the target gene is PCSK9. In some embodiments, the guide polynucleotide is encoded by any one of SEQ ID NOs: 573-587 and 1362-1376 or a sequence having at least 90%, 95%, 97%, 98%, or 99% sequence identity to any one of SEQ ID NOs: 573-587 and 1362-1376. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 573-587 and 1362-1376. In some embodiments, the guide polynucleotide is encoded by a sequence having at least about 80% identity to any one of SEQ ID NOs: 573-587 and 1362-1376. In some embodiments, the guide polynucleotide is encoded by a sequence having at least about 85% identity to any one of SEQ ID NOs: 573-587 and 1362-1376. In some embodiments, the guide polynucleotide is encoded by a sequence having at least about 90% identity to any one of SEQ ID NOs: 573-587 and 1362-1376. In some embodiments, the guide polynucleotide is encoded by a sequence having at least about 95% identity to any one of SEQ ID NOs: 573-587 and 1362-1376. In some embodiments, the guide polynucleotide is encoded by a sequence having at least about 96% identity to any one of SEQ ID NOs: 573-587 and 1362-1376. In some embodiments, the guide polynucleotide is encoded by a sequence having at least about 97% identity to any one of SEQ ID NOs: 573-587 and 1362-1376. In some embodiments, the guide polynucleotide is encoded by a sequence having at least about 98% identity to any one of SEQ ID NOs: 573-587 and 1362-1376. In some embodiments, the guide polynucleotide is encoded by a sequence having at least about 99% identity to any one of SEQ ID NOs: 573-587 and 1362-1376. In some embodiments, the guide polynucleotide is encoded by a sequence having 100% identity to any one of SEQ ID NOs: 573-587 and 1362-1376.

In some embodiments, the guide polynucleotide hybridizes or targets a sequence complementary to a target nucleic acid sequence within the PCSK9 gene or within an intron of an endogenous gene. In some embodiments, the guide polynucleotide hybridizes or targets a sequence complementary to any one of SEQ ID NOs: 573-587 and 1362-1376 or a sequence having at least 90%, 95%, 97%, 98%, or 99% sequence identity to any one of SEQ ID NOs: 573-587 and 1362-1376. In some embodiments, the guide polynucleotide hybridizes or targets a sequence complementary to a sequence having at least about 80% identity to any one of SEQ ID NOs: 573-587 and 1362-1376. In some embodiments, the guide polynucleotide hybridizes or targets a sequence complementary to a sequence having at least about 85% identity to any one of SEQ ID NOs: 573-587 and 1362-1376. In some embodiments, the guide polynucleotide hybridizes or targets a sequence complementary to a sequence having at least about 90% identity to any one of SEQ ID NOs: 573-587 and 1362-1376. In some embodiments, the guide polynucleotide hybridizes or targets a sequence complementary to a sequence having at least about 95% identity to any one of SEQ ID NOs: 573-587 and 1362-1376. In some embodiments, the guide polynucleotide hybridizes or targets a sequence complementary to a sequence having at least about 96% identity to any one of SEQ ID NOs: 573-587 and 1362-1376. In some embodiments, the guide polynucleotide hybridizes or targets a sequence complementary to a sequence having at least about 97% identity to any one of SEQ ID NOs: 573-587 and 1362-1376. In some embodiments, the guide polynucleotide hybridizes or targets a sequence complementary to a sequence having at least about 98% identity to any one of SEQ ID NOs: 573-587 and 1362-1376. In some embodiments, the guide polynucleotide hybridizes or targets a sequence complementary to a sequence having at least about 99% identity to any one of SEQ ID NOs: 573-587 and 1362-1376. In some embodiments, the guide polynucleotide hybridizes or targets a sequence complementary to a sequence having 100% identity to any one of SEQ ID NOs: 573-587 and 1362-1376.

In some embodiments, the guide polynucleotide hybridizes or targets a sequence within the PCSK9 gene or within an intron of an endogenous gene. In some embodiments, the guide polynucleotide hybridizes or targets a sequence according to any one of SEQ ID NOs: 588-602 and 1377-1391 or a sequence having at least 90%, 95%, 97%, 98%, or 99% sequence identity to any one of SEQ ID NOs: 588-602 and 1377-1391. In some embodiments, the guide polynucleotide hybridizes or targets a sequence having at least about 80% identity to any one of SEQ ID NOs: 588-602 and 1377-1391. In some embodiments, the guide polynucleotide hybridizes or targets a sequence having at least about 85% identity to any one of SEQ ID NOs: 588-602 and 1377-1391. In some embodiments, the guide polynucleotide hybridizes or targets a sequence having at least about 90% identity to any one of SEQ ID NOs: 588-602 and 1377-1391. In some embodiments, the guide polynucleotide hybridizes or targets a sequence having at least about 95% identity to any one of SEQ ID NOs: 588-602 and 1377-1391. In some embodiments, the guide polynucleotide hybridizes or targets a sequence having at least about 96% identity to any one of SEQ ID NOs: 588-602 and 1377-1391. In some embodiments, the guide polynucleotide hybridizes or targets a sequence having at least about 97% identity to any one of SEQ ID NOs: 588-602 and 1377-1391. In some embodiments, the guide polynucleotide hybridizes or targets a sequence having at least about 98% identity to any one of SEQ ID NOs: 588-602 and 1377-1391. In some embodiments, the guide polynucleotide hybridizes or targets a sequence having at least about 99% identity to any one of SEQ ID NOs: 588-602 and 1377-1391. In some embodiments, the guide polynucleotide hybridizes or targets a sequence having 100% identity to any one of SEQ ID NOs: 588-602 and 1377-1391.

In some embodiments, the target gene is VCP (e.g., VCP R155). In some embodiments, the guide polynucleotide is encoded by any one of SEQ ID NOs: 723-738 and 755-762 or a sequence having at least 90%, 95%, 97%, 98%, or 99% sequence identity to any one of SEQ ID NOs: 723-738 and 755-762. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 723-738 and 755-762. In some embodiments, the guide polynucleotide is encoded by a sequence having at least about 80% identity to any one of SEQ ID NOs: 723-738 and 755-762. In some embodiments, the guide polynucleotide is encoded by a sequence having at least about 85% identity to any one of SEQ ID NOs: 723-738 and 755-762. In some embodiments, the guide polynucleotide is encoded by a sequence having at least about 90% identity to any one of SEQ ID NOs: 723-738 and 755-762. In some embodiments, the guide polynucleotide is encoded by a sequence having at least about 95% identity to any one of SEQ ID NOs: 723-738 and 755-762. In some embodiments, the guide polynucleotide is encoded by a sequence having at least about 96% identity to any one of SEQ ID NOs: 723-738 and 755-762. In some embodiments, the guide polynucleotide is encoded by a sequence having at least about 97% identity to any one of SEQ ID NOs: 723-738 and 755-762. In some embodiments, the guide polynucleotide is encoded by a sequence having at least about 98% identity to any one of SEQ ID NOs: 723-738 and 755-762. In some embodiments, the guide polynucleotide is encoded by a sequence having at least about 99% identity to any one of SEQ ID NOs: 723-738 and 755-762. In some embodiments, the guide polynucleotide is encoded by a sequence having 100% identity to any one of SEQ ID NOs: 723-738 and 755-762.

In some embodiments, the guide polynucleotide hybridizes or targets a sequence complementary to a target nucleic acid sequence within the VCP gene or within an intron of an endogenous gene. In some embodiments, the guide polynucleotide hybridizes or targets a sequence complementary to any one of SEQ ID NOs: 723-738 and 755-762 or a sequence having at least 90%, 95%, 97%, 98%, or 99% sequence identity to any one of SEQ ID NOs: 723-738 and 755-762. In some embodiments, the guide polynucleotide hybridizes or targets a sequence complementary to a sequence having at least about 80% identity to any one of SEQ ID NOs: 723-738 and 755-762. In some embodiments, the guide polynucleotide hybridizes or targets a sequence complementary to a sequence having at least about 85% identity to any one of SEQ ID NOs: 723-738 and 755-762. In some embodiments, the guide polynucleotide hybridizes or targets a sequence complementary to a sequence having at least about 90% identity to any one of SEQ ID NOs: 723-738 and 755-762. In some embodiments, the guide polynucleotide hybridizes or targets a sequence complementary to a sequence having at least about 95% identity to any one of SEQ ID NOs: 723-738 and 755-762. In some embodiments, the guide polynucleotide hybridizes or targets a sequence complementary to a sequence having at least about 96% identity to any one of SEQ ID NOs: 723-738 and 755-762. In some embodiments, the guide polynucleotide hybridizes or targets a sequence complementary to a sequence having at least about 97% identity to any one of SEQ ID NOs: 723-738 and 755-762. In some embodiments, the guide polynucleotide hybridizes or targets a sequence complementary to a sequence having at least about 98% identity to any one of SEQ ID NOs: 723-738 and 755-762. In some embodiments, the guide polynucleotide hybridizes or targets a sequence complementary to a sequence having at least about 99% identity to any one of SEQ ID NOs: 723-738 and 755-762. In some embodiments, the guide polynucleotide hybridizes or targets a sequence complementary to a sequence having 100% identity to any one of SEQ ID NOs: 723-738 and 755-762.

In some embodiments, the guide polynucleotide hybridizes or targets a sequence within the VCP gene or within an intron of an endogenous gene. In some embodiments, the guide polynucleotide hybridizes or targets a sequence according to any one of SEQ ID NOs: 739-754 and 763-770 or a sequence having at least 90%, 95%, 97%, 98%, or 99% sequence identity to any one of SEQ ID NOs: 739-754 and 763-770. In some embodiments, the guide polynucleotide hybridizes or targets a sequence having at least about 80% identity to any one of SEQ ID NOs: 739-754 and 763-770. In some embodiments, the guide polynucleotide hybridizes or targets a sequence having at least about 85% identity to any one of SEQ ID NOs: 739-754 and 763-770. In some embodiments, the guide polynucleotide hybridizes or targets a sequence having at least about 90% identity to any one of SEQ ID NOs: 739-754 and 763-770. In some embodiments, the guide polynucleotide hybridizes or targets a sequence having at least about 95% identity to any one of SEQ ID NOs: 739-754 and 763-770. In some embodiments, the guide polynucleotide hybridizes or targets a sequence having at least about 96% identity to any one of SEQ ID NOs: 739-754 and 763-770. In some embodiments, the guide polynucleotide hybridizes or targets a sequence having at least about 97% identity to any one of SEQ ID NOs: 739-754 and 763-770. In some embodiments, the guide polynucleotide hybridizes or targets a sequence having at least about 98% identity to any one of SEQ ID NOs: 739-754 and 763-770. In some embodiments, the guide polynucleotide hybridizes or targets a sequence having at least about 99% identity to any one of SEQ ID NOs: 739-754 and 763-770. In some embodiments, the guide polynucleotide hybridizes or targets a sequence having 100% identity to any one of SEQ ID NOs: 739-754 and 763-770.

In some embodiments, the target locus is AAVS1. In some embodiments, the guide polynucleotide is encoded by any one of SEQ ID NOs: 928-949 and 1012-1049 or a sequence having at least 90%, 95%, 97%, 98%, or 99% sequence identity to any one of SEQ ID NOs: 928-949 and 1012-1049. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 928-949 and 1012-1049. In some embodiments, the guide polynucleotide is encoded by a sequence having at least about 80% identity to any one of SEQ ID NOs: 928-949 and 1012-1049. In some embodiments, the guide polynucleotide is encoded by a sequence having at least about 85% identity to any one of SEQ ID NOs: 928-949 and 1012-1049. In some embodiments, the guide polynucleotide is encoded by a sequence having at least about 90% identity to any one of SEQ ID NOs: 928-949 and 1012-1049. In some embodiments, the guide polynucleotide is encoded by a sequence having at least about 95% identity to any one of SEQ ID NOs: 928-949 and 1012-1049. In some embodiments, the guide polynucleotide is encoded by a sequence having at least about 96% identity to any one of SEQ ID NOs: 928-949 and 1012-1049. In some embodiments, the guide polynucleotide is encoded by a sequence having at least about 97% identity to any one of SEQ ID NOs: 928-949 and 1012-1049. In some embodiments, the guide polynucleotide is encoded by a sequence having at least about 98% identity to any one of SEQ ID NOs: 928-949 and 1012-1049. In some embodiments, the guide polynucleotide is encoded by a sequence having at least about 99% identity to any one of SEQ ID NOs: 928-949 and 1012-1049. In some embodiments, the guide polynucleotide is encoded by a sequence having 100% identity to any one of SEQ ID NOs: 928-949 and 1012-1049.

In some embodiments, the guide polynucleotide hybridizes or targets a sequence complementary to a target nucleic acid sequence within the AAVS1 locus or within an intron of an endogenous gene. In some embodiments, the guide polynucleotide hybridizes or targets a sequence complementary to any one of SEQ ID NOs: 928-949 and 1012-1049 or a sequence having at least 90%, 95%, 97%, 98%, or 99% sequence identity to any one of SEQ ID NOs: 928-949 and 1012-1049. In some embodiments, the guide polynucleotide hybridizes or targets a sequence complementary to a sequence having at least about 80% identity to any one of SEQ ID NOs: 928-949 and 1012-1049. In some embodiments, the guide polynucleotide hybridizes or targets a sequence complementary to a sequence having at least about 85% identity to any one of SEQ ID NOs: 928-949 and 1012-1049. In some embodiments, the guide polynucleotide hybridizes or targets a sequence complementary to a sequence having at least about 90% identity to any one of SEQ ID NOs: 928-949 and 1012-1049. In some embodiments, the guide polynucleotide hybridizes or targets a sequence complementary to a sequence having at least about 95% identity to any one of SEQ ID NOs: 928-949 and 1012-1049. In some embodiments, the guide polynucleotide hybridizes or targets a sequence complementary to a sequence having at least about 96% identity to any one of SEQ ID NOs: 928-949 and 1012-1049. In some embodiments, the guide polynucleotide hybridizes or targets a sequence complementary to a sequence having at least about 97% identity to any one of SEQ ID NOs: 928-949 and 1012-1049. In some embodiments, the guide polynucleotide hybridizes or targets a sequence complementary to a sequence having at least about 98% identity to any one of SEQ ID NOs: 928-949 and 1012-1049. In some embodiments, the guide polynucleotide hybridizes or targets a sequence complementary to a sequence having at least about 99% identity to any one of SEQ ID NOs: 928-949 and 1012-1049. In some embodiments, the guide polynucleotide hybridizes or targets a sequence complementary to a sequence having 100% identity to any one of SEQ ID NOs: 928-949 and 1012-1049.

In some embodiments, the guide polynucleotide hybridizes or targets a sequence within the AAVS1 locus or within an intron of an endogenous gene. In some embodiments, the guide polynucleotide hybridizes or targets a sequence according to any one of SEQ ID NOs: 950-971 and 1050-1087 or a sequence having at least 90%, 95%, 97%, 98%, or 99% sequence identity to any one of SEQ ID NOs: 950-971 and 1050-1087. In some embodiments, the guide polynucleotide hybridizes or targets a sequence having at least about 80% identity to any one of SEQ ID NOs: 950-971 and 1050-1087. In some embodiments, the guide polynucleotide hybridizes or targets a sequence having at least about 85% identity to any one of SEQ ID NOs: 950-971 and 1050-1087. In some embodiments, the guide polynucleotide hybridizes or targets a sequence having at least about 90% identity to any one of SEQ ID NOs: 950-971 and 1050-1087. In some embodiments, the guide polynucleotide hybridizes or targets a sequence having at least about 95% identity to any one of SEQ ID NOs: 950-971 and 1050-1087. In some embodiments, the guide polynucleotide hybridizes or targets a sequence having at least about 96% identity to any one of SEQ ID NOs: 950-971 and 1050-1087. In some embodiments, the guide polynucleotide hybridizes or targets a sequence having at least about 97% identity to any one of SEQ ID NOs: 950-971 and 1050-1087. In some embodiments, the guide polynucleotide hybridizes or targets a sequence having at least about 98% identity to any one of SEQ ID NOs: 950-971 and 1050-1087. In some embodiments, the guide polynucleotide hybridizes or targets a sequence having at least about 99% identity to any one of SEQ ID NOs: 950-971 and 1050-1087. In some embodiments, the guide polynucleotide hybridizes or targets a sequence having 100% identity to any one of SEQ ID NOs: 950-971 and 1050-1087.

In some embodiments, the target gene is GPR146. In some embodiments, the guide polynucleotide is encoded by any one of SEQ ID NOs: 1588-1656 or a sequence having at least 90%, 95%, 97%, 98%, or 99% sequence identity to any one of SEQ ID NOs: 1588-1656. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1588-1656. In some embodiments, the guide polynucleotide is encoded by a sequence having at least about 80% identity to any one of SEQ ID NOs: 1588-1656. In some embodiments, the guide polynucleotide is encoded by a sequence having at least about 85% identity to any one of SEQ ID NOs: 1588-1656. In some embodiments, the guide polynucleotide is encoded by a sequence having at least about 90% identity to any one of SEQ ID NOs: 1588-1656. In some embodiments, the guide polynucleotide is encoded by a sequence having at least about 95% identity to any one of SEQ ID NOs: 1588-1656. In some embodiments, the guide polynucleotide is encoded by a sequence having at least about 96% identity to any one of SEQ ID NOs: 1588-1656. In some embodiments, the guide polynucleotide is encoded by a sequence having at least about 97% identity to any one of SEQ ID NOs: 1588-1656. In some embodiments, the guide polynucleotide is encoded by a sequence having at least about 98% identity to any one of SEQ ID NOs: 1588-1656. In some embodiments, the guide polynucleotide is encoded by a sequence having at least about 99% identity to any one of SEQ ID NOs: 1588-1656. In some embodiments, the guide polynucleotide is encoded by a sequence having 100% identity to any one of SEQ ID NOs: 1588-1656.

In some embodiments, the guide polynucleotide hybridizes or targets a sequence complementary to a target nucleic acid sequence within the GPR146 gene or within an intron of an endogenous gene. In some embodiments, the guide polynucleotide hybridizes or targets a sequence complementary to any one of SEQ ID NOs: 1588-1656 or a sequence having at least 90%, 95%, 97%, 98%, or 99% sequence identity to any one of SEQ ID NOs: 1588-1656. In some embodiments, the guide polynucleotide hybridizes or targets a sequence complementary to a sequence having at least about 80% identity to any one of SEQ ID NOs: 1588-1656. In some embodiments, the guide polynucleotide hybridizes or targets a sequence complementary to a sequence having at least about 85% identity to any one of SEQ ID NOs: 1588-1656. In some embodiments, the guide polynucleotide hybridizes or targets a sequence complementary to a sequence having at least about 90% identity to any one of SEQ ID NOs: 1588-1656. In some embodiments, the guide polynucleotide hybridizes or targets a sequence complementary to a sequence having at least about 95% identity to any one of SEQ ID NOs: 1588-1656. In some embodiments, the guide polynucleotide hybridizes or targets a sequence complementary to a sequence having at least about 96% identity to any one of SEQ ID NOs: 1588-1656. In some embodiments, the guide polynucleotide hybridizes or targets a sequence complementary to a sequence having at least about 97% identity to any one of SEQ ID NOs: 1588-1656. In some embodiments, the guide polynucleotide hybridizes or targets a sequence complementary to a sequence having at least about 98% identity to any one of SEQ ID NOs: 1588-1656. In some embodiments, the guide polynucleotide hybridizes or targets a sequence complementary to a sequence having at least about 99% identity to any one of SEQ ID NOs: 1588-1656. In some embodiments, the guide polynucleotide hybridizes or targets a sequence complementary to a sequence having 100% identity to any one of SEQ ID NOs: 1588-1656.

In some embodiments, the guide polynucleotide hybridizes or targets a sequence within the GPR146 gene or within an intron of an endogenous gene. In some embodiments, the guide polynucleotide hybridizes or targets a sequence according to any one of SEQ ID NOs: 1657-1725 or a sequence having at least 90%, 95%, 97%, 98%, or 99% sequence identity to any one of SEQ ID NOs: 1657-1725. In some embodiments, the guide polynucleotide hybridizes or targets a sequence having at least about 80% identity to any one of SEQ ID NOs: 1657-1725. In some embodiments, the guide polynucleotide hybridizes or targets a sequence having at least about 85% identity to any one of SEQ ID NOs: 1657-1725.

In some embodiments, the guide polynucleotide hybridizes or targets a sequence having at least about 90% identity to any one of SEQ ID NOs: 1657-1725. In some embodiments, the guide polynucleotide hybridizes or targets a sequence having at least about 95% identity to any one of SEQ ID NOs: 1657-1725. In some embodiments, the guide polynucleotide hybridizes or targets a sequence having at least about 96% identity to any one of SEQ ID NOs: 1657-1725. In some embodiments, the guide polynucleotide hybridizes or targets a sequence having at least about 97% identity to any one of SEQ ID NOs: 1657-1725. In some embodiments, the guide polynucleotide hybridizes or targets a sequence having at least about 98% identity to any one of SEQ ID NOs: 1657-1725. In some embodiments, the guide polynucleotide hybridizes or targets a sequence having at least about 99% identity to any one of SEQ ID NOs: 1657-1725. In some embodiments, the guide polynucleotide hybridizes or targets a sequence having 100% identity to any one of SEQ ID NOs: 1657-1725.

In some embodiments, the target gene is APOA1. In some embodiments, the guide polynucleotide is encoded by any one of SEQ ID NOs: 1726-1744, 1764-1774, 1866-1961, and 2058-2088 or a sequence having at least 90%, 95%, 97%, 98%, or 99% sequence identity to any one of SEQ ID NOs: 1726-1744, 1764-1774, 1866-1961, and 2058-2088. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1726-1744, 1764-1774, 1866-1961, and 2058-2088. In some embodiments, the guide polynucleotide is encoded by a sequence having at least about 80% identity to any one of SEQ ID NOs: 1726-1744, 1764-1774, 1866-1961, and 2058-2088. In some embodiments, the guide polynucleotide is encoded by a sequence having at least about 85% identity to any one of SEQ ID NOs: 1726-1744, 1764-1774, 1866-1961, and 2058-2088. In some embodiments, the guide polynucleotide is encoded by a sequence having at least about 90% identity to any one of SEQ ID NOs: 1726-1744, 1764-1774, 1866-1961, and 2058-2088. In some embodiments, the guide polynucleotide is encoded by a sequence having at least about 95% identity to any one of SEQ ID NOs: 1726-1744, 1764-1774, 1866-1961, and 2058-2088. In some embodiments, the guide polynucleotide is encoded by a sequence having at least about 96% identity to any one of SEQ ID NOs: 1726-1744, 1764-1774, 1866-1961, and 2058-2088.

In some embodiments, the guide polynucleotide is encoded by a sequence having at least about 97% identity to any one of SEQ ID NOs: 1726-1744, 1764-1774, 1866-1961, and 2058-2088. In some embodiments, the guide polynucleotide is encoded by a sequence having at least about 98% identity to any one of SEQ ID NOs: 1726-1744, 1764-1774, 1866-1961, and 2058-2088. In some embodiments, the guide polynucleotide is encoded by a sequence having at least about 99% identity to any one of SEQ ID NOs: 1726-1744, 1764-1774, 1866-1961, and 2058-2088. In some embodiments, the guide polynucleotide is encoded by a sequence having 100% identity to any one of SEQ ID NOs: 1726-1744, 1764-1774, 1866-1961, and 2058-2088.

In some embodiments, the guide polynucleotide hybridizes or targets a sequence complementary to a target nucleic acid sequence within the APOA1 gene or within an intron of an endogenous gene. In some embodiments, the guide polynucleotide hybridizes or targets a sequence complementary to any one of SEQ ID NOs: 1726-1744, 1764-1774, 1866-1961, and 2058-2088 or a sequence having at least 90%, 95%, 97%, 98%, or 99% sequence identity to any one of SEQ ID NOs: 1726-1744, 1764-1774, 1866-1961, and 2058-2088. In some embodiments, the guide polynucleotide hybridizes or targets a sequence complementary to a sequence having at least about 80% identity to any one of SEQ ID NOs: 1726-1744, 1764-1774, 1866-1961, and 2058-2088. In some embodiments, the guide polynucleotide hybridizes or targets a sequence complementary to a sequence having at least about 85% identity to any one of SEQ ID NOs: 1726-1744, 1764-1774, 1866-1961, and 2058-2088. In some embodiments, the guide polynucleotide hybridizes or targets a sequence complementary to a sequence having at least about 90% identity to any one of SEQ ID NOs: 1726-1744, 1764-1774, 1866-1961, and 2058-2088. In some embodiments, the guide polynucleotide hybridizes or targets a sequence complementary to a sequence having at least about 95% identity to any one of SEQ ID NOs: 1726-1744, 1764-1774, 1866-1961, and 2058-2088. In some embodiments, the guide polynucleotide hybridizes or targets a sequence complementary to a sequence having at least about 96% identity to any one of SEQ ID NOs: 1726-1744, 1764-1774, 1866-1961, and 2058-2088. In some embodiments, the guide polynucleotide hybridizes or targets a sequence complementary to a sequence having at least about 97% identity to any one of SEQ ID NOs: 1726-1744, 1764-1774, 1866-1961, and 2058-2088. In some embodiments, the guide polynucleotide hybridizes or targets a sequence complementary to a sequence having at least about 98% identity to any one of SEQ ID NOs: 1726-1744, 1764-1774, 1866-1961, and 2058-2088. In some embodiments, the guide polynucleotide hybridizes or targets a sequence complementary to a sequence having at least about 99% identity to any one of SEQ ID NOs: 1726-1744, 1764-1774, 1866-1961, and 2058-2088. In some embodiments, the guide polynucleotide hybridizes or targets a sequence complementary to a sequence having 100% identity to any one of SEQ ID NOs: 1726-1744, 1764-1774, 1866-1961, and 2058-2088.

In some embodiments, the guide polynucleotide hybridizes or targets a sequence within the APOA1 gene or within an intron of an endogenous gene. In some embodiments, the guide polynucleotide hybridizes or targets a sequence according to any one of SEQ ID NOs: 1745-1763, 1775-1785, 1962-2057, and 2089-2119 or a sequence having at least 90%, 95%, 97%, 98%, or 99% sequence identity to any one of SEQ ID NOs: 1745-1763, 1775-1785, 1962-2057, and 2089-2119. In some embodiments, the guide polynucleotide hybridizes or targets a sequence having at least about 80% identity to any one of SEQ ID NOs: 1745-1763, 1775-1785, 1962-2057, and 2089-2119. In some embodiments, the guide polynucleotide hybridizes or targets a sequence having at least about 85% identity to any one of SEQ ID NOs: 1745-1763, 1775-1785, 1962-2057, and 2089-2119. In some embodiments, the guide polynucleotide hybridizes or targets a sequence having at least about 90% identity to any one of SEQ ID NOs: 1745-1763, 1775-1785, 1962-2057, and 2089-2119. In some embodiments, the guide polynucleotide hybridizes or targets a sequence having at least about 95% identity to any one of SEQ ID NOs: 1745-1763, 1775-1785, 1962-2057, and 2089-2119.

In some embodiments, the guide polynucleotide hybridizes or targets a sequence having at least about 96% identity to any one of SEQ ID NOs: 1745-1763, 1775-1785, 1962-2057, and 2089-2119. In some embodiments, the guide polynucleotide hybridizes or targets a sequence having at least about 97% identity to any one of SEQ ID NOs: 1745-1763, 1775-1785, 1962-2057, and 2089-2119. In some embodiments, the guide polynucleotide hybridizes or targets a sequence having at least about 98% identity to any one of SEQ ID NOs: 1745-1763, 1775-1785, 1962-2057, and 2089-2119. In some embodiments, the guide polynucleotide hybridizes or targets a sequence having at least about 99% identity to any one of SEQ ID NOs: 1745-1763, 1775-1785, 1962-2057, and 2089-2119. In some embodiments, the guide polynucleotide hybridizes or targets a sequence having 100% identity to any one of SEQ ID NOs: 1745-1763, 1775-1785, 1962-2057, and 2089-2119.

In some embodiments, the target gene is HAO1. In some embodiments, the guide polynucleotide is encoded by any one of SEQ ID NOs: 611-633, 1789-1826, and 1827-1865 or a sequence having at least 90%, 95%, 97%, 98%, or 99% sequence identity to any one of SEQ ID NOs: 611-633, 1789-1826, and 1827-1865. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 611-633, 1789-1826, and 1827-1865.

In some embodiments, the guide polynucleotide is encoded by a sequence having at least about 80% identity to any one of SEQ ID NOs: 611-633, 1789-1826, and 1827-1865. In some embodiments, the guide polynucleotide is encoded by a sequence having at least about 85% identity to any one of SEQ ID NOs: 611-633, 1789-1826, and 1827-1865. In some embodiments, the guide polynucleotide is encoded by a sequence having at least about 90% identity to any one of SEQ ID NOs: 611-633, 1789-1826, and 1827-1865. In some embodiments, the guide polynucleotide is encoded by a sequence having at least about 95% identity to any one of SEQ ID NOs: 611-633, 1789-1826, and 1827-1865. In some embodiments, the guide polynucleotide is encoded by a sequence having at least about 96% identity to any one of SEQ ID NOs: 611-633, 1789-1826, and 1827-1865. In some embodiments, the guide polynucleotide is encoded by a sequence having at least about 97% identity to any one of SEQ ID NOs: 611-633, 1789-1826, and 1827-1865. In some embodiments, the guide polynucleotide is encoded by a sequence having at least about 98% identity to any one of SEQ ID NOs: 611-633, 1789-1826, and 1827-1865. In some embodiments, the guide polynucleotide is encoded by a sequence having at least about 99% identity to any one of SEQ ID NOs: 611-633, 1789-1826, and 1827-1865. In some embodiments, the guide polynucleotide is encoded by a sequence having 100% identity to any one of SEQ ID NOs: 611-633, 1789-1826, and 1827-1865.

In some embodiments, the guide polynucleotide hybridizes or targets a sequence complementary to a target nucleic acid sequence within the HAO1 gene or within an intron of an endogenous gene. In some embodiments, the guide polynucleotide hybridizes or targets a sequence complementary to any one of SEQ ID NOs: 611-633, 1789-1826, and 1827-1865 or a sequence having at least 90%, 95%, 97%, 98%, or 99% sequence identity to any one of SEQ ID NOs: 611-633, 1789-1826, and 1827-1865. In some embodiments, the guide polynucleotide hybridizes or targets a sequence complementary to a sequence having at least about 80% identity to any one of SEQ ID NOs: 611-633, 1789-1826, and 1827-1865. In some embodiments, the guide polynucleotide hybridizes or targets a sequence complementary to a sequence having at least about 85% identity to any one of SEQ ID NOs: 611-633, 1789-1826, and 1827-1865. In some embodiments, the guide polynucleotide hybridizes or targets a sequence complementary to a sequence having at least about 90% identity to any one of SEQ ID NOs: 611-633, 1789-1826, and 1827-1865. In some embodiments, the guide polynucleotide hybridizes or targets a sequence complementary to a sequence having at least about 95% identity to any one of SEQ ID NOs: 611-633, 1789-1826, and 1827-1865. In some embodiments, the guide polynucleotide hybridizes or targets a sequence complementary to a sequence having at least about 96% identity to any one of SEQ ID NOs: 611-633, 1789-1826, and 1827-1865. In some embodiments, the guide polynucleotide hybridizes or targets a sequence complementary to a sequence having at least about 97% identity to any one of SEQ ID NOs: 611-633, 1789-1826, and 1827-1865. In some embodiments, the guide polynucleotide hybridizes or targets a sequence complementary to a sequence having at least about 98% identity to any one of SEQ ID NOs: 611-633, 1789-1826, and 1827-1865. In some embodiments, the guide polynucleotide hybridizes or targets a sequence complementary to a sequence having at least about 99% identity to any one of SEQ ID NOs: 611-633, 1789-1826, and 1827-1865. In some embodiments, the guide polynucleotide hybridizes or targets a sequence complementary to a sequence having 100% identity to any one of SEQ ID NOs: 611-633, 1789-1826, and 1827-1865.

In some embodiments, the guide polynucleotides (e.g., guide RNAs) comprise various structural elements including but not limited to: a spacer sequence which binds to the protospacer sequence (target sequence), a crRNA, and an optional tracrRNA. In some embodiments, the genome editing system comprises a CRISPR guide RNA. In some embodiments, the guide RNA comprises a crRNA comprising a spacer sequence. In some embodiments, the guide RNA additionally comprises a tracrRNA or a modified tracrRNA.

In some embodiments, the systems provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a crRNA is part or considered part of a guide RNA, or is comprised in a guide RNA, e.g., a crRNA:tracrRNA chimera.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur (S), such as a phosphorothioate inter-nucleoside linkage. In some embodiments, the guide RNA comprises greater than about 10%, 25%, 50%, 75%, or 90% modified inter-nucleoside linkers. In some embodiments, the guide RNA comprises 1, 2, 3, 4, 5, 6, 7, 8, 9, 10, or more than 10 modified inter-nucleoside linkers (e.g., phosphorothioate inter-nucleoside linkage).

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′—OH group naturally found in DNA and RNA nucleosides. In some embodiments, substituents are introduced at the 2′, 3′, 4′, 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In some embodiments, the guide RNA comprises greater than about 10%, 25%, 50%, 75%, or 90% modified sugars.

In some embodiments, the modified sugar is a bicyclic sugar. In some embodiments, the modified sugar comprises a 2′-O-methyl. In some embodiments, the modified sugar comprises a 2′-fluoro. In some embodiments, the modified sugar comprises a 2′-O-methoxyethyl group. In some embodiments, the guide RNA comprises 1, 2, 3, 4, 5, 6, 7, 8, 9, 10, or more than 10 modified sugars (e.g., comprising a 2′-O-methyl or 2′-fluoro).

In some embodiments, the guide RNA comprises both inter-nucleoside linker modifications and nucleoside modifications. In some embodiments, the guide RNA comprises greater than about 10%, 25%, 50%, 75%, or 90% modified inter-nucleoside linkers and greater than about 10%, 25%, 50%, 75%, or 90% modified sugars. In some embodiments, the guide RNA comprises 1, 2, 3, 4, 5, 6, 7, 8, 9, 10, or more than 10 modified inter-nucleoside linkers (e.g., phosphorothioate inter-nucleoside linkage) and 1, 2, 3, 4, 5, 6, 7, 8, 9, 10, or more than 10 modified sugars (e.g., comprising a 2′-O-methyl or 2′-fluoro).

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

### MG Endonuclease Systems

Described herein, in certain embodiments, are engineered nuclease systems comprising an engineered endonuclease and an engineered guide polynucleotide configured to form a complex with the endonuclease and to hybridize to a target nucleic acid sequence.

In some embodiments, the engineered nuclease system comprises an engineered endonuclease comprising a sequence having at least about 70% identity to any one of SEQ ID NOs: 1-27 and 771-862 and an engineered guide polynucleotide. In some embodiments, the engineered nuclease system comprises an engineered endonuclease comprising a sequence having at least about 75% identity to any one of SEQ ID NOs: 1-27 and 771-862 and an engineered guide polynucleotide. In some embodiments, the engineered nuclease system comprises an engineered endonuclease comprising a sequence having at least about 80% identity to any one of SEQ ID NOs: 1-27 and 771-862 and an engineered guide polynucleotide. In some embodiments, the engineered nuclease system comprises an engineered endonuclease comprising a sequence having at least about 85% identity to any one of SEQ ID NOs: 1-27 and 771-862 and an engineered guide polynucleotide. In some embodiments, the engineered nuclease system comprises an engineered endonuclease comprising a sequence having at least about 90% identity to any one of SEQ ID NOs: 1-27 and 771-862 and an engineered guide polynucleotide. In some embodiments, the engineered nuclease system comprises an engineered endonuclease comprising a sequence having at least about 95% identity to any one of SEQ ID NOs: 1-27 and 771-862 and an engineered guide polynucleotide. In some embodiments, the engineered nuclease system comprises an engineered endonuclease comprising a sequence having at least about 96% identity to any one of SEQ ID NOs: 1-27 and 771-862 and an engineered guide polynucleotide. In some embodiments, the engineered nuclease system comprises an engineered endonuclease comprising a sequence having at least about 97% identity to any one of SEQ ID NOs: 1-27 and 771-862 and an engineered guide polynucleotide. In some embodiments, the engineered nuclease system comprises an engineered endonuclease comprising a sequence having at least about 98% identity to any one of SEQ ID NOs: 1-27 and 771-862 and an engineered guide polynucleotide. In some embodiments, the engineered nuclease system comprises an engineered endonuclease comprising a sequence having at least about 99% identity to any one of SEQ ID NOs: 1-27 and 771-862 and an engineered guide polynucleotide. In some embodiments, the engineered nuclease system comprises an engineered endonuclease comprising 100% identity to any one of SEQ ID NOs: 1-27 and 771-862 and an engineered guide polynucleotide.

In some embodiments, the engineered nuclease system comprises an engineered endonuclease comprising a sequence having at least about 70% identity to any one of SEQ ID NOs: 109-110 and 2842-2854 and an engineered guide polynucleotide. In some embodiments, the engineered nuclease system comprises an engineered endonuclease comprising a sequence having at least about 75% identity to any one of SEQ ID NOs: 109-110 and 2842-2854 and an engineered guide polynucleotide. In some embodiments, the engineered nuclease system comprises an engineered endonuclease comprising a sequence having at least about 80% identity to any one of SEQ ID NOs: 109-110 and 2842-2854 and an engineered guide polynucleotide. In some embodiments, the engineered nuclease system comprises an engineered endonuclease comprising a sequence having at least about 85% identity to any one of SEQ ID NOs: 109-110 and 2842-2854 and an engineered guide polynucleotide. In some embodiments, the engineered nuclease system comprises an engineered endonuclease comprising a sequence having at least about 90% identity to any one of SEQ ID NOs: 109-110 and 2842-2854 and an engineered guide polynucleotide. In some embodiments, the engineered nuclease system comprises an engineered endonuclease comprising a sequence having at least about 95% identity to any one of SEQ ID NOs: 109-110 and 2842-2854 and an engineered guide polynucleotide. In some embodiments, the engineered nuclease system comprises an engineered endonuclease comprising a sequence having at least about 96% identity to any one of SEQ ID NOs: 109-110 and 2842-2854 and an engineered guide polynucleotide. In some embodiments, the engineered nuclease system comprises an engineered endonuclease comprising a sequence having at least about 97% identity to any one of SEQ ID NOs: 109-110 and 2842-2854 and an engineered guide polynucleotide. In some embodiments, the engineered nuclease system comprises an engineered endonuclease comprising a sequence having at least about 98% identity to any one of SEQ ID NOs: 109-110 and 2842-2854 and an engineered guide polynucleotide. In some embodiments, the engineered nuclease system comprises an engineered endonuclease comprising a sequence having at least about 99% identity to any one of SEQ ID NOs: 109-110 and 2842-2854 and an engineered guide polynucleotide. In some embodiments, the engineered nuclease system comprises an engineered endonuclease comprising 100% identity to any one of SEQ ID NOs: 109-110 and 2842-2854 and an engineered guide polynucleotide.

In some embodiments, the engineered guide polynucleotide is a single guide nucleic acid. In some embodiments, the engineered guide polynucleotide is a dual guide nucleic acid.

In some embodiments, the engineered guide polynucleotide is RNA. In some embodiments, the engineered endonuclease binds non-covalently to the engineered guide polynucleotide. In some embodiments, the endonuclease is covalently linked to the engineered guide polynucleotide.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 70% identity to any one of SEQ ID NOs: 67-86. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 75% identity to any one of SEQ ID NOs: 67-86. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 80% identity to any one of SEQ ID NOs: 67-86. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 85% identity to any one of SEQ ID NOs: 67-86. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 90% identity to any one of SEQ ID NOs: 67-86. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 95% identity to any one of SEQ ID NOs: 67-86. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 96% identity to any one of SEQ ID NOs: 67-86. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 97% identity to any one of SEQ ID NOs: 67-86. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 98% identity to any one of SEQ ID NOs: 67-86. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least about 99% identity to any one of SEQ ID NOs: 67-86. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising 100% identity to any one of SEQ ID NOs: 67-86. In some embodiments, the guide polynucleotide hybridizes or targets a sequence complementary to any one of SEQ ID NOs: 67-86 or a sequence having at least 90%, 95%, 97%, 98%, or 99% sequence identity to any one of SEQ ID NOs: 67-86.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 70% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 75% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 80% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 85% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 90% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 95% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 96% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 97% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 98% identity to any one of SEQ ID NOs: 119-138, 922-924, 972-991, 1088-1183, 1280-1320, 2390-2485, and 2582-2617. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least about 99% identity to any one of SEQ ID NOs: 119-138, 922-924, 972-991, 1088-1183, 1280-1320, 2390-2485, and 2582-2617. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising 100% identity to any one of SEQ ID NOs: 119-138, 922-924, 972-991, 1088-1183, 1280-1320, 2390-2485, and 2582-2617. In some embodiments, the guide polynucleotide hybridizes or targets a sequence complementary to any one of SEQ ID NOs: 119-138, 922-924, 972-991, 1088-1183, 1280-1320, 2390-2485, and 2582-2617 or a sequence having at least 90%, 95%, 97%, 98%, or 99% sequence identity to any one of SEQ ID NOs: 119-138, 922-924, 972-991, 1088-1183, 1280-1320, 2390-2485, and 2582-2617.

In some embodiments, the guide polynucleotide hybridizes or targets a sequence according to any one of SEQ ID NOs: 139-158, 925-927, 992-1011, 1184-1279, 1321-1361, 2486-2581, and 2618-2653 or a sequence having at least 90%, 95%, 97%, 98%, or 99% sequence identity to any one of SEQ ID NOs: 139-158, 925-927, 992-1011, 1184-1279, 1321-1361, 2486-2581, and 2618-2653. In some embodiments, the target nucleic acid sequence comprises a sequence having at least 90% sequence identity to any one of SEQ ID NOs: 139-158, 925-927, 992-1011, 1184-1279, 1321-1361, 2486-2581, and 2618-2653.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 70% identity to any one of SEQ ID NOs: 159-184. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 75% identity to any one of SEQ ID NOs: 159-184. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 80% identity to any one of SEQ ID NOs: 159-184. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 85% identity to any one of SEQ ID NOs: 159-184. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 90% identity to any one of SEQ ID NOs: 159-184. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 95% identity to any one of SEQ ID NOs: 159-184. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 96% identity to any one of SEQ ID NOs: 159-184. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 97% identity to any one of SEQ ID NOs: 159-184. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 98% identity to any one of SEQ ID NOs: 159-184. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least about 99% identity to any one of SEQ ID NOs: 159-184. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising 100% identity to any one of SEQ ID NOs: 159-184. In some embodiments, the guide polynucleotide hybridizes or targets a sequence complementary to any one of SEQ ID NOs: 159-184 or a sequence having at least 90%, 95%, 97%, 98%, or 99% sequence identity to any one of SEQ ID NOs: 159-184.

In some embodiments, the guide polynucleotide hybridizes or targets a sequence according to any one of SEQ ID NOs: 185-210 or a sequence having at least 90%, 95%, 97%, 98%, or 99% sequence identity to any one of SEQ ID NOs: 185-210. In some embodiments, the target nucleic acid sequence comprises a sequence having at least 90% sequence identity to any one of SEQ ID NOs: 185-210.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 70% identity to any one of SEQ ID NOs: 211-251. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 75% identity to any one of SEQ ID NOs: 211-251. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 80% identity to any one of SEQ ID NOs: 211-251. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 85% identity to any one of SEQ ID NOs: 211-251. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 90% identity to any one of SEQ ID NOs: 211-251. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 95% identity to any one of SEQ ID NOs: 211-251. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 96% identity to any one of SEQ ID NOs: 211-251. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 97% identity to any one of SEQ ID NOs: 211-251. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 98% identity to any one of SEQ ID NOs: 211-251. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least about 99% identity to any one of SEQ ID NOs: 211-251. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising 100% identity to any one of SEQ ID NOs: 211-251. In some embodiments, the guide polynucleotide hybridizes or targets a sequence complementary to any one of SEQ ID NOs: 211-251 or a sequence having at least 90%, 95%, 97%, 98%, or 99% sequence identity to any one of SEQ ID NOs: 211-251.

In some embodiments, the guide polynucleotide hybridizes or targets a sequence according to any one of SEQ ID NOs: 252-292 or a sequence having at least 90%, 95%, 97%, 98%, or 99% sequence identity to any one of SEQ ID NOs: 252-292. In some embodiments, the target nucleic acid sequence comprises a sequence having at least 90% sequence identity to any one of SEQ ID NOs: 252-292.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 70% identity to any one of SEQ ID NOs: 293-337. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 75% identity to any one of SEQ ID NOs: 293-337. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 80% identity to any one of SEQ ID NOs: 293-337. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 85% identity to any one of SEQ ID NOs: 293-337. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 90% identity to any one of SEQ ID NOs: 293-337. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 95% identity to any one of SEQ ID NOs: 293-337. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 96% identity to any one of SEQ ID NOs: 293-337. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 97% identity to any one of SEQ ID NOs: 293-337. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 98% identity to any one of SEQ ID NOs: 293-337. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least about 99% identity to any one of SEQ ID NOs: 293-337. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising 100% identity to any one of SEQ ID NOs: 293-337. In some embodiments, the guide polynucleotide hybridizes or targets a sequence complementary to any one of SEQ ID NOs: 293-337 or a sequence having at least 90%, 95%, 97%, 98%, or 99% sequence identity to any one of SEQ ID NOs: 293-337.

In some embodiments, the guide polynucleotide hybridizes or targets a sequence according to any one of SEQ ID NOs: 338-382 or a sequence having at least 90%, 95%, 97%, 98%, or 99% sequence identity to any one of SEQ ID NOs: 338-382. In some embodiments, the target nucleic acid sequence comprises a sequence having at least 90% sequence identity to any one of SEQ ID NOs: 338-382.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 70% identity to any one of SEQ ID NOs: 383-477, 1392-1489, 2120-2215, and 2312-2350. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 75% identity to any one of SEQ ID NOs: 383-477, 1392-1489, 2120-2215, and 2312-2350. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 80% identity to any one of SEQ ID NOs: 383-477, 1392-1489, 2120-2215, and 2312-2350. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 85% identity to any one of SEQ ID NOs: 383-477, 1392-1489, 2120-2215, and 2312-2350. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 90% identity to any one of SEQ ID NOs: 383-477, 1392-1489, 2120-2215, and 2312-2350. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 95% identity to any one of SEQ ID NOs: 383-477, 1392-1489, 2120-2215, and 2312-2350. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 96% identity to any one of SEQ ID NOs: 383-477, 1392-1489, 2120-2215, and 2312-2350. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 97% identity to any one of SEQ ID NOs: 383-477, 1392-1489, 2120-2215, and 2312-2350. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 98% identity to any one of SEQ ID NOs: 383-477, 1392-1489, 2120-2215, and 2312-2350. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least about 99% identity to any one of SEQ ID NOs: 383-477, 1392-1489, 2120-2215, and 2312-2350. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising 100% identity to any one of SEQ ID NOs: 383-477, 1392-1489, 2120-2215, and 2312-2350. In some embodiments, the guide polynucleotide hybridizes or targets a sequence complementary to any one of SEQ ID NOs: 383-477, 1392-1489, 2120-2215, and 2312-2350 or a sequence having at least 90%, 95%, 97%, 98%, or 99% sequence identity to any one of SEQ ID NOs: 383-477, 1392-1489, 2120-2215, and 2312-2350.

In some embodiments, the guide polynucleotide hybridizes or targets a sequence according to any one of SEQ ID NOs: 478-572, 1490-1587, 2216-2311, and 2351-2389 or a sequence having at least 90%, 95%, 97%, 98%, or 99% sequence identity to any one of SEQ ID NOs: 478-572, 1490-1587, 2216-2311, and 2351-2389. In some embodiments, the target nucleic acid sequence comprises a sequence having at least 90% sequence identity to any one of SEQ ID NOs: 478-572, 1490-1587, 2216-2311, and 2351-2389.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 70% identity to any one of SEQ ID NOs: 573-587 and 1362-1376. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 75% identity to any one of SEQ ID NOs: 573-587 and 1362-1376. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 80% identity to any one of SEQ ID NOs: 573-587 and 1362-1376. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 85% identity to any one of SEQ ID NOs: 573-587 and 1362-1376. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 90% identity to any one of SEQ ID NOs: 573-587 and 1362-1376. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 95% identity to any one of SEQ ID NOs: 573-587 and 1362-1376. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 96% identity to any one of SEQ ID NOs: 573-587 and 1362-1376. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 97% identity to any one of SEQ ID NOs: 573-587 and 1362-1376. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 98% identity to any one of SEQ ID NOs: 573-587 and 1362-1376. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least about 99% identity to any one of SEQ ID NOs: 573-587 and 1362-1376. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising 100% identity to any one of SEQ ID NOs: 573-587 and 1362-1376. In some embodiments, the guide polynucleotide hybridizes or targets a sequence complementary to any one of SEQ ID NOs: 573-587 and 1362-1376 or a sequence having at least 90%, 95%, 97%, 98%, or 99% sequence identity to any one of SEQ ID NOs: 573-587 and 1362-1376.

In some embodiments, the guide polynucleotide hybridizes or targets a sequence according to any one of SEQ ID NOs: 588-602 and 1377-1391 or a sequence having at least 90%, 95%, 97%, 98%, or 99% sequence identity to any one of SEQ ID NOs: 588-602 and 1377-1391. In some embodiments, the target nucleic acid sequence comprises a sequence having at least 90% sequence identity to any one of SEQ ID NOs: 588-602 and 1377-1391.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 70% identity to any one of SEQ ID NOs: 723-738 and 755-762. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 75% identity to any one of SEQ ID NOs: 723-738 and 755-762. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 80% identity to any one of SEQ ID NOs: 723-738 and 755-762. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 85% identity to any one of SEQ ID NOs: 723-738 and 755-762. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 90% identity to any one of SEQ ID NOs: 723-738 and 755-762. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 95% identity to any one of SEQ ID NOs: 723-738 and 755-762. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 96% identity to any one of SEQ ID NOs: 723-738 and 755-762. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 97% identity to any one of SEQ ID NOs: 723-738 and 755-762. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 98% identity to any one of SEQ ID NOs: 723-738 and 755-762. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least about 99% identity to any one of SEQ ID NOs: 723-738 and 755-762. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising 100% identity to any one of SEQ ID NOs: 723-738 and 755-762. In some embodiments, the guide polynucleotide hybridizes or targets a sequence complementary to any one of SEQ ID NOs: 723-738 and 755-762 or a sequence having at least 90%, 95%, 97%, 98%, or 99% sequence identity to any one of SEQ ID NOs: 723-738 and 755-762.

In some embodiments, the guide polynucleotide hybridizes or targets a sequence according to any one of SEQ ID NOs: 739-754 and 763-770 or a sequence having at least 90%, 95%, 97%, 98%, or 99% sequence identity to any one of SEQ ID NOs: 739-754 and 763-770. In some embodiments, the target nucleic acid sequence comprises a sequence having at least 90% sequence identity to any one of SEQ ID NOs: 739-754 and 763-770.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 70% identity to any one of SEQ ID NOs: 928-949 and 1012-1049. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 75% identity to any one of SEQ ID NOs: 928-949 and 1012-1049. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 80% identity to any one of SEQ ID NOs: 928-949 and 1012-1049. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 85% identity to any one of SEQ ID NOs: 928-949 and 1012-1049. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 90% identity to any one of SEQ ID NOs: 928-949 and 1012-1049. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 95% identity to any one of SEQ ID NOs: 928-949 and 1012-1049. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 96% identity to any one of SEQ ID NOs: 928-949 and 1012-1049. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 97% identity to any one of SEQ ID NOs: 928-949 and 1012-1049. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 98% identity to any one of SEQ ID NOs: 928-949 and 1012-1049. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least about 99% identity to any one of SEQ ID NOs: 928-949 and 1012-1049. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising 100% identity to any one of SEQ ID NOs: 928-949 and 1012-1049. In some embodiments, the guide polynucleotide hybridizes or targets a sequence complementary to any one of SEQ ID NOs: 928-949 and 1012-1049 or a sequence having at least 90%, 95%, 97%, 98%, or 99% sequence identity to any one of SEQ ID NOs: 928-949 and 1012-1049.

In some embodiments, the guide polynucleotide hybridizes or targets a sequence according to any one of SEQ ID NOs: 950-971 and 1050-1087 or a sequence having at least 90%, 95%, 97%, 98%, or 99% sequence identity to any one of SEQ ID NOs: 950-971 and 1050-1087. In some embodiments, the target nucleic acid sequence comprises a sequence having at least 90% sequence identity to any one of SEQ ID NOs: 950-971 and 1050-1087.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 70% identity to any one of SEQ ID NOs: 1588-1656. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 75% identity to any one of SEQ ID NOs: 1588-1656. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 80% identity to any one of SEQ ID NOs: 1588-1656. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 85% identity to any one of SEQ ID NOs: 1588-1656. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 90% identity to any one of SEQ ID NOs: 1588-1656. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 95% identity to any one of SEQ ID NOs: 1588-1656. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 96% identity to any one of SEQ ID NOs: 1588-1656. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 97% identity to any one of SEQ ID NOs: 1588-1656. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 98% identity to any one of SEQ ID NOs: 1588-1656. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least about 99% identity to any one of SEQ ID NOs: 1588-1656. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising 100% identity to any one of SEQ ID NOs: 1588-1656. In some embodiments, the guide polynucleotide hybridizes or targets a sequence complementary to any one of SEQ ID NOs: 1588-1656 or a sequence having at least 90%, 95%, 97%, 98%, or 99% sequence identity to any one of SEQ ID NOs: 1588-1656.

In some embodiments, the guide polynucleotide hybridizes or targets a sequence according to any one of SEQ ID NOs: 1657-1725 or a sequence having at least 90%, 95%, 97%, 98%, or 99% sequence identity to any one of SEQ ID NOs: 1657-1725. In some embodiments, the target nucleic acid sequence comprises a sequence having at least 90% sequence identity to any one of SEQ ID NOs: 1657-1725.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 70% identity to any one of SEQ ID NOs: 1726-1744, 1764-1774, 1866-1961 and 2058-2088. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 75% identity to any one of SEQ ID NOs: 1726-1744, 1764-1774, 1866-1961 and 2058-2088. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 80% identity to any one of SEQ ID NOs: 1726-1744, 1764-1774, 1866-1961 and 2058-2088. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 85% identity to any one of SEQ ID NOs: 1726-1744, 1764-1774, 1866-1961 and 2058-2088. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 90% identity to any one of SEQ ID NOs: 1726-1744, 1764-1774, 1866-1961 and 2058-2088. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 95% identity to any one of SEQ ID NOs: 1726-1744, 1764-1774, 1866-1961 and 2058-2088. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 96% identity to any one of SEQ ID NOs: 1726-1744, 1764-1774, 1866-1961 and 2058-2088. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 97% identity to any one of SEQ ID NOs: 1726-1744, 1764-1774, 1866-1961 and 2058-2088. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 98% identity to any one of SEQ ID NOs: 1726-1744, 1764-1774, 1866-1961 and 2058-2088. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least about 99% identity to any one of SEQ ID NOs: 1726-1744, 1764-1774, 1866-1961 and 2058-2088. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising 100% identity to any one of SEQ ID NOs: 1726-1744, 1764-1774, 1866-1961 and 2058-2088. In some embodiments, the guide polynucleotide hybridizes or targets a sequence complementary to any one of SEQ ID NOs: 1726-1744, 1764-1774, 1866-1961, and 2058-2088 or a sequence having at least 90%, 95%, 97%, 98%, or 99% sequence identity to any one of SEQ ID NOs: 1726-1744, 1764-1774, 1866-1961, and 2058-2088.

In some embodiments, the guide polynucleotide hybridizes or targets a sequence according to any one of SEQ ID NOs: 1745-1763, 1775-1785, 1962-2057, and 2089-2119 or a sequence having at least 90%, 95%, 97%, 98%, or 99% sequence identity to any one of SEQ ID NOs: 1745-1763, 1775-1785, 1962-2057, and 2089-2119. In some embodiments, the target nucleic acid sequence comprises a sequence having at least 90% sequence identity to any one of SEQ ID NOs: 1745-1763, 1775-1785, 1962-2057, and 2089-2119.

In some embodiments, the engineered nuclease system comprises an endonuclease comprising a sequence having at least about 70% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 70% identity to any one of SEQ ID NOs: 611-633, 1789-1826, and 1827-1865. In some embodiments, the engineered nuclease system comprises an endonuclease comprising a sequence having at least about 75% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 75% identity to any one of SEQ ID NOs: 611-633, 1789-1826, and 1827-1865. In some embodiments, the engineered nuclease system comprises an endonuclease comprising a sequence having at least about 80% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 80% identity to any one of SEQ ID NOs: 611-633, 1789-1826, and 1827-1865. In some embodiments, the engineered nuclease system comprises an endonuclease comprising a sequence having at least about 85% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 85% identity to any one of SEQ ID NOs: 611-633, 1789-1826, and 1827-1865. In some embodiments, the engineered nuclease system comprises an endonuclease comprising a sequence having at least about 90% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 90% identity to any one of SEQ ID NOs: 611-633, 1789-1826, and 1827-1865. In some embodiments, the engineered nuclease system comprises an endonuclease comprising a sequence having at least about 95% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 95% identity to any one of SEQ ID NOs: 611-633, 1789-1826, and 1827-1865. In some embodiments, the engineered nuclease system comprises an endonuclease comprising a sequence having at least about 96% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 96% identity to any one of SEQ ID NOs: 611-633, 1789-1826, and 1827-1865. In some embodiments, the engineered nuclease system comprises an endonuclease comprising a sequence having at least about 97% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 97% identity to any one of SEQ ID NOs: 611-633, 1789-1826, and 1827-1865. In some embodiments, the engineered nuclease system comprises an endonuclease comprising a sequence having at least about 98% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 98% identity to any one of SEQ ID NOs: 611-633, 1789-1826, and 1827-1865. In some embodiments, the engineered nuclease system comprises an endonuclease comprising a sequence having at least about 99% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least about 99% identity to any one of SEQ ID NOs: 611-633, 1789-1826, and 1827-1865. In some embodiments, the engineered nuclease system comprises an endonuclease comprising 100% identity to any one of SEQ ID NOs: 1-27 and 771-862 and an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising 100% identity to any one of SEQ ID NOs: 611-633, 1789-1826, and 1827-1865. In some embodiments, the guide polynucleotide hybridizes or targets a sequence complementary to any one of SEQ ID NOs: 611-633, 1789-1826, and 1827-1865 or a sequence having at least 90%, 95%, 97%, 98%, or 99% sequence identity to any one of SEQ ID NOs: 611-633, 1789-1826, and 1827-1865.

### Cells

Described herein, in certain embodiments, is a cell comprising engineered endonuclease systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a Streptococcus bacterium cell, a Streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the primary cell is a T cell. In some embodiments, the primary cell is a hematopoietic stem cell (HSC).

### Delivery and Vectors

Disclosed herein, in some embodiments, are nucleic acid sequences encoding an engineered nuclease system comprising an engineered endonuclease (e.g. a fusion endonuclease or a chimeric nuclease) and a guide polynucleotide, an engineered endonuclease, or a guide polynucleotide.

In some embodiments, the nucleic acid encoding the engineered endonuclease system is a DNA, for example a linear DNA, a plasmid DNA, or a minicircle DNA. In some embodiments, the nucleic acid encoding the engineered nuclease system is an RNA, for example a mRNA.

In some embodiments, the nucleic acid encoding the engineered endonuclease system is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3XFLAG, pSF-CMV-NEO—COOH-3XFLAG, pSF-CMV—PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEFla-mCherry-N1 vector, pEFla-tdTomato vector, pSF-CMV-FMDV-Hygro, pSF-CMV-PGK-Puro, pMCP-tag(m), pSF-CMV—PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV 11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the engineered endonuclease or the engineered endonuclease system is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the engineered endonuclease or the engineered endonuclease system is introduced into the cell in any suitable way, either stably or transiently. In some embodiments, the engineered endonuclease or the engineered endonuclease system is transfected into the cell. In some embodiments, the cell is transduced or transfected with a nucleic acid construct that encodes the engineered endonuclease or the engineered endonuclease system. For example, a cell is transduced (e.g., with a virus encoding the engineered endonuclease or the engineered endonuclease system), or transfected (e.g., with a plasmid encoding the engineered endonuclease or the engineered endonuclease system) with a nucleic acid that encodes the engineered endonuclease or the engineered endonuclease system, or the translated the engineered endonuclease or the engineered endonuclease system. In some embodiments, the transduction is a stable or transient transduction. In some embodiments, cells expressing the engineered endonuclease or the engineered endonuclease system or containing the engineered endonuclease or the engineered endonuclease system are transduced or transfected with one or more gRNA molecules, for example, when the engineered endonuclease or the engineered endonuclease system comprises a CRISPR nuclease. In some embodiments, a plasmid expressing the engineered endonuclease or the engineered endonuclease system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Lipid Nanoparticles

Disclosed herein, in certain embodiments, are lipid nanoparticles comprising an engineered endonuclease system of the disclosure for delivery of the engineered endonuclease systems into a cell.

In some embodiments, the lipid nanoparticle comprises the engineered endonuclease system. In some embodiments, the lipid nanoparticle comprises the engineered endonuclease or a nucleic acid encoding the engineered endonuclease and an engineered guide polynucleotide. In some embodiments, the lipid nanoparticle comprises the one or more components of the engineered endonuclease system. In some embodiments, the lipid nanoparticle comprises the engineered endonuclease or a nucleic acid encoding the engineered endonuclease. In some embodiments, the lipid nanoparticle comprises the engineered guide polynucleotide.

In some embodiments, the lipid nanoparticle is tethered to the engineered endonuclease system.

In some embodiments, the lipid nanoparticle is used to deliver the engineered endonuclease system or components thereof to a cell. In some embodiments, the cell is a specific cell type. In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the primary cell is a T cell. In some embodiments, the primary cell is a hematopoietic stem cell (HSC).

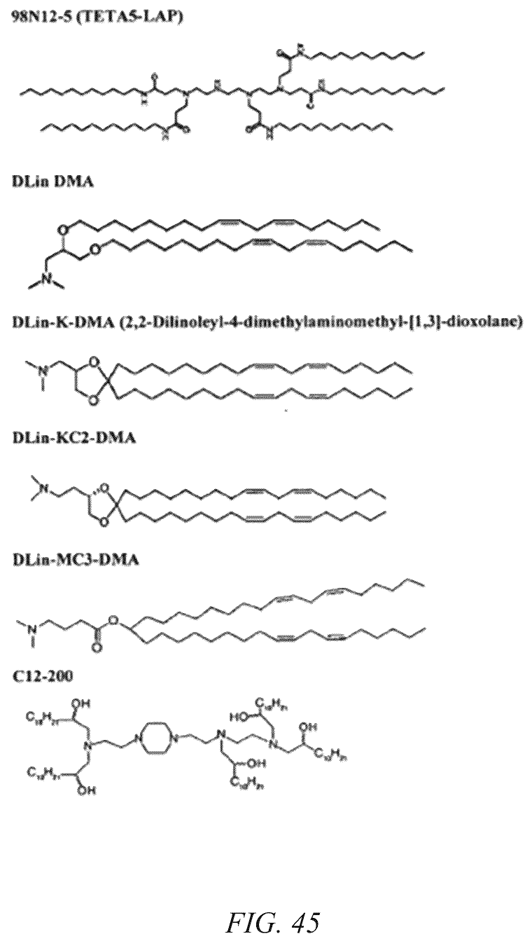

Lipid nanoparticles as described herein can be 4-component lipid nanoparticles. Such nanoparticles can be configured for delivery of RNA or other nucleic acids (e.g., synthetic RNA, mRNA, or in vitro-synthesized mRNA) and can be generally formulated as described in WO2012135805A2. Such nanoparticles can generally comprise: (a) a cationic lipid (e.g., any of the lipids described in FIG. 45), (b) a neutral lipid (e.g., DSPC or DOPE), (c) a sterol (e.g., cholesterol or a cholesterol analog), or (d) a PEG-modified lipid (e.g., PEG-DMG).

The cationic lipid referred to herein as “C12-200” is disclosed by Love et al., Proc Natl Acad Sci USA. 2010 107:1864-1869 and Liu and Huang, Molecular Therapy. 2010 669-670. Cationic lipid formulations can include particles comprising either 3 or 4 or more components in addition to polynucleotide, primary construct, or RNA (e.g., mRNA). As an example, formulations with certain cationic lipids, include, but are not limited to, 98N12-5 (or any of the other structures described in FIG. 45) and may contain 42% lipidoid, 48% cholesterol and 10% PEG (C14 or greater alkyl chain length). As another example, formulations with certain lipidoids include, but are not limited to, C12-200 and may contain 50% cationic lipid, 10% disteroylphosphatidyl choline, 38.5% cholesterol, and 1.5% PEG-DMG.

In some embodiments, lipid nanoparticles are formulated as described in U.S. Ser. No. 10/709,779. In some embodiments, the cationic lipid nanoparticle comprises a cationic lipid, a PEG-modified lipid, a sterol, and a non-cationic lipid. In some embodiments, the cationic lipid is selected from the group consisting of, or comprising any of, the cationic lipids depicted in FIG. 45. In some embodiments, the cationic lipid nanoparticle has a molar ratio of about 20-60% cationic lipid: about 5-25% non-cationic lipid: about 25-55% sterol; and about 0.5-15% PEG-modified lipid. In some embodiments, the cationic lipid nanoparticle comprises a molar ratio of about 50% cationic lipid, about 1.5% PEG-modified lipid, about 38.5% cholesterol, and about 10% non-cationic lipid. In some embodiments, the cationic lipid nanoparticle comprises a molar ratio of about 55% cationic lipid, about 2.5% PEG-modified lipid, about 32.5% cholesterol, and about 10% non-cationic lipid. In some embodiments, the cationic lipid is an ionizable cationic lipid, the non-cationic lipid is a neutral lipid, and the sterol is a cholesterol. In some embodiments, the cationic lipid nanoparticle has a molar ratio of 50:38.5:10:1.5 of cationic lipid: cholesterol: PEG2000-DMG:DSPC or DMG:DOPE. In some embodiments, lipid nanoparticles as described herein can comprise cholesterol, 1,2-dioleoyl-sn-glycero-3-phosphoethanolamine (DOPE), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), and DMG-PEG-2000 at molar ratios of 47.5:16:35:1.5.

### Methods of Use

Gene editing systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

Described herein, in some embodiments, are methods for gene editing using the engineered endonucleases (e.g., chimeric nucleases or fusion endonucleases) described herein. In some embodiments, the engineered nuclease is a class 2, type II endonuclease. In some embodiments, the type II endonuclease has a sequence having at least 55% (e.g., at least 60%, 65%, 70%, 75%, 80%, 85%, 90%, 95%, or 99%) identity to SEQ ID NO: 10.

Described herein, in certain embodiments, are methods for modifying a target nucleic acid comprising providing an engineered nuclease system disclosed herein to a cell. In some embodiments, the engineered nuclease system comprises an endonuclease and an engineered guide polynucleotide. In some embodiments, the target nucleic acid is double stranded. In some embodiments, the target nucleic acid is double stranded DNA. In some embodiments, the target nucleic acid is single stranded.

In some embodiments, the methods are used to introduce a modification in the genome of a cell. In some embodiments, the modification is an insertion, deletion, or mutation. In some embodiments, the methods are used to introduce site-directed insertions, deletions, and/or mutations in the genome of a cell (for example an insertion and a mutation). In some embodiments, the methods are used in combination with a nucleic acid template to facilitate site-directed insertions into the genome of a cell.

In some embodiments, the cell is a human cell. In some embodiments, the cell genome or a vector comprised in the cell is modified. In some embodiments, the cell genome is modified ex vivo. In some embodiments, the cell genome is modified in vivo. In some embodiments, methods described herein are for modifying an albumin gene. In some embodiments, methods described herein are for targeting an albumin gene in a cell. In some embodiments, the engineered guide polynucleotide (e.g., guide ribonucleic acid) is encoded by any one of SEQ ID NOs: 67-86 or a sequence having at least 90%, 95%, 97%, 98%, or 99% sequence identity to any one of SEQ ID NOs: 67-86. In some embodiments, the engineered guide polynucleotide is configured to hybridize to a sequence or target a sequence complementary to a sequence comprising any one of SEQ ID NOs: 67-86 or a sequence having at least 90%, 95%, 97%, 98%, or 99% sequence identity to any one of SEQ ID NOs: 67-86. In some embodiments, the engineered guide polynucleotide is configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least 90% sequence identity to any one of SEQ ID NOs: 67-86.

In some embodiments, methods described herein are for modifying a TRAC gene. In some embodiments, methods described herein are for targeting a TRAC gene in a cell. In some embodiments, the engineered guide polynucleotide (e.g., guide ribonucleic acid) is encoded by any one of SEQ ID NOs: 119-138, 922-924, 972-991, 1088-1183, 1280-1320, 2390-2485, and 2582-2617 or a sequence having at least 90%, 95%, 97%, 98%, or 99% sequence identity to any one of SEQ ID NOs: 119-138, 922-924, 972-991, 1088-1183, 1280-1320, 2390-2485, and 2582-2617. In some embodiments, the engineered guide polynucleotide is configured to hybridize to a sequence or target a sequence complementary to a sequence comprising any one of SEQ ID NOs: 119-138, 922-924, 972-991, 1088-1183, 1280-1320, 2390-2485, and 2582-2617 or a sequence having at least 90%, 95%, 97%, 98%, or 99% sequence identity to any one of SEQ ID NOs: 119-138, 922-924, 972-991, 1088-1183, 1280-1320, 2390-2485, and 2582-2617. In some embodiments, the engineered guide polynucleotide is configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least 90% sequence identity to any one of SEQ ID NOs: 119-138, 922-924, 972-991, 1088-1183, 1280-1320, 2390-2485, and 2582-2617. In some embodiments, the gRNA hybridizes or targets a sequence according to any one of SEQ ID NOs: 139-158, 925-927, 992-1011, 1184-1279, 1321-1361, 2486-2581, and 2618-2653 or a sequence having at least 90%, 95%, 97%, 98%, or 99% sequence identity to any one of SEQ ID NOs: 139-158, 925-927, 992-1011, 1184-1279, 1321-1361, 2486-2581, and 2618-2653.

In some embodiments, methods described herein are for modifying a B2M gene. In some embodiments, methods described herein are for targeting a B2M gene in a cell. In some embodiments, the engineered guide polynucleotide (e.g., guide ribonucleic acid) is encoded by any one of SEQ ID NOs: 159-184 or a sequence having at least 90%, 95%, 97%, 98%, or 99% sequence identity to any one of SEQ ID NOs: 159-184. In some embodiments, the engineered guide polynucleotide is configured to hybridize to a sequence or target a sequence complementary to a sequence comprising any one of SEQ ID NOs: 159-184 or a sequence having at least 90%, 95%, 97%, 98%, or 99% sequence identity to any one of SEQ ID NOs: 159-184. In some embodiments, the engineered guide polynucleotide is configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least 90% sequence identity to any one of SEQ ID NOs: 159-184. In some embodiments, the gRNA hybridizes or targets a sequence according to any one of SEQ ID NOs: 185-210 or a sequence having at least 90%, 95%, 97%, 98%, or 99% sequence identity to any one of SEQ ID NOs: 185-210.

In some embodiments, methods described herein are for modifying a TRBC1 gene. In some embodiments, methods described herein are for targeting a TRBC1 gene in a cell. In some embodiments, the engineered guide polynucleotide (e.g., guide ribonucleic acid) is encoded by any one of SEQ ID NOs: 211-251 or a sequence having at least 90%, 95%, 97%, 98%, or 99% sequence identity to any one of SEQ ID NOs: 211-251. In some embodiments, the engineered guide polynucleotide is configured to hybridize to a sequence or target a sequence complementary to a sequence comprising any one of SEQ ID NOs: 211-251 or a sequence having at least 90%, 95%, 97%, 98%, or 99% sequence identity to any one of SEQ ID NOs: 211-251. In some embodiments, the engineered guide polynucleotide is configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least 90% sequence identity to any one of SEQ ID NOs: 211-251. In some embodiments, the gRNA hybridizes or targets a sequence according to any one of SEQ ID NOs: 252-292 or a sequence having at least 90%, 95%, 97%, 98%, or 99% sequence identity to any one of SEQ ID NOs: 252-292.

In some embodiments, methods described herein are for modifying a TRBC2 gene. In some embodiments, methods described herein are for targeting a TRBC2 gene in a cell. In some embodiments, the engineered guide polynucleotide (e.g., guide ribonucleic acid) is encoded by any one of SEQ ID NOs: 293-337 or a sequence having at least 90%, 95%, 97%, 98%, or 99% sequence identity to any one of SEQ ID NOs: 293-337. In some embodiments, the engineered guide polynucleotide is configured to hybridize to a sequence or target a sequence complementary to a sequence comprising any one of SEQ ID NOs: 293-337 or a sequence having at least 90%, 95%, 97%, 98%, or 99% sequence identity to any one of SEQ ID NOs: 293-337. In some embodiments, the engineered guide polynucleotide is configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least 90% sequence identity to any one of SEQ ID NOs: 293-337. In some embodiments, the gRNA hybridizes or targets a sequence according to any one of SEQ ID NOs: 338-382 or a sequence having at least 90%, 95%, 97%, 98%, or 99% sequence identity to any one of SEQ ID NOs: 338-382.

In some embodiments, methods described herein are for modifying an ANGPTL3 gene. In some embodiments, methods described herein are for targeting an ANGPTL3 gene in a cell. In some embodiments, the engineered guide polynucleotide (e.g., guide ribonucleic acid) is encoded by any one of SEQ ID NOs: 383-477, 1392-1489, 2120-2215, and 2312-2350 or a sequence having at least 90%, 95%, 97%, 98%, or 99% sequence identity to any one of SEQ ID NOs: 383-477, 1392-1489, 2120-2215, and 2312-2350. In some embodiments, the engineered guide polynucleotide is configured to hybridize to a sequence or target a sequence complementary to a sequence comprising any one of SEQ ID NOs: 383-477, 1392-1489, 2120-2215, and 2312-2350 or a sequence having at least 90%, 95%, 97%, 98%, or 99% sequence identity to any one of SEQ ID NOs: 383-477, 1392-1489, 2120-2215, and 2312-2350. In some embodiments, the engineered guide polynucleotide is configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least 90% sequence identity to any one of SEQ ID NOs: 383-477, 1392-1489, 2120-2215, and 2312-2350. In some embodiments, the gRNA hybridizes or targets a sequence according to any one of SEQ ID NOs: 478-572, 1490-1587, 2216-2311, and 2351-2389 or a sequence having at least 90%, 95%, 97%, 98%, or 99% sequence identity to any one of SEQ ID NOs: 478-572, 1490-1587, 2216-2311, and 2351-2389.

In some embodiments, methods described herein are for modifying a PCSK9 gene. In some embodiments, methods described herein are for targeting a PCSK9 gene in a cell. In some embodiments, the engineered guide polynucleotide (e.g., guide ribonucleic acid) is encoded by any one of SEQ ID NOs: 573-587 and 1362-1376 or a sequence having at least 90%, 95%, 97%, 98%, or 99% sequence identity to any one of SEQ ID NOs: 573-587 and 1362-1376. In some embodiments, the engineered guide polynucleotide is configured to hybridize to a sequence or target a sequence complementary to a sequence comprising any one of SEQ ID NOs: 573-587 and 1362-1376 or a sequence having at least 90%, 95%, 97%, 98%, or 99% sequence identity to any one of SEQ ID NOs: 573-587 and 1362-1376. In some embodiments, the engineered guide polynucleotide is configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least 90% sequence identity to any one of SEQ ID NOs: 573-587 and 1362-1376. In some embodiments, the gRNA hybridizes or targets a sequence according to any one of SEQ ID NOs: 588-602 and 1377-1391 or a sequence having at least 90%, 95%, 97%, 98%, or 99% sequence identity to any one of SEQ ID NOs: 588-602 and 1377-1391.

In some embodiments, methods described herein are for modifying a VCP gene. In some embodiments, methods described herein are for targeting a VCP gene in a cell. In some embodiments, the engineered guide polynucleotide (e.g., guide ribonucleic acid) is encoded by any one of SEQ ID NOs: 723-738 and 755-762 or a sequence having at least 90%, 95%, 97%, 98%, or 99% sequence identity to any one of SEQ ID NOs: 723-738 and 755-762. In some embodiments, the engineered guide polynucleotide is configured to hybridize to a sequence or target a sequence complementary to a sequence comprising any one of SEQ ID NOs: 723-738 and 755-762 or a sequence having at least 90%, 95%, 97%, 98%, or 99% sequence identity to any one of SEQ ID NOs: 723-738 and 755-762. In some embodiments, the engineered guide polynucleotide is configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least 90% sequence identity to any one of SEQ ID NOs: 723-738 and 755-762. In some embodiments, the gRNA hybridizes or targets a sequence according to any one of SEQ ID NOs: 739-754 and 763-770 or a sequence having at least 90%, 95%, 97%, 98%, or 99% sequence identity to any one of SEQ ID NOs: 739-754 and 763-770.

In some embodiments, methods described herein are for modifying an AAVS1 locus. In some embodiments, methods described herein are for targeting an AAVS1 locus in a cell. In some embodiments, the engineered guide polynucleotide (e.g., guide ribonucleic acid) is encoded by any one of SEQ ID NOs: 928-949 and 1012-1049 or a sequence having at least 90%, 95%, 97%, 98%, or 99% sequence identity to any one of SEQ ID NOs: 928-949 and 1012-1049. In some embodiments, the engineered guide polynucleotide is configured to hybridize to a sequence or target a sequence complementary to a sequence comprising any one of SEQ ID NOs: 928-949 and 1012-1049 or a sequence having at least 90%, 95%, 97%, 98%, or 99% sequence identity to any one of SEQ ID NOs: 928-949 and 1012-1049. In some embodiments, the engineered guide polynucleotide is configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least 90% sequence identity to any one of SEQ ID NOs: 928-949 and 1012-1049. In some embodiments, the gRNA hybridizes or targets a sequence according to any one of SEQ ID NOs: 950-971 and 1050-1087 or a sequence having at least 90%, 95%, 97%, 98%, or 99% sequence identity to any one of SEQ ID NOs: 950-971 and 1050-1087.

In some embodiments, methods described herein are for modifying a GPR146 gene. In some embodiments, methods described herein are for targeting a GPR146 gene in a cell. In some embodiments, the engineered guide polynucleotide (e.g., guide ribonucleic acid) is encoded by any one of SEQ ID NOs: 1588-1656 or a sequence having at least 90%, 95%, 97%, 98%, or 99% sequence identity to any one of SEQ ID NOs: 1588-1656. In some embodiments, the engineered guide polynucleotide is configured to hybridize to a sequence or target a sequence complementary to a sequence comprising any one of SEQ ID NOs: 1588-1656 or a sequence having at least 90%, 95%, 97%, 98%, or 99% sequence identity to any one of SEQ ID NOs: 1588-1656. In some embodiments, the engineered guide polynucleotide is configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least 90% sequence identity to any one of SEQ ID NOs: 1588-1656. In some embodiments, the gRNA hybridizes or targets a sequence according to any one of SEQ ID NOs: 1657-1725 or a sequence having at least 90%, 95%, 97%, 98%, or 99% sequence identity to any one of SEQ ID NOs: 1657-1725.

In some embodiments, methods described herein are for modifying an APOA1 gene. In some embodiments, methods described herein are for targeting an APOA1 gene in a cell. In some embodiments, the engineered guide polynucleotide (e.g., guide ribonucleic acid) is encoded by any one of SEQ ID NOs: 1726-1744, 1764-1774, 1866-1961, and 2058-2088 or a sequence having at least 90%, 95%, 97%, 98%, or 99% sequence identity to any one of SEQ ID NOs: 1726-1744, 1764-1774, 1866-1961, and 2058-2088. In some embodiments, the engineered guide polynucleotide is configured to hybridize to a sequence or target a sequence complementary to a sequence comprising any one of SEQ ID NOs: 1726-1744, 1764-1774, 1866-1961, and 2058-2088 or a sequence having at least 90%, 95%, 97%, 98%, or 99% sequence identity to any one of SEQ ID NOs: 1726-1744, 1764-1774, 1866-1961, and 2058-2088. In some embodiments, the engineered guide polynucleotide is configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least 90% sequence identity to any one of SEQ ID NOs: 1726-1744, 1764-1774, 1866-1961, and 2058-2088. In some embodiments, the gRNA hybridizes or targets a sequence according to any one of SEQ ID NOs: 1745-1763, 1775-1785, 1962-2057, and 2089-2119 or a sequence having at least 90%, 95%, 97%, 98%, or 99% sequence identity to any one of SEQ ID NOs: 1745-1763, 1775-1785, 1962-2057, and 2089-2119.

In some embodiments, methods described herein are for modifying a HAO1 gene. In some embodiments, methods described herein are for targeting a HAO1 gene in a cell. In some embodiments, the engineered guide polynucleotide (e.g., guide ribonucleic acid) is encoded by any one of SEQ ID NOs: 611-633, 1789-1826, and 1827-1865 or a sequence having at least 90%, 95%, 97%, 98%, or 99% sequence identity to any one of SEQ ID NOs: 611-633, 1789-1826, and 1827-1865. In some embodiments, the engineered guide polynucleotide is configured to hybridize to a sequence or target a sequence complementary to a sequence comprising any one of SEQ ID NOs: 611-633, 1789-1826, and 1827-1865 or a sequence having at least 90%, 95%, 97%, 98%, or 99% sequence identity to any one of SEQ ID NOs: 611-633, 1789-1826, and 1827-1865. In some embodiments, the engineered guide polynucleotide is configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least 90% sequence identity to any one of SEQ ID NOs: 611-633, 1789-1826, and 1827-1865.

### Kits

In some embodiments, this disclosure provides kits comprising one or more nucleic acid constructs encoding the various components of the chimeric nuclease or gene editing system described herein, e.g., comprising a nucleotide sequence encoding the components of the chimeric nuclease or gene editing system capable of modifying a target DNA sequence. In some embodiments, the nucleotide sequence comprises a heterologous promoter that drives expression of the gene editing system components.

In some embodiments, any of the chimeric nuclease or gene editing systems disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1—Plasmids

Chimera sequences were codon optimized for E. coli expression, and synthesized and cloned into pET21 vector. To construct pET21-MG3-6+MG15-1(WP) and pET21-MG3-6+MG15-1(P), gene fragments were amplified from pMGX3-6 and pMGX15-1 using primers P441-P446. The resulting PCR products were purified and assembled into pAL3 (digested by ClaI and XhoI). DNA sequences of cloned chimeric genes were confirmed by Sanger sequencing.

### Example 2—Bioinformatic Analysis

CRISPR Type II endonucleases utilized herein were predicted to have nuclease activity based on the presence of putative HNH and RuvC catalytic residues. In addition, structural predictions suggested residues involved in guide, target, and recognition of and interaction with a PAM. Based on the location of important residues, the predicted domain architecture of Type II CRISPR endonucleases comprised three RuvC domains, an HNH endonuclease domain, a recognition domain and PAM interacting domain, among others. For genomic sequences encoding a full-length Type II endonuclease next to a CRISPR array, tracrRNA sequences were predicted, which were engineered to be used by the nuclease as single guide RNAs.

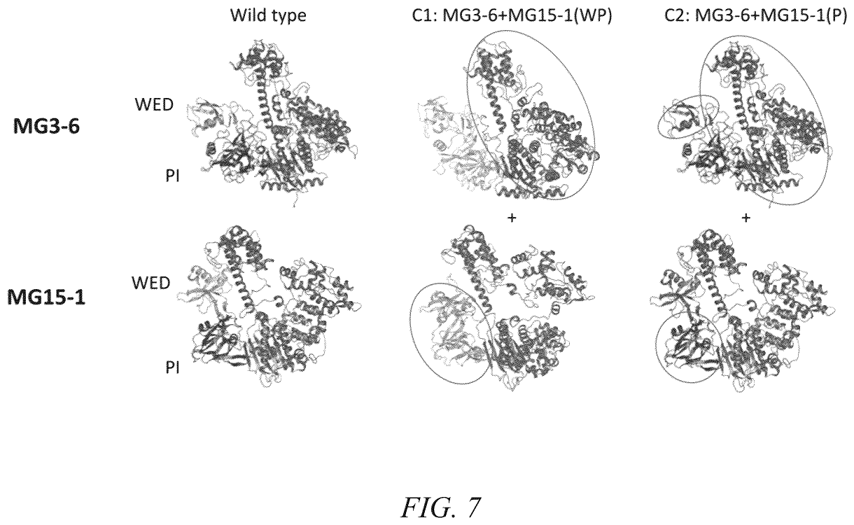

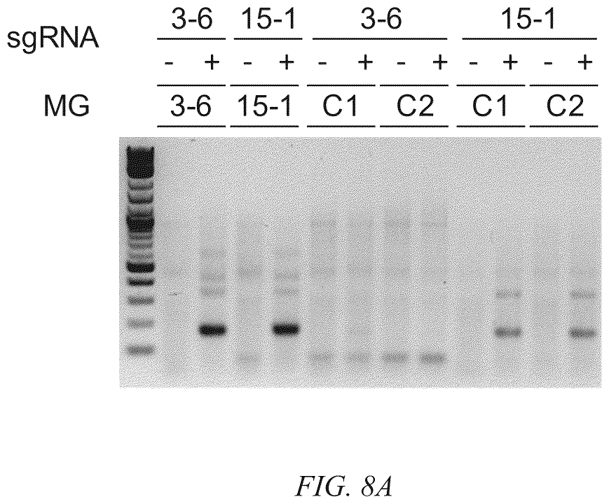

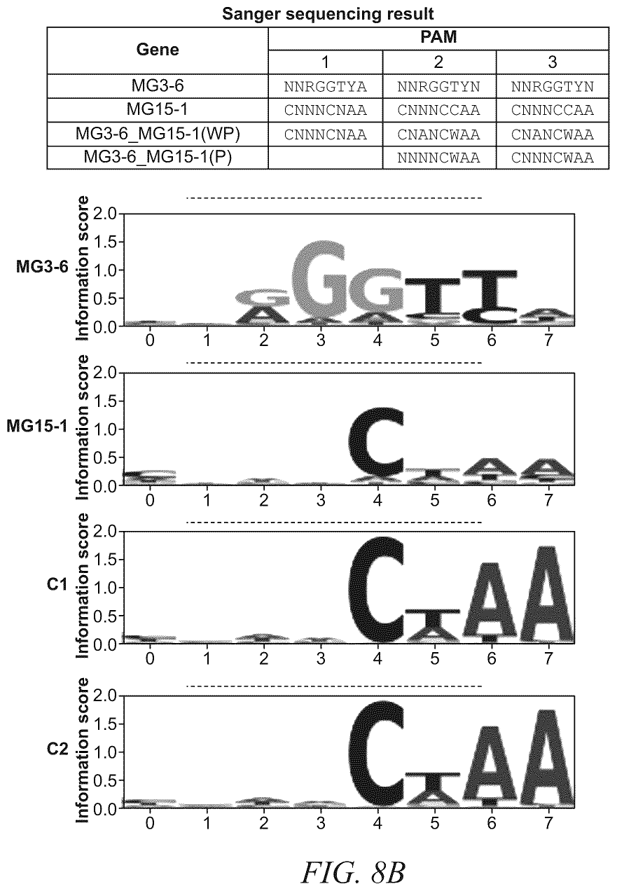

A multiple sequence alignment of selected RNA guided CRISPR Type II endonuclease sequences were performed (see FIG. 3A). Protein structures of MG3-6 and MG15-1 were predicted and displayed. Details of chimeric compositions are shown in Table 1B. Guided by predicted structural model information along with guide RNA optimization (see FIG. 7), protein variants recognizing non-canonical PAMs by concatenating domains from closely, as well as distantly related Type II CRISPR endonucleases were engineered.

TABLE 1B
Chimeric Compositions

| | | | Example |
|---|---|---|---|
| | | | Sequence |
| | | | (SEQ |
| Chimera | N-terminus | C-terminus | ID NO:) |
| MG3-6 + MG1-4 | MG3-6 (1-742) | MG1-4 (750-1025) | 1 |
| MG3-6 + MG1-5 | MG3-6 (1-742) | MG1-5 (789-1077) | 2 |
| MG3-6 + MG1-6 | MG3-6 (1-742) | MG1-6 (773-1059) | 3 |
| MG3-6 + MG1-7 | MG3-6 (1-742) | MG1-7 (775-1061) | 4 |
| MG3-6 + MG2-4 | MG3-6 (1-742) | MG2-4 (876-1201) | 5 |
| MG3-6 + MG2-7 | MG3-6 (1-742) | MG2-7 (817-1080) | 6 |
| MG3-6 + MG3-1 | MG3-6 (1-742) | MG3-1 (684-1050) | 7 |
| MG3-6 + MG3-2 | MG3-6 (1-742) | MG3-2 (755-1134) | 8 |
| MG3-6 + MG3-3 | MG3-6 (1-742) | MG3-3 (750-1132) | 9 |
| MG3-6 + MG3-4 | MG3-6 (1-742) | MG3-4 (743-1134) | 10 |
| MG3-6 + MG3-7 | MG3-6 (1-742) | MG3-7 (751-1131) | 11 |
| MG3-6 + MG3-8 | MG3-6 (1-742) | MG3-8 (741-1132) | 12 |
| MG3-6 + MG4-2 | MG3-6 (1-742) | MG4-2 (747-1043) | 13 |
| MG3-6 + MG4-5 | MG3-6 (1-742) | MG4-5 (747-1055) | 14 |
| MG3-6 + MG6-3 | MG3-6 (1-742) | MG6-3 (709-1027) | 15 |
| MG3-6 + MG14-1 | MG3-6 (1-742) | MG14-1 (756-1003) | 16 |
| MG3-6 + MG15-1 | MG3-6 (1-742) | MG15-1 (729-1082) | 17 |
| MG3-6 + MG16-1 | MG3-6 (1-742) | MG16-1 (787-1154) | 18 |
| MG3-6 + MG16-2 | MG3-6 (1-742) | MG16-2 (796-1227) | 19 |
| MG3-6 + MG18-1 | MG3-6 (1-742) | MG18-1 (997-1348) | 20 |
| MG3-6 + MG21-1 | MG3-6 (1-742) | MG21-1 (740-1098) | 21 |
| MG3-6 + MG22-1 | MG3-6 (1-742) | MG22-1 (1092- | 22 |
| | | 1521) | |
| MG3-6 + MG23-1 | MG3-6 (1-742) | MG23-1 (1008- | 23 |
| | | 1377) | |
| MG3-6 + SaCas9 | MG3-6 (1-742) | SaCas9 (706-1053) | 24 |
| MG3-6 + SpCas9 | MG3-6 (1-742) | SpCas9 (988-1368) | 25 |
| MG29-1 + | MG29-1 (1-560) | MG29-5 (556-856) | 109 |
| MG29-5 (WP) | | | |
| MG3-6 + MG15-1 | MG3-6 (1-840) | MG15-1 (818-1082) | 26 |
| (WP) | | | |
| MG3-6 + MG15-1(P) | MG3-6 (1-922) | MG15-1 (931-1082) | 27 |
| MG29-1 + | MG29-1 (1-560) | MG57-1 (633-945) | 110 |
| MG57-1 (WP) | | | |

### Example 3—In Vitro PAM Enrichment Assay

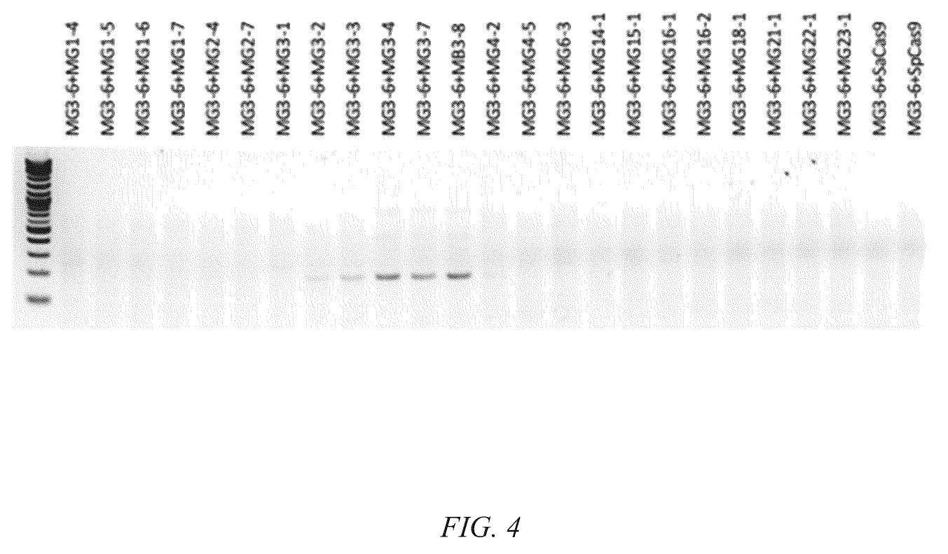

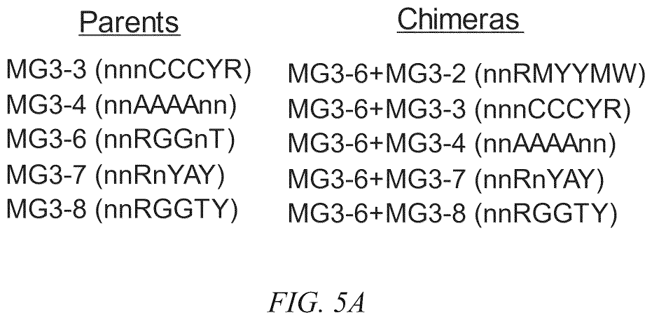

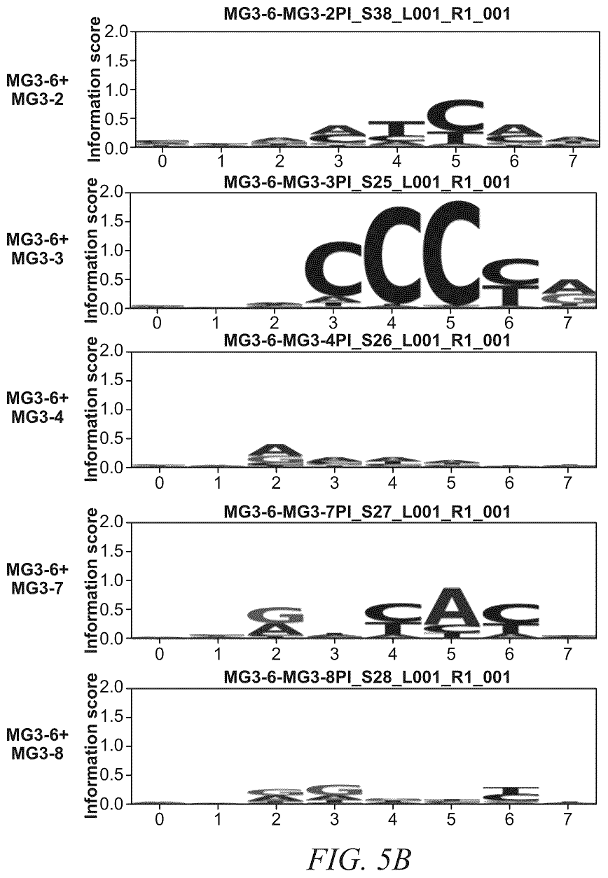

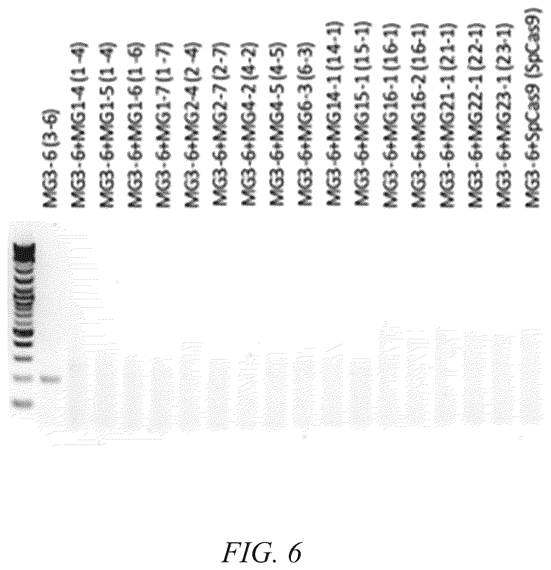

The PAM sequences of nucleases utilized herein were determined via expression in either an E. coli lysate-based expression system or reconstituted in vitro translation. The E. coli codon optimized protein sequence was transcribed and translated from a PCR fragment under control of a T7 promoter. This mixture was diluted into a reaction buffer (10 mM Tris pH 7.5, 100 mM NaCl, 10 mM MgCl₂) with protein-specific sgRNA and a PAM plasmid library (PAM library U67/U40). The library of plasmids contained a spacer sequence matching that in the single guide followed by 8N mixed bases, a subset of which were presumed to have the correct PAM. After 1-3 hours, the reaction was stopped and the DNA was recovered via a DNA clean-up kit. The DNA was subjected to a blunt-end ligation reaction which added adapter sequences to cleaved library plasmids while leaving intact circular plasmids unchanged. A PCR was performed with primers (LA065 and LA125) specific to the library and the adapter sequence and resolved on a gel to identify active protein complexes (see FIG. 4 and FIG. 6). The resulting PCR products were further amplified by PCR using high throughput sequencing primers with a cycling parameter of 8. Samples subjected to NGS analysis were quantified and pooled together. The NGS library was purified and quantified. Sequencing this library, which was a subset of the starting 8N library, revealed the sequences which contain the correct PAM (see FIGS. 5A and 5B).

### Example 4—Single Guide Design for In Vivo Targeting

The single guide (sgRNA) structures used herein comprised a structure of: 5′—22nt protospacer-repeat-tracr—3′. 20 single guides targeting mouse albumin intron 1 were designed. In some instances, guides were chemically synthesized and included a chemical modification of the guide.

### Example 5—In Vitro Transcription of mRNA

The coding sequences (CDS) encoding the chimeras (e.g., MG3-6+MG3-4 (SEQ ID NO: 10)) were codon-optimized for mouse and chemically synthesized. The CDS were cloned into mRNA production vector pMG010. The architecture of pMG010 comprised the sequence of elements: T7 promotor-5′UTR-start codon-nuclear localization signal 1-CDS-nuclear localization signal 2-stop codon-3′ UTR-107 nucleotide polyA tail (SEQ ID NO: 108). A plasmid pMG010 containing the MG3-6+MG 3-4 CDS was purified from a 200 ml bacterial culture. The vector was digested with SapI overnight in order to linearize the plasmid downstream of the polyA tail. The linearized vector was purified using phenol/chloroform DNA extraction. In vitro transcription was carried out using T7 RNA polymerase at 50° C. for 1 hour. In vitro transcribed mRNA was treated with DNase for 10 minutes at 37° C., and the mRNA was purified. mRNA was quantified by absorbance at 260 nm and its size and purity was assessed by automated electrophoresis and demonstrated to be of the expected size.

### Example 6—Transfection of Hepa1-6 Cells and Albumin Targeting

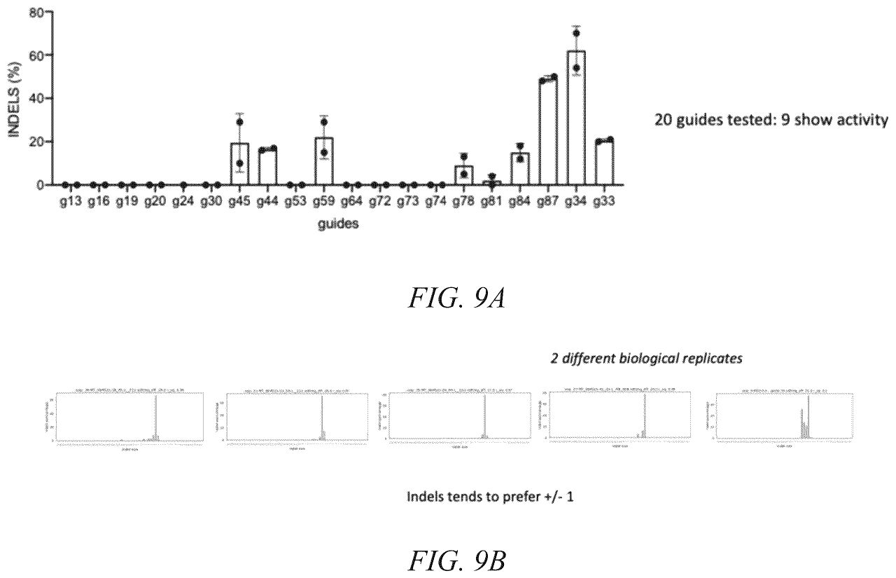

300 ng of mRNA and 350 ng of each single guide RNA (sgRNA) of SEQ ID NOs: 67-86 were co-transfected into Hepa1-6 cells as follows. One Day before transfection Hepa1-6 cells were seeded into 24 wells at a density to achieve 70% confluency 24 hours later. The following day MG3-6+MG3-4 chimera mRNA and a single guide were transfected into Hepa1-6 cells. Two days post transfection the media was aspirated, and genomic DNA was purified (see FIGS. 9A and 9B). The results indicate that the best performing sgRNAs were those designated g87 (SEQ ID NO:72) and g34 (SEQ ID NO: 70), with appreciable editing occurring also for gRNAs g45 (SEQ ID NO: 67), g44 (SEQ ID NO: 71), g59 (SEQ ID NO: 76), g78 (SEQ ID NO: 68), g84 (SEQ ID NO: 79), and g33 (SEQ ID NO: 80).

### Example 7—Sanger Sequencing of Genome Edited Samples

Primers flanking the regions of the genome targeted by the single guide RNAs (e.g., the albumin gene) were designed. PCR amplification using primers 57F (SEQ ID NO: 97) and 1072R (SEQ ID NO: 98) was performed resulting in a PCR product of 1016 bp. PCR products were purified and concentrated and 100 ng of PCR product subjected to Sanger sequencing using 8 pmoles of individual sequencing primers (132F, 282F, 446R, and 460F, SEQ ID NOs: 99-102). Sanger sequencing results were analyzed and data was plotted (see FIG. 9B).

### Example 8—MG3-6/3-4 Nuclease Guide Screen for Mouse HAO-1 Gene Using mRNA Transfection

Guide RNA for the MG3-6/3-4 nuclease targeting exons 1 to 4 of the mouse HAO-1 gene (encodes glycolate oxidase) were identified in silico by searching for the PAM sequence 3′ NNAAA(A/T)N 5′. A total of 23 guides with the fewest predicted off-target sites in the mouse genome were chemically synthesized as single guide RNAs. 300 ng mRNA and 120 ng single guide RNA were transfected into Hepa1-6 cells as follows. One day prior to transfection, Hepa1-6 cells that have been cultured for less than 10 days in DMEM, 10% FBS, 1×NEAA media, without Pen/Strep, were seeded into a TC-treated 24 well plate. Cells were counted, and the equivalent volume to 60,000 viable cells were added to each well. Additional pre-equilibrated media was added to each well to bring the total volume to 500 μL. On the day of transfection, 300 ng of the MG3-6-MG-3-4-encoding mRNA (SEQ ID NO: 108) and 120 ng of the sgRNA (scaffold sequence SEQ ID NO:34) were mixed together and transfected into the Hepa1-6 cells. Two days post transfection, the media was aspirated off of each well of Hepa1-6 cells and genomic DNA was purified by automated magnetic bead purification. The activity of the guides is summarized in Tables 2 and 3, while the primers used are summarized in Table 4.

TABLE 2
Average Activity of MG3-6/3-4 guides at mouse HAO1 delivered
by mRNA Transfection

| | | | | Editing |
|---|---|---|---|---|
| | | SEQ | | Activity |
| Guide | | ID | | (Average % |
| Name | PAM | NO: | Spacer Sequence | INDELs) |
| mH364-1 | GCAAATG | 611 | GTATGACTATTACAGGTCTGGG | 0 |
| mH364-2 | GAAAATG | 612 | AAATAGCAAAGTTTCTTACCTA | 0 |
| mH364-3 | AGAAAAT | 613 | TAAATAGCAAAGTTTCTTACCT | 0 |
| mH364-6 | CTAAAAC | 614 | ATTGGCATGCTGACTCTCTGTC | 0 |
| mH364-7 | AGAAAAG | 615 | GAGCTGGCCACTGTGCGAGGTA | 45.7 |
| mH364-9 | ACAAATA | 616 | CAGGTAAGGGGTGTCCACAGTC | 0 |
| mH364-10 | TGAAAAA | 617 | ATTCTATGTATCTATTCTAGGA | 0 |
| mH364-11 | GAAAAAC | 618 | TTCTATGTATCTATTCTAGGAT | 31 |
| mH364-15 | CCAAATC | 619 | AAATTTCCCTTAGGAGAAAATG | 0 |
| mH364-16 | GAAAATG | 620 | GTCTCCAAAATTTCCCTTAGGA | 10.7 |
| mH364-17 | AGAAAAT | 621 | TGTCTCCAAAATTTCCCTTAGG | 0 |
| mH364-18 | GGAAATT | 622 | TGATTTGGCATTTTCTCCTAAG | 0 |
| mH364-19 | CAAAATT | 623 | TCAGCAAGTCCACTGTTGTCTC | 0 |
| mH364-20 | CCAAAAT | 624 | TTCAGCAAGTCCACTGTTGTCT | 25.9 |
| mH364-22 | CAAAATG | 625 | AGTAGAGAAATGACAAACCTCT | 0 |
| mH364-23 | TCAAAAT | 626 | AAGTAGAGAAATGACAAACCTC | 20.7 |

TABLE 3
Results of testing MG3-6/3-4 guides with a more permissive PAM
design, at mouse HAO1 delivered by mRNA Transfection

| | | SEQ | | Editing | |
|---|---|---|---|---|---|
| Guide | | ID | | Activity | |
| Name | PAM | NO: | Spacer Sequence | (% INDELs) | R² |
| mH364-4 | AGAAACT | 627 | ACATCCAAGCATTTTCTAGGTA | 0 | 1 |
| mH364-5 | TAAAACA | 628 | TTGGCATGCTGACTCTCTGTCC | 0 | 1 |
| mH364-8 | ACAAAGA | 629 | CGCTGGATGCAACTGTACATCT | 0 | 0.99 |
| mH364-12 | AAAAACT | 630 | TCTATGTATCTATTCTAGGATG | 0 | 0.99 |
| mH364-13 | TGAAACC | 631 | TCTATTCTAGGATGAAAAACTT | 0 | 0.99 |
| mH364-14 | TCAAAGT | 632 | AGAAAATGCCAAATCATTGGTT | 0 | 0.99 |
| mH364-21 | GTAAAGG | 633 | ATTGACATCACTGCCTATTGTT | 0 | 1 |

TABLE 4
Primers designed for the mouse HAO1 gene, used for PCR at each
of the first four exons, and for sanger sequencing.

| | | | SEQ | |
|---|---|---|---|---|
| Target | | | ID | |
| Exon | Use | Primer Name | NO: | Primer Sequence |
| Mouse | Fwd PCR | PCR_mHE1_F_+233 | 634 | GTGACCAACCCTACCCGTTT |
| HAO1 | Rev PCR | PCR_mHE1_R_−553 | 635 | GCAAGCACCTACTGTCTCGT |
| Exon 1 | Sequencing | Seq_mHE1_F_+139 | 636 | GTCTAGGCATACAATGTTTGCTCA |
| Mouse | Fwd PCR | HAO1_E2_F5721 | 637 | CAACGAAGGTTCCCTCCAGG |
| HAO1 | Rev PCR | HAO1_E2_R6271 | 638 | GGAAGGGTGTTCGAGAAGGA |
| Exon 2 | Sequencing | 5938F Seq_HAO1_E2 | 639 | CTATGCAAGGAAAAGATTTGGCC |
| Mouse | Fwd PCR | HAO1_E3_F23198 | 640 | TGCCCTAGACAAGCTGACAC |
| HAO1 | Rev PCR | HAO1_E3_R23879 | 641 | CAGATTCTGGAAGTGGCCCA |
| Exon 3 | Sequencing | HAO1_E3_F23198 | 642 | Same as Fwd PCR Primer |
| Mouse | Fwd PCR | PCR_mHE4_F_+300 | 643 | GGCTGGCTGAAAATAGCATCC |
| HAO1 | Rev PCR | HAO1_E4_R31650 | 644 | AGGTTTGGTTCCCCTCACCT |
| Exon 4 | Sequencing | PCR_mHE4_R_−149 | 645 | TCTGCCATGAAGGCATATGGAC |

### Example 9—Guide Chemistry Optimization for the MG3-6/3-4 and MG3-6 Type II Nuclease

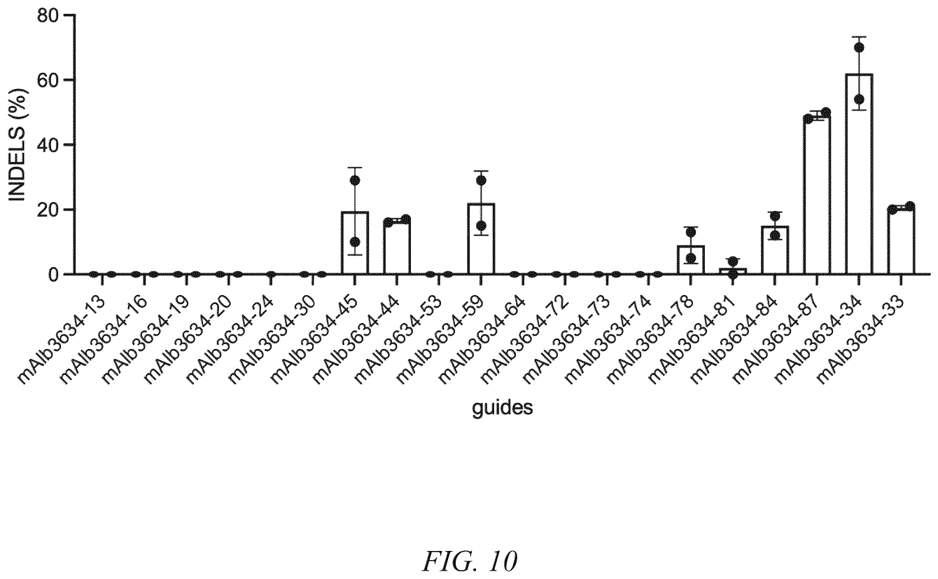

40 different chemically modified guides (named mAlb3634-34-0 to mAlb3634-34-44) were designed and the activity of 39 of these guides was tested. One guide, mH3634-34-32, failed RNA synthesis, thus it was not tested. The guide spacer sequence chosen as a model to insert various chemical modifications was mAlb3634-34 (targeting albumin intron 1) as it proved to be the most active guide in a guide screen in the mouse hepatocyte cell line Hepa1-6 cells (Table 5 and FIG. 10).

TABLE 5
Activity of chemically modified guides in Hepa1-6 cells

| | Editing Activity |
|---|---|
| Guide | (% INDELs) |
| mAlb3634-13 | BLOD |
| mAlb3634-16 | BLOD |
| mAlb3634-19 | BLOD |
| mAlb3634-20 | BLOD |
| mAlb3634-24 | BLOD |
| mAlb3634-30 | BLOD |
| mAlb3634-45 | 19.5 |
| mAlb3634-44 | 16.5 |
| mAlb3634-53 | BLOD |
| mAlb3634-59 | 22 |
| mAlb3634-64 | BLOD |
| mAlb3634-72 | BLOD |
| mAlb3634-73 | BLOD |
| mAlb3634-74 | BLOD |
| mAlb3634-78 | 9 |
| mAlb3634-81 | 2 |
| mAlb3634-84 | 15 |
| mAlb3634-87 | 49 |
| mAlb3634-34 | 62 |
| mAlb3634-33 | 20.5 |

BLOD = below limit of detection

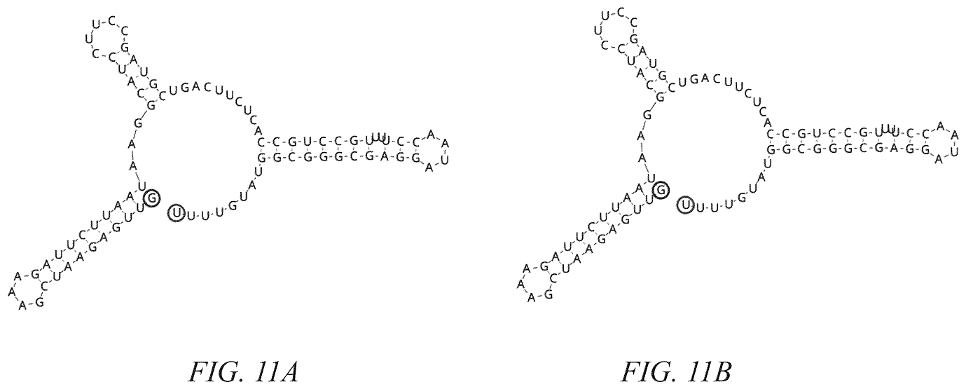

The sgRNA of MG3-6/3-4 comprises a spacer located at the 5′ end followed by the CRISPR repeat and the trans-activating CRISPR RNA (tracr). The CRISPR repeat and the tracr are identical to that of the MG3-6 nuclease (FIG. 11A, 11B). The CRISPR repeat and tracr form a structured RNA comprising 3 stem loops (FIG. 11A). Different areas of the stem loops were modified by replacing the 2′ hydroxyl of the ribose with methyl groups or replacing the phosphodiester backbone by a phosphorothioate (PS). Moreover, the spacer at the 5′ of the guide was modified with a mixture of 2′-O-methyl or 2′-fluorine bases and PS bonds. The different combinations of chemical modifications designed are called mAlb3634-34-0 to mAlb3634-34-44 and the respective sequences are shown in Table 6.

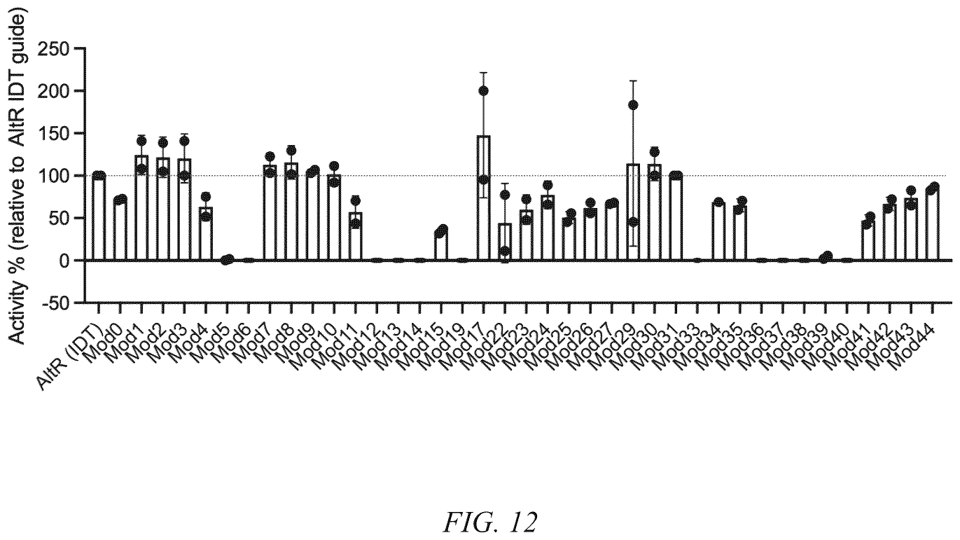

The editing activity of 39 single guides with the exact same base sequence but different chemical modifications was evaluated in Hepa1-6 cells by co-transfection of mRNA encoding MG3-6/3-4 and the guide; the editing activity is shown relative to the editing activity of guide control (AltR). The results are shown in Table 6 and FIG. 12.

TABLE 6
Sequences of chemically modified MG3-6/3-4 guides and their activity in
Hepa1-6 cells when co-transfected with MG3-6/3-4 mRNA

| | SEQ | | |
|---|---|---|---|
| | ID | | Activity |
| Guide | NO: | Sequence | % |
| mAlb3634-34-0 | 646 | rCrUrUrArGrGrUrCrArGrUrGrArArGrArGrArArGrArArGrUrUr | 71.8 |
| | | GrArGrArArUrCrGrArArArGrArUrUrCrUrUrArArUrArArGrGr | |
| | | CrArUrCrCrUrUrCrCrGrArUrGrCrUrGrArCrUrUrCrUrCrArCrC | |
| | | rGrUrCrCrGrUrUrUrUrCrCrArArUrArGrGrArGrCrGrGrGrCrGr | |
| | | GrUrArUrGrUrUrU | |
| mAlb3634-34-1 | 647 | mC*mU*mU*rArGrGrUrCrArGrUrGrArArGrArGrArArGrArAr | 124.5 |
| | | GrUrUrGrArGrArArUrCrGrArArArGrArUrUrCrUrUrArArUrAr | |
| | | ArGrGrCrArUrCrCrUrUrCrCrGrArUrGrCrUrGrArCrUrUrCrUrC | |
| | | rArCrCrGrUrCrCrGrUrUrUrUrCrCrArArUrArGrGrArGrCrGrGr | |
| | | GrCrGrGrUrArUrGrU*mU*mU*mU | |
| mAlb3634-34-2 | 648 | mC*mU*mU*rArGrGrUrCrArGrUrGrArArGrArGrArArGrArAr | 121.7 |
| | | GrUrUrGrArGrArArUrCrGrArArArGrArUrUrCrUrUrArArUrAr | |
| | | ArGrGrCrArUrCrCrUrUrCrCrGrArUrGrCrUrGrArCrUrUrCrUrC | |
| | | rArCrCrGrUrCrCrGrUrUrUrUrCrCrArArUrArGrGrArGrCrGrGr | |
| | | GrCrGrGrUrA*mU*mG*mU*mU*mU*mU | |
| mAlb3634-34-3 | 649 | mC*mU*mU*rArGrGrUrCrArGrUrGrArArGrArGrArArGrArAr | 120.5 |
| | | GrUrUrGrArGmAmAmUmCmGmAmAmAmGmAmUmUrCrUr | |
| | | UrArArUrArArGrGrCrArUrCrCrUrUrCrCrGrArUrGrCrUrGrArC | |
| | | rUrUrCrUrCrArCrCrGrUrCrCrGrUrUrUrUrCrCrArArUrArGrGr | |
| | | ArGrCrGrGrGrCrGrGrUrArUrGrU*mU*mU*mU | |
| mAlb3634-34-4 | 650 | mC*mU*mU*rArGrGrUrCrArGrUrGrArArGrArGrArArGrArAr | 63.3 |
| | | GrUrUrGrArG*mA*mA*mU*mC*mG*mA*mA*mA*mG*mA* | |
| | | mU*mUrCrUrUrArArUrArArGrGrCrArUrCrCrUrUrCrCrGrArUr | |
| | | GrCrUrGrArCrUrUrCrUrCrArCrCrGrUrCrCrGrUrUrUrUrCrCrA | |
| | | rArUrArGrGrArGrCrGrGrGrCrGrGrUrArUrGrU*mU*mU*mU | |
| mAlb3634-34-5 | 651 | mC*mU*mU*rArGrGrUrCrArGrUrGrArArGrArGrArArGrArAm | 0.8 |
| | | GmUmUmGmAmGmAmAmUmCmGmAmAmAmGmAmUmU | |
| | | mCmUmUmAmArUrArArGrGrCrArUrCrCrUrUrCrCrGrArUrGr | |
| | | CrUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-6 | 652 | mC*mU*mU*rArGrGrUrCrArGrUrGrArArGrArGrArArGrArAm | 0.0 |
| | | GmUmUmGmAmGmAmAmUmCrG*rA*rA*rA*mGmAmUmU | |
| | | mCmUmUmAmArUrArArGrGrCrArUrCrCrUrUrCrCrGrArUrGr | |
| | | CrUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-7 | 653 | mC*mU*mU*rArGrGrUrCrArGrUrGrArArGrArGrArArGrArAr | 113.0 |
| | | GrUrUrGrArGrArArUrCrGrArArArGrArUrUrCrUrUrArArUrAr | |
| | | AmGmGmCmAmUmCrCrUrUrCrCrGrArUrGrCrUrGrArCrUrUr | |
| | | CrUrCrArCrCrGrUrCrCrGrUrUrUrUrCrCrArArUrArGrGrArGrC | |
| | | rGrGrGrCrGrGrUrArUrGrU*mU*mU*mU | |
| mAlb3634-34-8 | 654 | mC*mU*mU*rArGrGrUrCrArGrUrGrArArGrArGrArArGrArAr | 115.6 |
| | | GrUrUrGrArGrArArUrCrGrArArArGrArUrUrCrUrUrArArUrAr | |
| | | AmGmGmCmAmUmCrC*rU*rU*rC*rC*rGrArUrGrCrUrGrArC | |
| | | rUrUrCrUrCrArCrCrGrUrCrCrGrUrUrUrUrCrCrArArUrArGrGr | |
| | | ArGrCrGrGrGrCrGrGrUrArUrGrU*mU*mU*mU | |
| mAlb3634-34-9 | 655 | mC*mU*mU*rArGrGrUrCrArGrUrGrArArGrArGrArArGrArAr | 105.0 |
| | | GrUrUrGrArGrArArUrCmGmAmAmArGrArUrUrCrUrUrArArU | |
| | | rArArGrGrCrArUrCmCmUmUmCmCrGrArUrGrCrUrGrArCrUr | |
| | | UrCrUrCrArCrCrGrUrCrCrGrUrUrUrUrCrCmAmAmUmArGrGr | |
| | | ArGrCrGrGrGrCrGrGrUrArUrGrU*mU*mU*mU | |
| mAlb3634-34-10 | 656 | mC*mU*mU*rArGrGrUrCrArGrUrGrArArGrArGrArArGrArAr | 101.6 |
| | | GrUrUrGrArGrArArUrCrG*rA*rA*rA*rGrArUrUrCrUrUrArAr | |
| | | UrArArGrGrCrArUrCrC*rU*rU*rC*rC*rGrArUrGrCrUrGrArCr | |
| | | UrUrCrUrCrArCrCrGrUrCrCrGrUrUrUrUrCrCrA*rA*rU*rA*rG | |
| | | rGrArGrCrGrGrGrCrGrGrUrArUrGrU*mU*mU*mU | |
| mAlb3634-34-11 | 657 | mC*mU*mU*rArGrGrUrCrArGrUrGrArArGrArGrArArGrArAr | 57.0 |
| | | GrUrUrGrArGrArArUrCrGrArArArGrArUrUrCrUrUrArArUrAr | |
| | | ArGrGrCrArUrCrCrUrUrCrCrGrArUrGrCrUrGrArCrUrUrCrUrC* | |
| | | mA*mC*mC*mG*mU*mC*mC*mG*mU*mU*mU*mU*mC* | |
| | | mC*mA*mA*mU*mArGrGrArGrCrGrGrGrCrGrGrUrA*mU*m | |
| | | G*mU*mU*mU*mU | |
| mAlb3634-34-12 | 658 | mC*mU*mU*rArGrGrUrCrArGrUrGrArArGrArGrArArGrArAm | 0.0 |
| | | GmUmUmGmAmGmAmAmUmCrG*rA*rA*rA*rGrArUrUrCrU | |
| | | rUrArArUrArArGrGrCrArUrCrCrUrUrCrCrGrArUrGrCrUrGrAr | |
| | | CrUrUrCrUrC*mA*mC*mC*mG*mU*mC*mC*mG*mU*mU* | |
| | | mU*mU*mC*mC*mA*mA*mU*mA*mG*mG*mA*mG*mC*m | |
| | | G*mG*mG*mC*mG*mG*mU*mA*mU*mG*mU*mU*mU*mU | |
| mAlb3634-34-13 | 659 | mC*mU*mU*rArGrGrUrCrArGrUrGrArArGrArGrArArGrArAm | 0.0 |
| | | GmUmUmGmAmGmAmAmUmCmGmAmAmAmGmAmUmU | |
| | | mCmUmUmAmArUrArArGrGrCrArUrCrCrUrUrCrCrGrArUrGr | |
| | | CrUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-14 | 670 | mC*mU*mU*rArGrGrUrCrArGrUrGrArArGrArGrArArGrArAr | 0.0 |
| | | GrUrUrGrArGmAmAmUmCmGmAmAmAmGmAmUmUrCrUr | |
| | | UrArArUrArArGrGrCrArUrCrCrUrUrCrCrGrArUrGrCrUrGrArC | |
| | | rUrUrCrUrC*mA*mC*mC*mG*mU*mC*mC*mG*mU*mU*m | |
| | | U*mU*mC*mC*mA*mA*mU*mA*mG*mG*mA*mG*mC*mG* | |
| | | mG*mG*mC*mG*mG*mU*mA*mU*mG*mU*mU*mU*mU | |
| mAlb3634-34-15 | 671 | mC*mU*mU*rArGrGrUrCrArGrUrGrArArGrArGrArArGrArAr | 34.5 |
| | | GrUrUrGrArGmAmAmUmCmGmAmAmAmGmAmUmUrCrUr | |
| | | UrArArUrArAmGmGmCmAmUmCrCrUrUrCrCrGrArUrGrCrUr | |
| | | GrArCrUrUrCmUmCmAmCmCmGmUmCmCmGmUmUmUmU | |
| | | mCmCmAmAmUmAmGmGmAmGmCmGmGmGmCmGmGmU | |
| | | mAmUmGmU*mU*mU*mU | |
| mAlb3634-34-19 | 672 | mC*mU*mU*rArGrGrUrCrArGrUrGrArArGrArGrArArGrArAr | 0.0 |
| | | GrUrUrGrArGrArArUrC*mG*mA*mA*mArGrArUrUrCrUrUrA | |
| | | rA*mU*mA*mArGrGrCrArUrC*mC*mU*mU*mC*mCrGrArUr | |
| | | GrCrU*mG*mA*mC*mU*mU*mC*mU*mCrArCrCrGrUrCrCr | |
| | | GrUrUrUrUrCrC*mA*mA*mU*mArGrGrArGrCrGrGrGrCrGrGr | |
| | | UrA*mU*mG*mU*mU*mU*mU | |
| mAlb3634-34-17 | 673 | mC*mU*mU*i2FAi2FGi2FGi2FUi2FCi2FAi2FGi2FUi2FGi2FAi | 147.7 |
| | | 2FAi2FGi2FAi2FGrArArGrArArGrUrUrGrArGrArArUrCrGrAr | |
| | | ArArGrArUrUrCrUrUrArArUrArArGrGrCrArUrCrCrUrUrCrCrG | |
| | | rArUrGrCrUrGrArCrUrUrCrUrCrArCrCrGrUrCrCrGrUrUrUrUr | |
| | | CrCrArArUrArGrGrArGrCrGrGrGrCrGrGrUrArUrGrU*mU*mU* | |
| | | mU | |
| mAlb3634-34-22 | 674 | mC*mU*mU*rArGrGrUrCrArGrUrGrArArGrArGrArArGrArAr | 44.2 |
| | | GrUrUrGrArGrArArUrCrG*rA*rA*rA*rGrArUrUrCrUrUrArAr | |
| | | UrArArGrGrCrArUrCrC*rU*U*rC*rC*rGrArUrGrCrUrGrArCr | |
| | | UrUrCrUrCmAmCmCmGmUmCmCmGmUmUmUmUmCmCrA* | |
| | | rA*rU*rA*mGmGmAmGmCmGmGmGmCmGmGmU*mA*m | |
| | | U*mG*mU*mU*mU*mU | |
| mAlb3634-34-23 | 675 | mC*mU*mU*rArGrGrUrCrArGrUrGrArArGrArGrArArGrArAr | 60.0 |
| | | GrUrUrGrArGrArArUrCrG*rA*rA*rA*rGrArUrUrCrUrUrArAr | |
| | | UrArArGrGrCrArUrCrC*rU*rU*rC*C*rGrArUrGrCrUrG*rA*r | |
| | | C*rU*rU*rC*U*rC*mAmCmCmGmUmCmCmGmUmUmUmU | |
| | | mCmCrA*rA*rU*rA*mGmGmAmGmCmGmGmGmCmGmGm | |
| | | U*mA*mU*mG*mU*mU*mU*mU | |
| mAlb3634-34-24 | 676 | mC*mU*mU*rArGrGrUrCrArGrUrGrArArGrArGrArArGrArAr | 77.4 |
| | | GrUrUrGrArGrArArUrCrG*rA*rA*rA*rGrArUrUrCrUrUrArAr | |
| | | UrArAmGmGmCmAmUmCrC*rU*rU*rC*rC*rGrArUrGrCrUrG | |
| | | rArCrUrUrCrUrCmAmCmCmGmUmCmCmGmUmUmUmUmC | |
| | | mCrA*rA*rU*rA*mGmGmAmGmCmGmGmGmCmGmGmU* | |
| | | mA*mU*mG*mU*mU*mU*mU | |
| mAlb3634-34-25 | 677 | mC*mU*mU*rArGrGrUrCrArGrUrGrArArGrArGrArArGrArAr | 50.5 |
| | | GrUrUrGrArGrArArUrCrG*rA*rA*rA*rGrArUrUrCrUrUrArAr | |
| | | UrArAmGmGmCmAmUmCrC*rU*rU*rC*rC*rGrArUrGrCrUrG* | |
| | | rA*rC*rU*rU*rC*rU*C*mAmCmCmGmUmCmCmGmUmUm | |
| | | UmUmCmCrA*rA*rU*rA*mGmGmAmGmCmGmGmGmCmG | |
| | | mGmU*mA*mU*mG*mU*mU*mU*mU | |
| mAlb3634-34-26 | 678 | mC*mU*mU*mA*rGrGrUrCrArGrUrGrArArGrArGrArArGrAr | 61.9 |
| | | ArGrUrUrGrArGrArArUrCrGrArArArGrArUrUrCrUrUrArArUr | |
| | | ArArGrGrCrArUrCrC*rU*rU*rC*rC*rGrArUrGrCrUrGrArCrUr | |
| | | UrCrUrCrArCrCrGrUrCrCrGrUrUrUrUrCrCrArArUrArGrGrArG | |
| | | rCrGrGrGrCrGrGrUrA*mU*mG*mU*mU*mU*mU | |
| mAlb3634-34-27 | 679 | mC*mU*mU*mA*rGrGrUrCrArGrUrGrArArGrArGrArArGrAr | 67.4 |
| | | ArGrUrUrGrArGrArArUrCrG*rA*rA*rA*rGrArUrUrCrUrUrAr | |
| | | ArUrArArGrGrCrArUrCrCrUrUrCrCrGrArUrGrCrUrGrArCrUrU | |
| | | rCrUrCmAmCmCmGmUmCmCmGmUmUmUmUmCmCrA*rA* | |
| | | rU*rA*rGrGrArGrCrGrGrGrCrGrGrUrA*mU*mG*mU*mU*m | |
| | | U*mU | |
| mAlb3634-34-29 | 680 | mC*i2FU*i2FU*i2FA*rGrGrUrCrArGrUrGrArArGrArGrArArGr | 114.4 |
| | | ArAmGmUmUmGmAmGmAmAmUmCrGrArArArGrArUrUrCr | |
| | | UrUrArArUrArArGrGrCrArUrCrCrUrUrCrCrGrArUrGrCrUrGrA | |
| | | rCrUrUrCrUrCrArCrCrGrUrCrCrGrUrUrUrUrCrCrArArUrArGr | |
| | | GrArGrCrGrGrGrCrGrGrUrA*mU*mG*mU*mU*mU*mU | |
| mAlb3634-34-30 | 681 | mC*i2FU*i2FU*i2FA*rGrGrUrCrArGrUrGrArArGrArGrArArGr | 113.9 |
| | | ArArGrUrUrGrArGrArArUrCrGrArArArGrArUrUrCrUrUrArAr | |
| | | UrArAmGmGmCmAmUmCrCrUrUrCrCrGrArUrGrCrUrGrArCr | |
| | | UrUrCrUrCrArCrCrGrUrCrCrGrUrUrUrUrCrCrArArUrArGrGrA | |
| | | rGrCrGrGrGrCrGrGrUrA*mU*mG*mU*mU*mU*mU | |
| mAlb3634-34-31 | 682 | mC*i2FU*i2FU*i2FA*rGrGrUrCrArGrUrGrArArGrArGrArArGr | 100.0 |
| | | ArArGrUrUrGrArGrArArUrCrGrArArArGrArUrUrCrUrUrArAr | |
| | | UrArArGrGrCrArUrCrCrUrUrCrCrGrArUrGrCrUrGrArCrUrUrC | |
| | | rUrCmAmCmCmGmUmCmCmGmUmUmUmUmCmCrArArUr | |
| | | ArGrGrArGrCrGrGrGrCrGrGrUrA*mU*mG*mU*mU*mU*mU | |
| mAlb3634-34-32 | 683 | mC*mU*mU*i2FA*i2FGi2FGi2FUi2FCi2FAi2FGi2FUi2FGi2F | NT |
| | | Ai2FAi2FGi2FAi2FGrArArGrArAmGmUmUmGmAmGmAmA | |
| | | mUmCrG*rA*rA*rA*mGmAmUmUrCrUrUrArArUrArAmGmG | |
| | | mCmAmUmCrC*rU*rU*rC*rC*rGrArUrGrCrUrGrArCrUrUrCr | |
| | | UrCmAmCmCmGmUmCmCmGmUmUmUmUmCmCrA*rA*rU* | |
| | | rA*mGmGmAmGmCmGmGmGmCmGmGmUmA*mU*mG*m | |
| | | U*mU*mU*mU | |
| mAlb3634-34-33 | 684 | mC*mU*mU*i2FA*i2FGi2FGi2FUi2FCi2FAi2FGi2FUi2FGi2F | 0.0 |
| | | Ai2FAi2FGi2FAi2FGrArArGrArAmGmUmUmGmAmGmAmA | |
| | | mUmCrG*rA*rA*rA*mGmAmUmUrCrUrUrArArUrArAmGmG | |
| | | mCmAmUmCrC*rU*U*rC*rC*rGrArUrGrCrUrGrArCrUrUrCr | |
| | | UrCrArCrCrGrUrCrCrGrUrUrUrUrCrCrA*A*U*rA*rGrGrArG | |
| | | rCrGrGrGrCrGrGrUrA*mU*mG*mU*mU*mU*mU | |
| mAlb3634-34-34 | 685 | mC*mU*mU*mA*i2FGi2FGi2FUi2FCi2FAi2FGi2FUi2FGi2FAi | 68.9 |
| | | 2FAi2FGi2FAi2FGrArArGrArArGrUrUrGrArGrArArUrCrG*rA* | |
| | | A*rA*rGrArUrUrCrUrUrArArUrArArGrGrCrArUrCrC*rU*rU* | |
| | | rC*rC*rGrArUrGrCrUrGrArCrUrUrCrUrCrArCrCrGrUrCrCrGr | |
| | | UrUrUrUrCrCrA*rA*rU*rA*rGrGrArGrCrGrGrGrCrGrGrUrA* | |
| | | mU*mG*mU*mU*mU*mU | |
| mAlb3634-34-35 | 686 | mC*mU*mU*mA*rGrGrUrCrArGrUrGrArArGrArGrArArGrAr | 65.0 |
| | | ArGrUrUrGrArGrArArUrCmG*mA*mA*mA*rGrArUrUrCrUrU | |
| | | rArArUrArArGrGrCrArUrCmC*mU*mU*mC*mC*rGrArUrGrC | |
| | | rUrGrArCrUrUrCrUrCrArCrCrGrUrCrCrGrUrUrUrUrCrCmA*m | |
| | | A*mU*mA*rGrGrArGrCrGrGrGrCrGrGrUrA*mU*mG*mU*m | |
| | | U*mU*mU | |
| mAlb3634-34-36 | 687 | mC*mU*mU*mA*rGrGrUrCrArGrUrGrArArGrArGrArArGrAr | 0.0 |
| | | AmGmUmUmGmAmGmAmAmUmCrG*rA*rA*rA*rGrArUrUr | |
| | | CrUrUrArArUrArAmGmGmCmAmUmCrC*rU*rU*rC*rC*rGrA | |
| | | rUrGrCrUrGrArCrUrUrCrUrCrArCrCrGrUrCrCrGrUrUrUrUrCr | |
| | | CrA*rA*rU*rA*rGrGrArGrCrGrGrGrCrGrGrUrA*mU*mG*mU* | |
| | | mU*mU*mU | |
| mAlb3634-34-37 | 688 | mC*mU*mU*mA*rGrGrUrCrArGrUrGrArArGrArGrArArGrAr | 0.0 |
| | | AmGmUmUmGmAmGmAmAmUmCrG*rA*rA*rA*rGrArUrUr | |
| | | CrUrUrArArUrArAmGmGmCmAmUmCrC*rU*rU*rC*rC*rGrA | |
| | | rUrGrCrUrGrArCrUrUrCrUrCmAmCmCmGmUmCmCmGmUm | |
| | | UmUmUmCmCrA*rA*rU*rA*rGrGrArGrCrGrGrGrCrGrGrUrA* | |
| | | mU*mG*mU*mU*mU*mU | |
| mAlb3634-34-38 | 689 | mC*mU*mU*mA*rGrGrUrCrArGrUrGrArArGrArGrArArGrAr | 0.0 |
| | | AmGmUmUmGmAmGmAmAmUmCrG*rA*rA*rA*rGrArUrUr | |
| | | CrUrUrArArUrArAmGmGmCmAmUmCrC*rU*rU*rC*rC*rGrA | |
| | | rUrGrCrUrGrArCrUrUrCrUrCrArCrCmGmUmCmCmGmUmUm | |
| | | UmUmCmCrA*rA*rU*rA*mGmGmAmGmCmGmGmGmCmGr | |
| | | GrUrA*mU*mG*mU*mU*mU*mU | |
| mAlb3634-34-39 | 690 | mC*mU*mU*mA*rGrGrUrCrArGrUrGrArArGrArGrArArGrAr | 3.7 |
| | | ArGrUrUrGrArGmAmAmUmCrG*rA*rA*rA*rGrArUrUrCrUrU | |
| | | rArArUrArArGrGrCrArUrCrC*rU*rU*rC*rC*rGrArUrGrCrUrG* | |
| | | rA*rC*rU*rU*rC*rU*rC*rArCrCrGrUrCrCrGrUrUrUrUrCrCrA* | |
| | | rA*rU*rA*rGrGrArGrCrGrGrGrCrGrGrUrA*mU*mG*mU*mU* | |
| | | mU*mU | |
| mAlb3634-34-40 | 691 | mC*mU*mU*mA*rGrGrUrCrArGrUrGrArArGrArGrArArGrAr | 0.0 |
| | | ArGrUrUrGrArGmAmAmUmCrG*rA*rA*rA*mGmAmUmUrCr | |
| | | UrUrArArUmAmAmGmGmCmAmUmCrC*rU*rU*rC*rC*mGm | |
| | | AmUmGmCrU*rG*rA*mCmUmUrCrUrCrArCrCrGrUrCrCrGrU | |
| | | rUrUrUrCrCrA*rA*rU*rA*rGrGrArGrCrGrGrGrCrGrGrUrA*m | |
| | | U*mG*mU*mU*mU*mU | |
| mAlb3634-34-41 | 692 | mC*mU*mU*mA*rGrGrUrCrArGrUrGrArArGrArGrArArGrAr | 47.1 |
| | | ArGrUrUrGrArGmAmAmUmCrG*rA*rA*rA*rGrArUrUrCrUrU | |
| | | rArArUrArAmGmGmCmAmUmCrC*rU*rU*rC*rC*rGrArUrGr | |
| | | CrUrGrArCrUrUrCrUrCmAmCmCmGmUmCmCmGmUmUmU | |
| | | mUmCmCrA*rA*rU*rA*rGrGrArGrCrGrGrGrCrGrGrUrA*mU* | |
| | | mG*mU*mU*mU*mU | |
| mAlb3634-34-42 | 693 | mC*mU*mU*mA*i2FGi2FGi2FUi2FCi2FAi2FGi2FUi2FGi2FAi | 66.7 |
| | | 2FAi2FGi2FAi2FGi2FAi2FAi2FGi2FArArGrUrUrGrArGrArArU | |
| | | rCrG*rA*rA*rA*rGrArUrUrCrUrUrArArUrArArGrGrCrArUrCr | |
| | | C*rU*rU*rC*rC*rGrArUrGrCrUrGrArCrUrUrCrUrCrArCrCrGr | |
| | | UrCrCrGrUrUrUrUrCrCrA*rA*rU*rA*rGrGrArGrCrGrGrGrCrG | |
| | | rGrUrA*mU*mG*mU*mU*mU*mU | |
| mAlb3634-34-43 | 694 | mC*mU*mU*rArGrGrUrCrArGrUrGrArArGrArGrArArGrArAr | 73.8 |
| | | GrUrUrGrArGrArArUrCrGrArArArGrArUrUrCrUrUrArArUrAr | |
| | | ArGrGrCrArUrCrCrUrUrCrCrGrArUrGrCrUrGrArCrUrUrCrUrC | |
| | | rArCrCrGrUrCrCrGrUrUrUrUrCrCrArArUrAmGmGmAmGmC | |
| | | mGmGmGmCmGmGmUmA*mU*mG*mU*mU*mU*mU | |
| mAlb3634-34-44 | 695 | mC*mU*mU*rArGrGrUrCrArGrUrGrArArGrArGrArArGrArAr | 84.9 |
| | | GrUrUrGrArGrArArUrCrGrArArArGrArUrUrCrUrUrArArUrAr | |
| | | ArGrGrCrArUrCrCrUrUrCrCrGrArUrGrCrUrGrArCrUrUrCrUrC | |
| | | mAmCmCmGmUmCmCmGmUmUmUmUmCmCrArArUrArGr | |
| | | GrArGrCrGrGrGrCrGrGrUrA*mU*mG*mU*mU*mU*mU | |

(r = native ribose base, m = 2′-O methyl modified base, F = 2′ Fluro modified base,
* = phosphorothioate bond)

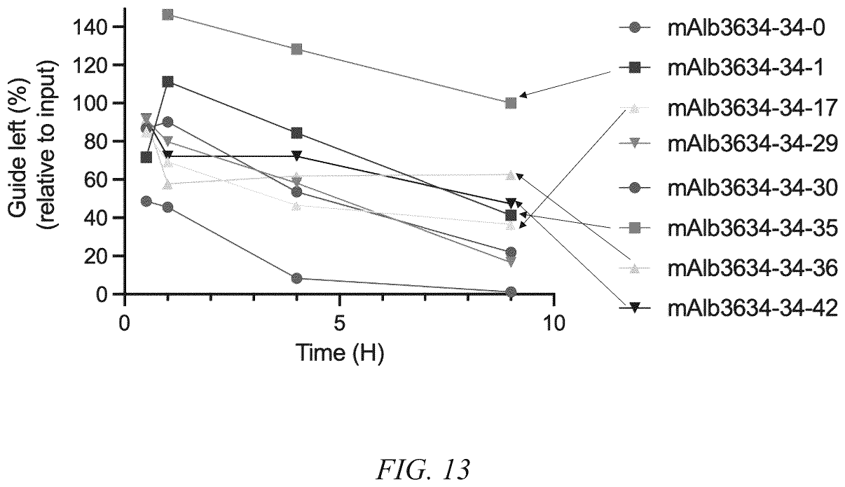

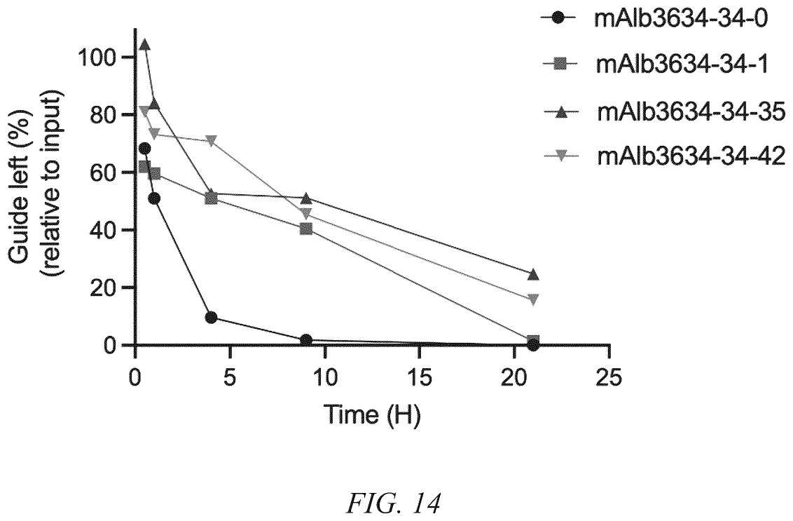

A guide with the same base sequence and an alternative chemical modification (AltR1/AltR2) was used as a control. The spacer sequence in these guides targets a 22-nucleotide region in albumin intron 1 of the mouse genome. Guide mAlb3634-34-0 (no chemical modifications) showed 72% activity relative to the AltR1/AltR2 guide. Guide mAlb3634-34-1 showed 124% activity relative to the AltR1/AltR2 guide, showing the importance of stability of guides for editing: mAlb3634-34-1 is more stable than mAlb3634-34-0 (FIG. 13 and FIG. 14). Importantly, mAlb3634-34-17 retained 147% of the activity relative to AltR1/AltR2. The incorporation of 2′-O-fluorines in the spacer greatly increased the stability of mAlb3634-34-35, and the guide retained 65% activity. mAlb3634-34-35 contains 2′-O-methyl and PS bonds in the loops of the three stem loops of the MG3-6/3-4 guide. Importantly, mAlb3634-34-42 retained 66% of activity and this guide contains as many fluorines in the spacer as mAlb3634-34-17, but it also contains PS bonds in all the loops present in the gRNA. mAlb3634-34-27 retained 67% activity and mAlb3634-34-29 retained 114% activity. Among the modifications these guides contain are PS bonds in the loop of the first stem loop and 2′-O-methyl groups in the first strand of the first stem loop for mAlb3634-34-27 and mAlb3634-34-29, respectively. When these 2 modifications were combined (2′-O-methyl in the first strand of the first stem loop and PS bonds in the loop of the first stem loop), the guides lost their activity (mAlb3634-34-33, mAlb3634-34-36, mAlb3634-34-38), showing the complexity of the gRNA/protein interaction and demonstrating that the results of simple extrapolations are difficult to predict.

In order to test the stability of these chemically modified guides compared to the guide with no chemical modification (native RNA), a stability assay using crude cell extracts was used. Crude cell extracts from mammalian cells were selected because they contain the mixture of nucleases that a guide RNA will be exposed to when delivered to mammalian cells in vitro or in vivo. Hepa1-6 cells were collected by adding 3 mL of cold PBS per 15 cm dish of confluent cells and releasing the cells from the surface of the dish using a cell scraper. The cells were pelleted at 200 g for 10 minutes and frozen at −80° C. for future use. For the stability assays, cells were resuspended in 4 volumes of cold PBS (e.g., for a 100 mg pellet, cells were resuspended in 400 μL of cold PBS). Triton X-100 was added to a concentration of 0.2% (v/v), cells were vortexed for 10 seconds, put on ice for 10 minutes, and vortexed again for 10 seconds. Triton X-100 is a mild non-ionic detergent that disrupts cell membranes but does not inactivate or denature proteins at the concentration used. Stability reactions were set up on ice and comprised 20 μl of cell crude extract with 2 pmoles of each guide (1 μL of a 2 μM stock). Six reactions were set up per guide comprising: input, 0.5 hour, 1 hour, 4 hours, 9 hours, and in some cases 21 hours (The time in hours referring to the length of time each sample was incubated). Samples were incubated at 37° C. from 0.5 hours up to 21 hours while the input control was left on ice for 5 minutes. After each incubation period, the reaction was stopped by adding 300 μL of a mixture of phenol and guanidine thiocyanate (Tri Reagent), which immediately denatures all proteins and efficiently inhibits ribonucleases and facilitates the subsequent recovery of RNA. After adding Tri Reagent, the samples were vortexed for 15 seconds and stored at −20° C. RNA was extracted from the samples and eluted in 100 μL of nuclease-free water. Detection of the modified guide was performed, and primers and probes were designed to specifically detect the sequence in the mAlb3634-34 sgRNA, which is the same for all of the guides. Data was plotted as a function of percentage of sgRNA remaining in relation to the input sample (Tables 7 and 8; FIG. 13 and FIG. 14).

TABLE 7
Stability of MG3-6/3-4 chemically modified guides
over 9 hours at 37° C.

| | Percentage (%) guide left | | | |
|---|---|---|---|---|
| Time | mAlb3634- | mAlb3634- | mAlb3634- | mAlb3634- |
| (H) | 34-0 | 34-1 | 34-17 | 34-29 |
| 0.5 | 48.6 | 71.6 | 84.9 | 91.3 |
| 1 | 45.5 | 111.3 | 69.2 | 79.8 |
| 4 | 8.3 | 84.3 | 46.6 | 58.2 |
| 9 | 1.2 | 41.3 | 36.6 | 16.5 |
| Time | mAlb3634- | mAlb3634- | mAlb3634- | mAlb3634- |
| (H) | 34-30 | 34-35 | 34-36 | 34-42 |
| 0.5 | 86.7 | ND | 91.7 | 91.7 |
| 1 | 90.1 | 146.4 | 57.8 | 72.1 |
| 4 | 53.5 | 128.3 | 61.9 | 72.1 |
| 9 | 21.9 | 100 | 62.6 | 47.3 |

TABLE 8
Stability of MG3-6/3-4 chemically modified guides
over 21 hours at 37° C.

| | Percentage guide left | | | |
|---|---|---|---|---|
| Time | mAlb3634- | mAlb3634- | mAlb3634- | mAlb3634- |
| (H) | 34-0 | 34-1 | 34-35 | 34-42 |
| 0.5 | 68.3 | 61.9 | 104.6 | 80.9 |
| 1 | 51.0 | 59.6 | 84.0 | 73.2 |
| 4 | 9.6 | 51.0 | 52.6 | 70.7 |
| 9 | 1.7 | 40.4 | 51.2 | 45.3 |
| 21 | 0.03 | 1.4 | 24.8 | 15.6 |

The stability assays showed that introducing three 2′-O-methyls and three PS bonds in the 5′ and 3′ end of the guides significantly improved stability (FIG. 13 and FIG. 14). Adding extra 2′-fluors to the 5′ and 3′ modifications, as in mAlb3634-17 and mAlb3634-42, did not show an apparent advantage at early time points (up to 9 hours) as shown in FIG. 13, but a slight improvement in stability was apparent when the stability assays were run for 21 hours (FIG. 14). Including 2-O-methyl and PS bonds in all the loops of the stem loops (mAlb3634-35) gave an apparent larger increment in stability compared to the guide with chemical modifications on the 5′ and 3′ ends (mAlb3634-1), as seen in FIG. 13. However, when these results were repeated and at longer time points, this increment became less apparent at earlier time points and was became apparent at longer time points up to 21 hours, as seen in FIG. 14. Including 2′-O-methyl in the first strand of distinct stem loops did not provide an advantage in stability for up to 9 hours, as shown by comparing mAlb3634-0 and mAlb3634-29 and mAlb3634-30. mAlb3634-36, which has a combination of 2′-O-methyl in the first strand of all stem loops and PS bonds in the loops of all stem loops, showed an apparent increased stability at 9 hr when compared to end modified guide (mAlb3634-0). However, this guide was not active when tested via mRNA transfection in Hepa1-6 cells. In general, adding extra modifications (e.g., 2′-O-methyl, 2′-O-fluor or PS bonds) to the end modified guide did not confer a large advantage in stability at earlier time points up to 9 hours (FIG. 13), and a small increase in stability was apparent at longer time points (FIG. 14). The large size (110 nt) and highly structured nature of this gRNA may make it inherently more stable than shorter or less structured guide RNA and thereby limit the benefit of chemical modifications on stability. Modifying the 5′ and 3′ ends of the guide appears to provide a good level of protection against nucleases. However adding the extra modifications in the guides might provide more benefit in vivo, as these types of modifications may reduce immunogenicity.

### Example 10—Protein Recombination of Type V-A Nucleases

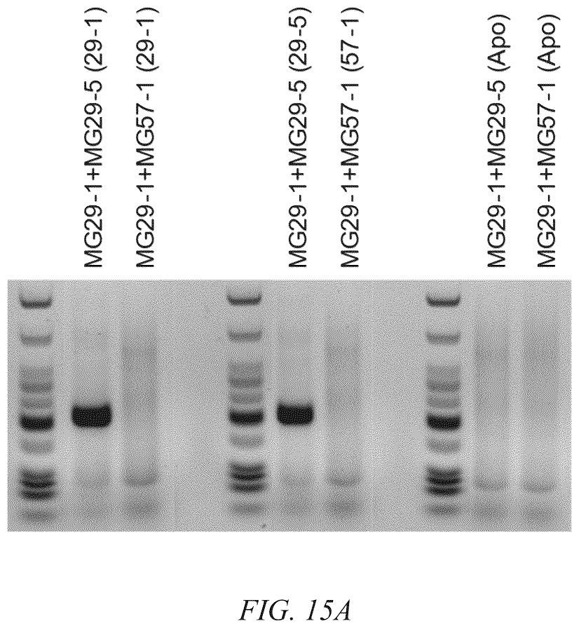

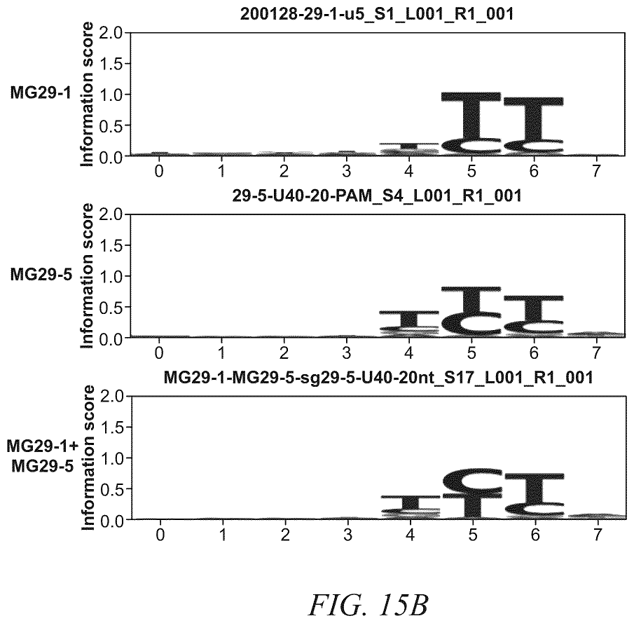

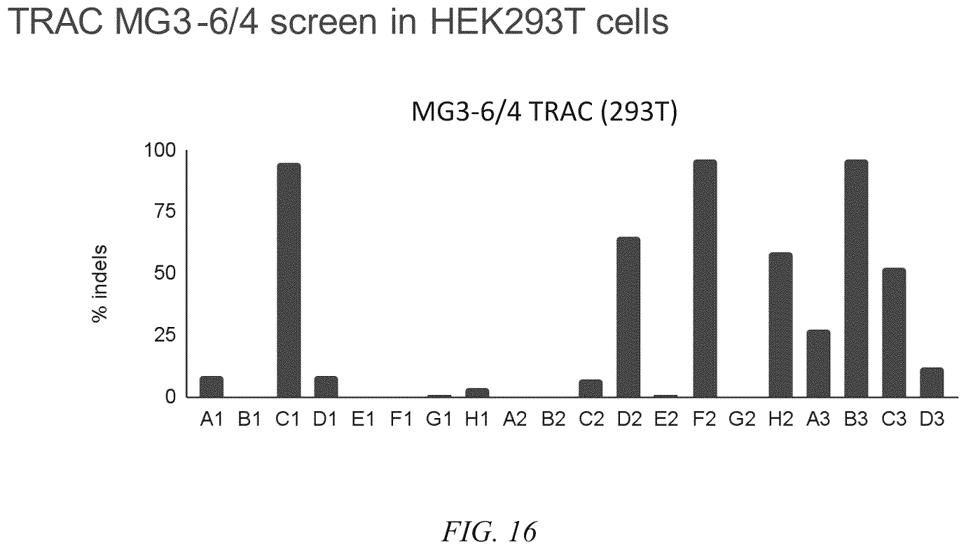

To expand the capability of rapid PAM exchange beyond type II nucleases, three type V-A nucleases were chosen for protein recombination. The breakpoint was chosen based on the predicted structural information (Table 1B). Similar to type II enzyme recombinants, the type V chimera showed activity when proteins were recombined from a closely related family. In vitro PAM enrichment and NGS analysis revealed a consistent result that the PAM of a chimera is inherited from C-terminal parent. It may be possible to avoid potential structural disruptions of protein recombination from distantly related families by utilizing breakpoint optimization (FIGS. 15A-15B).

### Example 11—Analysis of Gene-Editing Outcomes at the DNA Level for TRAC in HEK293T Cells

Nucleofection of MG3-6/4 RNPs (104 pmol protein/300 pmol guide) comprising sgRNAs described below in Table 9A and SEQ ID NOs: 119-158 was performed into HEK293T cells (200,000 cells/well) by electroporation. Three days post-transfection, cells were harvested and genomic DNA was prepared. PCR primers appropriate for use in NGS-based DNA sequencing were generated, optimized, and used to amplify the individual target sequences for each guide RNA. The amplicons were sequenced and analyzed to determine gene editing (FIG. 16). The results show that sgRNAs C1, F2, and B3 were most effective at inducing indels, with appreciable editing also occurring for sgRNAs D2, H2, A3, and C3.

TABLE 9A
gRNAs and Targeting Sequences Used in Example 11

| | SEQ | | |
|---|---|---|---|
| | ID | | |
| Category | NO: | Name | Sequence |
| MG3-6/3- | 119 | MG3- | mG*mC*mC*rGrUrGrUrArCrCrArGrCrUrGrArGrArGrArCrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | A1 | rCrCrGrUrUrUrUrCrCrArArUrArGrGrArGrCrGrGrGrCrGrGrUrAr |
| | | | UrGrU*mU*mU*mU |
| MG3-6/3- | 120 | MG3- | mA*mU*mU*rCrArCrCrGrArUrUrUrUrGrArUrUrCrUrCrArArGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | B1 | rCrCrGrUrUrUrUrCrCrArArUrArGrGrArGrCrGrGrGrCrGrGrUrAr |
| | | | UrGrU*mU*mU*mU |
| MG3-6/3- | 121 | MG3- | mG*mA*mU*rUrCrUrGrArUrGrUrGrUrArUrArUrCrArCrArGrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | C1 | rCrCrGrUrUrUrUrCrCrArArUrArGrGrArGrCrGrGrGrCrGrGrUrAr |
| | | | UrGrU*mU*mU*mU |
| MG3-6/3- | 122 | MG3- | mA*mA*mC*rArGrUrGrCrUrGrUrGrGrCrCrUrGrGrArGrCrArGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | D1 | rCrCrGrUrUrUrUrCrCrArArUrArGrGrArGrCrGrGrGrCrGrGrUrAr |
| | | | UrGrU*mU*mU*mU |
| MG3-6/3- | 123 | MG3- | mG*mG*mC*rUrGrGrGrGrArArGrArArGrGrUrGrUrCrUrUrCrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | E1 | rCrCrGrUrUrUrUrCrCrArArUrArGrGrArGrCrGrGrGrCrGrGrUrAr |
| | | | UrGrU*mU*mU*mU |
| MG3-6/3- | 124 | MG3- | mG*mU*mU*rUrUrGrUrCrUrGrUrGrArUrArUrArCrArCrArUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | F1 | rCrCrGrUrUrUrUrCrCrArArUrArGrGrArGrCrGrGrGrCrGrGrUrAr |
| | | | UrGrU*mU*mU*mU |
| MG3-6/3- | 125 | MG3- | mU*mU*mA*rCrUrUrUrGrUrGrArCrArCrArUrUrUrGrUrUrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | G1 | rCrCrGrUrUrUrUrCrCrArArUrArGrGrArGrCrGrGrGrCrGrGrUrAr |
| | | | UrGrU*mU*mU*mU |
| MG3-6/3- | 126 | MG3- | mU*mU*mG*rUrGrArCrArCrArUrUrUrGrUrUrUrGrArGrArArGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | H1 | rCrCrGrUrUrUrUrCrCrArArUrArGrGrArGrCrGrGrGrCrGrGrUrAr |
| | | | UrGrU*mU*mU*mU |
| MG3-6/3- | 127 | MG3- | mU*mG*mU*rGrArCrArCrArUrUrUrGrUrUrUrGrArGrArArUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | A2 | rCrCrGrUrUrUrUrCrCrArArUrArGrGrArGrCrGrGrGrCrGrGrUrAr |
| | | | UrGrU*mU*mU*mU |
| MG3-6/3- | 128 | MG3- | mA*mU*mU*rUrGrUrUrUrGrArGrArArUrCrArArArArUrCrGrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | B2 | rCrCrGrUrUrUrUrCrCrArArUrArGrGrArGrCrGrGrGrCrGrGrUrAr |
| | | | UrGrU*mU*mU*mU |
| MG3-6/3- | 129 | MG3- | mU*mU*mC*rCrUrGrUrGrArUrGrUrCrArArGrCrUrGrGrUrCrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | C2 | rCrCrGrUrUrUrUrCrCrArArUrArGrGrArGrCrGrGrGrCrGrGrUrAr |
| | | | UrGrU*mU*mU*mU |
| MG3-6/3- | 130 | MG3- | mU*mC*mC*rUrGrUrGrArUrGrUrCrArArGrCrUrGrGrUrCrGrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | D2 | rCrCrGrUrUrUrUrCrCrArArUrArGrGrArGrCrGrGrGrCrGrGrUrAr |
| | | | UrGrU*mU*mU*mU |
| MG3-6/3- | 131 | MG3- | mG*mUmC*rArArGrCrUrGrGrUrCrGrArGrArArArArGrCrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | E2 | rCrCrGrUrUrUrUrCrCrArArUrArGrGrArGrCrGrGrGrCrGrGrUrAr |
| | | | UrGrU*mU*mU*mU |
| MG3-6/3- | 132 | MG3- | mA*mG*mC*rUrUrGrArCrArUrCrArCrArGrGrArArCrUrUrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | F2 | rCrCrGrUrUrUrUrCrCrArArUrArGrGrArGrCrGrGrGrCrGrGrUrAr |
| | | | UrGrU*mU*mU*mU |
| MG3-6/3- | 133 | MG3- | mG*mA*mC*rArUrCrArCrArGrGrArArCrUrUrUrCrUrArArArGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | G2 | rCrCrGrUrUrUrUrCrCrArArUrArGrGrArGrCrGrGrGrCrGrGrUrAr |
| | | | UrGrU*mU*mU*mU |
| MG3-6/3- | 134 | MG3- | mUmU*mA*rCrArGrArUrArCrGrArArCrCrUrArArArCrUrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | H2 | rCrCrGrUrUrUrUrCrCrArArUrArGrGrArGrCrGrGrGrCrGrGrUrAr |
| | | | UrGrU*mU*mU*mU |
| MG3-6/3- | 135 | MG3- | mA*mA*mA*rArCrCrUrGrUrCrArGrUrGrArUrUrGrGrGrUrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | A3 | rCrCrGrUrUrUrUrCrCrArArUrArGrGrArGrCrGrGrGrCrGrGrUrAr |
| | | | UrGrU*mU*mU*mU |
| MG3-6/3- | 136 | MG3- | mG*mA*mU*rUrGrGrGrUrUrCrCrGrArArUrCrCrUrCrCrUrCrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | B3 | rCrCrGrUrUrUrUrCrCrArArUrArGrGrArGrCrGrGrGrCrGrGrUrAr |
| | | | UrGrU*mU*mU*mU |
| MG3-6/3- | 137 | MG3- | mG*mG*mA*rArCrCrCrArArUrCrArCrUrGrArCrArGrGrUrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | C3 | rCrCrGrUrUrUrUrCrCrArArUrArGrGrArGrCrGrGrGrCrGrGrUrAr |
| | | | UrGrU*mU*mU*mU |
| MG3-6/3 | 138 | MG3- | mU*mUmG*rArArArGrUrUrUrArGrGrUrUrCrGrUrArUrCrUrGrUrU |
| 4 sgRNA | | 6/3-4 | rGrArGrArArUrCrGrArArArGrArUrUrCrUrUrArArUrArArGrGrCr |
| targeting | | TRAC | ArUrCrCrUrUrCrCrGrArUrGrCrUrGrArCrUrUrCrUrCrArCrCrGrU |
| TRAC | | D3 | rCrCrGrUrUrUrUrCrCrArArUrArGrGrArGrCrGrGrGrCrGrGrUrAr |
| | | | UrGrU*mU*mU*mU |
| DNA | 139 | MG3- | GCCGTGTACCAGCTGAGAGACT |
| sequence | | 6/3-4 | |
| of TRAC | | TRAC | |
| target site | | A1 | |
| DNA | 140 | MG3- | ATTCACCGATTTTGATTCTCAA |
| sequence | | 6/3-4 | |
| of TRAC | | TRAC | |
| target site | | B1 | |
| DNA | 141 | MG3- | GATTCTGATGTGTATATCACAG |
| sequence | | 6/3-4 | |
| of TRAC | | TRAC | |
| target site | | C1 | |
| DNA | 142 | MG3- | AACAGTGCTGTGGCCTGGAGCA |
| sequence | | 6/3-4 | |
| of TRAC | | TRAC | |
| target site | | D1 | |
| DNA | 143 | MG3- | GGCTGGGGAAGAAGGTGTCTTC |
| sequence | | 6/3-4 | |
| of TRAC | | TRAC | |
| target site | | E1 | |
| DNA | 144 | MG3- | GTTTTGTCTGTGATATACACAT |
| sequence | | 6/3-4 | |
| of TRAC | | TRAC | |
| target site | | F1 | |
| DNA | 145 | MG3- | TTACTTTGTGACACATTTGTTT |
| sequence | | 6/3-4 | |
| of TRAC | | TRAC | |
| target site | | G1 | |
| DNA | 146 | MG3- | TTGTGACACATTTGTTTGAGAA |
| sequence | | 6/3-4 | |
| of TRAC | | TRAC | |
| target site | | H1 | |
| DNA | 147 | MG3- | TGTGACACATTTGTTTGAGAAT |
| sequence | | 6/3-4 | |
| of TRAC | | TRAC | |
| target site | | A2 | |
| DNA | 148 | MG3- | ATTTGTTTGAGAATCAAAATCG |
| sequence | | 6/3-4 | |
| of TRAC | | TRAC | |
| target site | | B2 | |
| DNA | 149 | MG3- | TTCCTGTGATGTCAAGCTGGTC |
| sequence | | 6/3-4 | |
| of TRAC | | TRAC | |
| target site | | C2 | |
| DNA | 150 | MG3- | TCCTGTGATGTCAAGCTGGTCG |
| sequence | | 6/3-4 | |
| of TRAC | | TRAC | |
| target site | | D2 | |
| DNA | 151 | MG3- | GTCAAGCTGGTCGAGAAAAGCT |
| sequence | | 6/3-4 | |
| of TRAC | | TRAC | |
| target site | | E2 | |
| DNA | 152 | MG3- | AGCTTGACATCACAGGAACTTT |
| sequence | | 6/3-4 | |
| of TRAC | | TRAC | |
| target site | | F2 | |
| DNA | 153 | MG3- | GACATCACAGGAACTTTCTAAA |
| sequence | | 6/3-4 | |
| of TRAC | | TRAC | |
| target site | | G2 | |
| DNA | 154 | MG3- | TTACAGATACGAACCTAAACTT |
| sequence | | 6/3-4 | |
| of TRAC | | TRAC | |
| target site | | H2 | |
| DNA | 155 | MG3- | AAAACCTGTCAGTGATTGGGTT |
| sequence | | 6/3-4 | |
| of TRAC | | TRAC | |
| target site | | A3 | |
| DNA | 156 | MG3- | GATTGGGTTCCGAATCCTCCTC |
| sequence | | 6/3-4 | |
| of TRAC | | TRAC | |
| target site | | B3 | |
| DNA | 157 | MG3- | GGAACCCAATCACTGACAGGTT |
| sequence | | 6/3-4 | |
| of TRAC | | TRAC | |
| target site | | C3 | |
| DNA | 158 | MG3- | TTGAAAGTTTAGGTTCGTATCT |
| sequence | | 6/3-4 | |
| of TRAC | | TRAC | |
| target site | | D3 | |

(r = native ribose base, m = 2′-O methyl modified base, F = 2′ Fluro modified base,
* = phosphorothioate bond)

### Example 12—Analysis of Gene-Editing Outcomes at the DNA Level for B32M in HEK293T Cells

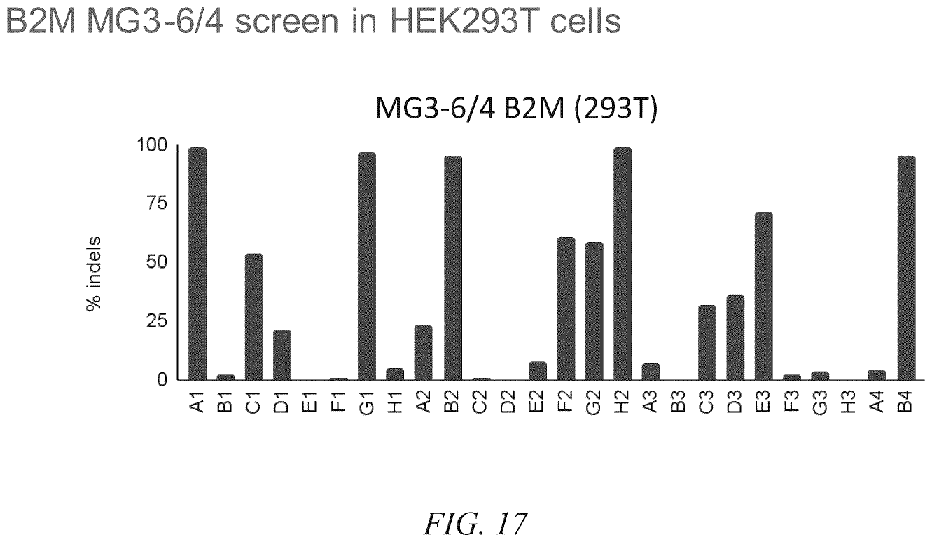

Nucleofection of MG3-6/4 RNPs (104 pmol protein/300 pmol guide) comprising sgRNAs described below in Table 9B and SEQ ID NOs: 159-210 was performed into HEK293T cells (200,000 cells) using electroporation. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing (FIG. 17). The results show that sgRNAs A1, GU, B2, H2, and B4 were the most effective for inducing editing, with appreciable editing also being detected for sgRNAs C, D1, A2, H1, E2, F2, G2, A3, C3, and D3.

TABLE 9B
gRNAs and Targeting Sequences Used
in Example 12

| | SEQ | | |
|---|---|---|---|
| | ID | | |
| Category | NO: | Name | Sequence |
| MG3-6/3- | 159 | MG3- | mU*mC*mA*rCrGrCrUrGrGrArU |
| 4 sgRNA | | 6/3-4 | rArGrCrCrUrCrCrArGrGrCrGr |
| targeting | | B2M | UrUrGrArGrArArUrCrGrArArA |
| B2M | | A1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 160 | MG3- | mG*mG*mU*rUrUrArCrUrCrArC |
| 4 sgRNA | | 6/3-4 | rGrUrCrArUrCrCrArGrCrArGr |
| targeting | | B2M | UrUrGrArGrArArUrCrGrArArA |
| B2M | | B1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 161 | MG3- | mA*mC*mU*rCrArCrGrUrCrArU |
| 4 sgRNA | | 6/3-4 | rCrCrArGrCrArGrArGrArArGr |
| targeting | | B2M | UrUrGrArGrArArUrCrGrArArA |
| B2M | | C1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 162 | MG3- | mUmC*mA*rUrCrCrArGrCrArGr |
| 4 sgRNA | | 6/3-4 | ArGrArArUrGrGrArArArGrGrU |
| targeting | | B2M | rUrGrArGrArArUrCrGrArArAr |
| B2M | | D1 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 163 | MG3- | mA*mG*mA*rGrArArUrGrGrArA |
| 4 sgRNA | | 6/3-4 | rArGrUrCrArArArUrUrUrCrGr |
| targeting | | B2M | UrUrGrArGrArArUrCrGrArArA |
| B2M | | E1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 164 | MG3- | mC *mG*mA*rCrArUrUrGrArAr |
| 4 sgRNA | | 6/3-4 | GrUrUrGrArCrUrUrArCrUrGrG |
| targeting | | B2M | rUrUrGrArGrArArUrCrGrArAr |
| B2M | | F1 | ArGrArUrUrCrUrUrArArUrArA |
| | | | rGrGrCrArUrCrCrUrUrCrCrGr |
| | | | ArUrGrCrUrGrArCrUrUrCrUrC |
| | | | rArCrCrGrUrCrCrGrUrUrUrUr |
| | | | CrCrArArUrArGrGrArGrCrGrG |
| | | | rGrCrGrGrUrArUrGrU*mU*mU* |
| | | | mU |
| MG3-6/3- | 165 | MG3- | mU*mU*mG*rArCrUrUrArCrUrG |
| 4 sgRNA | | 6/3-4 | rArArGrArArUrGrGrArGrArGr |
| targeting | | B2M | UrUrGrArGrArArUrCrGrArArA |
| B2M | | G1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 166 | MG3- | mUmU*mA*rCrUrGrArArGrArAr |
| 4 sgRNA | | 6/3-4 | UrGrGrArGrArGrArGrArArGrU |
| targeting | | B2M | rUrGrArGrArArUrCrGrArArAr |
| B2M | | H1 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 167 | MG3- | mU*mA*mC*rUrGrArArGrArArU |
| 4 sgRNA | | 6/3-4 | rGrGrArGrArGrArGrArArUrGr |
| targeting | | B2M | UrUrGrArGrArArUrCrGrArArA |
| B2M | | A2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 168 | MG3- | mA*mC*mU*rGrArArGrArArUrG |
| 4 sgRNA | | 6/3-4 | rGrArGrArGrArGrArArUrUrGr |
| targeting | | B2M | UrUrGrArGrArArUrCrGrArArA |
| B2M | | B2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 169 | MG3- | mU*mC*mU*rUrUrCrUrArUrCrU |
| 4 sgRNA | | 6/3-4 | rCrUrUrGrUrArCrUrArCrArGr |
| targeting | | B2M | UrUrGrArGrArArUrCrGrArArA |
| B2M | | C2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 170 | MG3- | mU*mA*mC*rUrArCrArCrUrGrA |
| 4 sgRNA | | 6/3-4 | rArUrUrCrArCrCrCrCrCrArGr |
| targeting | | B2M | UrUrGrArGrArArUrCrGrArArA |
| B2M | | D2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 171 | MG3- | mA*mC*mU*rArCrArCrUrGrArA |
| 4 sgRNA | | 6/3-4 | rUrUrCrArCrCrCrCrCrArCrGr |
| targeting | | B2M | UrUrGrArGrArArUrCrGrArArA |
| B2M | | E2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 172 | MG3- | mC*mU*mA*rCrArCrUrGrArArU |
| 4 sgRNA | | 6/3-4 | rUrCrArCrCrCrCrCrArCrUrGr |
| targeting | | B2M | UrUrGrArGrArArUrCrGrArArA |
| B2M | | F2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 173 | MG3- | mA*mU*mA*rCrUrCrArUrCrUrU |
| 4 sgRNA | | 6/3-4 | rUrUrUrCrArGrUrGrGrGrGrGr |
| targeting | | B2M | UrUrGrArGrArArUrCrGrArArA |
| B2M | | G2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 174 | MG3- | mG*mA*mA*rUrUrCrArGrUrGrU |
| 4 sgRNA | | 6/3-4 | rArGrUrArCrArArGrArGrArGr |
| targeting | | B2M | UrUrGrArGrArArUrCrGrArArA |
| B2M | | H2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 175 | MG3- | mG*mA*mG*rArUrArGrArArArG |
| 4 sgRNA | | 6/3-4 | rArCrCrArGrUrCrCrUrUrGrGr |
| targeting | | B2M | UrUrGrArGrArArUrCrGrArArA |
| B2M | | A3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 176 | MG3- | mC*mA*mG*rUrCrCrUrUrGrCrU |
| 4 sgRNA | | 6/3-4 | rGrArArArGrArCrArArGrUrGr |
| targeting | | B2M | UrUrGrArGrArArUrCrGrArArA |
| B2M | | B3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 177 | MG3- | mA*mG*mU*rCrArArCrUrUrCrA |
| 4 sgRNA | | 6/3-4 | rArUrGrUrCrGrGrArUrGrGrGr |
| targeting | | B2M | UrUrGrArGrArArUrCrGrArArA |
| B2M | | C3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 178 | MG3- | mA*mA*mA*rCrCrCrArGrArCrA |
| 4 sgRNA | | 6/3-4 | rCrArUrArGrCrArArUrUrCrGr |
| targeting | | B2M | UrUrGrArGrArArUrCrGrArArA |
| B2M | | D3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 179 | MG3- | mA*mA*mC*rCrCrArGrArCrArC |
| 4 sgRNA | | 6/3-4 | rArUrArGrCrArArUrUrCrArGr |
| targeting | | B2M | UrUrGrArGrArArUrCrGrArArA |
| B2M | | E3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 180 | MG3- | mC*mU*mG*rCrUrGrGrArUrGrA |
| 4 sgRNA | | 6/3-4 | rCrGrUrGrArGrUrArArArCrGr |
| targeting | | B2M | UrUrGrArGrArArUrCrGrArArA |
| B2M | | F3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 181 | MG3- | mA*mC*mC*rUrGrArArUrCrUrU |
| 4 sgRNA | | 6/3-4 | rUrGrGrArGrUrArCrCrUrGrGr |
| targeting | | B2M | UrUrGrArGrArArUrCrGrArArA |
| B2M | | G3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 182 | MG3- | mU*mG*mC*rUrGrCrUrUrArCrA |
| 4 sgRNA | | 6/3-4 | rUrGrUrCrUrCrGrArUrCrUrGr |
| targeting | | B2M | UrUrGrArGrArArUrCrGrArArA |
| B2M | | H3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 183 | MG3- | mG*mC*mU*rGrCrUrUrArCrArU |
| 4 sgRNA | | 6/3-4 | rGrUrCrUrCrGrArUrCrUrArGr |
| targeting | | B2M | UrUrGrArGrArArUrCrGrArArA |
| B2M | | A4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 184 | MG3- | mC *mU*mG*rCrUrUrArCrArUr |
| 4 sgRNA | | 6/3-4 | GrUrCrUrCrGrArUrCrUrArUrG |
| targeting | | B2M | rUrUrGrArGrArArUrCrGrArAr |
| B2M | | B4 | ArGrArUrUrCrUrUrArArUrArA |
| | | | rGrGrCrArUrCrCrUrUrCrCrGr |
| | | | ArUrGrCrUrGrArCrUrUrCrUrC |
| | | | rArCrCrGrUrCrCrGrUrUrUrUr |
| | | | CrCrArArUrArGrGrArGrCrGrG |
| | | | rGrCrGrGrUrArUrGrU*mU*mU* |
| | | | mU |
| DNA | 185 | MG3- | TCACGCTGGATAGCCTCCAGGC |
| sequence | | 6/3-4 | |
| of B2M | | B2M | |
| target | | A1 | |
| site | | | |
| DNA | 186 | MG3- | GGTTTACTCACGTCATCCAGCA |
| sequence | | 6/3-4 | |
| of B2M | | B2M | |
| target | | B1 | |
| site | | | |
| DNA | 187 | MG3- | ACTCACGTCATCCAGCAGAGAA |
| sequence | | 6/3-4 | |
| of B2M | | B2M | |
| target | | C1 | |
| site | | | |
| DNA | 188 | MG3- | TCATCCAGCAGAGAATGGAAAG |
| sequence | | 6/3-4 | |
| of B2M | | B2M | |
| target | | D1 | |
| site | | | |
| DNA | 189 | MG3- | AGAGAATGGAAAGTCAAATTTC |
| sequence | | 6/3-4 | |
| of B2M | | B2M | |
| target | | E1 | |
| site | | | |
| DNA | 190 | MG3- | CGACATTGAAGTTGACTTACTG |
| sequence | | 6/3-4 | |
| of B2M | | B2M | |
| target | | F1 | |
| site | | | |
| DNA | 191 | MG3- | TTGACTTACTGAAGAATGGAGA |
| sequence | | 6/3-4 | |
| of B2M | | B2M | |
| target | | G1 | |
| site | | | |
| DNA | 192 | MG3- | TTACTGAAGAATGGAGAGAGAA |
| sequence | | 6/3-4 | |
| of B2M | | B2M | |
| target | | H1 | |
| site | | | |
| DNA | 193 | MG3- | TACTGAAGAATGGAGAGAGAAT |
| sequence | | 6/3-4 | |
| of B2M | | B2M | |
| target | | A2 | |
| site | | | |
| DNA | 194 | MG3- | ACTGAAGAATGGAGAGAGAATT |
| sequence | | 6/3-4 | |
| of B2M | | B2M | |
| target | | B2 | |
| site | | | |
| DNA | 195 | MG3- | TCTTTCTATCTCTTGTACTACA |
| sequence | | 6/3-4 | |
| of B2M | | B2M | |
| target | | C2 | |
| site | | | |
| DNA | 196 | MG3- | TACTACACTGAATTCACCCCCA |
| sequence | | 6/3-4 | |
| of B2M | | B2M | |
| target | | D2 | |
| site | | | |
| DNA | 197 | MG3- | ACTACACTGAATTCACCCCCAC |
| sequence | | 6/3-4 | |
| of B2M | | B2M | |
| target | | E2 | |
| site | | | |
| DNA | 198 | MG3- | CTACACTGAATTCACCCCCACT |
| sequence | | 6/3-4 | |
| of B2M | | B2M | |
| target | | F2 | |
| site | | | |
| DNA | 199 | MG3- | ATACTCATCTTTTTCAGTGGGG |
| sequence | | 6/3-4 | |
| of B2M | | B2M | |
| target | | G2 | |
| site | | | |
| DNA | 200 | MG3- | GAATTCAGTGTAGTACAAGAGA |
| sequence | | 6/3-4 | |
| of B2M | | B2M | |
| target | | H2 | |
| site | | | |
| DNA | 201 | MG3- | GAGATAGAAAGACCAGTCCTTG |
| sequence | | 6/3-4 | |
| of B2M | | B2M | |
| target | | A3 | |
| site | | | |
| DNA | 202 | MG3- | CAGTCCTTGCTGAAAGACAAGT |
| sequence | | 6/3-4 | |
| of B2M | | B2M | |
| target | | B3 | |
| site | | | |
| DNA | 203 | MG3- | AGTCAACTTCAATGTCGGATGG |
| sequence | | 6/3-4 | |
| of B2M | | B2M | |
| target | | C3 | |
| site | | | |
| DNA | 204 | MG3- | AAACCCAGACACATAGCAATTC |
| sequence | | 6/3-4 | |
| of B2M | | B2M | |
| target | | D3 | |
| site | | | |
| DNA | 205 | MG3- | AACCCAGACACATAGCAATTCA |
| sequence | | 6/3-4 | |
| of B2M | | B2M | |
| target | | E3 | |
| site | | | |
| DNA | 206 | MG3- | CTGCTGGATGACGTGAGTAAAC |
| sequence | | 6/3-4 | |
| of B2M | | B2M | |
| target | | F3 | |
| site | | | |
| DNA | 207 | MG3- | ACCTGAATCTTTGGAGTACCTG |
| sequence | | 6/3-4 | |
| of B2M | | B2M | |
| target | | G3 | |
| site | | | |
| DNA | 208 | MG3- | TGCTGCTTACATGTCTCGATCT |
| sequence | | 6/3-4 | |
| of B2M | | B2M | |
| target | | H3 | |
| site | | | |
| DNA | 209 | MG3- | GCTGCTTACATGTCTCGATCTA |
| sequence | | 6/3-4 | |
| of B2M | | B2M | |
| target | | A4 | |
| site | | | |
| DNA | 210 | MG3- | CTGCTTACATGTCTCGATCTAT |
| sequence | | 6/3-4 | |
| of B2M | | B2M | |
| target | | B4 | |
| site | | | |

(r = native ribose base, m = 2′-O methyl modified base, F = 2′ Fluro modified base, * = phosphorothioate bond)

### Example 13—Analysis of Gene-Editing Outcomes at the DNA and Phenotypic Levels for TRAC in T Cells

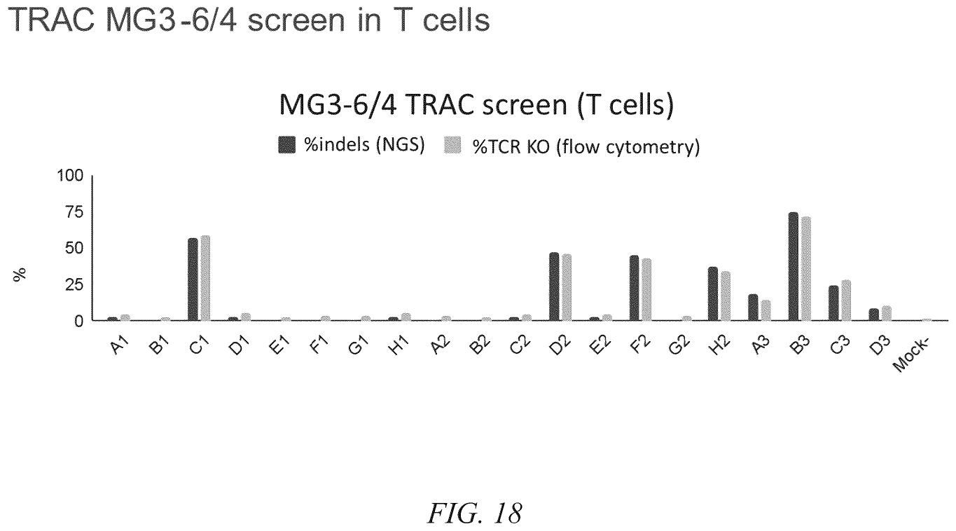

Primary T cells were purified from PMBCs using a negative selection. Nucleofection of MG3-6/4 RNPs (104 pmol protein/120 pmol guide) comprising selected sgRNAs described in Table 9A and SEQ ID NOs: 119-158 was performed into T cells (200,000) using electroporation. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing. For analysis by flow cytometry, 3 days post-nucleofection, 100,000 T cells were stained with anti-CD3 antibody for 30 minutes at 4° C. and analyzed by flow cytometry (FIG. 18). Results indicated that sgRNAs C1, D2, F2, H2, A3, B3, C3, and D3 showed appreciable editing, with the most editing performed by sgRNAs C1 and B3.

### Example 14—Analysis of Gene-Editing Outcomes at the DNA Level for B2M in T Cells

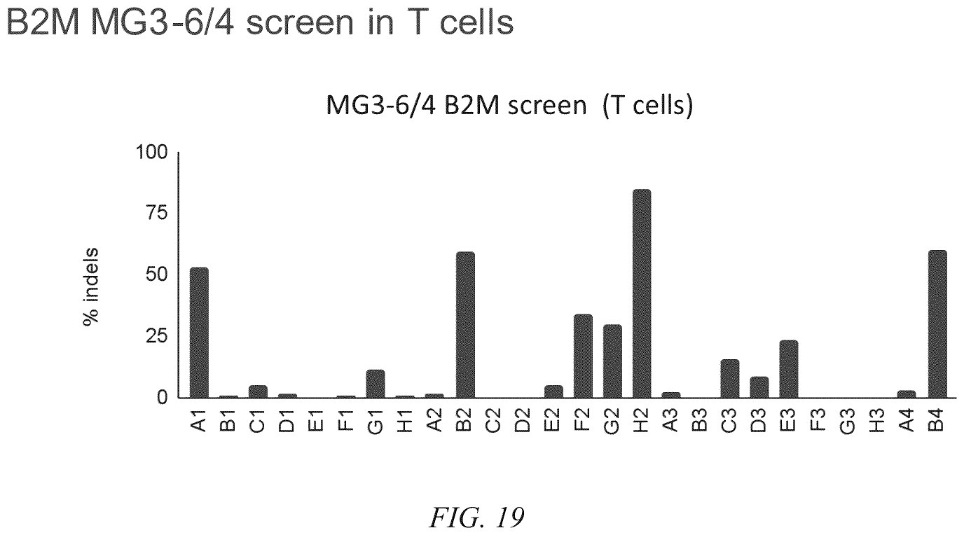

Primary T cells were purified from PMBCs using a negative selection. Nucleofection of MG3-6/4 RNPs (104 pmol protein/120 pmol guide) comprising selected sgRNAs described in Table 9B and SEQ ID NOs: 159-210 was performed into T cells (200,000) using electroporation. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing (FIG. 19).

### Example 15—Analysis of Gene-Editing Outcomes at the Phenotypic Level for TRBC1 and TRBC2 in T Cells

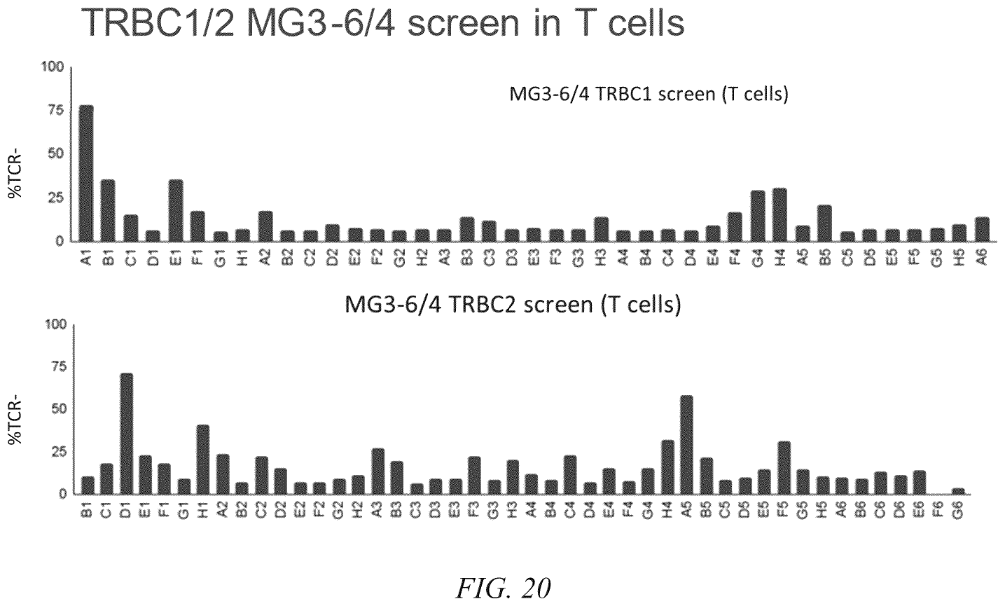

Primary T cells were purified from PBMCs using a negative selection. Nucleofection of MG3-6/4 RNPs (104 pmol protein/120 pmol guide) comprising sgRNAs described below in Table 9C below and SEQ ID NOs: 211-382 was performed into T cells (200,000) using electroporation. For analysis by flow cytometry, 3 days post-nucleofection, 100,000 T cells were stained with anti-CD3 antibody for 30 minutes at 4° C. and analyzed by flow cytometry (FIG. 20). The results show, the highest-performing sgRNAs for TRBC1 were A1, B1, E1, G4, H4, and B5 (FIG. 20). Similarly, the highest performing sgRNAs for TRBC2 were D1, H1, and A5.

TABLE 9C
gRNAs and Targeting Sequences Used
in Example 15

| | SEQ | | |
|---|---|---|---|
| | ID | | |
| Category | NO: | Name | Sequence |
| MG3-6/3- | 211 | MG3- | mC*mA*mG*rArArGrCrArGrArG |
| 4 sgRNA | | 6/3-4 | rArUrCrUrCrCrCrArCrArCrGr |
| targeting | A1 | TRBC1 | UrUrGrArGrArArUrCrGrArArA |
| TRBC1 | | | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 212 | MG3- | mC*mC*mA*rCrGrUrGrGrArGrC |
| 4 sgRNA | | 6/3- | rUrGrArGrCrUrGrGrUrGrGrGr |
| targeting | 4 | TRBC1 | UrUrGrArGrArArUrCrGrArArA |
| TRBC1 | | B1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 213 | MG3- | mA*mG*mU*rCrCrArGrUrUrCrU |
| 4 sgRNA | | 6/3- | rArCrGrGrGrCrUrCrUrCrGrGr |
| targeting | 4 | TRBC1 | UrUrGrArGrArArUrCrGrArArA |
| TRBC1 | | C1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 214 | MG3- | mG*mA*mU*rUrArGrGrUrGrArG |
| 4 sgRNA | | 6/3- | rArCrCrArGrCrUrArCrCrArGr |
| targeting | 4 | TRBC1 | UrUrGrArGrArArUrCrGrArArA |
| TRBC1 | | D1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 215 | MG3- | mA*mU*mU*rArGrGrUrGrArGrA |
| 4 sgRNA | | 6/3- | rCrCrArGrCrUrArCrCrArGrGr |
| targeting | 4 | TRBC1 | UrUrGrArGrArArUrCrGrArArA |
| TRBC1 | | E1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 216 | MG3- | mU*mU*mA*rGrGrUrGrArGrArC |
| 4 sgRNA | | 6/3- | rCrArGrCrUrArCrCrArGrGrGr |
| targeting | 4 | TRBC1 | UrUrGrArGrArArUrCrGrArArA |
| TRBC1 | | F1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 217 | MG3- | mU*mG*mA*rGrArCrCrArGrCrU |
| 4 sgRNA | | 6/3- | rArCrCrArGrGrGrArArArArGr |
| targeting | 4 | TRBC1 | UrUrGrArGrArArUrCrGrArArA |
| TRBC1 | | G1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 218 | MG3- | mC*mA*mG*rGrUrArGrCrArGrA |
| 4 sgRNA | | 6/3- | rCrArArGrArCrUrArGrArUrGr |
| targeting | 4 | TRBC1 | UrUrGrArGrArArUrCrGrArArA |
| TRBC1 | | H1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 219 | MG3- | mA*mG*mG*rUrArGrCrArGrArC |
| 4 sgRNA | | 6/3- | rArArGrArCrUrArGrArUrCrGr |
| targeting | 4 | TRBC1 | UrUrGrArGrArArUrCrGrArArA |
| TRBC1 | | A2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3 | 220 | MG3- | mA*mG*mC*rArGrArCrArArGrA |
| 4 sgRNA | | 6/3- | rCrUrArGrArUrCrCrArArArGr |
| targeting | 4 | TRBC1 | UrUrGrArGrArArUrCrGrArArA |
| TRBC1 | | B2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 221 | MG3- | mG*mG*mA*rArCrCrArGrCrGrC |
| 4 sgRNA | | 6/3- | rArCrArCrCrArUrGrArArGrGr |
| targeting | 4 | TRBC1 | UrUrGrArGrArArUrCrGrArArA |
| TRBC1 | | C2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 222 | MG3- | mG*mUmG*rGrCrUrGrArCrArUr |
| 4 sgRNA | | 6/3- | CrUrGrCrArUrGrGrCrArGrGrU |
| targeting | 4 | TRBC1 | rUrGrArGrArArUrCrGrArArAr |
| TRBC1 | | D2 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3 | 223 | MG3- | mG*mG*mC*rCrUrGrGrGrArGrU |
| 4 sgRNA | | 6/3- | rCrUrGrUrGrCrCrArArCrUrGr |
| targeting | 4 | TRBC1 | UrUrGrArGrArArUrCrGrArArA |
| TRBC1 | | E2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 224 | MG3- | mC*mU*mG*rArCrUrUrUrArCrU |
| 4 sgRNA | | 6/3-4 | rUrUrUrArArUrUrGrCrCrUrGr |
| targeting | | TRBC1 | UrUrGrArGrArArUrCrGrArArA |
| TRBC1 | | F2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 225 | MG3- | mU*mG*mA*rCrUrUrUrArCrUrU |
| 4 sgRNA | | 6/3- | rUrUrArArUrUrGrCrCrUrArGr |
| targeting | 4 | TRBC1 | UrUrGrArGrArArUrCrGrArArA |
| TRBC1 | | G2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 226 | MG3- | mG*mA*mC*rUrUrUrArCrUrUrU |
| 4 sgRNA | | 6/3- | rUrArArUrUrGrCrCrUrArUrGr |
| targeting | 4 | TRBC1 | UrUrGrArGrArArUrCrGrArArA |
| TRBC1 | | H2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 227 | MG3- | mG*mG*mG*rArArGrGrArGrArA |
| 4 sgRNA | | 6/3- | rGrCrUrGrGrArGrUrCrArCrGr |
| targeting | 4 | TRBC1 | UrUrGrArGrArArUrCrGrArArA |
| TRBC1 | | A3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 228 | MG3- | mG*mG*mA*rArGrGrArGrArArG |
| 4 sgRNA | | 6/3- | rCrUrGrGrArGrUrCrArCrCrGr |
| targeting | 4 | TRBC1 | UrUrGrArGrArArUrCrGrArArA |
| TRBC1 | | B3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 229 | MG3- | mA*mA*mC*rUrCrCrUrGrGrCrU |
| 4 sgRNA | | 6/3- | rCrUrUrArArUrArArCrCrCrGr |
| targeting | 4 | TRBC1 | UrUrGrArGrArArUrCrGrArArA |
| TRBC1 | | C3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 230 | MG3- | mA*mA*mC*rUrUrUrCrUrCrUrU |
| 4 sgRNA | | 6/3- | rCrUrGrCrArGrGrUrCrArArGr |
| targeting | 4 | TRBC1 | UrUrGrArGrArArUrCrGrArArA |
| TRBC1 | | D3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 231 | MG3- | mA*mC*mU*rCrCrArCrUrUrCrC |
| 4 sgRNA | | 6/3- | rArGrGrGrCrUrGrCrCrUrUrGr |
| targeting | 4 | TRBC1 | UrUrGrArGrArArUrCrGrArArA |
| TRBC1 | | E3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 232 | MG3- | mC*mUmC*rCrArCrUrUrCrCrAr |
| 4 sgRNA | | 6/3- | GrGrGrCrUrGrCrCrUrUrCrGrU |
| targeting | 4 | TRBC1 | rUrGrArGrArArUrCrGrArArAr |
| TRBC1 | | F3 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 233 | MG3- | mU*mC*mC*rUrUrUrCrUrCrUrU |
| 4 sgRNA | | 6/3- | rGrArCrCrUrGrCrArGrArArGr |
| targeting | 4 | TRBC1 | UrUrGrArGrArArUrCrGrArArA |
| TRBC1 | | G3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 234 | MG3- | mA*mG*mC*rCrArGrGrArGrUrU |
| 4 sgRNA | | 6/3- | rGrUrGrArGrGrArUrUrGrArGr |
| targeting | 4 | TRBC1 | UrUrGrArGrArArUrCrGrArArA |
| TRBC1 | | H3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 235 | MG3- | mA*mG*mU*rArGrUrArGrGrGrC |
| 4 sgRNA | | 6/3- | rCrCrArUrUrGrArCrCrArCrGr |
| targeting | 4 | TRBC1 | UrUrGrArGrArArUrCrGrArArA |
| TRBC1 | | A4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 236 | MG3- | mU*mG*mC*rArArGrUrUrArUrC |
| 4 sgRNA | | 6/3- | rUrUrCrUrGrArGrGrCrArCrGr |
| targeting | 4 | TRBC1 | UrUrGrArGrArArUrCrGrArArA |
| TRBC1 | | B4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 237 | MG3- | mA*mG*mU*rUrArUrCrUrUrCrU |
| 4 sgRNA | | 6/3- | rGrArGrGrCrArCrCrUrGrArGr |
| targeting | 4 | TRBC1 | UrUrGrArGrArArUrCrGrArArA |
| TRBC1 | | C4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 238 | MG3- | mG*mU*mU*rArUrCrUrUrCrUrG |
| 4 sgRNA | | 6/3-4 | rArGrGrCrArCrCrUrGrArArGr |
| targeting | | TRBC1 | UrUrGrArGrArArUrCrGrArArA |
| TRBC1 | | D4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 239 | MG3- | mUmC*mA*rArGrArArCrCrArUr |
| 4 sgRNA | | 6/3- | GrArGrArGrArGrGrGrArGrGrU |
| targeting | 4 | TRBC1 | rUrGrArGrArArUrCrGrArArAr |
| TRBC1 | | E4 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 240 | MG3- | mC*mA*mA*rGrArArCrCrArUrG |
| 4 sgRNA | | 6/3- | rArGrArGrArGrGrGrArGrArGr |
| targeting | 4 | TRBC1 | UrUrGrArGrArArUrCrGrArArA |
| TRBC1 | | F4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 241 | MG3- | mU*mU*mA*rCrCrCrGrArGrGrU |
| 4 sgRNA | | 6/3-4 | rArArArGrCrCrArCrArGrUrGr |
| targeting | | TRBC1 | UrUrGrArGrArArUrCrGrArArA |
| TRBC1 | | G4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 242 | MG3- | mC*mC*mG*rArGrGrUrArArArG |
| 4 sgRNA | | 6/3- | rCrCrArCrArGrUrCrUrGrArGr |
| targeting | 4 | TRBC1 | UrUrGrArGrArArUrCrGrArArA |
| TRBC1 | | H4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 243 | MG3- | mC*mA*mG*rUrCrUrGrArArArG |
| 4 sgRNA | | 6/3- | rArArArGrCrArGrGrGrArGrGr |
| targeting | 4 | TRBC1 | UrUrGrArGrArArUrCrGrArArA |
| TRBC1 | | A5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 244 | MG3- | mA*mG*mU*rCrUrGrArArArGrA |
| 4 sgRNA | | 6/3- | rArArGrCrArGrGrGrArGrArGr |
| targeting | 4 | TRBC1 | UrUrGrArGrArArUrCrGrArArA |
| TRBC1 | | B5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 245 | MG3- | mG*mU*mC*rUrGrArArArGrArA |
| 4 sgRNA | | 6/3-4 | rArGrCrArGrGrGrArGrArGrGr |
| targeting | | TRBC1 | UrUrGrArGrArArUrCrGrArArA |
| TRBC1 | | C5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 246 | MG3- | mG*mA*mA*rArGrArArArGrCrA |
| 4 sgRNA | | 6/3- | rGrGrGrArGrArGrGrArArArGr |
| targeting | 4 | TRBC1 | UrUrGrArGrArArUrCrGrArArA |
| TRBC1 | | D5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 247 | MG3- | mG*mA*mG*rArCrCrUrUrArUrU |
| 4 sgRNA | | 6/3-4 | rUrUrCrArUrArGrGrCrArArGr |
| targeting | | TRBC1 | UrUrGrArGrArArUrCrGrArArA |
| TRBC1 | | E5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 248 | MG3- | mG*mA*mU*rGrArGrArGrUrUrA |
| 4 sgRNA | | 6/3- | rCrArCrArGrGrCrCrArCrArGr |
| targeting | 4 | TRBC1 | UrUrGrArGrArArUrCrGrArArA |
| TRBC1 | | F5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 249 | MG3- | mA*mG*mC*rUrGrCrUrUrGrGrC |
| 4 sgRNA | | 6/3- | rUrCrUrGrUrUrGrGrGrCrUrGr |
| targeting | 4 | TRBC1 | UrUrGrArGrArArUrCrGrArArA |
| TRBC1 | | G5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 250 | MG3- | mU*mG*mU*rUrGrGrGrCrUrGrA |
| 4 sgRNA | | 6/3- | rGrArArUrCrUrGrGrGrArGrGr |
| targeting | 4 | TRBC1 | UrUrGrArGrArArUrCrGrArArA |
| TRBC1 | | H5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 251 | MG3- | mG*mG*mA*rArCrArCrCrUrUrG |
| 4 sgRNA | | 6/3- | rUrUrCrArGrGrUrCrCrUrCrGr |
| targeting | 4 | TRBC1 | UrUrGrArGrArArUrCrGrArArA |
| TRBC1 | | A6 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| DNA | 252 | MG3- | CAGAAGCAGAGATCTCCCACAC |
| sequence | | 6/3-4 | |
| of | | TRBC1 | |
| TRBC1 | | A1 | |
| target | | | |
| site | | | |
| DNA | 253 | MG3- | CCACGTGGAGCTGAGCTGGTGG |
| sequence | | 6/3-4 | |
| of | | TRBC1 | |
| TRBC1 | | B1 | |
| target | | | |
| site | | | |
| DNA | 254 | MG3- | AGTCCAGTTCTACGGGCTCTCG |
| sequence | | 6/3-4 | |
| of | | TRBC1 | |
| TRBC1 | | C1 | |
| target | | | |
| site | | | |
| DNA | 255 | MG3- | GATTAGGTGAGACCAGCTACCA |
| sequence | | 6/3-4 | |
| of | | TRBC1 | |
| TRBC1 | | D1 | |
| target | | | |
| site | | | |
| DNA | 256 | MG3- | ATTAGGTGAGACCAGCTACCAG |
| sequence | | 6/3-4 | |
| of | | TRBC1 | |
| TRBC1 | | E1 | |
| target | | | |
| site | | | |
| DNA | 257 | MG3- | TTAGGTGAGACCAGCTACCAGG |
| sequence | | 6/3-4 | |
| of | | TRBC1 | |
| TRBC1 | | F1 | |
| target | | | |
| site | | | |
| DNA | 258 | MG3- | TGAGACCAGCTACCAGGGAAAA |
| sequence | | 6/3-4 | |
| of | | TRBC1 | |
| TRBC1 | | G1 | |
| target | | | |
| site | | | |
| DNA | 259 | MG3- | CAGGTAGCAGACAAGACTAGAT |
| sequence | | 6/3-4 | |
| of | | TRBC1 | |
| TRBC1 | | H1 | |
| target | | | |
| site | | | |
| DNA | 260 | MG3- | AGGTAGCAGACAAGACTAGATC |
| sequence | | 6/3-4 | |
| of | | TRBC1 | |
| TRBC1 | | A2 | |
| target | | | |
| site | | | |
| DNA | 261 | MG3- | AGCAGACAAGACTAGATCCAAA |
| sequence | | 6/3-4 | |
| of | | TRBC1 | |
| TRBC1 | | B2 | |
| target | | | |
| site | | | |
| DNA | 262 | MG3- | GGAACCAGCGCACACCATGAAG |
| sequence | | 6/3-4 | |
| of | | TRBC1 | |
| TRBC1 | | C2 | |
| target | | | |
| site | | | |
| DNA | 263 | MG3- | GTGGCTGACATCTGCATGGCAG |
| sequence | | 6/3-4 | |
| of | | TRBC1 | |
| TRBC1 | | D2 | |
| target | | | |
| site | | | |
| DNA | 264 | MG3- | GGCCTGGGAGTCTGTGCCAACT |
| sequence | | 6/3-4 | |
| of | | TRBC1 | |
| TRBC1 | | E2 | |
| target | | | |
| site | | | |
| DNA | 265 | MG3- | CTGACTTTACTTTTAATTGCCT |
| sequence | | 6/3-4 | |
| of | | TRBC1 | |
| TRBC1 | | F2 | |
| target | | | |
| site | | | |
| DNA | 266 | MG3- | TGACTTTACTTTTAATTGCCTA |
| sequence | | 6/3-4 | |
| of | | TRBC1 | |
| TRBC1 | | G2 | |
| target | | | |
| site | | | |
| DNA | 267 | MG3- | GACTTTACTTTTAATTGCCTAT |
| sequence | | 6/3-4 | |
| of | | TRBC1 | |
| TRBC1 | | H2 | |
| target | | | |
| site | | | |
| DNA | 268 | MG3- | GGGAAGGAGAAGCTGGAGTCAC |
| sequence | | 6/3-4 | |
| of | | TRBC1 | |
| TRBC1 | | A3 | |
| target | | | |
| site | | | |
| DNA | 269 | MG3- | GGAAGGAGAAGCTGGAGTCACC |
| sequence | | 6/3-4 | |
| of | | TRBC1 | |
| TRBC1 | | B3 | |
| target | | | |
| site | | | |
| DNA | 270 | MG3- | AACTCCTGGCTCTTAATAACCC |
| sequence | | 6/3-4 | |
| of | | TRBC1 | |
| TRBC1 | | C3 | |
| target | | | |
| site | | | |
| DNA | 271 | MG3- | AACTTTCTCTTCTGCAGGTCAA |
| sequence | | 6/3-4 | |
| of | | TRBC1 | |
| TRBC1 | | D3 | |
| target | | | |
| site | | | |
| DNA | 272 | MG3- | ACTCCACTTCCAGGGCTGCCTT |
| sequence | | 6/3-4 | |
| of | | TRBC1 | |
| TRBC1 | | E3 | |
| target | | | |
| site | | | |
| DNA | 273 | MG3- | CTCCACTTCCAGGGCTGCCTTC |
| sequence | | 6/3-4 | |
| of | | TRBC1 | |
| TRBC1 | | F3 | |
| target | | | |
| site | | | |
| DNA | 274 | MG3- | TCCTTTCTCTTGACCTGCAGAA |
| sequence | | 6/3-4 | |
| of | | TRBC1 | |
| TRBC1 | | G3 | |
| target | | | |
| site | | | |
| DNA | 275 | MG3- | AGCCAGGAGTTGTGAGGATTGA |
| sequence | | 6/3-4 | |
| of | | TRBC1 | |
| TRBC1 | | H3 | |
| target | | | |
| site | | | |
| DNA | 276 | MG3- | AGTAGTAGGGCCCATTGACCAC |
| sequence | | 6/3-4 | |
| of | | TRBC1 | |
| TRBC1 | | A4 | |
| target | | | |
| site | | | |
| DNA | 277 | MG3- | TGCAAGTTATCTTCTGAGGCAC |
| sequence | | 6/3-4 | |
| of | | TRBC1 | |
| TRBC1 | | B4 | |
| target | | | |
| site | | | |
| DNA | 278 | MG3- | AGTTATCTTCTGAGGCACCTGA |
| sequence | | 6/3-4 | |
| of | | TRBC1 | |
| TRBC1 | | C4 | |
| target | | | |
| site | | | |
| DNA | 279 | MG3- | GTTATCTTCTGAGGCACCTGAA |
| sequence | | 6/3-4 | |
| of | | TRBC1 | |
| TRBC1 | | D4 | |
| target | | | |
| site | | | |
| DNA | 280 | MG3- | TCAAGAACCATGAGAGAGGGAG |
| sequence | | 6/3-4 | |
| of | | TRBC1 | |
| TRBC1 | | E4 | |
| target | | | |
| site | | | |
| DNA | 281 | MG3- | CAAGAACCATGAGAGAGGGAGA |
| sequence | | 6/3-4 | |
| of | | TRBC1 | |
| TRBC1 | | F4 | |
| target | | | |
| site | | | |
| DNA | 282 | MG3- | TTACCCGAGGTAAAGCCACAGT |
| sequence | | 6/3-4 | |
| of | | TRBC1 | |
| TRBC1 | | G4 | |
| target | | | |
| site | | | |
| DNA | 283 | MG3- | CCGAGGTAAAGCCACAGTCTGA |
| sequence | | 6/3-4 | |
| of | | TRBC1 | |
| TRBC1 | | H4 | |
| target | | | |
| site | | | |
| DNA | 284 | MG3- | CAGTCTGAAAGAAAGCAGGGAG |
| sequence | | 6/3-4 | |
| of | | TRBC1 | |
| TRBC1 | | A5 | |
| target | | | |
| site | | | |
| DNA | 285 | MG3- | AGTCTGAAAGAAAGCAGGGAGA |
| sequence | | 6/3-4 | |
| of | | TRBC1 | |
| TRBC1 | | B5 | |
| target | | | |
| site | | | |
| DNA | 286 | MG3- | GTCTGAAAGAAAGCAGGGAGAG |
| sequence | | 6/3-4 | |
| of | | TRBC1 | |
| TRBC1 | | C5 | |
| target | | | |
| site | | | |
| DNA | 287 | MG3- | GAAAGAAAGCAGGGAGAGGAAA |
| sequence | | 6/3-4 | |
| of | | TRBC1 | |
| TRBC1 | | D5 | |
| target | | | |
| site | | | |
| DNA | 288 | MG3- | GAGACCTTATTTTCATAGGCAA |
| sequence | | 6/3-4 | |
| of | | TRBC1 | |
| TRBC1 | | E5 | |
| target | | | |
| site | | | |
| DNA | 289 | MG3- | GATGAGAGTTACACAGGCCACA |
| sequence | | 6/3-4 | |
| of | | TRBC1 | |
| TRBC1 | | F5 | |
| target | | | |
| site | | | |
| DNA | 290 | MG3- | AGCTGCTTGGCTCTGTTGGGCT |
| sequence | | 6/3-4 | |
| of | | TRBC1 | |
| TRBC1 | | G5 | |
| target | | | |
| site | | | |
| DNA | 291 | MG3- | TGTTGGGCTGAGAATCTGGGAG |
| sequence | | 6/3-4 | |
| of | | TRBC1 | |
| TRBC1 | | H5 | |
| target | | | |
| site | | | |
| DNA | 292 | MG3- | GGAACACCTTGTTCAGGTCCTC |
| sequence | | 6/3-4 | |
| of | | TRBC1 | |
| TRBC1 | | A6 | |
| target | | | |
| site | | | |
| MG3-6/3- | 293 | MG3- | mA*mC*mC*rUrCrUrUrCrCrCrU |
| 4 sgRNA | | 6/3-4 | rUrUrCrCrArGrArGrGrArCrGr |
| targeting | | TRBC2 | UrUrGrArGrArArUrCrGrArArA |
| TRBC2 | | A1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 294 | MG3- | mC*mC*mU*rCrUrUrCrCrCrUrU |
| 4 sgRNA | | 6/3-4 | rUrCrCrArGrArGrGrArCrCrGr |
| targeting | | TRBC2 | UrUrGrArGrArArUrCrGrArArA |
| TRBC2 | | B1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 295 | MG3- | mC*mU*mC*rUrUrCrCrCrUrUrU |
| 4 sgRNA | | 6/3-4 | rCrCrArGrArGrGrArCrCrUrGr |
| targeting | | TRBC2 | UrUrGrArGrArArUrCrGrArArA |
| TRBC2 | | C1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 296 | MG3- | mC*mA*mG*rArArGrCrArGrArG |
| 4 sgRNA | | 6/3-4 | rArUrCrUrCrCrCrArCrArCrGr |
| targeting | | TRBC2 | UrUrGrArGrArArUrCrGrArArA |
| TRBC2 | | D1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 297 | MG3- | mC*mC*mA*rCrGrUrGrGrArGrC |
| 4 sgRNA | | 6/3-4 | rUrGrArGrCrUrGrGrUrGrGrGr |
| targeting | | TRBC2 | UrUrGrArGrArArUrCrGrArArA |
| TRBC2 | | E1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 298 | MG3- | mA*mG*mU*rCrCrArGrUrUrCrU |
| 4 sgRNA | | 6/3-4 | rArCrGrGrGrCrUrCrUrCrGrGr |
| targeting | | TRBC2 | UrUrGrArGrArArUrCrGrArArA |
| TRBC2 | | F1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3 | 299 | MG3- | mG*mA*mU*rUrArGrGrUrGrArG |
| 4 sgRNA | | 6/3-4 | rArCrCrArGrCrUrArCrCrArGr |
| targeting | | TRBC2 | UrUrGrArGrArArUrCrGrArArA |
| TRBC2 | | G1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 300 | MG3- | mA*mU*mU*rArGrGrUrGrArGrA |
| 4 sgRNA | | 6/3-4 | rCrCrArGrCrUrArCrCrArGrGr |
| targeting | | TRBC2 | UrUrGrArGrArArUrCrGrArArA |
| TRBC2 | | H1 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 301 | MG3- | mU*mU*mA*rGrGrUrGrArGrArC |
| 4 sgRNA | | 6/3-4 | rCrArGrCrUrArCrCrArGrGrGr |
| targeting | | TRBC2 | UrUrGrArGrArArUrCrGrArArA |
| TRBC2 | | A2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 302 | MG3- | mUmG*mA*rGrArCrCrArGrCrUr |
| 4 sgRNA | | 6/3-4 | ArCrCrArGrGrGrArArArArGrU |
| targeting | | TRBC2 | rUrGrArGrArArUrCrGrArArAr |
| TRBC2 | | B2 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 303 | MG3- | mU*mA*mG*rCrGrGrArCrArArG |
| 4 sgRNA | | 6/3-4 | rArCrUrArGrArUrCrCrArGrGr |
| targeting | | TRBC2 | UrUrGrArGrArArUrCrGrArArA |
| TRBC2 | | C2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 304 | MG3- | mC*mC*mC*rCrCrArCrCrArArG |
| 4 sgRNA | | 6/3-4 | rArArGrCrArUrArGrArGrGrGr |
| targeting | | TRBC2 | UrUrGrArGrArArUrCrGrArArA |
| TRBC2 | | D2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 305 | MG3- | mUmC*mU*rGrCrUrCrUrCrGrAr |
| 4 sgRNA | | 6/3-4 | ArCrCrArGrGrGrCrArUrGrGrU |
| targeting | | TRBC2 | rUrGrArGrArArUrCrGrArArAr |
| TRBC2 | | E2 | GrArUrUrCrUrUrArArUrArArG |
| | | | rGrCrArUrCrCrUrUrCrCrGrAr |
| | | | UrGrCrUrGrArCrUrUrCrUrCrA |
| | | | rCrCrGrUrCrCrGrUrUrUrUrCr |
| | | | CrArArUrArGrGrArGrCrGrGrG |
| | | | rCrGrGrUrArUrGrU*mU*mU*mU |
| MG3-6/3- | 306 | MG3- | mG*mG*mA*rArCrArUrCrArCrA |
| 4 sgRNA | | 6/3-4 | rCrArUrGrGrGrCrArUrArArGr |
| targeting | | TRBC2 | UrUrGrArGrArArUrCrGrArArA |
| TRBC2 | | F2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 307 | MG3- | mC*mC*mU*rArArUrArUrArUrC |
| 4 sgRNA | | 6/3-4 | rCrUrArUrCrArCrCrUrCrArGr |
| targeting | | TRBC2 | UrUrGrArGrArArUrCrGrArArA |
| TRBC2 | | G2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 308 | MG3- | mA*mC*mC*rArUrArArUrGrArA |
| 4 sgRNA | | 6/3-4 | rGrCrCrArGrArCrUrGrGrGrGr |
| targeting | | TRBC2 | UrUrGrArGrArArUrCrGrArArA |
| TRBC2 | | H2 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 309 | MG3- | mC*mC*mA*rUrArArUrGrArArG |
| 4 sgRNA | | 6/3-4 | rCrCrArGrArCrUrGrGrGrGrGr |
| targeting | | TRBC2 | UrUrGrArGrArArUrCrGrArArA |
| TRBC2 | | A3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 310 | MG3- | mC*mA*mU*rArArUrGrArArGrC |
| 4 sgRNA | | 6/3-4 | rCrArGrArCrUrGrGrGrGrArGr |
| targeting | | TRBC2 | UrUrGrArGrArArUrCrGrArArA |
| TRBC2 | | B3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 311 | MG3- | mG*mC*mC*rArGrArCrUrGrGrG |
| 4 sgRNA | | 6/3-4 | rGrArGrArArArArUrGrCrArGr |
| targeting | | TRBC2 | UrUrGrArGrArArUrCrGrArArA |
| TRBC2 | | C3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 312 | MG3- | mG*mG*mA*rGrArArArArUrGrC |
| 4 sgRNA | | 6/3-4 | rArGrGrGrArArUrArUrCrArGr |
| targeting | | TRBC2 | UrUrGrArGrArArUrCrGrArArA |
| TRBC2 | | D3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 313 | MG3- | mG*mG*mA*rGrArCrArArCrCrA |
| 4 sgRNA | | 6/3-4 | rGrCrGrArGrCrCrCrUrArCrGr |
| targeting | | TRBC2 | UrUrGrArGrArArUrCrGrArArA |
| TRBC2 | | E3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 314 | MG3- | mU*mA*mC*rUrCrCrUrGrCrUrG |
| 4 sgRNA | | 6/3-4 | rUrGrCrCrArUrArGrCrCrCrGr |
| targeting | | TRBC2 | UrUrGrArGrArArUrCrGrArArA |
| TRBC2 | | F3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 315 | MG3- | mC*mU*mG*rUrGrCrCrArUrArG |
| 4 sgRNA | | 6/3-4 | rCrCrCrCrUrGrArArArCrCrGr |
| targeting | | TRBC2 | UrUrGrArGrArArUrCrGrArArA |
| TRBC2 | | G3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 316 | MG3- | mU*mG*mU*rGrCrCrArUrArGrC |
| 4 sgRNA | | 6/3-4 | rCrCrCrUrGrArArArCrCrCrGr |
| targeting | | TRBC2 | UrUrGrArGrArArUrCrGrArArA |
| TRBC2 | | H3 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 317 | MG3- | mG*mU*mG*rCrCrArUrArGrCrC |
| 4 sgRNA | | 6/3-4 | rCrCrUrGrArArArCrCrCrUrGr |
| targeting | | TRBC2 | UrUrGrArGrArArUrCrGrArArA |
| TRBC2 | | A4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 318 | MG3- | mU*mG*mU*rUrCrUrCrUrCrUrU |
| 4 sgRNA | | 6/3-4 | rCrCrArCrArGrGrUrCrArArGr |
| targeting | | TRBC2 | UrUrGrArGrArArUrCrGrArArA |
| TRBC2 | | B4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 319 | MG3- | mG*mA*mA*rArGrGrArUrUrCrC |
| 4 sgRNA | | 6/3-4 | rArGrArGrGrCrUrArGrCrUrGr |
| targeting | | TRBC2 | UrUrGrArGrArArUrCrGrArArA |
| TRBC2 | | C4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 320 | MG3- | mG*mG*mA*rUrGrGrUrUrUrUrG |
| 4 sgRNA | | 6/3-4 | rGrArGrCrUrArGrCrCrUrCrGr |
| targeting | | TRBC2 | UrUrGrArGrArArUrCrGrArArA |
| TRBC2 | | D4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 321 | MG3- | mC*mC*mC*rUrGrGrUrUrCrGrA |
| 4 sgRNA | | 6/3-4 | rGrArGrCrArGrArGrArCrGrGr |
| targeting | | TRBC2 | UrUrGrArGrArArUrCrGrArArA |
| TRBC2 | | E4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrU |
| | | | rCrCrGrUrUrUrUrCrCrArArUr |
| | | | ArGrGrArGrCrGrGrGrCrGrGrU |
| | | | rArUrGrU*mU*mU*mU |
| MG3-6/3- | 322 | MG3- | mA*mG*mC*rArGrArGrArCrGrG |
| 4 sgRNA | | 6/3-4 | rCrGrArArArGrArUrArGrArGr |
| targeting | | TRBC2 | UrUrGrArGrArArUrCrGrArArA |
| TRBC2 | | F4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 323 | MG3- | mG*mC*mA*rGrArGrArCrGrGrC |
| 4 sgRNA | | 6/3-4 | rGrArArArGrArUrArGrArGrGr |
| targeting | | TRBC2 | UrUrGrArGrArArUrCrGrArArA |
| TRBC2 | | G4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 324 | MG3- | mC*mA*mG*rArGrArCrGrGrCrG |
| 4 sgRNA | | 6/3-4 | rArArArGrArUrArGrArGrArGr |
| targeting | | TRBC2 | UrUrGrArGrArArUrCrGrArArA |
| TRBC2 | | H4 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 325 | MG3- | mU*mU*mA*rCrCrGrGrArGrGrU |
| 4 sgRNA | | 6/3-4 | rGrArArGrCrCrArCrArGrUrGr |
| targeting | | TRBC2 | UrUrGrArGrArArUrCrGrArArA |
| TRBC2 | | A5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 326 | MG3- | mC*mG*mG*rArGrGrUrGrArArG |
| 4 sgRNA | | 6/3-4 | rCrCrArCrArGrUrCrUrGrArGr |
| targeting | | TRBC2 | UrUrGrArGrArArUrCrGrArArA |
| TRBC2 | | B5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 327 | MG3- | mG*mG*mA*rGrGrUrGrArArGrC |
| 4 sgRNA | | 6/3-4 | rCrArCrArGrUrCrUrGrArArGr |
| targeting | | TRBC2 | UrUrGrArGrArArUrCrGrArArA |
| TRBC2 | | C5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 328 | MG3- | mA*mC*mA*rGrUrCrUrGrArArA |
| 4 sgRNA | | 6/3-4 | rGrArArArArCrArGrGrGrGrGr |
| targeting | | TRBC2 | UrUrGrArGrArArUrCrGrArArA |
| TRBC2 | | D5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 329 | MG3- | mC*mA*mG*rUrCrUrGrArArArG |
| 4 sgRNA | | 6/3-4 | rArArArArCrArGrGrGrGrArGr |
| targeting | | TRBC2 | UrUrGrArGrArArUrCrGrArArA |
| TRBC2 | | E5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 330 | MG3- | mA*mG*mU*rCrUrGrArArArGrA |
| 4 sgRNA | | 6/3-4 | rArArArCrArGrGrGrGrArArGr |
| targeting | | TRBC2 | UrUrGrArGrArArUrCrGrArArA |
| TRBC2 | | F5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 331 | MG3- | mG*mU*mC*rUrGrArArArGrArA |
| 4 sgRNA | | 6/3-4 | rArArCrArGrGrGrGrArArGrGr |
| targeting | | TRBC2 | UrUrGrArGrArArUrCrGrArArA |
| TRBC2 | | G5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 332 | MG3- | mA*mC*mA*rGrGrGrGrArArGrA |
| 4 sgRNA | | 6/3-4 | rArArArArUrGrGrArUrGrArGr |
| targeting | | TRBC2 | UrUrGrArGrArArUrCrGrArArA |
| TRBC2 | | H5 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 333 | MG3- | mG*mC*mG*rArArGrUrGrGrUrC |
| 4 sgRNA | | 6/3-4 | rArCrUrArUrGrArUrCrUrUrGr |
| targeting | | TRBC2 | UrUrGrArGrArArUrCrGrArArA |
| TRBC2 | | A6 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 334 | MG3- | mUmUmA*rGrGrArArArCrCrArG |
| 4 sgRNA | | 6/3-4 | rGrArCrCrCrCrArGrArArGrUr |
| targeting | | TRBC2 | UrGrArGrArArUrCrGrArArArG |
| TRBC2 | | B6 | rArUrUrCrUrUrArArUrArArGr |
| | | | GrCrArUrCrCrUrUrCrCrGrArU |
| | | | rGrCrUrGrArCrUrUrCrUrCrAr |
| | | | CrCrGrUrCrCrGrUrUrUrUrCrC |
| | | | rArArUrArGrGrArGrCrGrGrGr |
| | | | CrGrGrUrArUrGrU*mU*mU*mU |
| MG3-6/3- | 335 | MG3- | mU*mA*mU*rGrGrCrUrGrGrUrC |
| 4 sgRNA | | 6/3-4 | rCrUrCrArGrGrGrArGrArCrGr |
| targeting | | TRBC2 | UrUrGrArGrArArUrCrGrArArA |
| TRBC2 | | C6 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 336 | MG3- | mC*mU*mA*rArGrGrUrGrUrCrA |
| 4 sgRNA | | 6/3-4 | rGrGrArUrCrUrGrArArGrGrGr |
| targeting | | TRBC2 | UrUrGrArGrArArUrCrGrArArA |
| TRBC2 | | D6 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| MG3-6/3- | 337 | MG3- | mG*mG*mA*rArCrArCrGrUrUrU |
| 4 sgRNA | | 6/3-4 | rUrUrCrArGrGrUrCrCrUrCrGr |
| targeting | | TRBC2 | UrUrGrArGrArArUrCrGrArArA |
| TRBC2 | | E6 | rGrArUrUrCrUrUrArArUrArAr |
| | | | GrGrCrArUrCrCrUrUrCrCrGrA |
| | | | rUrGrCrUrGrArCrUrUrCrUrCr |
| | | | ArCrCrGrUrCrCrGrUrUrUrUrC |
| | | | rCrArArUrArGrGrArGrCrGrGr |
| | | | GrCrGrGrUrArUrGrU*mU*mU*m |
| | | | U |
| DNA | 338 | MG3- | ACCTCTTCCCTTTCCAGAGGAC |
| sequence | | 6/3-4 | |
| of | | TRBC2 | |
| TRBC2 | | A1 | |
| target | | | |
| site | | | |
| DNA | 339 | MG3- | CCTCTTCCCTTTCCAGAGGACC |
| sequence | | 6/3-4 | |
| of | | TRBC2 | |
| TRBC2 | | B1 | |
| target | | | |
| site | | | |
| DNA | 340 | MG3- | CTCTTCCCTTTCCAGAGGACCT |
| sequence | | 6/3-4 | |
| of | | TRBC2 | |
| TRBC2 | | C1 | |
| target | | | |
| site | | | |
| DNA | 341 | MG3- | CAGAAGCAGAGATCTCCCACAC |
| sequence | | 6/3-4 | |
| of | | TRBC2 | |
| TRBC2 | | D1 | |
| target | | | |
| site | | | |
| DNA | 342 | MG3- | CCACGTGGAGCTGAGCTGGTGG |
| sequence | | 6/3-4 | |
| of | | TRBC2 | |
| TRBC2 | | E1 | |
| target | | | |
| site | | | |
| DNA | 343 | MG3- | AGTCCAGTTCTACGGGCTCTCG |
| sequence | | 6/3-4 | |
| of | | TRBC2 | |
| TRBC2 | | F1 | |
| target | | | |
| site | | | |
| DNA | 344 | MG3- | GATTAGGTGAGACCAGCTACCA |
| sequence | | 6/3-4 | |
| of | | TRBC2 | |
| TRBC2 | | G1 | |
| target | | | |
| site | | | |
| DNA | 345 | MG3- | ATTAGGTGAGACCAGCTACCAG |
| sequence | | 6/3-4 | |
| of | | TRBC2 | |
| TRBC2 | | H1 | |
| target | | | |
| site | | | |
| DNA | 346 | MG3- | TTAGGTGAGACCAGCTACCAGG |
| sequence | | 6/3-4 | |
| of | | TRBC2 | |
| TRBC2 | | A2 | |
| target | | | |
| site | | | |
| DNA | 347 | MG3- | TGAGACCAGCTACCAGGGAAAA |
| sequence | | 6/3-4 | |
| of | | TRBC2 | |
| TRBC2 | | B2 | |
| target | | | |
| site | | | |
| DNA | 348 | MG3- | TAGCGGACAAGACTAGATCCAG |
| sequence | | 6/3-4 | |
| of | | TRBC2 | |
| TRBC2 | | C2 | |
| target | | | |
| site | | | |
| DNA | 349 | MG3- | CCCCCACCAAGAAGCATAGAGG |
| sequence | | 6/3-4 | |
| of | | TRBC2 | |
| TRBC2 | | D2 | |
| target | | | |
| site | | | |
| DNA | 350 | MG3- | TCTGCTCTCGAACCAGGGCATG |
| sequence | | 6/3-4 | |
| of | | TRBC2 | |
| TRBC2 | | E2 | |
| target | | | |
| site | | | |
| DNA | 351 | MG3- | GGAACATCACACATGGGCATAA |
| sequence | | 6/3-4 | |
| of | | TRBC2 | |
| TRBC2 | | F2 | |
| target | | | |
| site | | | |
| DNA | 352 | MG3- | CCTAATATATCCTATCACCTCA |
| sequence | | 6/3-4 | |
| of | | TRBC2 | |
| TRBC2 | | G2 | |
| target | | | |
| site | | | |
| DNA | 353 | MG3- | ACCATAATGAAGCCAGACTGGG |
| sequence | | 6/3-4 | |
| of | | TRBC2 | |
| TRBC2 | | H2 | |
| target | | | |
| site | | | |
| DNA | 354 | MG3- | CCATAATGAAGCCAGACTGGGG |
| sequence | | 6/3-4 | |
| of | | TRBC2 | |
| TRBC2 | | A3 | |
| target | | | |
| site | | | |
| DNA | 355 | MG3- | CATAATGAAGCCAGACTGGGGA |
| sequence | | 6/3-4 | |
| of | | TRBC2 | |
| TRBC2 | | B3 | |
| target | | | |
| site | | | |
| DNA | 356 | MG3- | GCCAGACTGGGGAGAAAATGCA |
| sequence | | 6/3-4 | |
| of | | TRBC2 | |
| TRBC2 | | C3 | |
| target | | | |
| site | | | |
| DNA | 357 | MG3- | GGAGAAAATGCAGGGAATATCA |
| sequence | | 6/3-4 | |
| of | | TRBC2 | |
| TRBC2 | | D3 | |
| target | | | |
| site | | | |
| DNA | 358 | MG3- | GGAGACAACCAGCGAGCCCTAC |
| sequence | | 6/3-4 | |
| of | | TRBC2 | |
| TRBC2 | | E3 | |
| target | | | |
| site | | | |
| DNA | 359 | MG3- | TACTCCTGCTGTGCCATAGCCC |
| sequence | | 6/3-4 | |
| of | | TRBC2 | |
| TRBC2 | | F3 | |
| target | | | |
| site | | | |
| DNA | 360 | MG3- | CTGTGCCATAGCCCCTGAAACC |
| sequence | | 6/3-4 | |
| of | | TRBC2 | |
| TRBC2 | | G3 | |
| target | | | |
| site | | | |
| DNA | 361 | MG3- | TGTGCCATAGCCCCTGAAACCC |
| sequence | | 6/3-4 | |
| of | | TRBC2 | |
| TRBC2 | | H3 | |
| target | | | |
| site | | | |
| DNA | 362 | MG3- | GTGCCATAGCCCCTGAAACCCT |
| sequence | | 6/3-4 | |
| of | | TRBC2 | |
| TRBC2 | | A4 | |
| target | | | |
| site | | | |
| DNA | 363 | MG3- | TGTTCTCTCTTCCACAGGTCAA |
| sequence | | 6/3-4 | |
| of | | TRBC2 | |
| TRBC2 | | B4 | |
| target | | | |
| site | | | |
| DNA | 364 | MG3- | GAAAGGATTCCAGAGGCTAGCT |
| sequence | | 6/3-4 | |
| of | | TRBC2 | |
| TRBC2 | | C4 | |
| target | | | |
| site | | | |
| DNA | 365 | MG3- | GGATGGTTTTGGAGCTAGCCTC |
| sequence | | 6/3-4 | |
| of | | TRBC2 | |
| TRBC2 | | D4 | |
| target | | | |
| site | | | |
| DNA | 366 | MG3- | CCCTGGTTCGAGAGCAGAGACG |
| sequence | | 6/3-4 | |
| of | | TRBC2 | |
| TRBC2 | | E4 | |
| target | | | |
| site | | | |
| DNA | 367 | MG3- | AGCAGAGACGGCGAAAGATAGA |
| sequence | | 6/3-4 | |
| of | | TRBC2 | |
| TRBC2 | | F4 | |
| target | | | |
| site | | | |
| DNA | 368 | MG3- | GCAGAGACGGCGAAAGATAGAG |
| sequence | | 6/3-4 | |
| of | | TRBC2 | |
| TRBC2 | | G4 | |
| target | | | |
| site | | | |
| DNA | 369 | MG3- | CAGAGACGGCGAAAGATAGAGA |
| sequence | | 6/3-4 | |
| of | | TRBC2 | |
| TRBC2 | | H4 | |
| target | | | |
| site | | | |
| DNA | 370 | MG3- | TTACCGGAGGTGAAGCCACAGT |
| sequence | | 6/3-4 | |
| of | | TRBC2 | |
| TRBC2 | | A5 | |
| target | | | |
| site | | | |
| DNA | 371 | MG3- | CGGAGGTGAAGCCACAGTCTGA |
| sequence | | 6/3-4 | |
| of | | TRBC2 | |
| TRBC2 | | B5 | |
| target | | | |
| site | | | |
| DNA | 372 | MG3- | GGAGGTGAAGCCACAGTCTGAA |
| sequence | | 6/3-4 | |
| of | | TRBC2 | |
| TRBC2 | | C5 | |
| target | | | |
| site | | | |
| DNA | 373 | MG3- | ACAGTCTGAAAGAAAACAGGGG |
| sequence | | 6/3-4 | |
| of | | TRBC2 | |
| TRBC2 | | D5 | |
| target | | | |
| site | | | |
| DNA | 374 | MG3- | CAGTCTGAAAGAAAACAGGGGA |
| sequence | | 6/3-4 | |
| of | | TRBC2 | |
| TRBC2 | | E5 | |
| target | | | |
| site | | | |
| DNA | 375 | MG3- | AGTCTGAAAGAAAACAGGGGAA |
| sequence | | 6/3-4 | |
| of | | TRBC2 | |
| TRBC2 | | F5 | |
| target | | | |
| site | | | |
| DNA | 376 | MG3- | GTCTGAAAGAAAACAGGGGAAG |
| sequence | | 6/3-4 | |
| of | | TRBC2 | |
| TRBC2 | | G5 | |
| target | | | |
| site | | | |
| DNA | 377 | MG3- | ACAGGGGAAGAAAAATGGATGA |
| sequence | | 6/3-4 | |
| of | | TRBC2 | |
| TRBC2 | | H5 | |
| target | | | |
| site | | | |
| DNA | 378 | MG3- | GCGAAGTGGTCACTATGATCTT |
| sequence | | 6/3-4 | |
| of | | TRBC2 | |
| TRBC2 | | A6 | |
| target | | | |
| site | | | |
| DNA | 379 | MG3- | TTAGGAAACCAGGACCCCAGAA |
| sequence | | 6/3-4 | |
| of | | TRBC2 | |
| TRBC2 | | B6 | |
| target | | | |
| site | | | |
| DNA | 380 | MG3- | TATGGCTGGTCCTCAGGGAGAC |
| sequence | | 6/3-4 | |
| of | | TRBC2 | |
| TRBC2 | | C6 | |
| target | | | |
| site | | | |
| DNA | 381 | MG3- | CTAAGGTGTCAGGATCTGAAGG |
| sequence | | 6/3-4 | |
| of | | TRBC2 | |
| TRBC2 | | D6 | |
| target | | | |
| site | | | |
| DNA | 382 | MG3- | GGAACACGTTTTTCAGGTCCTC |
| sequence | | 6/3-4 | |
| of | | TRBC2 | |
| TRBC2 | | E6 | |
| target | | | |
| site | | | |

(r = native ribose base, m = 2'-O methyl modified base, F = 2' Fluro modified base, * = phosphorothioate bond)

### Example 16—Analysis of Gene-Editing Outcomes at the DNA Level for ANGPTL3 in Hep3B Cells

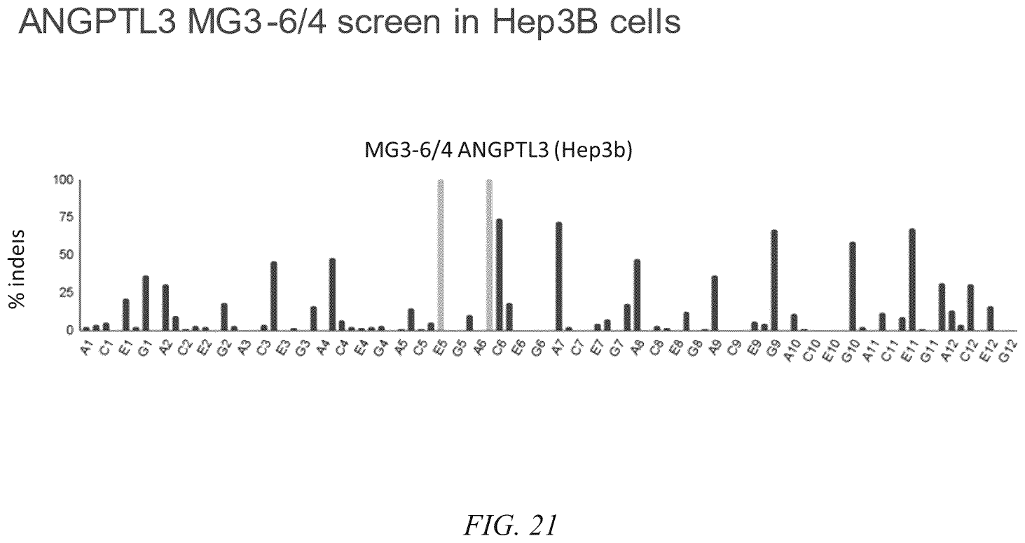

Nucleofection of MG3-6/4 RNPs (104 pmol protein/120 pmol guide) comprising sgRNAs described below in Table 9D below and SEQ ID NOs: 383-572 was performed into Hep3B cells (100,000) using electroporation. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed determine gene editing (FIG. 21). The results show that sgRNA E5, C6, A7, A8, A9, G9, G10, Eli, A12, and C12 are the highest performing sgRNAs in this assay.

TABLE 9D
gRNAs and Targeting Sequences Used in Example 16

| | SEQ | | |
|---|---|---|---|
| | ID | | |
| Category | NO: | Name | Sequence |
| MG3-6/3- | 383 | MG3- | mU*mU*mG*rUrUrCrCrUrCrUrArGrUrUrArUrUrUrCrCrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A1 | rCrCrGrUrUrUrUrCrCrArArUrArGrGrArGrCrGrGrGrCrGrGrUrAr |
| | | | UrGrU*mU*mU*mU |
| MG3-6/3- | 384 | MG3- | mA*mU*mU*rUrGrArUrUrCrUrCrUrArUrCrUrCrCrArGrArGrGrUrU |
| 4 sgRNA | | 6/3-4 | rGrArGrArArUrCrGrArArArGrArUrUrCrUrUrArArUrArArGrGrCr |
| targeting | | ANGPTL | ArUrCrCrUrUrCrCrGrArUrGrCrUrGrArCrUrUrCrUrCrArCrCrGrU |
| ANGPTL3 | | B1 | rCrCrGrUrUrUrUrCrCrArArUrArGrGrArGrCrGrGrGrCrGrGrUrAr |
| | | | UrGrU*mU*mU*mU |
| MG3-6/3- | 385 | MG3- | mU*mU*mU*rGrArUrUrCrUrCrUrArUrCrUrCrCrArGrArGrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C1 | rCrCrGrUrUrUrUrCrCrArArUrArGrGrArGrCrGrGrGrCrGrGrUrAr |
| | | | UrGrU*mU*mU*mU |
| MG3-6/3- | 386 | MG3- | mA*mA*mG*rArUrUrUrGrCrUrArUrGrUrUrArGrArCrGrArUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D1 | rCrCrGrUrUrUrUrCrCrArArUrArGrGrArGrCrGrGrGrCrGrGrUrAr |
| | | | UrGrU*mU*mU*mU |
| MG3-6/3- | 387 | MG3- | mA*mG*mA*rUrUrUrGrCrUrArUrGrUrUrArGrArCrGrArUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E1 | rCrCrGrUrUrUrUrCrCrArArUrArGrGrArGrCrGrGrGrCrGrGrUrAr |
| | | | UrGrU*mU*mU*mU |
| MG3-6/3 | 388 | MG3- | mG*mA*mU*rUrUrGrCrUrArUrGrUrUrArGrArCrGrArUrGrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F1 | rCrCrGrUrUrUrUrCrCrArArUrArGrGrArGrCrGrGrGrCrGrGrUrAr |
| | | | UrGrU*mU*mU*mU |
| MG3-6/3- | 389 | MG3- | mA*mC*mU*rUrUrGrUrCrCrArUrArArGrArCrGrArArGrGrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G1 | rCrCrGrUrUrUrUrCrCrArArUrArGrGrArGrCrGrGrGrCrGrGrUrAr |
| | | | UrGrU*mU*mU*mU |
| MG3-6/3- | 390 | MG3- | mA*mG*mG*rGrCrCrArArArUrUrArArUrGrArCrArUrArUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H1 | rCrCrGrUrUrUrUrCrCrArArUrArGrGrArGrCrGrGrGrCrGrGrUrAr |
| | | | UrGrU*mU*mU*mU |
| MG3-6/3- | 391 | MG3- | mG*mG*mG*rCrCrArArArUrUrArArUrGrArCrArUrArUrUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A2 | rCrCrGrUrUrUrUrCrCrArArUrArGrGrArGrCrGrGrGrCrGrGrUrAr |
| | | | UrGrU*mU*mU*mU |
| MG3-6/3- | 392 | MG3- | mU*mA*mU*rGrArUrCrUrArUrCrGrCrUrGrCrArArArCrCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B2 | rCrCrGrUrUrUrUrCrCrArArUrArGrGrArGrCrGrGrGrCrGrGrUrAr |
| | | | UrGrU*mU*mU*mU |
| MG3-6/3- | 393 | MG3- | mA*mU*mG*rArUrCrUrArUrCrGrCrUrGrCrArArArCrCrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C2 | rCrCrGrUrUrUrUrCrCrArArUrArGrGrArGrCrGrGrGrCrGrGrUrAr |
| | | | UrGrU*mU*mU*mU |
| MG3-6/3- | 394 | MG3- | mC*mA*mA*rArCrCrArGrUrGrArArArUrCrArArArGrArArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D2 | rCrCrGrUrUrUrUrCrCrArArUrArGrGrArGrCrGrGrGrCrGrGrUrAr |
| | | | UrGrU*mU*mU*mU |
| MG3-6/3- | 395 | MG3- | mA*mA*mA*rCrCrArGrUrGrArArArUrCrArArArGrArArGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E2 | rCrCrGrUrUrUrUrCrCrArArUrArGrGrArGrCrGrGrGrCrGrGrUrAr |
| | | | UrGrU*mU*mU*mU |
| MG3-6/3- | 396 | MG3- | mA*mC*mA*rArGrUrCrArArArArArUrGrArArGrArGrGrUrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F2 | rCrCrGrUrUrUrUrCrCrArArUrArGrGrArGrCrGrGrGrCrGrGrUrAr |
| | | | UrGrU*mU*mU*mU |
| MG3-6/3- | 397 | MG3- | mG*mA*mA*rUrArUrGrUrCrArCrUrUrGrArArCrUrCrArArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G2 | rCrCrGrUrUrUrUrCrCrArArUrArGrGrArGrCrGrGrGrCrGrGrUrAr |
| | | | UrGrU*mU*mU*mU |
| MG3-6/3- | 398 | MG3- | mU*mC*mA*rCrUrUrGrArArCrUrCrArArCrUrCrArArArArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H2 | rCrCrGrUrUrUrUrCrCrArArUrArGrGrArGrCrGrGrGrCrGrGrUrAr |
| | | | UrGrU*mU*mU*mU |
| MG3-6/3- | 399 | MG3- | mU*mC*mA*rArArArCrUrUrGrArArArGrCrCrUrCrCrUrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A3 | rCrCrGrUrUrUrUrCrCrArArUrArGrGrArGrCrGrGrGrCrGrGrUrAr |
| | | | UrGrU*mU*mU*mU |
| MG3-6/3- | 400 | MG3- | mC*mA*mA*rArArCrUrUrGrArArArGrCrCrUrCrCrUrArGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B3 | rCrCrGrUrUrUrUrCrCrArArUrArGrGrArGrCrGrGrGrCrGrGrUrAr |
| | | | UrGrU*mU*mU*mU |
| MG3-6/3- | 401 | MG3- | mA*mA*mA*rArCrUrUrGrArArArGrCrCrUrCrCrUrArGrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C3 | rCrCrGrUrUrUrUrCrCrArArUrArGrGrArGrCrGrGrGrCrGrGrUrAr |
| | | | UrGrU*mU*mU*mU |
| MG3-6/3- | 402 | MG3- | mA*mA*mA*rCrUrUrGrArArArGrCrCrUrCrCrUrArGrArArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D3 | rCrCrGrUrUrUrUrCrCrArArUrArGrGrArGrCrGrGrGrCrGrGrUrAr |
| | | | UrGrU*mU*mU*mU |
| MG3-6/3- | 403 | MG3- | mA*mA*mC*rUrUrGrArArArGrCrCrUrCrCrUrArGrArArGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E3 | rCrCrGrUrUrUrUrCrCrArArUrArGrGrArGrCrGrGrGrCrGrGrUrAr |
| | | | UrGrU*mU*mU*mU |
| MG3-6/3- | 404 | MG3- | mG*mU*mU*rCrUrGrGrArGrUrUrUrCrArGrGrUrUrGrArUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F3 | rCrCrGrUrUrUrUrCrCrArArUrArGrGrArGrCrGrGrGrCrGrGrUrAr |
| | | | UrGrU*mU*mU*mU |
| MG3-6/3- | 405 | MG3- | mC*mA*mC*rUrGrGrUrUrUrGrCrArGrCrGrArUrArGrArUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G3 | rCrCrGrUrUrUrUrCrCrArArUrArGrGrArGrCrGrGrGrCrGrGrUrAr |
| | | | UrGrU*mU*mU*mU |
| MG3-6/3- | 406 | MG3- | mA*mC*mU*rGrGrUrUrUrGrCrArGrCrGrArUrArGrArUrCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H3 | rCrCrGrUrUrUrUrCrCrArArUrArGrGrArGrCrGrGrGrCrGrGrUrAr |
| | | | UrGrU*mU*mU*mU |
| MG3-6/3- | 407 | MG3- | mC*mG*mA*rUrArGrArUrCrArUrArArArArArGrArCrUrGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A4 | rCrCrGrUrUrUrUrCrCrArArUrArGrGrArGrCrGrGrGrCrGrGrUrAr |
| | | | UrGrU*mU*mU*mU |
| MG3-6/3- | 408 | MG3- | mC*mC*mC*rArArCrUrGrArArGrGrArGrGrCrCrArUrUrGrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B4 | rCrCrGrUrUrUrUrCrCrArArUrArGrGrArGrCrGrGrGrCrGrGrUrAr |
| | | | UrGrU*mU*mU*mU |
| MG3-6/3- | 409 | MG3- | mC*mC*mA*rArCrUrGrArArGrGrArGrGrCrCrArUrUrGrGrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C4 | rCrCrGrUrUrUrUrCrCrArArUrArGrGrArGrCrGrGrGrCrGrGrUrAr |
| | | | UrGrU*mU*mU*mU |
| MG3-6/3- | 410 | MG3- | mC*mU*mU*rGrArUrUrUrUrGrGrCrUrCrUrGrGrArGrArUrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D4 | rCrCrGrUrUrUrUrCrCrArArUrArGrGrArGrCrGrGrGrCrGrGrUrAr |
| | | | UrGrU*mU*mU*mU |
| MG3-6/3- | 411 | MG3- | mU*mU*mU*rUrGrGrCrUrCrUrGrGrArGrArUrArGrArGrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E4 | rCrCrGrUrUrUrUrCrCrArArUrArGrGrArGrCrGrGrGrCrGrGrUrAr |
| | | | UrGrU*mU*mU*mU |
| MG3-6/3- | 412 | MG3- | mU*mC*mU*rGrGrArGrArUrArGrArGrArArUrCrArArArUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F4 | rCrCrGrUrUrUrUrCrCrArArUrArGrGrArGrCrGrGrGrCrGrGrUrAr |
| | | | UrGrU*mU*mU*mU |
| MG3-6/3- | 413 | MG3- | mG*mA*mA*rUrUrGrUrCrUrUrGrArUrCrArArUrUrCrUrGrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G4 | rCrCrGrUrUrUrUrCrCrArArUrArGrGrArGrCrGrGrGrCrGrGrUrAr |
| | | | UrGrU*mU*mU*mU |
| MG3-6/3- | 414 | MG3- | mA*mA*mU*rUrGrUrCrUrUrGrArUrCrArArUrUrCrUrGrGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H4 | rCrCrGrUrUrUrUrCrCrArArUrArGrGrArGrCrGrGrGrCrGrGrUrAr |
| | | | UrGrU*mU*mU*mU |
| MG3-6/3- | 415 | MG3- | mG*mG*mA*rGrGrArArArUrArArCrUrArGrArGrGrArArCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A5 | rCrCrGrUrUrUrUrCrCrArArUrArGrGrArGrCrGrGrGrCrGrGrUrAr |
| | | | UrGrU*mU*mU*mU |
| MG3-6/3- | 416 | MG3- | mG*mA*mG*rGrArArArUrArArCrUrArGrArGrGrArArCrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B5 | rCrCrGrUrUrUrUrCrCrArArUrArGrGrArGrCrGrGrGrCrGrGrUrAr |
| | | | UrGrU*mU*mU*mU |
| MG3-6/3- | 417 | MG3- | mA*mC*mU*rCrUrCrUrArUrArUrCrCrArGrArCrUrUrUrUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C5 | rCrCrGrUrUrUrUrCrCrArArUrArGrGrArGrCrGrGrGrCrGrGrUrAr |
| | | | UrGrU*mU*mU*mU |
| MG3-6/3- | 418 | MG3- | mC*mU*mC*rUrCrUrArUrArUrCrCrArGrArCrUrUrUrUrGrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D5 | rCrCrGrUrUrUrUrCrCrArArUrArGrGrArGrCrGrGrGrCrGrGrUrAr |
| | | | UrGrU*mU*mU*mU |
| MG3-6/3- | 419 | MG3- | mU*mC*mU*rCrUrArUrArUrCrCrArGrArCrUrUrUrUrGrUrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E5 | rCrCrGrUrUrUrUrCrCrArArUrArGrGrArGrCrGrGrGrCrGrGrUrAr |
| | | | UrGrU*mU*mU*mU |
| MG3-6/3- | 420 | MG3- | mA*mA*mC*rArArUrUrArArArCrCrArArCrArGrCrArUrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F5 | rCrCrGrUrUrUrUrCrCrArArUrArGrGrArGrCrGrGrGrCrGrGrUrAr |
| | | | UrGrU*mU*mU*mU |
| MG3-6/3- | 421 | MG3- | mA*mU*mU*rArArArCrCrArArCrArGrCrArUrArGrUrCrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G5 | rCrCrGrUrUrUrUrCrCrArArUrArGrGrArGrCrGrGrGrCrGrGrUrAr |
| | | | UrGrU*mU*mU*mU |
| MG3-6/3- | 422 | MG3- | mA*mA*mC*rCrArArCrArGrCrArUrArGrUrCrArArArUrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H5 | rCrCrGrUrUrUrUrCrCrArArUrArGrGrArGrCrGrGrGrCrGrGrUrAr |
| | | | UrGrU*mU*mU*mU |
| MG3-6/3- | 423 | MG3- | mA*mC*mC*rArArCrArGrCrArUrArGrUrCrArArArUrArArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A6 | rCrCrGrUrUrUrUrCrCrArArUrArGrGrArGrCrGrGrGrCrGrGrUrAr |
| | | | UrGrU*mU*mU*mU |
| MG3-6/3- | 424 | MG3- | mG*mA*mU*rGrCrUrArUrUrArUrCrUrUrGrUrUrUrUrUrCrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B6 | rCrCrGrUrUrUrUrCrCrArArUrArGrGrArGrCrGrGrGrCrGrGrUrAr |
| | | | UrGrU*mU*mU*mU |
| MG3-6/3- | 425 | MG3- | mA*mG*mG*rArCrUrArGrUrArUrUrCrArArGrArArCrCrCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C6 | rCrCrGrUrUrUrUrCrCrArArUrArGrGrArGrCrGrGrGrCrGrGrUrAr |
| | | | UrGrU*mU*mU*mU |
| MG3-6/3- | 426 | MG3- | mG*mG*mA*rCrUrArGrUrArUrUrCrArArGrArArCrCrCrArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D6 | rCrCrGrUrUrUrUrCrCrArArUrArGrGrArGrCrGrGrGrCrGrGrUrAr |
| | | | UrGrU*mU*mU*mU |
| MG3-6/3- | 427 | MG3- | mA*mA*mG*rArArCrUrArCrUrCrCrCrUrUrUrCrUrUrCrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E6 | rCrCrGrUrUrUrUrCrCrArArUrArGrGrArGrCrGrGrGrCrGrGrUrAr |
| | | | UrGrU*mU*mU*mU |
| MG3-6/3- | 428 | MG3- | mA*mC*mU*rArCrUrCrCrCrUrUrUrCrUrUrCrArGrUrUrGrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F6 | rCrCrGrUrUrUrUrCrCrArArUrArGrGrArGrCrGrGrGrCrGrGrUrAr |
| | | | UrGrU*mU*mU*mU |
| MG3-6/3- | 429 | MG3- | mC*mU*mA*rCrUrCrCrCrUrUrUrCrUrUrCrArGrUrUrGrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G6 | rCrCrGrUrUrUrUrCrCrArArUrArGrGrArGrCrGrGrGrCrGrGrUrAr |
| | | | UrGrU*mU*mU*mU |
| MG3-6/3- | 430 | MG3- | mC*mC*mU*rUrUrCrUrUrCrArGrUrUrGrArArUrGrArArArUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H6 | rCrCrGrUrUrUrUrCrCrArArUrArGrGrArGrCrGrGrGrCrGrGrUrAr |
| | | | UrGrU*mU*mU*mU |
| MG3-6/3- | 431 | MG3- | mG*mG*mU*rGrCrUrCrUrUrGrGrCrUrUrGrGrArArGrArUrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A7 | rCrCrGrUrUrUrUrCrCrArArUrArGrGrArGrCrGrGrGrCrGrGrUrAr |
| | | | UrGrU*mU*mU*mU |
| MG3-6/3- | 432 | MG3- | mG*mU*mG*rCrUrCrUrUrGrGrCrUrUrGrGrArArGrArUrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B7 | rCrCrGrUrUrUrUrCrCrArArUrArGrGrArGrCrGrGrGrCrGrGrUrAr |
| | | | UrGrU*mU*mU*mU |
| MG3-6/3- | 433 | MG3- | mA*mU*mA*rGrArGrArArArUrUrUrCrUrGrUrGrGrGrUrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C7 | rCrCrGrUrUrUrUrCrCrArArUrArGrGrArGrCrGrGrGrCrGrGrUrAr |
| | | | UrGrU*mU*mU*mU |
| MG3-6/3- | 434 | MG3- | mG*mA*mA*rUrArCrUrArGrUrCrCrUrUrCrUrGrArGrCrUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D7 | rCrCrGrUrUrUrUrCrCrArArUrArGrGrArGrCrGrGrGrCrGrGrUrAr |
| | | | UrGrU*mU*mU*mU |
| MG3-6/3- | 435 | MG3- | mU*mU*mA*rUrUrGrArUrUrCrUrArGrGrCrArUrUrCrCrUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E7 | rCrCrGrUrUrUrUrCrCrArArUrArGrGrArGrCrGrGrGrCrGrGrUrAr |
| | | | UrGrU*mU*mU*mU |
| MG3-6/3- | 436 | MG3- | mG*mU*mC*rUrArCrUrGrUrGrArUrGrUrUrArUrArUrCrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F7 | rCrCrGrUrUrUrUrCrCrArArUrArGrGrArGrCrGrGrGrCrGrGrUrAr |
| | | | UrGrU*mU*mU*mU |
| MG3-6/3- | 437 | MG3- | mC*mU*mG*rArUrArUrArArCrArUrCrArCrArGrUrArGrArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G7 | rCrCrGrUrUrUrUrCrCrArArUrArGrGrArGrCrGrGrGrCrGrGrUrAr |
| | | | UrGrU*mU*mU*mU |
| MG3-6/3- | 438 | MG3- | mU*mG*mA*rUrArUrArArCrArUrCrArCrArGrUrArGrArCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H7 | rCrCrGrUrUrUrUrCrCrArArUrArGrGrArGrCrGrGrGrCrGrGrUrAr |
| | | | UrGrU*mU*mU*mU |
| MG3-6/3- | 439 | MG3- | mG*mA*mU*rArUrArArCrArUrCrArCrArGrUrArGrArCrArUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A8 | rCrCrGrUrUrUrUrCrCrArArUrArGrGrArGrCrGrGrGrCrGrGrUrAr |
| | | | UrGrU*mU*mU*mU |
| MG3-6/3- | 440 | MG3- | mC*mA*mC*rUrUrGrUrArUrGrUrUrCrArCrCrUrCrUrGrUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B8 | rCrCrGrUrUrUrUrCrCrArArUrArGrGrArGrCrGrGrGrCrGrGrUrAr |
| | | | UrGrU*mU*mU*mU |
| MG3-6/3 | 441 | MG3- | mU*mA*mU*rArArArUrGrGrUrGrGrUrArCrArUrUrCrArGrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C8 | rCrCrGrUrUrUrUrCrCrArArUrArGrGrArGrCrGrGrGrCrGrGrUrAr |
| | | | UrGrU*mU*mU*mU |
| MG3-6/3- | 442 | MG3- | mU*mG*mG*rUrArCrArUrUrCrArGrCrArGrGrArArUrGrCrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D8 | rCrCrGrUrUrUrUrCrCrArArUrArGrGrArGrCrGrGrGrCrGrGrUrAr |
| | | | UrGrU*mU*mU*mU |
| MG3-6/3- | 443 | MG3- | mG*mU*mC*rCrArUrGrGrArCrArUrUrArArUrUrCrArArCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E8 | rCrCrGrUrUrUrUrCrCrArArUrArGrGrArGrCrGrGrGrCrGrGrUrAr |
| | | | UrGrU*mU*mU*mU |
| MG3-6/3- | 444 | MG3- | mU*mU*mC*rArArCrArUrCrGrArArUrArGrArUrGrGrArUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F8 | rCrCrGrUrUrUrUrCrCrArArUrArGrGrArGrCrGrGrGrCrGrGrUrAr |
| | | | UrGrU*mU*mU*mU |
| MG3-6/3- | 445 | MG3- | mA*mU*mA*rGrArUrGrGrArUrCrArCrArArArArCrUrUrCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G8 | rCrCrGrUrUrUrUrCrCrArArUrArGrGrArGrCrGrGrGrCrGrGrUrAr |
| | | | UrGrU*mU*mU*mU |
| MG3-6/3- | 446 | MG3- | mU*mU*mC*rArArUrGrArArArCrGrUrGrGrGrArGrArArCrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H8 | rCrCrGrUrUrUrUrCrCrArArUrArGrGrArGrCrGrGrGrCrGrGrUrAr |
| | | | UrGrU*mU*mU*mU |
| MG3-6/3- | 447 | MG3- | mA*mG*mU*rCrCrCrCrUrUrArCrCrArUrCrArArGrCrCrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A9 | rCrCrGrUrUrUrUrCrCrArArUrArGrGrArGrCrGrGrGrCrGrGrUrAr |
| | | | UrGrU*mU*mU*mU |
| MG3-6/3- | 448 | MG3- | mU*mU*mU*rGrUrGrArUrCrCrArUrCrUrArUrUrCrGrArUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B9 | rCrCrGrUrUrUrUrCrCrArArUrArGrGrArGrCrGrGrGrCrGrGrUrAr |
| | | | UrGrU*mU*mU*mU |
| MG3-6/3- | 449 | MG3- | mU*mG*mA*rArUrUrArArUrGrUrCrCrArUrGrGrArCrUrArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C9 | rCrCrGrUrUrUrUrCrCrArArUrArGrGrArGrCrGrGrGrCrGrGrUrAr |
| | | | UrGrU*mU*mU*mU |
| MG3-6/3- | 450 | MG3- | mU*mU*mU*rArCrGrArArUrUrGrArGrUrUrGrGrArArGrArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D9 | rCrCrGrUrUrUrUrCrCrArArUrArGrGrArGrCrGrGrGrCrGrGrUrAr |
| | | | UrGrU*mU*mU*mU |
| MG3-6/3- | 451 | MG3- | mG*mG*mC*rArArUrGrUrCrCrCrCrArArUrGrCrArArUrCrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E9 | rCrCrGrUrUrUrUrCrCrArArUrArGrGrArGrCrGrGrGrCrGrGrUrAr |
| | | | UrGrU*mU*mU*mU |
| MG3-6/3- | 452 | MG3- | mG*mC*mA*rArUrGrUrCrCrCrCrArArUrGrCrArArUrCrCrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F9 | rCrCrGrUrUrUrUrCrCrArArUrArGrGrArGrCrGrGrGrCrGrGrUrAr |
| | | | UrGrU*mU*mU*mU |
| MG3-6/3- | 453 | MG3- | mG*mU*mU*rUrUrCrUrArCrUrUrGrGrGrArUrCrArCrArArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G9 | rCrCrGrUrUrUrUrCrCrArArUrArGrGrArGrCrGrGrGrCrGrGrUrAr |
| | | | UrGrU*mU*mU*mU |
| MG3-6/3- | 454 | MG3- | mC*mC*mU*rUrUrUrGrCrUrUrUrGrUrGrArUrCrCrCrArArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H9 | rCrCrGrUrUrUrUrCrCrArArUrArGrGrArGrCrGrGrGrCrGrGrUrAr |
| | | | UrGrU*mU*mU*mU |
| MG3-6/3- | 455 | MG3- | mC*mU*mU*rUrUrGrCrUrUrUrGrUrGrArUrCrCrCrArArGrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A10 | rCrCrGrUrUrUrUrCrCrArArUrArGrGrArGrCrGrGrGrCrGrGrUrAr |
| | | | UrGrU*mU*mU*mU |
| MG3-6/3- | 456 | MG3- | mU*mU*mG*rUrGrArUrCrCrCrArArGrUrArGrArArArArCrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B10 | rCrCrGrUrUrUrUrCrCrArArUrArGrGrArGrCrGrGrGrCrGrGrUrAr |
| | | | UrGrU*mU*mU*mU |
| MG3-6/3- | 457 | MG3- | mA*mG*mU*rUrGrGrUrUrUrCrGrUrGrArUrUrUrCrCrCrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C10 | rCrCrGrUrUrUrUrCrCrArArUrArGrGrArGrCrGrGrGrCrGrGrUrAr |
| | | | UrGrU*mU*mU*mU |
| MG3-6/3- | 458 | MG3- | mG*mU*mU*rGrGrUrUrUrCrGrUrGrArUrUrUrCrCrCrArArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D10 | rCrCrGrUrUrUrUrCrCrArArUrArGrGrArGrCrGrGrGrCrGrGrUrAr |
| | | | UrGrU*mU*mU*mU |
| MG3-6/3 | 459 | MG3- | mG*mU*mU*rUrCrGrUrGrArUrUrUrCrCrCrArArGrUrArArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E10 | rCrCrGrUrUrUrUrCrCrArArUrArGrGrArGrCrGrGrGrCrGrGrUrAr |
| | | | UrGrU*mU*mU*mU |
| MG3-6/3- | 460 | MG3- | mU*mU*mC*rCrArGrUrCrUrUrCrCrArArCrUrCrArArUrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F10 | rCrCrGrUrUrUrUrCrCrArArUrArGrGrArGrCrGrGrGrCrGrGrUrAr |
| | | | UrGrU*mU*mU*mU |
| MG3-6/3- | 461 | MG3- | mA*mG*mU*rArUrArUrCrUrUrCrUrCrUrArGrGrCrCrCrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G10 | rCrCrGrUrUrUrUrCrCrArArUrArGrGrArGrCrGrGrGrCrGrGrUrAr |
| | | | UrGrU*mU*mU*mU |
| MG3-6/3- | 462 | MG3- | mG*mU*mA*rUrArUrCrUrUrCrUrCrUrArGrGrCrCrCrArArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H10 | rCrCrGrUrUrUrUrCrCrArArUrArGrGrArGrCrGrGrGrCrGrGrUrAr |
| | | | UrGrU*mU*mU*mU |
| MG3-6/3- | 463 | MG3- | mU*mC*mU*rArGrGrCrCrCrArArCrCrArArArArUrUrCrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A11 | rCrCrGrUrUrUrUrCrCrArArUrArGrGrArGrCrGrGrGrCrGrGrUrAr |
| | | | UrGrU*mU*mU*mU |
| MG3-6/3- | 464 | MG3- | mC*mU*mA*rGrGrCrCrCrArArCrCrArArArArUrUrCrUrCrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B11 | rCrCrGrUrUrUrUrCrCrArArUrArGrGrArGrCrGrGrGrCrGrGrUrAr |
| | | | UrGrU*mU*mU*mU |
| MG3-6/3- | 465 | MG3- | mG*mC*mC*rCrArArCrCrArArArArUrUrCrUrCrCrUrGrArArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C11 | rCrCrGrUrUrUrUrCrCrArArUrArGrGrArGrCrGrGrGrCrGrGrUrAr |
| | | | UrGrU*mU*mU*mU |
| MG3-6/3- | 466 | MG3- | mU*mG*mG*rUrGrGrUrGrGrCrArUrGrArUrGrArGrUrGrUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D11 | rCrCrGrUrUrUrUrCrCrArArUrArGrGrArGrCrGrGrGrCrGrGrUrAr |
| | | | UrGrU*mU*mU*mU |
| MG3-6/3- | 467 | MG3- | mG*mG*mU*rGrGrUrGrGrCrArUrGrArUrGrArGrUrGrUrGrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E11 | rCrCrGrUrUrUrUrCrCrArArUrArGrGrArGrCrGrGrGrCrGrGrUrAr |
| | | | UrGrU*mU*mU*mU |
| MG3-6/3- | 468 | MG3- | mU*mG*mA*rUrGrArGrUrGrUrGrGrArGrArArArArCrArArCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F11 | rCrCrGrUrUrUrUrCrCrArArUrArGrGrArGrCrGrGrGrCrGrGrUrAr |
| | | | UrGrU*mU*mU*mU |
| MG3-6/3- | 469 | MG3- | mU*mG*mU*rGrGrArGrArArArArCrArArCrCrUrArArArUrGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G11 | rCrCrGrUrUrUrUrCrCrArArUrArGrGrArGrCrGrGrGrCrGrGrUrAr |
| | | | UrGrU*mU*mU*mU |
| MG3-6/3- | 470 | MG3- | mG*mG*mU*rArArArUrArUrArArCrArArArCrCrArArGrArGrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | H11 | rCrCrGrUrUrUrUrCrCrArArUrArGrGrArGrCrGrGrGrCrGrGrUrAr |
| | | | UrGrU*mU*mU*mU |
| MG3-6/3- | 471 | MG3- | mG*mA*mA*rGrArGrGrArUrUrArUrCrUrUrGrGrArArGrUrCrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | A12 | rCrCrGrUrUrUrUrCrCrArArUrArGrGrArGrCrGrGrGrCrGrGrUrAr |
| | | | UrGrU*mU*mU*mU |
| MG3-6/3- | 472 | MG3- | mA*mA*mG*rArGrGrArUrUrArUrCrUrUrGrGrArArGrUrCrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | B12 | rCrCrGrUrUrUrUrCrCrArArUrArGrGrArGrCrGrGrGrCrGrGrUrAr |
| | | | UrGrU*mU*mU*mU |
| MG3-6/3- | 473 | MG3- | mU*mC*mA*rArArArUrGrGrArArGrGrUrUrArUrArCrUrCrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | C12 | rCrCrGrUrUrUrUrCrCrArArUrArGrGrArGrCrGrGrGrCrGrGrUrAr |
| | | | UrGrU*mU*mU*mU |
| MG3-6/3- | 474 | MG3- | mC*mA*mA*rArArUrGrGrArArGrGrUrUrArUrArCrUrCrUrArGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | D12 | rCrCrGrUrUrUrUrCrCrArArUrArGrGrArGrCrGrGrGrCrGrGrUrAr |
| | | | UrGrU*mU*mU*mU |
| MG3-6/3- | 475 | MG3- | mA*mU*mG*rUrUrGrArUrCrCrArUrCrCrArArCrArGrArUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | E12 | rCrCrGrUrUrUrUrCrCrArArUrArGrGrArGrCrGrGrGrCrGrGrUrAr |
| | | | UrGrU*mU*mU*mU |
| MG3-6/3- | 476 | MG3- | mC*mA*mU*rCrCrArArCrArGrArUrUrCrArGrArArArGrCrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | F12 | rCrCrGrUrUrUrUrCrCrArArUrArGrGrArGrCrGrGrGrCrGrGrUrAr |
| | | | UrGrU*mU*mU*mU |
| MG3-6/3- | 477 | MG3- | mG*mC*mC*rUrCrArGrUrUrCrArUrUrCrArArArGrCrUrUrUrGrUrU |
| 4 sgRNA | | 6/3-4 | rGrArGrArArUrCrGrArArArGrArUrUrCrUrUrArArUrArArGrGrCr |
| targeting | | ANGPTL3 | ArUrCrCrUrUrCrCrGrArUrGrCrUrGrArCrUrUrCrUrCrArCrCrGrU |
| ANGPTL3 | | G12 | rCrCrGrUrUrUrUrCrCrArArUrArGrGrArGrCrGrGrGrCrGrGrUrAr |
| | | | UrGrU*mU*mU*mU |
| DNA | 478 | MG3- | TTGTTCCTCTAGTTATTTCCTC |
| sequence | | 6/3-4 | |
| of | | ANGPTL3 | |
| ANGPTL3 | | A1 | |
| target | | | |
| site | | | |
| DNA | 479 | MG3- | ATTTGATTCTCTATCTCCAGAG |
| sequence | | 6/3-4 | |
| of | | ANGPTL3 | |
| ANGPTL3 | | B1 | |
| target | | | |
| site | | | |
| DNA | 480 | MG3- | TTTGATTCTCTATCTCCAGAGC |
| sequence | | 6/3-4 | |
| of | | ANGPTL3 | |
| ANGPTL3 | | C1 | |
| target | | | |
| site | | | |
| DNA | 481 | MG3- | AAGATTTGCTATGTTAGACGAT |
| sequence | | 6/3-4 | |
| of | | ANGPTL3 | |
| ANGPTL3 | | D1 | |
| target | | | |
| site | | | |
| DNA | 482 | MG3- | AGATTTGCTATGTTAGACGATG |
| sequence | | 6/3-4 | |
| of | | ANGPTL3 | |
| ANGPTL3 | | E1 | |
| target | | | |
| site | | | |
| DNA | 483 | MG3- | GATTTGCTATGTTAGACGATGT |
| sequence | | 6/3-4 | |
| of | | ANGPTL3 | |
| ANGPTL3 | | F1 | |
| target | | | |
| site | | | |
| DNA | 484 | MG3- | ACTTTGTCCATAAGACGAAGGG |
| sequence | | 6/3-4 | |
| of | | ANGPTL3 | |
| ANGPTL3 | | G1 | |
| target | | | |
| site | | | |
| DNA | 485 | MG3- | AGGGCCAAATTAATGACATATT |
| sequence | | 6/3-4 | |
| of | | ANGPTL3 | |
| ANGPTL3 | | H1 | |
| target | | | |
| site | | | |
| DNA | 486 | MG3- | GGGCCAAATTAATGACATATTT |
| sequence | | 6/3-4 | |
| of | | ANGPTL3 | |
| ANGPTL3 | | A2 | |
| target | | | |
| site | | | |
| DNA | 487 | MG3- | TATGATCTATCGCTGCAAACCA |
| sequence | | 6/3-4 | |
| of | | ANGPTL3 | |
| ANGPTL3 | | B2 | |
| target | | | |
| site | | | |
| DNA | 488 | MG3- | ATGATCTATCGCTGCAAACCAG |
| sequence | | 6/3-4 | |
| of | | ANGPTL3 | |
| ANGPTL3 | | C2 | |
| target | | | |
| site | | | |
| DNA | 489 | MG3- | CAAACCAGTGAAATCAAAGAAG |
| sequence | | 6/3-4 | |
| of | | ANGPTL3 | |
| ANGPTL3 | | D2 | |
| target | | | |
| site | | | |
| DNA | 490 | MG3- | AAACCAGTGAAATCAAAGAAGA |
| sequence | | 6/3-4 | |
| of | | ANGPTL3 | |
| ANGPTL3 | | E2 | |
| target | | | |
| site | | | |
| DNA | 491 | MG3- | ACAAGTCAAAAATGAAGAGGTA |
| sequence | | 6/3-4 | |
| of | | ANGPTL3 | |
| ANGPTL3 | | F2 | |
| target | | | |
| site | | | |
| DNA | 492 | MG3- | GAATATGTCACTTGAACTCAAC |
| sequence | | 6/3-4 | |
| of | | ANGPTL3 | |
| ANGPTL3 | | G2 | |
| target | | | |
| site | | | |
| DNA | 493 | MG3- | TCACTTGAACTCAACTCAAAAC |
| sequence | | 6/3-4 | |
| of | | ANGPTL3 | |
| ANGPTL3 | | H2 | |
| target | | | |
| site | | | |
| DNA | 494 | MG3- | TCAAAACTTGAAAGCCTCCTAG |
| sequence | | 6/3-4 | |
| of | | ANGPTL3 | |
| ANGPTL3 | | A3 | |
| target | | | |
| site | | | |
| DNA | 495 | MG3- | CAAAACTTGAAAGCCTCCTAGA |
| sequence | | 6/3-4 | |
| of | | ANGPTL3 | |
| ANGPTL3 | | B3 | |
| target | | | |
| site | | | |
| DNA | 496 | MG3- | AAAACTTGAAAGCCTCCTAGAA |
| sequence | | 6/3-4 | |
| of | | ANGPTL3 | |
| ANGPTL3 | | C3 | |
| target | | | |
| site | | | |
| DNA | 497 | MG3- | AAACTTGAAAGCCTCCTAGAAG |
| sequence | | 6/3-4 | |
| of | | ANGPTL3 | |
| ANGPTL3 | | D3 | |
| target | | | |
| site | | | |
| DNA | 498 | MG3- | AACTTGAAAGCCTCCTAGAAGA |
| sequence | | 6/3-4 | |
| of | | ANGPTL3 | |
| ANGPTL3 | | E3 | |
| target | | | |
| site | | | |
| DNA | 499 | MG3- | GTTCTGGAGTTTCAGGTTGATT |
| sequence | | 6/3-4 | |
| of | | ANGPTL3 | |
| ANGPTL3 | | F3 | |
| target | | | |
| site | | | |
| DNA | 500 | MG3- | CACTGGTTTGCAGCGATAGATC |
| sequence | | 6/3-4 | |
| of | | ANGPTL3 | |
| ANGPTL3 | | G3 | |
| target | | | |
| site | | | |
| DNA | 501 | MG3- | ACTGGTTTGCAGCGATAGATCA |
| sequence | | 6/3-4 | |
| of | | ANGPTL3 | |
| ANGPTL3 | | H3 | |
| target | | | |
| site | | | |
| DNA | 502 | MG3- | CGATAGATCATAAAAAGACTGA |
| sequence | | 6/3-4 | |
| of | | ANGPTL3 | |
| ANGPTL3 | | A4 | |
| target | | | |
| site | | | |
| DNA | 503 | MG3- | CCCAACTGAAGGAGGCCATTGG |
| sequence | | 6/3-4 | |
| of | | ANGPTL3 | |
| ANGPTL3 | | B4 | |
| target | | | |
| site | | | |
| DNA | 504 | MG3- | CCAACTGAAGGAGGCCATTGGC |
| sequence | | 6/3-4 | |
| of | | ANGPTL3 | |
| ANGPTL3 | | C4 | |
| target | | | |
| site | | | |
| DNA | 505 | MG3- | CTTGATTTTGGCTCTGGAGATA |
| sequence | | 6/3-4 | |
| of | | ANGPTL3 | |
| ANGPTL3 | | D4 | |
| target | | | |
| site | | | |
| DNA | 506 | MG3- | TTTTGGCTCTGGAGATAGAGAA |
| sequence | | 6/3-4 | |
| of | | ANGPTL3 | |
| ANGPTL3 | | E4 | |
| target | | | |
| site | | | |
| DNA | 507 | MG3- | TCTGGAGATAGAGAATCAAATG |
| sequence | | 6/3-4 | |
| of | | ANGPTL3 | |
| ANGPTL3 | | F4 | |
| target | | | |
| site | | | |
| DNA | 508 | MG3- | GAATTGTCTTGATCAATTCTGG |
| sequence | | 6/3-4 | |
| of | | ANGPTL | |
| ANGPTL3 | | G4 | |
| target | | | |
| site | | | |
| DNA | 509 | MG3- | AATTGTCTTGATCAATTCTGGA |
| sequence | | 6/3-4 | |
| of | | ANGPTL3 | |
| ANGPTL3 | | H4 | |
| target | | | |
| site | | | |
| DNA | 510 | MG3- | GGAGGAAATAACTAGAGGAACA |
| sequence | | 6/3-4 | |
| of | | ANGPTL3 | |
| ANGPTL3 | | A5 | |
| target | | | |
| site | | | |
| DNA | 511 | MG3- | GAGGAAATAACTAGAGGAACAA |
| sequence | | 6/3-4 | |
| of | | ANGPTL3 | |
| ANGPTL3 | | B5 | |
| target | | | |
| site | | | |
| DNA | 512 | MG3- | ACTCTCTATATCCAGACTTTTG |
| sequence | | 6/3-4 | |
| of | | ANGPTL3 | |
| ANGPTL3 | | C5 | |
| target | | | |
| site | | | |
| DNA | 513 | MG3- | CTCTCTATATCCAGACTTTTGT |
| sequence | | 6/3-4 | |
| of | | ANGPTL3 | |
| ANGPTL3 | | D5 | |
| target | | | |
| site | | | |
| DNA | 514 | MG3- | TCTCTATATCCAGACTTTTGTA |
| sequence | | 6/3-4 | |
| of | | ANGPTL3 | |
| ANGPTL3 | | E5 | |
| target | | | |
| site | | | |
| DNA | 515 | MG3- | AACAATTAAACCAACAGCATAG |
| sequence | | 6/3-4 | |
| of | | ANGPTL3 | |
| ANGPTL3 | | F5 | |
| target | | | |
| site | | | |
| DNA | 516 | MG3- | ATTAAACCAACAGCATAGTCAA |
| sequence | | 6/3-4 | |
| of | | ANGPTL3 | |
| ANGPTL3 | | G5 | |
| target | | | |
| site | | | |
| DNA | 517 | MG3- | AACCAACAGCATAGTCAAATAA |
| sequence | | 6/3-4 | |
| of | | ANGPTL3 | |
| ANGPTL3 | | H5 | |
| target | | | |
| site | | | |
| DNA | 518 | MG3- | ACCAACAGCATAGTCAAATAAA |
| sequence | | 6/3-4 | |
| of | | ANGPTL3 | |
| ANGPTL3 | | A6 | |
| target | | | |
| site | | | |
| DNA | 519 | MG3- | GATGCTATTATCTTGTTTTTCT |
| sequence | | 6/3-4 | |
| of | | ANGPTL3 | |
| ANGPTL3 | | B6 | |
| target | | | |
| site | | | |
| DNA | 520 | MG3- | AGGACTAGTATTCAAGAACCCA |
| sequence | | 6/3-4 | |
| of | | ANGPTL3 | |
| ANGPTL3 | | C6 | |
| target | | | |
| site | | | |
| DNA | 521 | MG3- | GGACTAGTATTCAAGAACCCAC |
| sequence | | 6/3-4 | |
| of | | ANGPTL3 | |
| ANGPTL3 | | D6 | |
| target | | | |
| site | | | |
| DNA | 522 | MG3- | AAGAACTACTCCCTTTCTTCAG |
| sequence | | 6/3-4 | |
| of | | ANGPTL3 | |
| ANGPTL3 | | E6 | |
| target | | | |
| site | | | |
| DNA | 523 | MG3- | ACTACTCCCTTTCTTCAGTTGA |
| sequence | | 6/3-4 | |
| of | | ANGPTL3 | |
| ANGPTL3 | | F6 | |
| target | | | |
| site | | | |
| DNA | 524 | MG3- | CTACTCCCTTTCTTCAGTTGAA |
| sequence | | 6/3-4 | |
| of | | ANGPTL3 | |
| ANGPTL3 | | G6 | |
| target | | | |
| site | | | |
| DNA | 525 | MG3- | CCTTTCTTCAGTTGAATGAAAT |
| sequence | | 6/3-4 | |
| of | | ANGPTL3 | |
| ANGPTL3 | | H6 | |
| target | | | |
| site | | | |
| DNA | 526 | MG3- | GGTGCTCTTGGCTTGGAAGATA |
| sequence | | 6/3-4 | |
| of | | ANGPTL3 | |
| ANGPTL3 | | A7 | |
| target | | | |
| site | | | |
| DNA | 527 | MG3- | GTGCTCTTGGCTTGGAAGATAG |
| sequence | | 6/3-4 | |
| of | | ANGPTL3 | |
| ANGPTL3 | | B7 | |
| target | | | |
| site | | | |
| DNA | 528 | MG3- | ATAGAGAAATTTCTGTGGGTTC |
| sequence | | 6/3-4 | |
| of | | ANGPTL3 | |
| ANGPTL3 | | C7 | |
| target | | | |
| site | | | |
| DNA | 529 | MG3- | GAATACTAGTCCTTCTGAGCTG |
| sequence | | 6/3-4 | |
| of | | ANGPTL3 | |
| ANGPTL3 | | D7 | |
| target | | | |
| site | | | |
| DNA | 530 | MG3- | TTATTGATTCTAGGCATTCCTG |
| sequence | | 6/3-4 | |
| of | | ANGPTL3 | |
| ANGPTL3 | | E7 | |
| target | | | |
| site | | | |
| DNA | 531 | MG3- | GTCTACTGTGATGTTATATCAG |
| sequence | | 6/3-4 | |
| of | | ANGPTL3 | |
| ANGPTL3 | | F7 | |
| target | | | |
| site | | | |
| DNA | 532 | MG3- | CTGATATAACATCACAGTAGAC |
| sequence | | 6/3-4 | |
| of | | ANGPTL3 | |
| ANGPTL3 | | G7 | |
| target | | | |
| site | | | |
| DNA | 533 | MG3- | TGATATAACATCACAGTAGACA |
| sequence | | 6/3-4 | |
| of | | ANGPTL3 | |
| ANGPTL3 | | H7 | |
| target | | | |
| site | | | |
| DNA | 534 | MG3- | GATATAACATCACAGTAGACAT |
| sequence | | 6/3-4 | |
| of | | ANGPTL3 | |
| ANGPTL3 | | A8 | |
| target | | | |
| site | | | |
| DNA | 535 | MG3- | CACTTGTATGTTCACCTCTGTT |
| sequence | | 6/3-4 | |
| of | | ANGPTL3 | |
| ANGPTL3 | | B8 | |
| target | | | |
| site | | | |
| DNA | 536 | MG3- | TATAAATGGTGGTACATTCAGC |
| sequence | | 6/3-4 | |
| of | | ANGPTL3 | |
| ANGPTL3 | | C8 | |
| target | | | |
| site | | | |
| DNA | 537 | MG3- | TGGTACATTCAGCAGGAATGCC |
| sequence | | 6/3-4 | |
| of | | ANGPTL3 | |
| ANGPTL3 | | D8 | |
| target | | | |
| site | | | |
| DNA | 538 | MG3- | GTCCATGGACATTAATTCAACA |
| sequence | | 6/3-4 | |
| of | | ANGPTL3 | |
| ANGPTL3 | | E8 | |
| target | | | |
| site | | | |
| DNA | 539 | MG3- | TTCAACATCGAATAGATGGATC |
| sequence | | 6/3-4 | |
| of | | ANGPTL3 | |
| ANGPTL3 | | F8 | |
| target | | | |
| site | | | |
| DNA | 540 | MG3- | ATAGATGGATCACAAAACTTCA |
| sequence | | 6/3-4 | |
| of | | ANGPTL3 | |
| ANGPTL3 | | G8 | |
| target | | | |
| site | | | |
| DNA | 541 | MG3- | TTCAATGAAACGTGGGAGAACT |
| sequence | | 6/3-4 | |
| of | | ANGPTL3 | |
| ANGPTL3 | | H8 | |
| target | | | |
| site | | | |
| DNA | 542 | MG3- | AGTCCCCTTACCATCAAGCCTC |
| sequence | | 6/3-4 | |
| of | | ANGPTL3 | |
| ANGPTL3 | | A9 | |
| target | | | |
| site | | | |
| DNA | 543 | MG3- | TTTGTGATCCATCTATTCGATG |
| sequence | | 6/3-4 | |
| of | | ANGPTL3 | |
| ANGPTL3 | | B9 | |
| target | | | |
| site | | | |
| DNA | 544 | MG3- | TGAATTAATGTCCATGGACTAC |
| sequence | | 6/3-4 | |
| of | | ANGPTL3 | |
| ANGPTL3 | | C9 | |
| target | | | |
| site | | | |
| DNA | 545 | MG3- | TTTACGAATTGAGTTGGAAGAC |
| sequence | | 6/3-4 | |
| of | | ANGPTL3 | |
| ANGPTL3 | | D9 | |
| target | | | |
| site | | | |
| DNA | 546 | MG3- | GGCAATGTCCCCAATGCAATCC |
| sequence | | 6/3-4 | |
| of | | ANGPTL3 | |
| ANGPTL3 | | E9 | |
| target | | | |
| site | | | |
| DNA | 547 | MG3- | GCAATGTCCCCAATGCAATCCC |
| sequence | | 6/3-4 | |
| of | | ANGPTL3 | |
| ANGPTL3 | | F9 | |
| target | | | |
| site | | | |
| DNA | 548 | MG3- | GTTTTCTACTTGGGATCACAAA |
| sequence | | 6/3-4 | |
| of | | ANGPTL3 | |
| ANGPTL3 | | G9 | |
| target | | | |
| site | | | |
| DNA | 549 | MG3- | CCTTTTGCTTTGTGATCCCAAG |
| sequence | | 6/3-4 | |
| of | | ANGPTL3 | |
| ANGPTL3 | | H9 | |
| target | | | |
| site | | | |
| DNA | 550 | MG3- | CTTTTGCTTTGTGATCCCAAGT |
| sequence | | 6/3-4 | |
| of | | ANGPTL3 | |
| ANGPTL3 | | A10 | |
| target | | | |
| site | | | |
| DNA | 551 | MG3- | TTGTGATCCCAAGTAGAAAACA |
| sequence | | 6/3-4 | |
| of | | ANGPTL3 | |
| ANGPTL3 | | B10 | |
| target | | | |
| site | | | |
| DNA | 552 | MG3- | AGTTGGTTTCGTGATTTCCCAA |
| sequence | | 6/3-4 | |
| of | | ANGPTL3 | |
| ANGPTL3 | | C10 | |
| target | | | |
| site | | | |
| DNA | 553 | MG3- | GTTGGTTTCGTGATTTCCCAAG |
| sequence | | 6/3-4 | |
| of | | ANGPTL3 | |
| ANGPTL3 | | D10 | |
| target | | | |
| site | | | |
| DNA | 554 | MG3- | GTTTCGTGATTTCCCAAGTAAA |
| sequence | | 6/3-4 | |
| of | | ANGPTL3 | |
| ANGPTL3 | | E10 | |
| target | | | |
| site | | | |
| DNA | 555 | MG3- | TTCCAGTCTTCCAACTCAATTC |
| sequence | | 6/3-4 | |
| of | | ANGPTL3 | |
| ANGPTL3 | | F10 | |
| target | | | |
| site | | | |
| DNA | 556 | MG3- | AGTATATCTTCTCTAGGCCCAA |
| sequence | | 6/3-4 | |
| of | | ANGPTL3 | |
| ANGPTL3 | | G10 | |
| target | | | |
| site | | | |
| DNA | 557 | MG3- | GTATATCTTCTCTAGGCCCAAC |
| sequence | | 6/3-4 | |
| of | | ANGPTL3 | |
| ANGPTL3 | | H10 | |
| target | | | |
| site | | | |
| DNA | 558 | MG3- | TCTAGGCCCAACCAAAATTCTC |
| sequence | | 6/3-4 | |
| of | | ANGPTL3 | |
| ANGPTL3 | | A11 | |
| target | | | |
| site | | | |
| DNA | 559 | MG3- | CTAGGCCCAACCAAAATTCTCC |
| sequence | | 6/3-4 | |
| of | | ANGPTL3 | |
| ANGPTL3 | | B11 | |
| target | | | |
| site | | | |
| DNA | 560 | MG3- | GCCCAACCAAAATTCTCCTGAA |
| sequence | | 6/3-4 | |
| of | | ANGPTL3 | |
| ANGPTL3 | | C11 | |
| target | | | |
| site | | | |
| DNA | 561 | MG3- | TGGTGGTGGCATGATGAGTGTG |
| sequence | | 6/3-4 | |
| of | | ANGPTL3 | |
| ANGPTL3 | | D11 | |
| target | | | |
| site | | | |
| DNA | 562 | MG3- | GGTGGTGGCATGATGAGTGTGG |
| sequence | | 6/3-4 | |
| of | | ANGPTL3 | |
| ANGPTL3 | | E11 | |
| target | | | |
| site | | | |
| DNA | 563 | MG3- | TGATGAGTGTGGAGAAAACAAC |
| sequence | | 6/3-4 | |
| of | | ANGPTL3 | |
| ANGPTL3 | | F11 | |
| target | | | |
| site | | | |
| DNA | 564 | MG3- | TGTGGAGAAAACAACCTAAATG |
| sequence | | 6/3-4 | |
| of | | ANGPTL3 | |
| ANGPTL3 | | G11 | |
| target | | | |
| site | | | |
| DNA | 565 | MG3- | GGTAAATATAACAAACCAAGAG |
| sequence | | 6/3-4 | |
| of | | ANGPTL3 | |
| ANGPTL3 | | H11 | |
| target | | | |
| site | | | |
| DNA | 566 | MG3- | GAAGAGGATTATCTTGGAAGTC |
| sequence | | 6/3-4 | |
| of | | ANGPTL3 | |
| ANGPTL3 | | A12 | |
| target | | | |
| site | | | |
| DNA | 567 | MG3- | AAGAGGATTATCTTGGAAGTCT |
| sequence | | 6/3-4 | |
| of | | ANGPTL3 | |
| ANGPTL3 | | B12 | |
| target | | | |
| site | | | |
| DNA | 568 | MG3- | TCAAAATGGAAGGTTATACTCT |
| sequence | | 6/3-4 | |
| of | | ANGPTL3 | |
| ANGPTL3 | | C12 | |
| target | | | |
| site | | | |
| DNA | 569 | MG3- | CAAAATGGAAGGTTATACTCTA |
| sequence | | 6/3-4 | |
| of | | ANGPTL3 | |
| ANGPTL3 | | D12 | |
| target | | | |
| site | | | |
| DNA | 570 | MG3- | ATGTTGATCCATCCAACAGATT |
| sequence | | 6/3-4 | |
| of | | ANGPTL3 | |
| ANGPTL3 | | E12 | |
| target | | | |
| site | | | |
| DNA | 571 | MG3- | CATCCAACAGATTCAGAAAGCT |
| sequence | | 6/3-4 | |
| of | | ANGPTL3 | |
| ANGPTL3 | | F12 | |
| target | | | |
| site | | | |
| DNA | 572 | MG3- | GCCTCAGTTCATTCAAAGCTTT |
| sequence | | 6/3-4 | |
| of | | ANGPTL3 | |
| ANGPTL3 | | G12 | |
| target | | | |
| site | | | |

(r = native ribose base, m = 2′-O methyl modified base, F = 2′ Fluro modified base, * = phosphorothioate bond)

### Example 17—Analysis of Gene-Editing Outcomes at the DNA Level for PCSK9 in Hep3B Cells

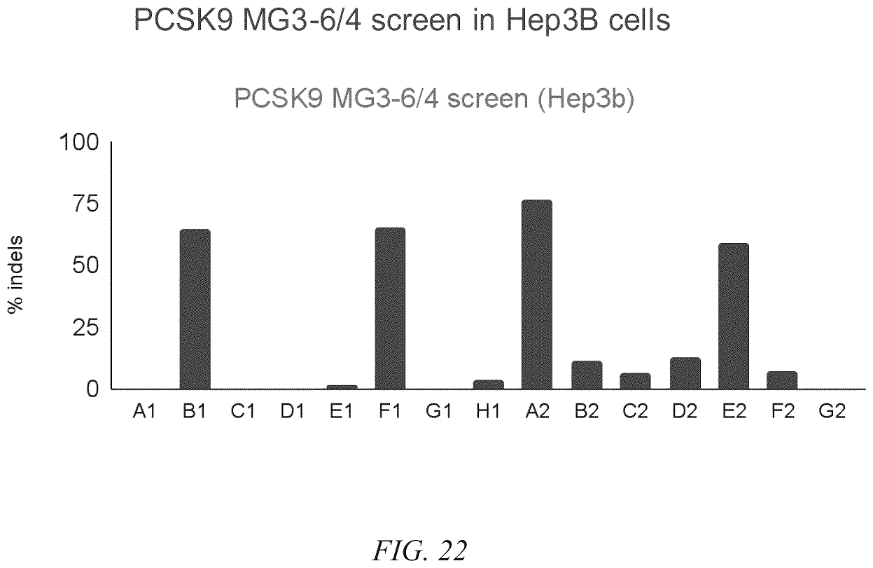

Nucleofection of MG3-6/4 RNPs (104 pmol protein/120 pmol guide) comprising sgRNAs described below in Table 9E below and SEQ ID NOs: 573-602 was performed into Hep3B cells (100,000) using electroporation. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing (FIG. 22). Results indicate that the highest editing performance was achieved with sgRNAs B1, F1, A2, and E2, with appreciable editing also occurring with D2, C2, B2, H1, and F2.

TABLE 9E
gRNAs and Targeting Sequences Used in Example 17

| | SEQ | | |
|---|---|---|---|
| | ID | | |
| Category | NO: | Name | Sequence |
| MG3-6/3- | 573 | MG3- | mA*mC*mC*rCrCrUrCrCrArCrGrGrUrArCrCrGrGrGrCrGrGrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | A1 | rCrCrGrUrUrUrUrCrCrArArUrArGrGrArGrCrGrGrGrCrGrGrUrAr |
| | | | UrGrU*mU*mU*mU |
| MG3-6/3- | 574 | MG3- | mA*mC*mC*rArGrCrArUrArCrArGrArGrUrGrArCrCrArCrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | B1 | rCrCrGrUrUrUrUrCrCrArArUrArGrGrArGrCrGrGrGrCrGrGrUrAr |
| | | | UrGrU*mU*mU*mU |
| MG3-6/3- | 575 | MG3- | mC*mC*mA*rGrCrArUrArCrArGrArGrUrGrArCrCrArCrCrGrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | C1 | rCrCrGrUrUrUrUrCrCrArArUrArGrGrArGrCrGrGrGrCrGrGrUrAr |
| | | | UrGrU*mU*mU*mU |
| MG3-6/3- | 576 | MG3- | mC*mA*mG*rGrGrUrCrArUrGrGrUrCrArCrCrGrArCrUrUrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | D1 | rCrCrGrUrUrUrUrCrCrArArUrArGrGrArGrCrGrGrGrCrGrGrUrAr |
| | | | UrGrU*mU*mU*mU |
| MG3-6/3- | 577 | MG3- | mC*mC*mU*rCrCrCrArGrGrCrCrUrGrGrArGrUrUrUrArUrUrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | E1 | rCrCrGrUrUrUrUrCrCrArArUrArGrGrArGrCrGrGrGrCrGrGrUrAr |
| | | | UrGrU*mU*mU*mU |
| MG3-6/3- | 578 | MG3- | mC*mU*mC*rCrCrArGrGrCrCrUrGrGrArGrUrUrUrArUrUrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | F1 | rCrCrGrUrUrUrUrCrCrArArUrArGrGrArGrCrGrGrGrCrGrGrUrAr |
| | | | UrGrU*mU*mU*mU |
| MG3-6/3- | 579 | MG3- | mC*mA*mG*rGrCrUrGrGrArCrCrArGrCrUrGrGrCrUrUrUrUrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | G1 | rCrCrGrUrUrUrUrCrCrArArUrArGrGrArGrCrGrGrGrCrGrGrUrAr |
| | | | UrGrU*mU*mU*mU |
| MG3-6/3- | 580 | MG3- | mG*mG*mU*rGrGrCrCrCrCrArArCrUrGrUrGrArUrGrArCrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | H1 | rCrCrGrUrUrUrUrCrCrArArUrArGrGrArGrCrGrGrGrCrGrGrUrAr |
| | | | UrGrU*mU*mU*mU |
| MG3-6/3- | 581 | MG3- | mG*mC*mC*rCrCrGrCrCrGrCrUrUrCrCrCrArCrUrCrCrUrGrGrUrU |
| 4 sgRNA | | 6/3-4 | rGrArGrArArUrCrGrArArArGrArUrUrCrUrUrArArUrArArGrGrCr |
| | | | ArUrCrCrUrUrCrCrGrArUrGrCrUrGrArCrUrUrCrUrCrArCrCrGrU |
| targeting | | PCSK9 | rCrCrGrUrUrUrUrCrCrArArUrArGrGrArGrCrGrGrGrCrGrGrUrAr |
| PCSK9 | | A2 | UrGrU*mU*mU*mU |
| MG3-6/3- | 582 | MG3- | mA*mG*mU*rGrUrGrCrUrGrArCrCrArUrArCrArGrUrCrCrUrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | B2 | rCrCrGrUrUrUrUrCrCrArArUrArGrGrArGrCrGrGrGrCrGrGrUrAr |
| | | | UrGrU*mU*mU*mU |
| MG3-6/3- | 583 | MG3- | mC*mC*mU*rGrCrArArArArCrArGrCrUrGrCrCrArArCrCrUrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | C2 | rCrCrGrUrUrUrUrCrCrArArUrArGrGrArGrCrGrGrGrCrGrGrUrAr |
| | | | UrGrU*mU*mU*mU |
| MG3-6/3- | 584 | MG3- | mC*mU*mG*rCrArArArArCrArGrCrUrGrCrCrArArCrCrUrGrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | D2 | rCrCrGrUrUrUrUrCrCrArArUrArGrGrArGrCrGrGrGrCrGrGrUrAr |
| | | | UrGrU*mU*mU*mU |
| MG3-6/3- | 585 | MG3- | mA*mA*mU*rGrGrCrGrUrArGrArCrArCrCrCrUrCrArCrCrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | E2 | rCrCrGrUrUrUrUrCrCrArArUrArGrGrArGrCrGrGrGrCrGrGrUrAr |
| | | | UrGrU*mU*mU*mU |
| MG3-6/3- | 586 | MG3- | mUmC*mC*rUrGrCrUrGrCrCrArUrGrCrCrCrCrArGrGrUrCrGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | F2 | rCrCrGrUrUrUrUrCrCrArArUrArGrGrArGrCrGrGrGrCrGrGrUrAr |
| | | | UrGrU*mU*mU*mU |
| MG3-6/3- | 587 | MG3- | mU*mG*mG*rArArUrGrCrArArArGrUrCrArArGrGrArGrCrArGrUrU |
| 4 sgRNA | | 6/3-4 | rGrArGrArArUrCrGrArArArGrArUrUrCrUrUrArArUrArArGrGrCr |
| targeting | | PCSK9 | ArUrCrCrUrUrCrCrGrArUrGrCrUrGrArCrUrUrCrUrCrArCrCrGrU |
| PCSK9 | | G2 | rCrCrGrUrUrUrUrCrCrArArUrArGrGrArGrCrGrGrGrCrGrGrUrAr |
| | | | UrGrU*mU*mU*mU |
| DNA | 588 | MG3- | ACCCCTCCACGGTACCGGGCGG |
| sequence | | 6/3-4 | |
| of | | PCSK9 | |
| PCSK9 | | A1 | |
| target site | | | |
| DNA | 589 | MG3- | ACCAGCATACAGAGTGACCACC |
| sequence | | 6/3-4 | |
| of | | PCSK9 | |
| PCSK9 | | B1 | |
| target site | | | |
| DNA | 590 | MG3- | CCAGCATACAGAGTGACCACCG |
| sequence | | 6/3-4 | |
| of | | PCSK9 | |
| PCSK9 | | C1 | |
| target site | | | |
| DNA | 591 | MG3- | CAGGGTCATGGTCACCGACTTC |
| sequence | | 6/3-4 | |
| of | | PCSK9 | |
| PCSK9 | | D1 | |
| target site | | | |
| DNA | 592 | MG3- | CCTCCCAGGCCTGGAGTTTATT |
| sequence | | 6/3-4 | |
| of | | PCSK9 | |
| PCSK9 | | E1 | |
| target site | | | |
| DNA | 593 | MG3- | CTCCCAGGCCTGGAGTTTATTC |
| sequence | | 6/3-4 | |
| of | | PCSK9 | |
| PCSK9 | | F1 | |
| target site | | | |
| DNA | 594 | MG3- | CAGGCTGGACCAGCTGGCTTTT |
| sequence | | 6/3-4 | |
| of | | PCSK9 | |
| PCSK9 | | G1 | |
| target site | | | |
| DNA | 595 | MG3- | GGTGGCCCCAACTGTGATGACC |
| sequence | | 6/3-4 | |
| of | | PCSK9 | |
| PCSK9 | | H1 | |
| target site | | | |
| DNA | 596 | MG3- | GCCCCGCCGCTTCCCACTCCTG |
| sequence | | 6/3-4 | |
| of | | PCSK9 | |
| PCSK9 | | A2 | |
| target site | | | |
| DNA | 597 | MG3- | AGTGTGCTGACCATACAGTCCT |
| sequence | | 6/3-4 | |
| of | | PCSK9 | |
| PCSK9 | | B2 | |
| target site | | | |
| DNA | 598 | MG3- | CCTGCAAAACAGCTGCCAACCT |
| sequence | | 6/3-4 | |
| of | | PCSK9 | |
| PCSK9 | | C2 | |
| target site | | | |
| DNA | 599 | MG3- | CTGCAAAACAGCTGCCAACCTG |
| sequence | | 6/3-4 | |
| of | | PCSK9 | |
| PCSK9 | | D2 | |
| target site | | | |
| DNA | 600 | MG3- | AATGGCGTAGACACCCTCACCC |
| sequence | | 6/3-4 | |
| of | | PCSK9 | |
| PCSK9 | | E2 | |
| target site | | | |
| DNA | 601 | MG3- | TCCTGCTGCCATGCCCCAGGTC |
| sequence | | 6/3-4 | |
| of | | PCSK9 | |
| PCSK9 | | F2 | |
| target site | | | |
| DNA | 602 | MG3- | TGGAATGCAAAGTCAAGGAGCA |
| sequence | | 6/3-4 | |
| of | | PCSK9 | |
| PCSK9 | | G2 | |
| target site | | | |
| bond) | | | |

(r = native ribose base, m = 2′-O methyl modified base, F = 2′ Fluro modified base, * = phosphorothioate

### Example 18—In Vivo Gene Editing in the Liver of Mice by the Chimeric Nuclease MG3-6/3-4 Delivered by Systemic Administration of a Lipid Nanoparticle

To evaluate the ability of the MG3-6/3-4 chimeric Type II nuclease to edit the genome in vivo in a living animal, a lipid nanoparticle was used to deliver an mRNA encoding the MG3-6/3-4 nuclease (e.g., RNA version of SEQ ID NO: 603) and single guide RNAs (sgRNA) that target different parts of the coding sequence of the mouse HAO-1 gene (e.g., described in Table 10). The HAO-1 gene encodes glycolate oxidase which is an enzyme involved in glycolate metabolism and is expressed primarily in hepatocytes in the liver. A screen of sgRNAs that target the HAO-1 coding sequence was performed in the mouse liver cell line Hepa1-6 to identify active guides. The sgRNAs mH364-7 and mH364-20, which exhibited 46% and 26% editing in Hepa1-6 cells when transfected with the mRNA encoding the MG3-6/3-4 nuclease, were selected for testing in mice. mH364-7 targets exon 2 and mH364-20 targets exon 4.

A number of chemical modifications of the native RNA structure were incorporated into these sgRNAs. These chemical modifications were selected based on their ability to improve the stability of the sgRNA in vitro when incubated in extracts from mammalian cells without negatively impacting editing activity. For initial testing in mice, sgRNAs mH364-7 and mH364-20 incorporating chemistry 1 and chemistry 35 were selected for testing and designated as mH364-7-1, mH364-20-1, mH364-7-35, mH364-20-35. The sequences of these guides including the chemical modifications are shown below in Table 10.

TABLE 10
Sequences and chemical modifications of guide RNA tested in vivo in mice

| Guide name | Sequence | SEQ ID NO: |
|---|---|---|
| mH364-7-1 | mG*mA*mG*CUGGCCACUGUGCGAGGUAGUU | 1795 |
| | GAGAAUCGAAAGAUUCUUAAUAAGGCAUCC | |
| | UUCCGAUGCUGACUUCUCACCGUCCGUUUUC | |
| | CAAUAGGAGCGGGCGGUAUGU*mU*mU*mU | |
| mH364-20-1 | mU*mU*mC*AGCAAGUCCACUGUUGUCUGUU | 1808 |
| | GAGAAUCGAAAGAUUCUUAAUAAGGCAUCC | |
| | UUCCGAUGCUGACUUCUCACCGUCCGUUUUC | |
| | CAAUAGGAGCGGGCGGUAUGU*mU*mU*mU | |
| mH364-7-35 | mG*mA*mG*mC*UGGCCACUGUGCGAGGUAG | 1816 |
| | UUGAGAAUCmG*mA*mA*mA*GAUUCUUAAU | |
| | AAGGCAUCmC*mU*mU*mC*mC*GAUGCUGAC | |
| | UUCUCACCGUCCGUUUUCCmA*mA*mU*mA*G | |
| | GAGCGGGCGGUA*mU*mG*mU*mU*mU*mU | |
| mH364-20-35 | mU*mU*mC*mA*GCAAGUCCACUGUUGUCUG | 1826 |
| | UUGAGAAUCmG*mA*mA*mA*GAUUCUUAAU | |
| | AAGGCAUCmC*mU*mU*mC*mC*GAUGCUGAC | |
| | UUCUCACCGUCCGUUUUCCmA*mA*mU*mA*G | |
| | GAGCGGGCGGUA*mU*mG*mU*mU*mU*mU | |

m: 2′-O methyl modified base, *: phosphorothioate backbone

The mRNA encoding the MG3-6/3-4 nuclease was generated by in vitro transcription of a linearized plasmid template using T7 RNA polymerase, nucleotides, and enzymes.

The DNA sequence (SEQ ID NO: 603) that was transcribed into RNA comprised the following elements in order from 5′ to 3′: the T7 RNA polymerase promoter, a 5′ untranslated region (5′ UTR), a nuclear localization signal, a short linker, the coding sequence for the MG3-6/3-4 nuclease, a short linker, a nuclear localization signal, and a 3′ untranslated region and an approximately 100 nucleotide polyA tail (SEQ ID NO: 2865) (not included in SEQ ID No: 603).

The protein sequence encoded in the synthetic mRNA encoded in this MG3-6/3-4 cassette comprises the following elements from 5′ to 3′: the nuclear localization signal from SV40, a five amino acid linker (GGGGS (SEQ ID NO: 2864)), the protein coding sequence of the MG3-6/3-4 nuclease from which the initiating methionine codon was removed, a 3 amino acid linker (SGG) and the nuclear localization signal from nucleoplasmin. The DNA sequence of the protein coding region of this cassette was modified to reflect the codon usage in humans. An approximately 100-nucleotide polyA tail (SEQ ID NO: 2865) was encoded in the plasmid used for in vitro transcription and the mRNA was co-transcriptionally capped. Uridine in the mRNA was replaced with N1-methyl pseudouridine.

The lipid nanoparticle (LNP) formulation used to deliver the MG3-6/3-4 mRNA and the guide RNA comprised 4 lipid components. The four lipid components were dissolved in ethanol and mixed in an appropriate molar ratio to make the lipid working mix. The mRNA and the guide RNA were either mixed prior to formulation at a 1:1 mass ratio or formulated in separate LNP that were later co-injected into mice at a 1:1 mass ratio of the two RNA's. In either case, the RNA was diluted in 100 mM Sodium Acetate (pH 4.0) to make the RNA working stock. The lipid working stock and the RNA working stock were mixed in a microfluidics device at a flow rate ratio of 1:3, respectively and a flow rate of 12 mLs/min. The LNP were dialyzed against phosphate buffered saline (PBS) for 2 to 16 hours and then concentrated until the desired reduced volume was achieved. The concentration of RNA in the LNP formulation was measured using the Ribogreen reagent. The diameter and polydispersity (PDI) of the LNP were determined by dynamic light scattering. Representative LNP had diameters ranged from 65 nm to 120 nm with PDI of 0.05 to 0.20. LNP were injected intravenously into 8- to 12-week-old C57B16 wild type mice via the tail vein (0.1 mL per mouse) at a total RNA dose of 1 mg RNA per kg body weight. Eleven days post-dosing, 3 of the 5 mice in each group were sacrificed and the liver was collected and homogenized using a bead beater in a digestion buffer. Genomic DNA was purified from the resulting homogenate and quantified by measuring the absorbance at 260 nm. Genomic DNA purified from mice injected with buffer alone was used as a control. At 28 days post-dosing, the remaining 2 mice in each group were sacrificed and the liver was collected and homogenized using a bead beater in a digestion buffer. Genomic DNA was purified from the resulting homogenate and quantified by measuring the absorbance at 260 nm. Genomic DNA purified from mice injected with buffer alone was used as a control.

The liver genomic DNA was then PCR amplified using a first set of primers flanking the region targeted by the two guides. The PCR primers used are shown below in Table 11.

TABLE 11
Sequences of PCR primers and Next Generation Sequencing primers used to
analyze in vivo genome editing in mice

| Primer Set | | | |
|---|---|---|---|
| Name | Purpose | Left Primer Sequence | Right Primer Sequence |
| mHAO1-NGS- | Amplify the | GTAAAGAAAAACAAG | ATCTGTCAACTTCTG |
| P4 | target site in | GAATGTAAT (SEQ ID | TTTTAGGAC (SEQ ID |
| | HAO1 exon 2 | NO: 2866) | NO: 2868) |
| | for guide | | |
| | mH364-7 | | |
| mHAO1-NGS- | Amplify the | GCAAAGTAGAGAAATG | ACCAAGTCAGATATA |
| P5 | target site in | ACAAACC (SEQ ID NO: | AACTGTCT (SEQ ID |
| | HAO1 exon 4 | 2867) | NO: 2869) |
| | for guide | | |
| | mH364-20 | | |

The 5′ end of these primers comprise conserved regions complementary to the PCR primers used in the second PCR, followed by 5 Ns in order to give sequence diversity and improve sequencing quality, and end with sequences complementary to the target region in the mouse genome. PCR was performed using 100 ng of genomic DNA and an annealing temperature of 60° C. for a total of 30 cycles. This was followed by a 2nd round of 10 cycles of PCR using primers designed to add unique dual barcodes for next generation sequencing. Each sample was sequenced to a depth of greater than 10,000 reads using 150 bp paired end reads. Reads were merged to generate a single 250 bp sequence from which Indel percentage and INDEL profile was calculated.

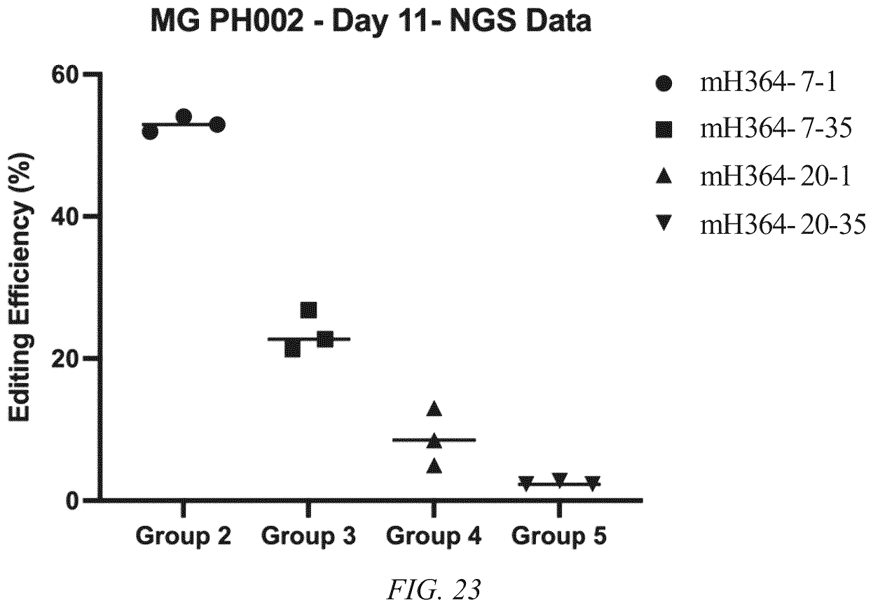

The results of the NGS analysis of INDELS from mice at day 11 post dosing are shown in Table 12 for individual mice and are summarized in FIG. 23.

TABLE 12
Genome editing at the HAO-1 locus by MG3-6/3-4 in
the whole liver of wild type mice at day 11 post
LNP dosing analyzed by next generation sequencing.

| | | | | | | Mean |
|---|---|---|---|---|---|---|
| | | Total | | % of | | total |
| Animal | | NGS | Indel | Indels | Mean | OOF |
| # | Guide RNA | reads | % | OOF | INDELS | % |
| 1 | PBS control | 210962 | 0.09 | 100 | 0.2 | 0.2 |
| 2 | PBS control | 259982 | 0.29 | 99.87 | | |
| 3 | PBS control | 211193 | 0.08 | 100 | | |
| 6 | 364mHA-G7-1 | 164396 | 54.06 | 87.02 | 53.0 | 46.0 |
| 7 | 364mHA-G7-1 | 163409 | 51.93 | 85.9 | | |
| 8 | 364mHA-G7-1 | 183054 | 52.94 | 87.6 | | |
| 11 | 364mHA-G7-35 | 38835 | 22.71 | 91.57 | 23.6 | 21.1 |
| 12 | 364mHA-G7-35 | 269963 | 26.83 | 89.59 | | |
| 13 | 364mHA-G7-35 | 190007 | 21.32 | 87.11 | | |
| 16 | 364mHA-G20-1 | 227766 | 8.53 | 88.62 | 8.9 | 7.5 |
| 17 | 364mHA-G20-1 | 202915 | 5.01 | 90.36 | | |
| 18 | 364mHA-G20-1 | 236757 | 13.06 | 80.52 | | |
| 21 | 364mHA-G20-35 | 177059 | 2.78 | 80.98 | 2.5 | 2.0 |
| 22 | 364mHA-G20-35 | 163515 | 2.29 | 67.62 | | |
| 23 | 364mHA-G20-35 | 136634 | 2.31 | 89.32 | | |

Data for individual mice is shown. All mice that received guide RNA LNP also received LNP encapsulating the MG3-6/3-4 mRNA. % of indels OOF is the percentage of all the INDELS that resulted in a sequence where the HAO1 coding sequence is out of frame. The mean total OOF % is the average percentage of all alleles in which the HAO1 coding sequence is out of frame. The total number of NGS sequencing reads is given.

Group 2 mice received LNP encapsulating guide RNA mH364-7-1. Group 3 mice received LNP encapsulating guide RNAmH364-7-35. Group 4 mice received LNP encapsulating guide RNA mH364-20-1. Group 5 mice received LNP encapsulating guide RNAmH364-20-35. All mice in groups 2 to 5 also received LNP encapsulating the MG3-6/3-4 mRNA that was mixed with the guide RNA containing LNP at a 1:1 RNA mass ratio prior to injection. No INDELS were detected in the liver of mice injected with PBS buffer (see Table 12). Mice injected with LNPs encapsulating guide 364mHA-G7-1 and MG3-6/3-4 mRNA exhibited INDELS at the target site in HAO-1 at a mean frequency of 53.0%. Mice injected with LNPs encapsulating guide 364mHA-G7-35 and MG3-6/3-4 mRNA exhibited INDELS at the target site in HAO-1 at a mean frequency of 23.6%. Mice injected with LNPs encapsulating guide 364mHA-G20-1 and MG3-6/3-4 mRNA exhibited INDELS at the target site in HAO-1 at a mean frequency of 8.9%. Mice injected with LNPs encapsulating guide 364mHA-G20-35 and MG3-6/3-4 mRNA exhibited indels at the target site in HAO-1 at a mean frequency of 2.5%. These data demonstrate that the guides with spacer 7 (364mHA-G7-1 and 364mHA-G7-35) are significantly more potent in vivo than the guides with spacer 20 (364mHA-G20-1 and 364mHA-G20-35) when guides with the same chemical modifications are compared. This is consistent with the higher level of editing observed with these 2 guide sequences in Hepa1-6 cells by mRNA-based transfection (mH364-7 exhibited 46% INDELS and mH364-20 26% INDELS in Hepa1-6 cells). Guide chemistry #1 resulted in higher levels of editing than chemistry #35 for both guide 7 (2.2-fold higher editing with chemistry #1) and guide 20 (3.5-fold higher editing with chemistry #1). These data demonstrate that the MG3-6/3-4 nuclease can edit in vivo in mice at the target site specified by the sgRNA. Moreover, an sgRNA with a set of chemical modifications designated chemistry #1 was able to promote editing at 53% of the genomic DNA in whole liver when delivered using an LNP. The LNP used in these studies is taken up via binding of apolipoprotein E (apoE) to the LNP which is a ligand for binding to the low-density lipoprotein receptor.

The liver is composed of a number of different cell types. In the liver of mice, the hepatocytes make up about 52% of all cells (and 35% of hepatocytes contain two nuclei), with Kupffer cells (18%), Ito cells (8%), and endothelial cells (22%) making up the remaining cells. By extrapolation, without wishing to be bound by theory, about 60% [((52+(0.35×52))/(48+(52+(0.35×52)))] of the total nuclei in the mouse liver are predicted to be derived from hepatocytes. Because the LDL receptor is expressed mainly on hepatocytes in the liver the LNP used in the mouse studies described herein is expected to be taken up primarily by hepatocytes. Because hepatocyte nuclei make up about 60% of all nuclei in the whole liver of mice, it can be predicted that if all the hepatocyte nuclei were edited, the level of INDELS measured in the whole liver are predicted to be about 60%. The finding that LNP delivery of MG3-6/3-4 was able to achieve INDEL rates of 53% suggests that the majority of hepatocyte nuclei were edited.

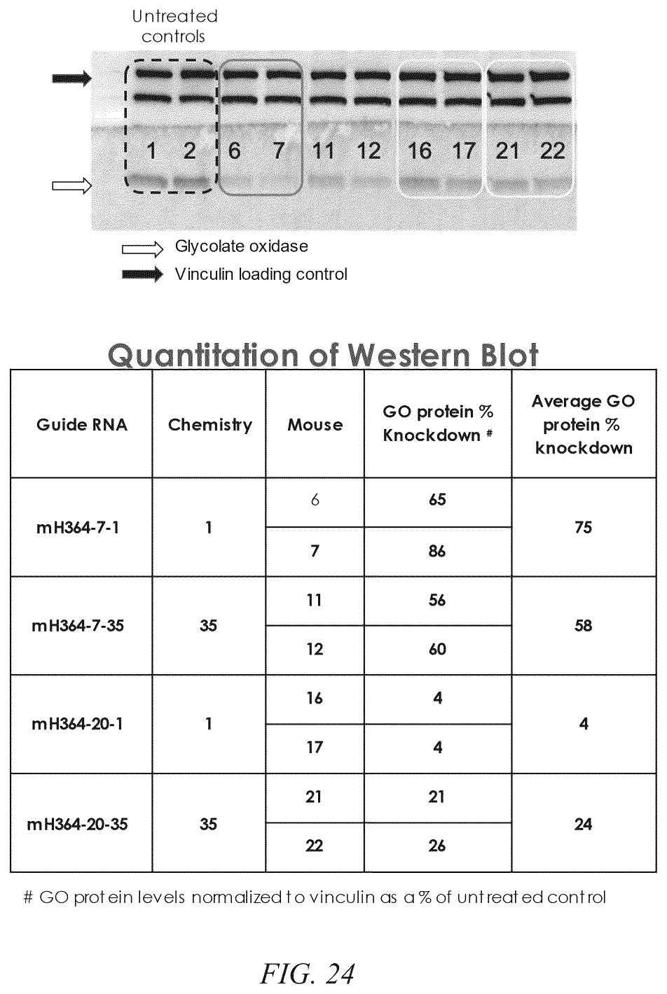

The HAO1 gene encodes the protein glycolate oxidase (GO), an intracellular enzyme involved in glycolate metabolism. To determine if the observed gene editing in the HAO1 gene resulted in a reduction in the expression of the GO protein in the liver, total protein from a separate lobe of the liver was extracted from mice in the same study. The GO protein was detected using a Western blot assay with commercially available antibodies against the mouse GO protein. The protein vinculin was used as a loading control on the Western blot, as Vinculin levels are predicted to not be impacted by gene editing of the HAO1 gene. As shown in FIG. 24, the level of GO protein was significantly reduced in the livers of mice treated with LNP encapsulating MG3-6/3-4 mRNA and sgRNA targeting HAO1. Quantification of the Western blot using image analysis software and normalization of GO to the level of vinculin demonstrated that GO levels were reduced by an average of 75%, 58%, 4%, and 24% in mice treated with sgRNA mH364-7-1, mH364-7-35, mH364-20-1, and mH364-20-35, respectively. The degree of GO protein reduction correlates with the INDEL frequency in these groups of mice (see Table 12). These data demonstrate that the MG3-6/3-4 nuclease combined with an appropriately designed sgRNA can be used to create indels in a gene of interest in vivo in a living mammal and reduce (knockdown) the production of the protein encoded by that gene. Reducing the expression of specific genes can be therapeutically beneficial in specific diseases. In the case of the HAO1 gene that encodes the GO protein, reduction of the levels of GO protein in the liver is expected to be beneficial in patients with the hereditary disease primary hyperoxaluria type I. Thus, the MG3-6/3-4 nuclease, together with an appropriate sgRNA containing appropriate chemical modifications targeting the HAO1 gene, is a potential approach for the treatment of primary hyperoxaluria type I.

### Example 19—Comparison of MG3-6/3-4 Gene Editing Efficiency in Mice Using the Same Guide RNA Sequence with Four Different Chemical Modifications

The impact of chemical modifications to the sgRNA upon in vivo editing efficiency was further investigated by testing 4 different guide chemistries introduced into the same guide RNA sequence. Guide RNA 7 that targets the mouse HAO1 gene was synthesized with chemical modifications #1, #35, #42, or #45. The sequences of these guides are shown below in Table 13.

TABLE 13
Sequences of MG3-6/3-4 sgRNA guide 7 targeting mouse HAO1

| Guide name | Sequence | SEQ ID NO: |
|---|---|---|
| mH364-7-1 | mG*mA*mG*CUGGCCACUGUGCGAGGUAGUUGA | 1821 |
| | GAAUCGAAAGAUUCUUAAUAAGGCAUCCUUCCG | |
| | AUGCUGACUUCUCACCGUCCGUUUUCCAAUAGG | |
| | AGCGGGCGGUAUGU*mU*mU*mU | |
| mH364-7-35 | mG*mA*mG*mC*UGGCCACUGUGCGAGGUAGUU | 1822 |
| | GAGAAUCmG*mA*mA*mA*GAUUCUUAAUAAGG | |
| | CAUCmC*mU*mU*mC*mC*GAUGCUGACUUCUCA | |
| | CCGUCCGUUUUCCmA*mA*mU*mA*GGAGCGGGC | |
| | GGUA*mU*mG*mU*mU*mU*mU | |
| mH364-7-42 | mG*mA*mG*mC*fUfGfGfCfCfAfCfUfGfUfGfCfGfAf | 1823 |
| | GfGfUAGUUGAGAAUCG*A*A*A*GAUUCUUAAUA | |
| | AGGCAUCC*U*U*C*C*GAUGCUGACUUCUCACC | |
| | GUCCGUUUUCCA*A*U*A*GGAGCGGGCGGUA*m | |
| | U*mG*mU*mU*mU*mU | |
| mH364-7-45 | mG*mA*mG*mC*fUfGfGfCfCfAfCfUfGfUfGfCfGfAf | 1824 |
| | GfGfUAGUUGAGAAUCmG*mA*mA*mA*GAUUCU | |
| | UAAUAAGGCAUCmC*mU*mU*mC*mC*GAUGCUG | |
| | ACUUCUCACCGUCCGUUUUCCmA*mA*mU*mA*G | |
| | GAGCGGGCGGUA*mU*mG*mU*mU*mU*mU | |

m: 2′-O methyl modified base, *: phosphorothioate backbone

The mRNA encoding MG3-6/3-4 nuclease was generated by in vitro transcription of a linearized plasmid template using T7 RNA polymerase, nucleotides, and enzymes. The DNA sequence that was transcribed into RNA comprised the following elements in order from 5′ to 3′: the T7 RNA polymerase promoter, a 5′ untranslated region (5′ UTR), a nuclear localization signal, a short linker, the coding sequence for the MG3-6/3-4 nuclease, a short linker, a nuclear localization signal, and a 3′ untranslated region (SEQ ID NO: 603) and an approximately 100 nucleotide polyA tail (SEQ ID NO: 2865) (not included in SEQ ID No: 603).

The protein sequence encoded in the synthetic mRNA encoded in this MG3-6/3-4 cassette comprises the following elements from 5′ to 3′: the nuclear localization signal from SV40, a five amino acid linker (GGGGS (SEQ ID NO: 2864)), the protein coding sequence of the MG3-6/3-4 nuclease from which the initiating methionine codon was removed, a 3 amino acid linker (SGG), and the nuclear localization signal from nucleoplasmin. The DNA sequence of the protein coding region of this cassette was modified to reflect the codon usage in humans. An approximately 100 nucleotide polyA tail (SEQ ID NO: 2865) was encoded in the plasmid used for in vitro transcription, and the mRNA was co-transcriptionally capped. Uridine in the mRNA was replaced with N1-methyl pseudouridine. The lipid nanoparticle (LNP) formulation used to deliver the MG3-6/3-4 mRNA and the guide RNA is based on LNP formulations as described above. The four lipid components were dissolved in ethanol and mixed in an appropriate molar ratio to make the lipid working mix. The mRNA and the guide RNA were either mixed prior to formulation at a 1:1 mass ratio or formulated in separate LNP that were later co-injected into mice at a 1:1 mass ratio of the two RNA's. In either case, the RNA was diluted in 100 mM Sodium Acetate (pH 4.0) to make the RNA working stock. The lipid working stock and the RNA working stock were mixed in a microfluidics device at a flow rate ratio of 1:3, respectively, and a flow rate of 12 mLs/min. The LNP were dialyzed against phosphate buffered saline (PBS) for 2 to 16 hours and then concentrated until the reduced volume was achieved. The concentration of RNA in the LNP formulation was measured using the Ribogreen reagent. The diameter and polydispersity (PDI) of the LNP were determined by dynamic light scattering. Representative LNP had diameters ranged from 65 nm to 120 nm with PDI of 0.05 to 0.20. LNP were injected intravenously into 8- to 12-week-old C57B16 wild type mice via the tail vein (0.1 mL per mouse) at a total RNA dose of 1 mg RNA per kg body weight. Ten days post-dosing, 3 of the 5 mice in each group were sacrificed and the liver was collected and homogenized using a bead beater in a digestion buffer. Genomic DNA was purified from the resulting homogenate and quantified by measuring the absorbance at 260 nm. Genomic DNA purified from mice injected with buffer alone was used as a control. At 28 days post-dosing, the remaining 2 mice in each group were sacrificed and the liver was collected and homogenized using a bead beater in a digestion buffer. Genomic DNA was purified from the resulting homogenate and quantified by measuring the absorbance at 260 nm. Genomic DNA purified from mice injected with buffer alone was used as a control.

The liver genomic DNA was then PCR amplified using a first set of primers flanking the region targeted by the two guides. The PCR primers used are shown in Table 11. The 5′ end of these primers comprise conserved regions complementary to the PCR primers used in the second PCR, followed by 5 Ns in order to give sequence diversity and improve sequencing quality, and end with sequences complementary to the target region in the mouse genome. PCR was performed on 100 ng of genomic DNA and an annealing temperature of 60° C. for a total of 30 cycles. This was followed by a 2nd round of 10 cycles of PCR using primers designed to add unique dual barcodes for next generation sequencing. Each sample was sequenced to a depth of greater than 10,000 reads using 150 bp paired end reads. Reads were merged to generate a single 250 bp sequence from which Indel percentage and INDEL profile was calculated.

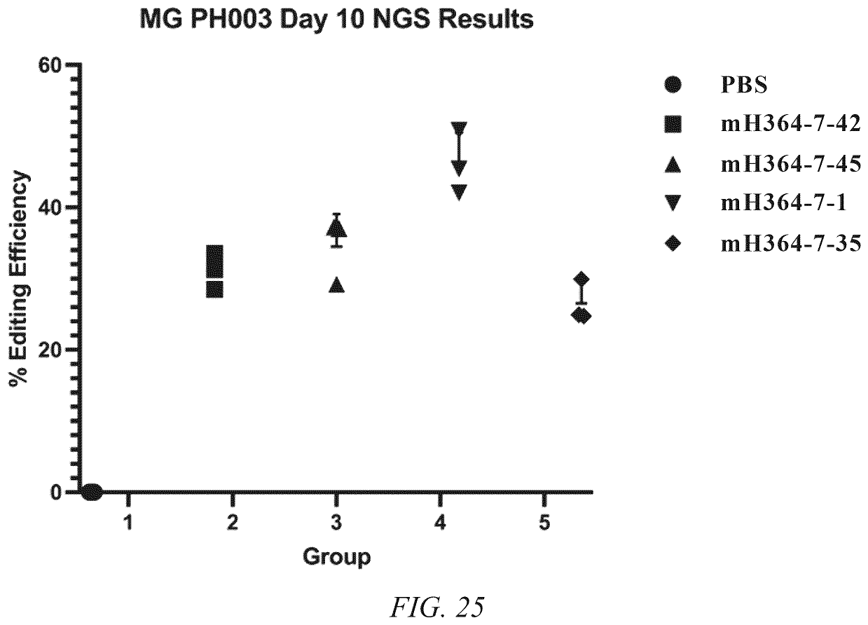

The editing results are summarized in FIG. 25 and tabulated in Table 14.

TABLE 14
Genome editing frequencies in the HAO1 gene in the whole
liver of individual mice treated with LNP encapsulating
MG3-6/3-4 mRNA and guide RNA 7 targeting the
HAO-1 gene with chemical modifications 42 (mH364-7-42),
45 (mH364-7-45), 1 (mH364-7-1), and 35 (mH364-7-35)

| | mH364 Guide 7 | | INDEL | Mean Group | |
|---|---|---|---|---|---|
| DAY | chemistry | Mouse | % | INDELS | Stdev |
| 10 | PBS control | 1 | 0.01 | 0.0 | 0.0 |
| 10 | PBS control | 2 | 0.01 | | |
| 10 | PBS control | 3 | 0.01 | | |
| 28 | PBS control | 4 | 0.02 | | |
| 28 | PBS control | 5 | 0.02 | | |
| 10 | 42 | 6 | 33.54 | 32.4 | 2.5 |
| 10 | 42 | 7 | 28.48 | | |
| 10 | 42 | 8 | 31.3 | | |
| 28 | 42 | 9 | 34.43 | | |
| 28 | 42 | 10 | 34.19 | | |
| 10 | 45 | 11 | 29.22 | 32.1 | 5.8 |
| 10 | 45 | 12 | 37.04 | | |
| 10 | 45 | 13 | 37.24 | | |
| 28 | 45 | 14 | 33.57 | | |
| 28 | 45 | 15 | 23.63 | | |
| 10 | 1 | 16 | 42.04 | 46.1 | 3.1 |
| 10 | 1 | 17 | 45.38 | | |
| 10 | 1 | 18 | 50.8 | | |
| 28 | 1 | 19 | 46.31 | | |
| 28 | 1 | 20 | 45.98 | | |
| 10 | 35 | 21 | 24.95 | 26.6 | 2.3 |
| 10 | 35 | 22 | 29.93 | | |
| 10 | 35 | 23 | 24.75 | | |
| 28 | 35 | 24 | 28.14 | | |
| 28 | 35 | 25 | 25.22 | | |

Control mice injected with PBS buffer did not contain measurable INDELS at the target site for guide 7. The mean INDEL frequency in mice that received LNP containing guides mH364-7-1, mH364-7-35, mH364-7-42, and mH364-7-45 was 46.1%, 26.6%, 32.4%, and 32.1%, respectively, demonstrating that guide RNA chemistry #1 was the most potent followed by #42 and #45, with chemistry #35 being the least potent. These data suggest that chemical modifications to the bases and backbone at the 5′ and 3′ ends of the guide RNA provided the highest in vivo potency amongst the chemistries tested. Additional modifications of internal bases did not improve in vivo potency. These findings are in contrast with published data for the spCas9 sgRNA where modifications of bases or the backbone at both the ends of the sgRNA and at internal sequences was required for optimal in vivo editing and modifications of just the 5′ and 3′ ends of the sgRNA enabled low levels of editing (20% INDELS) in the liver using delivery in a similar LNP.

Total RNA was purified from a separate lobe of the liver from the same mice described in Table 14 and used to measure level of HAO-1 mRNA by digital droplet PCR (dd-PCR). The PBS injected mice were used as controls and the levels of HAO-1 mRNA in the livers of edited mice were compared to these controls. The dd-PCR assay was designed and optimized using molecular biology techniques. ddPCR is a highly accurate method for determining the absolute copy number of a specific nucleic acid in a complex mixture. The total liver RNA was first converted to cDNA by reverse transcription then quantified in the dd-PCR assay using GAPDH as an internal control to normalize between samples. As shown in Table 15A, the level of HAO1 mRNA in the individual mice treated with LNP encapsulating MG3-6/3-4 mRNA and sgRNA targeting the mouse HAO1 gene was decreased, and the magnitude of decrease was correlated with the INDEL frequency.

TABLE 15A
HAO1 mRNA levels in the whole liver of individual mice treated
with LNP encapsulating MG3-6/3-4 mRNA and guide RNA 7 targeting
the HAO-1 gene with chemical modifications 42 (mH364-7-42), 45
(mH364-7-45), 1 (mH364-7-1), and 35 (mH364-7-35).

| | mH364 | | | Mean Group | |
|---|---|---|---|---|---|
| Harvest | Guide 7 | | % Decrease in | % decrease in | |
| Day | chemistry | Mouse | HAO mRNA | HAO mRNA | Stdev |
| 10 | 42 | 6 | 47.4 | 35.5 | 8.8 |
| 10 | 42 | 7 | 42.4 | | |
| 10 | 42 | 8 | 29.0 | | |
| 28 | 42 | 9 | 29.6 | | |
| 28 | 42 | 10 | 28.9 | | |
| 10 | 45 | 11 | 20.3 | 38.0 | 10.2 |
| 10 | 45 | 12 | 38.6 | | |
| 10 | 45 | 13 | 41.8 | | |
| 28 | 45 | 14 | 45.9 | | |
| 28 | 45 | 15 | 43.2 | | |
| 10 | 1 | 16 | 57.0 | 60.0 | 3.9 |
| 10 | 1 | 17 | 54.7 | | |
| 10 | 1 | 18 | 62.5 | | |
| 28 | 1 | 19 | 63.1 | | |
| 28 | 1 | 20 | 62.6 | | |
| 10 | 35 | 21 | 18.3 | 23.4 | 20.8 |
| 10 | 35 | 22 | −2.5 | | |
| 10 | 35 | 23 | 14.8 | | |
| 28 | 35 | 24 | 52.6 | | |
| 28 | 35 | 25 | 33.8 | | |

The same mice as in Table 10 were analyzed

The largest reduction in HAO1 mRNA was seen in the group of mice treated with sgRNA mH364-7-1, while the smallest reduction of HAO-1 mRNA was observed in mice treated with sgRNA mH364-7-35. A reduction in HAO1 mRNA can occur when frameshift mutations are introduced into the coding sequence of a gene via a mechanism called nonsense mediated decay. The observation of reduced HAO-1 mRNA in the liver of mice edited at the HAO-1 gene with MG3-6/3-4 is consistent with the presence of INDELS that result in a high rate of frame shifts as shown in Tables 15A-15B.

TABLE 15B
Analysis of the frequency of edits that result in frame shifts in the
liver of mice treated with LNP encapsulating MG3-6/3-4 mRNA
and sgRNA number 7 (G7) that targets the HAO-1 gene

| | Mean | Stdev of | Mean OOF % | Stdev OFF % |
|---|---|---|---|---|
| Treatment | INDELS | INDELS | total | total |
| PBS control | 0.0 | 0.0 | 0.0 | 0.0 |
| mH364-7-42 | 31.1 | 2.1 | 28.6 | 1.7 |
| mH364-7-45 | 34.5 | 3.7 | 31.2 | 3.2 |
| mH364-7-1 | 46.1 | 3.6 | 41.9 | 3.4 |
| mH364-7-35 | 26.5 | 2.4 | 24.3 | 2.5 |

The out of frame percentage (OOF %) was calculated by analyzing the NGS data

In Table 15B, the mean frequency of INDELS that result in a frame shift in the HAO1 coding sequence were determined from the NGS data. This analysis shows that the majority of the INDELS resulted in a frameshift for all four of the sgRNA tested.

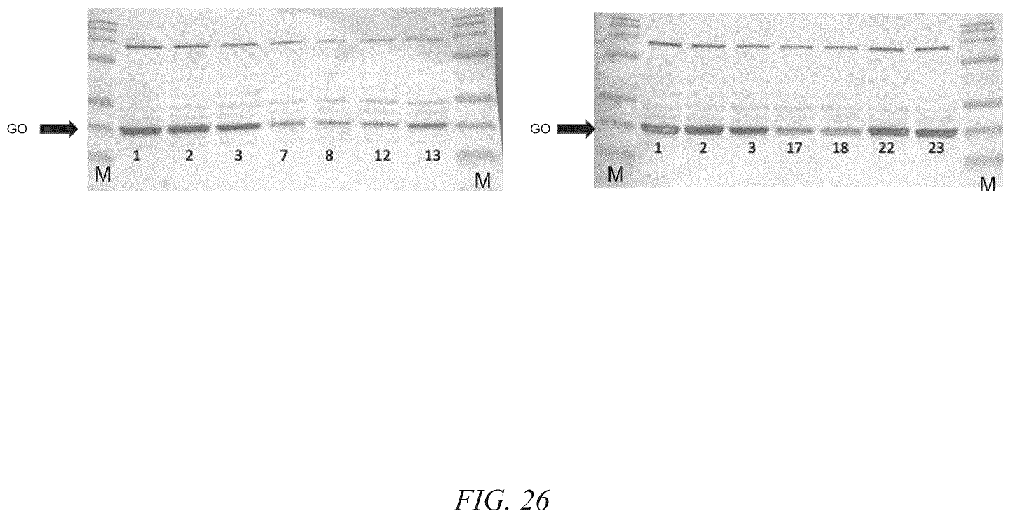

The HAO1 gene encodes the protein glycolate oxidase (GO) that is an intracellular enzyme involved in glycolate metabolism. To determine if the observed gene editing in the HAO1 gene resulted in a reduction in the expression of the GO protein in the liver, total protein from a separate lobe of the liver was extracted from mice in the same study described in FIG. 25 and Tables 14 to 15B. The GO protein was detected using a Western blot assay with commercially available antibodies against the mouse GO protein. Equal amounts of protein were loaded on the Western blot. As shown in FIG. 26, the level of GO protein was reduced in the livers of mice treated with LNP encapsulating MG3-6/3-4 mRNA and sgRNA targeting HAO1. Guides mH364-7-42 (mice 7, 8), mH364-7-45 (mice 12, 13), and mH364-7-1 (mice 17, 18) resulted in clear reductions in GO protein. Guide mH364-7-35 (mice 22, 23) which had the lowest levels of INDELS among the 4 guides tested, did not appreciably reduce GO protein levels. These data demonstrate that the MG3-6/3-4 nuclease combined with an appropriately designed sgRNA can be used to create INDELS in a gene of interest in vivo in a living mammal and reduce (knockdown) the production of the protein encoded by that gene. Reducing the expression of specific genes can be therapeutically beneficial in specific diseases. In the case of the HAO1 gene that encodes the GO protein, reduction of the levels of GO protein in the liver is expected to be beneficial in patients with the hereditary disease primary hyperoxaluria type I. Thus the MG3-6/3-4 nuclease, together with an appropriate sgRNA containing appropriate chemical modifications targeting the HAO1 gene, is a potential approach for the treatment of primary hyperoxaluria type I.

### Example 20—MG3-6/3-4 Nuclease Guide Screen for Human HAO-1 Gene Using mRNA Transfection of Hep3B Cells

Guide RNA for the MG3-6/3-4 nuclease targeting exons 1 to 4 of the human HAO-1 gene (encoding glycolate oxidase) were identified in silico by searching for the PAM sequence 5′ NNAAA(A/T)N 3′. A total of 20 guides with the fewest predicted off-target sites in the human genome were chemically synthesized as single guide RNAs with end-modifications designated AltR1/AltR2 by inserting targeting sequences in a guide scaffold for MG3-6/3-4 (e.g., SEQ ID NO: 722 or SEQ ID NO:863).

### Hep3B Transfection Protocol

300 ng mRNA and 120 ng single guide RNA were transfected into the human hepatoma cell line Hep3B as follows.

One day prior to transfection, Hep3B cells that had been cultured for less than 10 days in EMEM-10% FBS-2 mM glutamine-1% NEAA media, without Pen/Step, were seeded into a TC-treated 24 well plate. Cells were counted, and a volume containing 60,000 viable cells were added to each well. Additional pre-equilibrated media was added to each well to bring the total volume to 500 μL.

On the day of transfection, 300 ng of the MG3-6/3-4 mRNA and 120 ng of the sgRNA were transfected into the Hep3B cells.

Two days post transfection, the media was aspirated from each well of Hep3B cells and genomic DNA was purified by automated magnetic bead purification.

### PCR Amplification and Editing Analysis by Sanger Sequencing

HAO-1 gene sequences targeted by the single guides were amplified by PCR from purified genomic DNA using exon-specific primers (Table 16 below).

TABLE 16
Primers designed for the human HAO1 gene, used for PCR amplification of
the first four exons, and for Sanger sequencing.

| | | | | SEQ |
|---|---|---|---|---|
| Target | | | | ID |
| Exon | Use | Primer Name | Primer Sequence | NO: |
| Human | Fwd PCR | PCR_hHe1_F_ + 490 | TTTCATGGATGCCCCGTTCA | 2870 |
| HAO1 | Rev PCR | PCR_hHe1_R_ − 412 | ACGAAAAGCCAGCAGGAAGA | 2871 |
| Exon 1 | Sequencing | Seq_hHe1_R_ − 121 | AGCCCCAAGAACTTTTCCCT | 2872 |
| Human | Fwd PCR | PCR_hHe2_F_ + 391 | TGCATCAGTGGTTGTCAGGG | 2873 |
| HAO1 | Rev PCR | PCR_hHe2_R_ − 387 | CCTAGCTGTGACTTTGGGCA | 2874 |
| Exon 2 | Sequencing | Seq_hHe2_R_ − 152 | TGGAAAGAAGAGGAGCAGGAC | 2875 |
| Human | Fwd PCR | PCR_hHe3_F_ + 238 | AGGCTGGATGTTCAGGTTCTT | 2876 |
| HAO1 | Rev PCR | PCR_hHe3_R _− 212 | TCCCAAAGCCAAAGCCCTTA | 2877 |
| Exon 3 | Sequencing | Seq_hHe3_F_ + 186 | AGCAGAAATAACTCCAGTAGC | 2878 |
| | | | CA | |
| Human | Fwd PCR | PCR_hHe4_F_ + 324 | GCTGGCTGAAAATCGTGTCAA | 2879 |
| HAO1 | Rev PCR | PCR_hHe4_R_ − 348 | TCCTTGGGGCTTCTCTTTGG | 2880 |
| Exon 4 | Sequencing | Seq_hHe4_F_ + 174 | ACTGATTAAGACCACTAGAGTA | 2881 |
| | | | TCACA | |

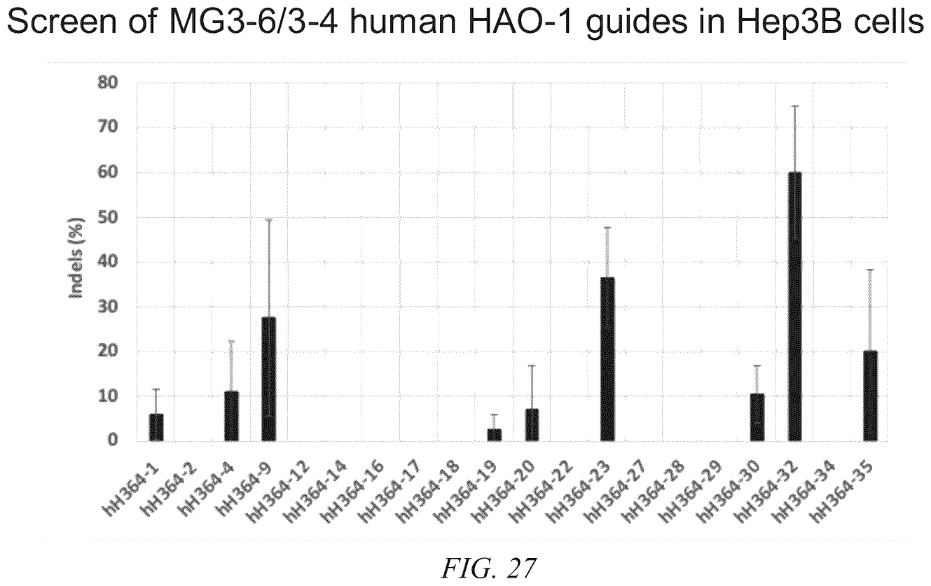

PCR products were purified and concentrated and 40 ng of PCR product subjected to Sanger sequencing. The Sanger sequencing chromatograms were analyzed for insertions and deletions (INDELS) at the predicted target site for each guide. The presence of INDELS at the target site is the consequence of the generation of double strand breaks in the DNA which are then repaired by the error prone cellular repair machinery which introduces insertions and deletions. From this screen, guides hH364-9, 23, 32, and 35 were identified as the most active guides to be assayed for editing in PHH cells (FIG. 27 and Table 17 below).

TABLE 17
Editing activity of MG3-6/3-4 guides targeting the human HAO1 gene
delivered by mRNA transfection, based on MG3-6/3-4 guide

| | | | | Editing | Editing |
|---|---|---|---|---|---|
| | | | | Activity | Activity |
| | | SEQ | | in Hep3B | in PHH |
| Guide | | ID | Spacer Sequence | (Average | (Average |
| Name | PAM | NO: | (Targeting sequence) | % INDELs) | % INDELs) |
| hH364-1 | ACAAATT | 2882 | CATGTTGTTCATAATCATTGAT | 6.0 | |
| hH364-2 | CTAAATC | 2883 | ATCAATGATTATGAACAACATG | 0.0 | |
| hH364-4 | GCAAATG | 2884 | ATATGACTATTACAGGTCTGGG | 11.0 | |
| hH364-9 | AGAAAAT | 2885 | ATATTGCAGCATTTTCCAGGTA | 27.5 | 25.7 |
| hH364-12 | CTAAAAC | 2886 | ATTGGCATGCTGACCCTCTGTC | 0.0 | |
| hH364-14 | ACAAATA | 2887 | CAGGTAAGGTGTGTCCACTGTC | 0.0 | |
| hH364-16 | TAAAATA | 2888 | CTGGTTTCAAAATTTTTCATCC | 0.0 | |
| hH364-17 | CTAAAAT | 2889 | ACTGGTTTCAAAATTTTTCATC | 0.0 | |
| hH364- | TGAAAAA | 2890 | TTATGCATTTCTTATTTTAGGA | 0.0 | |
| 18 | | | | | |
| hH364- | GAAAAAT | 2891 | TATGCATTTCTTATTTTAGGAT | 2.5 | |
| 19 | | | | | |
| hH364- | AAAAATT | 2892 | ATGCATTTCTTATTTTAGGATG | 7.0 | |
| 20 | | | | | |
| hH364- | CAAAATT | 2893 | GAAAATGATAAAGTACTGGTTT | 0.0 | |
| 22 | | | | | |
| hH364- | TCAAAAT | 2894 | AGAAAATGATAAAGTACTGGTT | 36.5 | 26.3 |
| 23 | | | | | |
| hH364- | GGAAAAT | 2895 | GTACTTTATCATTTTCTCCTGA | 0.0 | |
| 27 | | | | | |
| hH364- | GAAAATT | 2896 | TACTTTATCATTTTCTCCTGAG | 0.0 | |
| 28 | | | | | |
| hH364- | CAAAATT | 2897 | GCAGCAAGTCCACTGTCGTCTC | 0.0 | |
| 29 | | | | | |
| hH364- | CCAAAAT | 2898 | TGCAGCAAGTCCACTGTCGTCT | 10.5 | |
| 30 | | | | | |
| hH364- | TCAAATG | 2899 | CCATCTATCAGCTGGGAAGATA | 60.0 | 25.0 |
| 32 | | | | | |
| hH364- | CAAAATG | 2900 | GTAGAGAAATAAACGAACCTCT | 0.0 | |
| 34 | | | | | |
| hH364- | TCAAAAT | 2901 | AGTAGAGAAATAAACGAACCTC | 20.0 | 26.3 |
| 35 | | | | | |

### Example 21—MG3-6/3-4 Nuclease Guide Screen for Human HAO-1 Gene Using mRNA Transfection of Primary Human Hepatocytes (PHH)

The four MG3-6/3-4 single guide RNAs with the highest editing activity in Hep3B cells (guides 9, 23, 32, and 35) were chemically synthesized with end-modifications (Table 18 below), purified by HPLC, and transfected into Primary Human Hepatocytes.

TABLE 18
Sequences of chemically modified MG3-6/3-4 guides assayed in Primary
Human Hepatocytes (m = 2′-O methyl modified base, * = phosphorothioate bond)

| | | SEQ ID |
|---|---|---|
| Guide | Sequence | NO: |
| hH364-9 | mA*mU*mA*UUGCAGCAUUUUCCAGGUAGUUGAGAAUCGA | 1862 |
| | AAGAUUCUUAAUAAGGCAUCCUUCCGAUGCUGACUUCUCA | |
| | CCGUCCGUUUUCCAAUAGGAGCGGGCGGUAUGU*mU*mU*m | |
| | U | |
| hH364-23 | mA*mG*mA*AAAUGAUAAAGUACUGGUUGUUGAGAAUCGA | 1863 |
| | AAGAUUCUUAAUAAGGCAUCCUUCCGAUGCUGACUUCUCA | |
| | CCGUCCGUUUUCCAAUAGGAGCGGGCGGUAUGU*mU*mU*m | |
| | U | |
| hH364-32 | mC*mC*mA*UCUAUCAGCUGGGAAGAUAGUUGAGAAUCGA | 1864 |
| | AAGAUUCUUAAUAAGGCAUCCUUCCGAUGCUGACUUCUCA | |
| | CCGUCCGUUUUCCAAUAGGAGCGGGCGGUAUGU*mU*mU*m | |
| | U | |
| hH364-35 | mA*mG*mU*AGAGAAAUAAACGAACCUCGUUGAGAAUCGA | 1865 |
| | AAGAUUCUUAAUAAGGCAUCCUUCCGAUGCUGACUUCUCA | |
| | CCGUCCGUUUUCCAAUAGGAGCGGGCGGUAUGU*mU*mU*m | |
| | U | |

Notations for chemical modifications: m = 2′O-Methyl ribonucleotide (e.g., mC = cytosine ribonucleotide with 2′-O Methyl in place of 2′ hydroxyl); f = 2′Fluorine ribonucleotide (e.g., fC = cytosine ribonucleotide with 2′ fluorine in place of 2′ hydroxyl); * = phosphorothioate bond. All other bases are native ribonucleotides.

### Primary Human Hepatocyte Transfection Protocol

779.76 ng of MG3-6/3-4 mRNA and 470.24 ng of MG3-6/3-4 single guide RNA with appropriate targeting sequence (1:20 molar ratio of mRNA:guide RNA) were transfected into primary human hepatocyte (PHH) cells as follows.

One day prior to transfection, PHH cells were thawed and seeded in into collagen-treated 12 well plates at 1,000,000 viable cells per well.

On the day of transfection, 779.76 ng of the MG3-6/3-4 mRNA and 470.24 ng of the sgRNA were mixed, and transfected into the PHH cells. Following the transfection, media was replaced every day until harvest.

Three days post transfection, the genomic DNA of the transfected cells was purified by automated magnetic bead purification.

### PCR Amplification and Editing Analysis by Next-Generation Sequencing (NGS)

The region of the HAO-1 gene targeted by each specific sgRNA was PCR amplified with Q5 high fidelity DNA polymerase and gene specific primers (Table 19 below) with adapters complementary to the barcoded primers used for next generation sequencing (NGS) for a total of 29 cycles.

TABLE 19
Primers used to amplify each human HAO1 guide target site and for NGS

| Guide | Primer | Fwd with Miseq | SEQ | Rev with Miseq | SEQ ID |
|---|---|---|---|---|---|
| | Set Name | adapter | ID NO: | adapter | NO: |
| hH364-9 | hHAO1- | GCTCTTCCGATCTN | 2902 | GCTCTTCCGATCTN | 2906 |
| | NGS-P8 | NNNNCCCAAGAAC | | NNNNCCCCCGGCTA | |
| | | TTTTCCCTCTC | | ATTTGTATCA | |
| hH364-23 | hHAO1- | GCTCTTCCGATCTN | 2903 | GCTCTTCCGATCTN | 2907 |
| | NGS-P9 | NNNNGATGTCAGT | | NNNNGAGCAGTGA | |
| | | CTTCTCAGCCA | | ACAGCCAATTG | |
| hH364-32 | hHAO1- | GCTCTTCCGATCTN | 2904 | GCTCTTCCGATCTN | 2908 |
| | NGS-P4 | NNNNTATGAACGT | | NNNNTCTCCTGAGG | |
| | | ATCCACAAAGGAT | | AAAATTTTGGAG | |
| hH364-35 | hHAO1- | GCTCTTCCGATCTN | 2905 | GCTCTTCCGATCTN | 2909 |
| | NGS-P10 | NNNNGAGTTGGAA | | NNNNTGGAGACGA | |
| | | TGTCTCTCTCAA | | CAGTGGACTTG | |

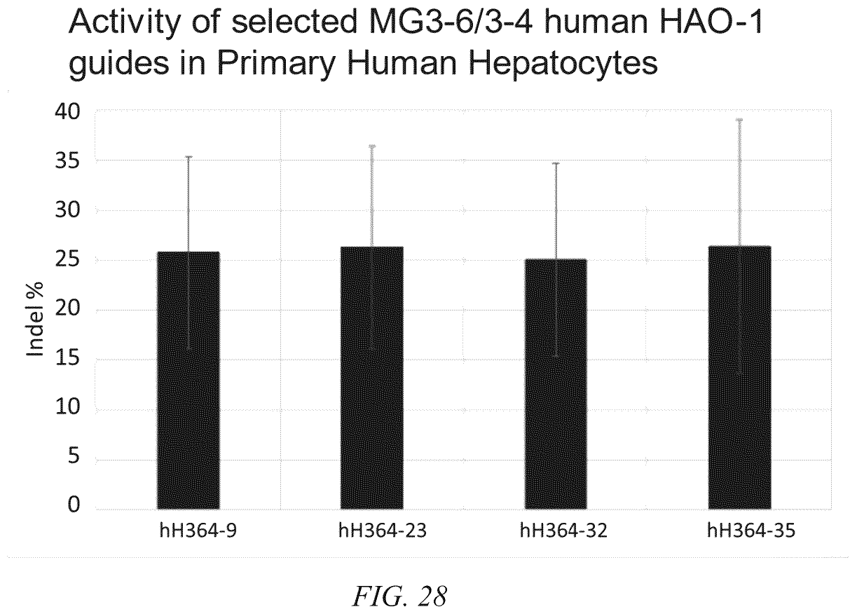

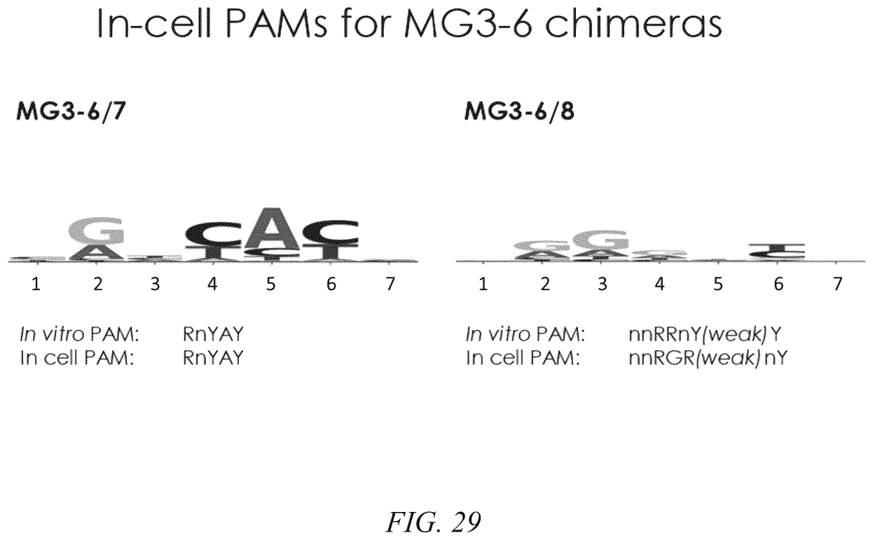

The product of this first PCR reaction was PCR amplified using the barcoded primers for NGS using a total of 10 cycles. The resulting product was subjected to NGS the results were analyzed to generate the percentage of sequencing reads that contain insertions or deletions (indels) at the targeted site in the HAO-1 gene. The results indicate that all four guides edited the HAO-1 gene in PHH with similar activities (FIG. 28 and Table 17). PHH are a surrogate for editing hepatocytes in vivo. These data demonstrate that the MG3-6/3-4 nuclease and an appropriate single guide RNA has utility in generating insertions and deletions (indels) in the coding sequence of the HAO-1 gene. Indel profile obtained from the NGS sequencing was used to determine the percentage of indels that result in a frame shift (i.e., where the number of bases inserted or deleted are not 3 or a multiple of 3). For guides 9, 23, 32, and 35, the percentage of indels that were out of frame in edited PHH was 87.1%, 89.8%, 77.8%, and 82.0%, respectively.

### Example 22—Demonstration of Mammalian Cell Editing and Determination of PAM Sequences for Two MG3-6 Chimeric Enzymes

A lentiviral library was created in which randomized seven base pair regions were flanked by the sequences of target sites for active guide RNAs. A population of 293 cells was created where each cell was transduced by, on average, five lentiviral particles from the library. The appropriate guide RNAs and the matching mRNA encoding chimeric enzymes (where the PAM-interacting domains of MG3-7 and MG3-8 were used to replace the analogous domain in MG3-6) were electroporated into the 293 cell population (2,000,000 cells, 2500 ng mRNA and 1200 pmol guide RNA). Indel formation at the target sites was monitored and the consensus PAM sequence was compiled and is shown in FIG. 29.

### Example 23—In Vivo Editing Activity of MG3-6/3-4 Single Guide RNA with Different Chemical Modifications

To evaluate the ability of the MG3-6/3-4 Type II nuclease to edit the genome in vivo in a living animal, lipid nanoparticles were used to deliver an mRNA encoding the MG3-6/-34 nuclease and one of six guide RNAs with the same nucleotide sequence but different RNA chemistries. The sequences of these guides are shown in Table 20.

TABLE 20
Sequences and chemical modifications of guide RNAs tested in vivo in mice

| Guide | | SEQ ID |
|---|---|---|
| RNA | Sequence | NO: |
| mH364-7-1 | mG*mA*mG*CUGGCCACUGUGCGAGGUAGUUGAGAAUCG | 1812 |
| | AAAGAUUCUUAAUAAGGCAUCCUUCCGAUGCUGACUUC | |
| | UCACCGUCCGUUUUCCAAUAGGAGCGGGCGGUAUGU*mU | |
| | *mU*mU | |
| mH364-7-2 | mG*mA*mG*CUGGCCACUGUGCGAGGUAGUUGAGAAUCG | 1813 |
| | AAAGAUUCUUAAUAAGGCAUCCUUCCGAUGCUGACUUC | |
| | UCACCGUCCGUUUUCCAAUAGGAGCGGGCGGUA*mU*mG | |
| | *mU*mU*mU*mU | |
| mH364-7-9 | mG*mA*mG*CUGGCCACUGUGCGAGGUAGUUGAGAAUCm | 1814 |
| | GmAmAmAGAUUCUUAAUAAGGCAUCmCmUmUmCmCGAU | |
| | GCUGACUUCUCACCGUCCGUUUUCCmAmAmUmAGGAGC | |
| | GGGCGGUAUGU*mU*mU*mU | |
| mH364-7-10 | mG*mA*mG*CUGGCCACUGUGCGAGGUAGUUGAGAAUCG | 1815 |
| | *A*A*A*GAUUCUUAAUAAGGCAUCC*U*U*C*C*GAUGCU | |
| | GACUUCUCACCGUCCGUUUUCCA*A*U*A*GGAGCGGGCG | |
| | GUAUGU*mU*mU*mU | |
| mH364-7-46 | mG*mA*mG*CUGGCCACUGUGCGAGGUAGUUGAGAAUCm | 1819 |
| | GmAmAmAGAUUCUUAAUAAGGCAUCmCmUmUmCmCGAU | |
| | GCUGACUUCUCACCGUCCGUUUUCCmAmAmUmAGGAGC | |
| | GGGCGGUA*mU*mG*mU*mU*mU*mU | |
| mH364-7-47 | mG*mA*mG*CUGGCCACUGUGCGAGGUAGUUGAGAAUCG | 1820 |
| | *A*A*A*GAUUCUUAAUAAGGCAUCC*U*U*C*C*GAUGCU | |
| | GACUUCUCACCGUCCGUUUUCCA*A*U*A*GGAGCGGGCG | |
| | GUA*mU*mG*mU*mU*mU*mU | |

Notations for chemical modifications: m = 2′O-Methyl ribonucleotide (e.g., mC = cytosine ribonucleotide with 2′-O Methyl in place of 2′ hydroxyl); f = 2′Fluorine ribonucleotide (e.g., fC = cytosine ribonucleotide with 2′ fluorine in place of 2′ hydroxyl); * = phosphorothioate bond. All other bases are native ribonucleotides.

The spacer sequence in these guides targets the mouse HAO-1 gene. Guide mH364-7-1 has end modifications. Guide mH364-7-2 has additional methyl and phosphorothioate modifications at the 3′ end. Guide mH364-7-9 has end modifications plus methyl groups in all three loops. Guide mH364-7-10 has end modifications plus phosphorothioate linkages in all three loops. Guide mH364-7-46 has 5′ and 3′ end modifications the same as mH364-7-2 and methyl groups in all three loops. Guide mH364-7-47 has 5′ and 3′ end modifications the same as mH364-7-2, plus the phosphorothioate linkages in all three loops the same as in mH364-7-10. Because the chemical modifications in the sgRNA impact sgRNA stability, which is critical for in vivo potency, it was important to evaluate the impact of these changes on in vivo editing.

Preparation of MG3-613-4 mRNA

The mRNA encoding MG3-6/3-4 was generated by in vitro transcription of a linearized plasmid template using T7 RNA polymerase. The protein coding sequence of the MG3-6/3-4 cassette comprises the following elements from 5′ to 3′: the nuclear localization signal from SV40, a five amino acid linker (GGGGS (SEQ ID NO: 2864)), the protein coding sequence of the MG3-6/3-4 nuclease from which the initiating methionine codon was removed, a 3 amino acid linker (SGG), and the nuclear localization signal from nucleoplasmin. The DNA sequence of this cassette was codon optimized for human. An approximately 100 nucleotide polyA tail (SEQ ID NO: 2865) was encoded in the plasmid used for in vitro transcription and the mRNA was co-transcriptionally capped. Uridine in the mRNA was replaced with N1-methyl pseudouridine.

### Preparation of Lipid Nanoparticles

The lipid nanoparticle (LNP) formulation used to deliver the MG3-6/3-4 mRNA and the guide RNA is based on LNP formulations as described above. The four lipid components were dissolved in ethanol and mixed in an appropriate molar ratio to make the lipid working stock. The RNAs were diluted in 100 mM Sodium Acetate (pH 4.0) to make the RNA working stocks. The lipid working stock and the RNA working stocks were mixed in a microfluidics device at a flow rate ratio of 1:3, respectively, and a flow rate of 12 mL/min. The LNP were dialyzed against phosphate buffered saline (PBS) for 2 to 16 hours and then concentrated. The concentration of RNA in the LNP formulation was measured using the Ribogreen reagent. The diameter and polydispersity (PDI) of the LNP were determined by dynamic light scattering. Representative LNP had diameters ranging from 70 nm to 84 nm and PDI of 0.098 to 0.150.

### Mouse Dosing and Harvesting

LNP for mRNA and sgRNA were mixed at 1:1 mass ratio and injected intravenously into 7-week-old C57B16 wild type mice via the tail vein (0.1 mL per mouse) at a total RNA dose of 0.5 mg RNA per kg body weight. Seven days after dosing, the mice were sacrificed, and the left lateral, medial, and right lateral lobes of the liver were collected for preparation of DNA, RNA, and protein, respectively. Blood was collected by exsanguination via cardiac puncture and collected onto BD microtainer (heparin coated). Samples were kept on wet ice for no longer than 30 minutes prior to centrifugation. Samples were centrifuged at 2,000 G for 10 minutes and the plasma transferred to 1.5 mL Cryotubes and stored at −80° C.

### Genomic DNA Preparation and Editing Analysis by Next-Generation Sequencing (NGS)

The left lateral lobe of the liver (100 mg) was homogenized using a Bead Ruptor in the digestion buffer. Genomic DNA was purified from the resulting homogenate and quantified by measuring the absorbance at 260 nm. Genomic DNA purified from mice injected with PBS buffer alone was used as a control. The region of the HAO-1 gene targeted by each specific sgRNA was PCR amplified with Q5 high fidelity DNA polymerase and gene specific primers (Table 21) with adapters complementary to the barcoded primers used for next generation sequencing (NGS) for a total of 29 cycles. The product of this first PCR reaction was PCR amplified using the barcoded primers for NGS for a total of 10 cycles. The resulting product was subjected to NGS, and the results were analyzed to generate the percentage of sequencing reads that contain insertions or deletions (indels) at the targeted site in the HAO-1 gene.

TABLE 21
Primers used to amplify HAO1 guide target site and for NGS

| Guide | Primer Set Name | Fwd with Miseq adapter | Rev with Miseq adapter |
|---|---|---|---|
| mH364-7 | mHAO1-NGS-P4 | GCTCTTCCGATCTNNNNNG | GCTCTTCCGATCTNNN |
| (spacer 7) | | TAAAGAAAAACAAGGAAT | NNATCTGTCAACTTCT |
| | | GTAAT (SEQ ID NO: 2910) | GTTTTAGGAC (SEQ ID |
| | | | NO: 2911) |

### RNA Preparation and Analysis by RT-ddPCR

The medial lobe of the liver was stored in storage buffer to preserve the integrity of the RNA prior to homogenization. A maximum of 10 mg of tissue was transferred to a 2 mL tube containing 1.4 mm ceramic beads and homogenized using a Bead Ruptor following the soft tissue homogenization protocol: 5.00 m/s for 10-15 sec. Homogenized tissue was then processed and treated with an additional 45 minute on-column DNase I digestion treatment. The RNA products were quantified by measuring the absorbance at 260 nm. Isolated RNA samples then underwent an additional DNase treatment and reverse transcription. The cDNA product was then quantified by measuring the absorbance at 260 nm.

HAO1 mRNA levels were quantified using a custom ddPCR probe-based assay that was multiplexed with the housekeeping gene GAPDH. HAO1 was labeled with a HEX probe, while GAPDH was labeled with a FAM probe. Both probes were flanked by primers specific to the respective genes, creating amplicons of about 115 bp. The template material for the assay was 25 ng of cDNA product from the process above mixed with 900 nM of each primer and 250 nM of probe per gene assay along with 10 μL of ddPCR Supermix for Probes (No dUTP) raised to a final volume of 20 μL with nuclease-free water. The ddPCR was performed and analyzed. Results were divided into four sections: negative droplets (no fluorescence), single positive droplets (HEX or FAM positive fluorescence), and double positive droplets (both HEX and FAM fluorescence). Copies per p L of HAO1 and GAPDH were calculated for each sample. The ratio of HAO1 to GAPDH was compared for mice treated with mH29-29 against mice treated with buffer alone.

TABLE 22A
Primers and probes used in the amplification HAO1 and GAPDH ddPCR
assays

| Target | Probe | Probe | Fwd Primer | Rev primer |
|---|---|---|---|---|
| Gene | Fluorescence | Sequence | Sequence | sequence |
| HAO1 | HEX | AGTG+GGTG+C+C | GGGGAGAAAGG | CTCACCAATGT |
| | | A+GAAT+GTGAA | TGTTCAAGATGT | CTTGTCGATGA |
| | | (SEQ ID NO: 2912) | (SEQ ID NO: 2914) | (SEQ ID NO: |
| | | | | 2916) |
| GAPDH | FAM | +C+CATGCCATC | GCTTAG (SEQ ID | CTTCTGGG (SEQ |
| | | (SEQ ID NO: 2913) | NO: 2915) | ID NO: 2917) |
| | | CATGACCA+CAGT | GCACCACCAACT | CCATCCACAGT |

Notations for chemical modifications: + = Locked Nucleic Acid (LNA) base modification.

### Results and Conclusions

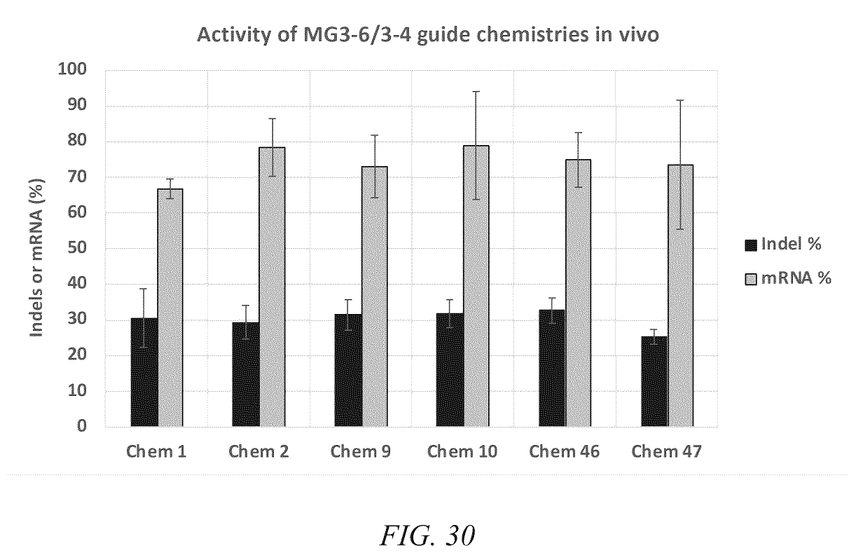

The NGS analysis of mouse liver genomic DNA showed that editing at the target site of the sgRNA in the HAO-1 gene of the groups dosed with LNP encapsulating MG3-6/3-4 mRNA and each of the six sgRNA ranged from 25% to 33% of total liver genomic DNA (FIG. 30). There were minor differences between most of the groups, indicating that guide RNAs with chemistries 1, 2, 9, 10, and 46 edited with similar efficiency, and that chemistry 47 may be somewhat less effective.

The GAPDH-normalized levels of HAO1 mRNA in the livers of mice from the groups treated with LNP encapsulating the mH364-7 sgRNA and MG3-6/3-4 mRNA ranged from 67% to 79% of the control group (FIG. 30). Editing of the HAO-1 gene that introduces INDELS into the coding sequence may result in a reduction in the amount of HAO-1 mRNA expressed as a result of nonsense mediated mRNA decay. The magnitude of reduction in HAO-1 mRNA correlated with the genome editing efficiency. Taken together, the genome editing and HAO-1 mRNA knockdown data demonstrate that the different sgRNA chemistries 1, 2, 9, 10, 46, and 47 had minor effects upon in vivo potency.

### Example 24—Analysis of Gene-Editing Outcomes at the DNA Level for VCP in K562 Cells

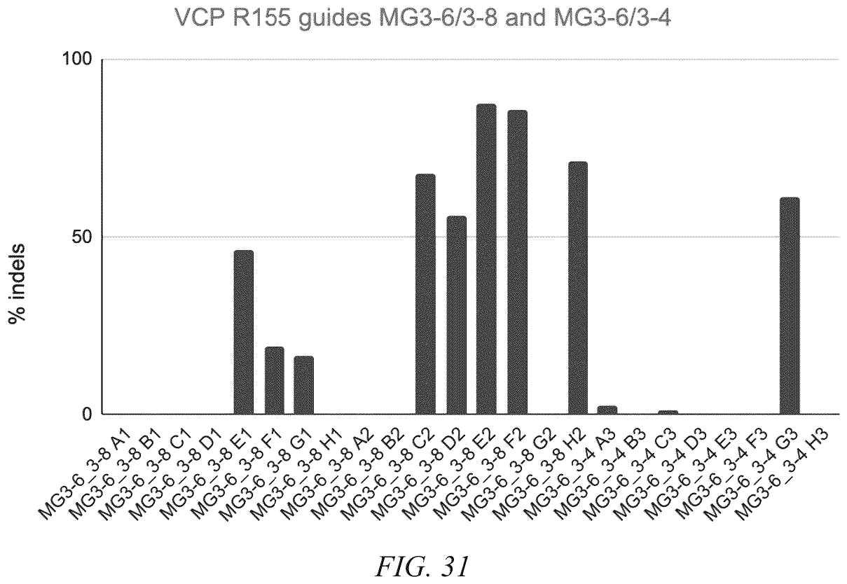

Nucleofection of either MG3-6/3-8 or MG3-6/3-4 mRNA and guide (500 ng mRNA, 150 pmol guide) was performed into K562 cells (200,000) using electroporation. Guides have a single mismatch targeting R155. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing (FIG. 31).

TABLE 22B
Guide Sequences for Editing VCP as Described in Example 24

| SEQ | | |
|---|---|---|
| ID | Guide | |
| NO: | RNA | Sequence |
| 723 | MG3- | mG*mC*mA*rCrGrCrArUrCrCrCrArCrCrArCrGrGrArCrArArGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | A1 | |
| 724 | MG3- | mG*mC*mA*rCrGrCrArUrCrCrCrArCrCrArUrGrGrArCrArArGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | B1 | |
| 725 | MG3- | mA*mC*mG*rCrArUrCrCrCrArCrCrArCrGrGrArCrArArGrArGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | C1 | |
| 726 | MG3- | mA*mC*mG*rCrArUrCrCrCrArCrCrArUrGrGrArCrArArGrArGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | D1 | |
| 727 | MG3- | mC*mG*mG*rArCrArArGrArArArArArUrGrUrCrUrCrCrUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | E1 | |
| 728 | MG3- | mU*mG*mG*rArCrArArGrArArArArArUrGrUrCrUrCrCrUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP F1 | CrGrGrUrArUrGrU*mU*mU*mU |
| 729 | MG3- | mG*mG*mA*rGrArCrArUrUrUrUrUrCrUrUrGrUrCrCrGrUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | G1 | |
| 730 | MG3- | mG*mG*mA*rGrArCrArUrUrUrUrUrCrUrUrGrUrCrCrArUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | H1 | |
| 731 | MG3- | mA*mG*mA*rCrArUrUrUrUrUrCrUrUrGrUrCrCrGrUrGrGrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | A2 | |
| 732 | MG3- | mA*mG*mA*rCrArUrUrUrUrUrCrUrUrGrUrCrCrArUrGrGrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | B2 | |
| 733 | MG3- | mU*mG*mU*rCrCrGrUrGrGrUrGrGrGrArUrGrCrGrUrGrCrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | C2 | |
| 734 | MG3- | mU*mG*mU*rCrCrArUrGrGrUrGrGrGrArUrGrCrGrUrGrCrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | D2 | |
| 735 | MG3- | mG*mU*mC*rCrGrUrGrGrUrGrGrGrArUrGrCrGrUrGrCrUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | E2 | |
| 736 | MG3- | mG*mU*mC*rCrArUrGrGrUrGrGrGrArUrGrCrGrUrGrCrUrGrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP F2 | CrGrGrUrArUrGrU*mU*mU*mU |
| 737 | MG3- | mU*mC*mC*rGrUrGrGrUrGrGrGrArUrGrCrGrUrGrCrUrGrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | G2 | |
| 738 | MG3- | mU*mC*mC*rArUrGrGrUrGrGrGrArUrGrCrGrUrGrCrUrGrUrGrUrUrGrArGrArArUr |
| | 6/3-8- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | H2 | |
| 739 | MG3 | GCACGCATCCCACCACGGACAA |
| | 6/3-8- | |
| | human | |
| | VCP | |
| | A1 | |
| 740 | MG3- | GCACGCATCCCACCATGGACAA |
| | 6/3-8- | |
| | human | |
| | VCP | |
| | B1 | |
| 741 | MG3- | ACGCATCCCACCACGGACAAGA |
| | 6/3-8- | |
| | human | |
| | VCP | |
| | C1 | |
| 742 | MG3- | ACGCATCCCACCATGGACAAGA |
| | 6/3-8- | |
| | human | |
| | VCP | |
| | D1 | |
| 743 | MG3- | CGGACAAGAAAAATGTCTCCTG |
| | 6/3-8- | |
| | human | |
| | VCP | |
| | E1 | |
| 744 | MG3- | TGGACAAGAAAAATGTCTCCTG |
| | 6/3-8- | |
| | human | |
| | VCP F1 | |
| 745 | MG3- | GGAGACATTTTTCTTGTCCGTG |
| | 6/3-8- | |
| | human | |
| | VCP | |
| | G1 | |
| 746 | MG3- | GGAGACATTTTTCTTGTCCATG |
| | 6/3-8- | |
| | human | |
| | VCP | |
| | H1 | |
| 747 | MG3- | AGACATTTTTCTTGTCCGTGGT |
| | 6/3-8- | |
| | human | |
| | VCP | |
| | A2 | |
| 748 | MG3- | AGACATTTTTCTTGTCCATGGT |
| | 6/3-8- | |
| | human | |
| | VCP | |
| | B2 | |
| 749 | MG3- | TGTCCGTGGTGGGATGCGTGCT |
| | 6/3-8- | |
| | human | |
| | VCP | |
| | C2 | |
| 750 | MG3- | TGTCCATGGTGGGATGCGTGCT |
| | 6/3-8- | |
| | human | |
| | VCP | |
| | D2 | |
| 751 | MG3- | GTCCGTGGTGGGATGCGTGCTG |
| | 6/3-8- | |
| | human | |
| | VCP | |
| | E2 | |
| 752 | MG3- | GTCCATGGTGGGATGCGTGCTG |
| | 6/3-8- | |
| | human | |
| | VCP F2 | |
| 753 | MG3- | TCCGTGGTGGGATGCGTGCTGT |
| | 6/3-8- | |
| | human | |
| | VCP | |
| | G2 | |
| 754 | MG3- | TCCATGGTGGGATGCGTGCTGT |
| | 6/3-8- | |
| | human | |
| | VCP | |
| | H2 | |
| 755 | MG3 | mC*mA*mG*rCrArCrGrCrArUrCrCrCrArCrCrArCrGrGrArCrGrUrUrGrArGrArArUrC |
| | 6/3-4- | rGrArArArGrArUrUrCrUrUrArArUrArArGrGrCrArUrCrCrUrUrCrCrGrArUrGrCrUrGr |
| | human | ArCrUrUrCrUrCrArCrCrGrUrCrCrGrUrUrUrUrCrCrArArUrArGrGrArGrCrGrGrGrCr |
| | VCP | GrGrUrArUrGrU*mU*mU*mU |
| | A3 | |
| 756 | MG3- | mC*mA*mG*rCrArCrGrCrArUrCrCrCrArCrCrArUrGrGrArCrGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | B3 | |
| 757 | MG3- | mA*mG*mC*rArCrGrCrArUrCrCrCrArCrCrArCrGrGrArCrArGrUrUrGrArGrArArUr |
| | 6/3-4 | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | C3 | |
| 758 | MG3- | mA*mG*mC*rArCrGrCrArUrCrCrCrArCrCrArUrGrGrArCrArGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | D3 | |
| 759 | MG3- | mG*mC*mA*rCrGrCrArUrCrCrCrArCrCrArCrGrGrArCrArArGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | E3 | |
| 760 | MG3- | mG*mC*mA*rCrGrCrArUrCrCrCrArCrCrArUrGrGrArCrArArGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP F3 | CrGrGrUrArUrGrU*mU*mU*mU |
| 761 | MG3- | mC*mA*mC*rGrCrArUrCrCrCrArCrCrArCrGrGrArCrArArGrGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | G3 | |
| 762 | MG3- | mC*mA*mC*rGrCrArUrCrCrCrArCrCrArUrGrGrArCrArArGrGrUrUrGrArGrArArUr |
| | 6/3-4- | CrGrArArArGrArUrUrCrUrUrArArUrArArGrGrCrArUrCrCrUrUrCrCrGrArUrGrCrUr |
| | human | GrArCrUrUrCrUrCrArCrCrGrUrCrCrGrUrUrUrUrCrCrArArUrArGrGrArGrCrGrGrGr |
| | VCP | CrGrGrUrArUrGrU*mU*mU*mU |
| | H3 | |
| 763 | MG3- | CAGCACGCATCCCACCACGGAC |
| | 6/3-4- | |
| | human | |
| | VCP | |
| | A3 | |
| 764 | MG3- | CAGCACGCATCCCACCATGGAC |
| | 6/3-4- | |
| | human | |
| | VCP | |
| | B3 | |
| 765 | MG3- | AGCACGCATCCCACCACGGACA |
| | 6/3-4- | |
| | human | |
| | VCP | |
| | C3 | |
| 766 | MG3- | AGCACGCATCCCACCATGGACA |
| | 6/3-4- | |
| | human | |
| | VCP | |
| | D3 | |
| 767 | MG3- | GCACGCATCCCACCACGGACAA |
| | 6/3-4- | |
| | human | |
| | VCP | |
| | E3 | |
| 768 | MG3- | GCACGCATCCCACCATGGACAA |
| | 6/3-4- | |
| | human | |
| | VCP F3 | |
| 769 | MG3- | CACGCATCCCACCACGGACAAG |
| | 6/3-4- | |
| | human | |
| | VCP | |
| | G3 | |
| 770 | MG3- | CACGCATCCCACCATGGACAAG |
| | 6/3-4- | |
| | human | |
| | VCP | |
| | H3 | |

Notations for chemical modifications: m = 2′O-Methyl ribonucleotide (e.g., mC = cytosine ribonucleotide with 2′-O Methyl in place of 2′ hydroxyl); f = 2′Fluorine ribonucleotide (e.g., fC = cytosine ribonucleotide with 2′ fluorine in place of 2′ hydroxyl); * = phosphorothioate bond. All other bases are native ribonucleotides.

### Example 25—In Vitro Transcription of mRNA

Sequences for chimera mRNA were codon optimized for human expression, then synthesized and cloned into a high copy ampicillin plasmid. Synthesized constructs encoding T7 promoter, UTRs, chimera ORF, and NLS sequences were digested from the backbone with HindII and BamHI, and ligated into a pUC19 plasmid backbone (Seq ID NO: 1788). The complete chimera mRNA plasmid comprises an ORI, ampicillin resistance cassette, the synthesized construct, and an encoded polyA tail. Chimera mRNA was synthesized via in vitro transcription (IVT) using the linearized chimera mRNA plasmid. This plasmid was linearized and purified with Phenol:Chloroform:Isoamyl Alcohol (25:24:1, v/v), precipitated in ethanol, and resuspended in nuclease-free water at an adjusted concentration of 500 ng/μL. The IVT reaction to generate chimera mRNA was performed at 50° C. for 1 hr. After 1 hr, the IVT reaction was stopped and plasmid DNA was digested with the addition of 250 U/mL DNaseI and incubated for 10 min at 37° C. Chimera mRNA was purified and transcript concentration was determined by UV and further analyzed by capillary gel electrophoresis.

### Example 26—Protein Recombination of Type II Endonucleases

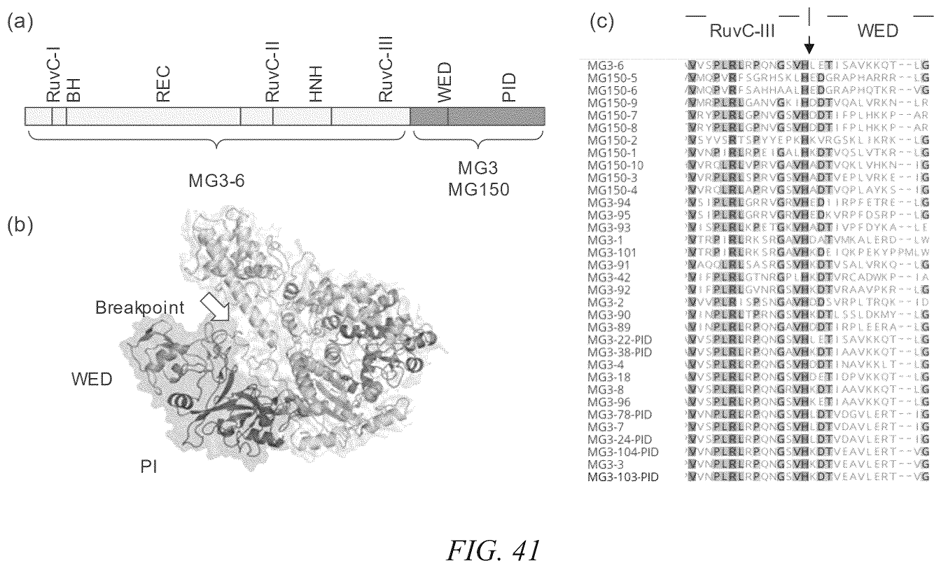

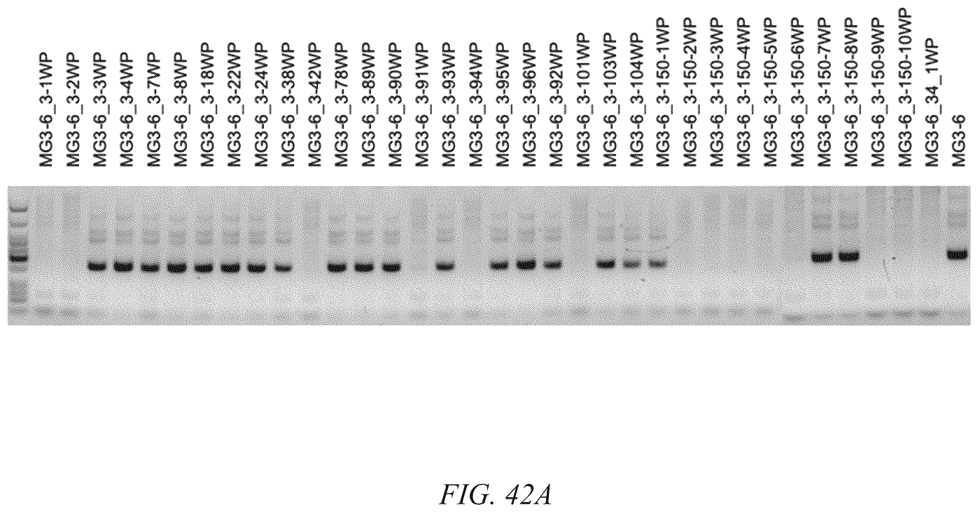

MG3-6 is a highly active and specific double-stranded DNA (dsDNA) cleaving enzyme. Engineering of the enzyme to accommodate a more generic or different preference for PAM (protospacer adjacent motif, a short stretch of DNA sequence required for DNA recognition) would further increase the utility of the enzyme to target additional sequences in DNA. A set of MG3-6 chimeras were generated by swapping domains including partial RuvC (e.g., RuvC-III, denoted as R), WED (denoted as W), and PAM-interacting domain (denoted as PI) domains. For simplicity, these chimeric systems were individually designated MG3-6 followed by _MG number and RWP. To expand the compatibility of PAM in the genome, the WED and PI domain (PAM interaction domain) of MG3-6 were swapped with domains from MG3 and MG150 (MG3-like family) proteins. The breakpoint was chosen in the unstructured loop between RuvC-III and WED domains (see FIG. 41 panels (a) and (b)). To determine the specific amino acid position for structural recombination, homologous sequences of MG3 and MG150 were analyzed, and WED and PI domains were swapped right after histidine, corresponding to residue 838 of MG3-6 (see FIG. 41 panel (c)) (chimeric sequences provided as SEQ ID NOs: 774-807).

Activities of chimeric proteins were characterized by an automated in vitro PAM enrichment, where active enzymes digested dsDNA and subsequent processing yielded DNA fragments as products, according to the following procedure. Chimera sequences were codon-optimized for E. coli expression, synthesized, and cloned into pAL3 vector (SEQ ID NO: 920). The PAM sequence was determined via expression in either an E. coli lysate-based expression system or reconstituted via in vitro translation. The E. coli codon-optimized protein sequence was transcribed and translated from a PCR fragment under control of a T7 promoter. This mixture was diluted into a reaction buffer (10 mM Tris pH 7.5, 100 mM NaCl, 10 mM MgCl₂) with protein-specific sgRNA and a PAM plasmid library (PAM library U67/U40). Specifically, MG3-6 sgRNA was used for MG3-6 chimera recombining with MG3 and MG150 members (SEQ ID NO: 863). The guide was synthesized. The library of plasmids contained a spacer sequence matching that in the single guide followed by 8N mixed bases, a subset of which would have the preferred PAM (SEQ ID NO: 921). After 1-3 h, the reaction was stopped, and the DNA was recovered. The cleaved DNA products from Type II chimera's digestion were subjected to a blunt-end ligation reaction which added adapter sequences to cleaved library plasmids while leaving intact circular plasmids unchanged. A PCR was performed with primers (LA065 and LA125) (SEQ ID NOs: 1786-1787) specific to the library and the adapter sequence, and the product was resolved on a gel to identify active protein complexes. The resulting PCR products were further amplified by PCR using high throughput sequencing primers. Samples subjected to NGS analysis were quantified and pooled together. The NGS library was purified and quantified. Sequencing this library, which was a subset of the starting 8N library, reveals the sequences which contain the correct PAM.

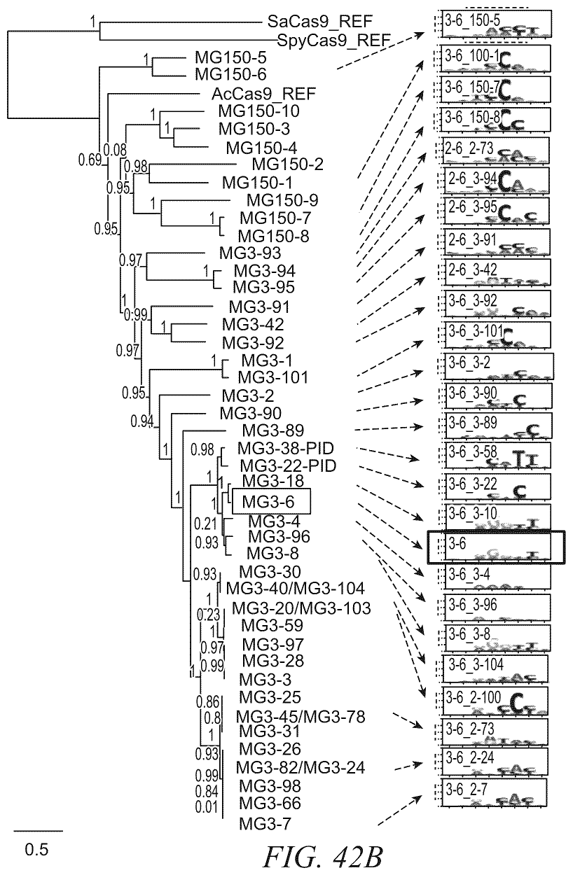

The agarose gel result suggested that 20 out 33 recombined MG3-6 with MG3 and MG150 were active, a hit of 60% with this breakpoint (see FIG. 42A). A lower success rate of MG3-6 recombining with MG150 may be due to structural disruption. While bands for MG3-6_3-2WP, MG3-6_3-42WP, MG3-6_3-91WP, MG3-6_3-94WP, MG3-6_3-101WP were not observed on the agarose gel, seqlogos were acquired with sensitive NGS analysis. It was observed that closely related PIDs may share similar PAMs. For example, MG3-6_150-1WP, MG3-6_150-7WP, and MG3-6_150-8WP exhibited sequence preferences of CC on the fourth and fifth positions in an 8N library. With this protein engineering approach, the MG3-6 PAM (nnRGRYYn) was expanded to a variety of PAM sequences (see FIG. 42B).

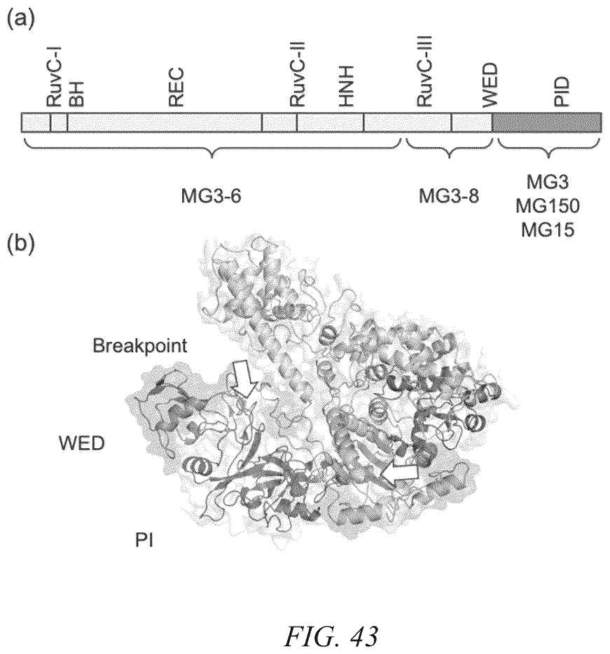

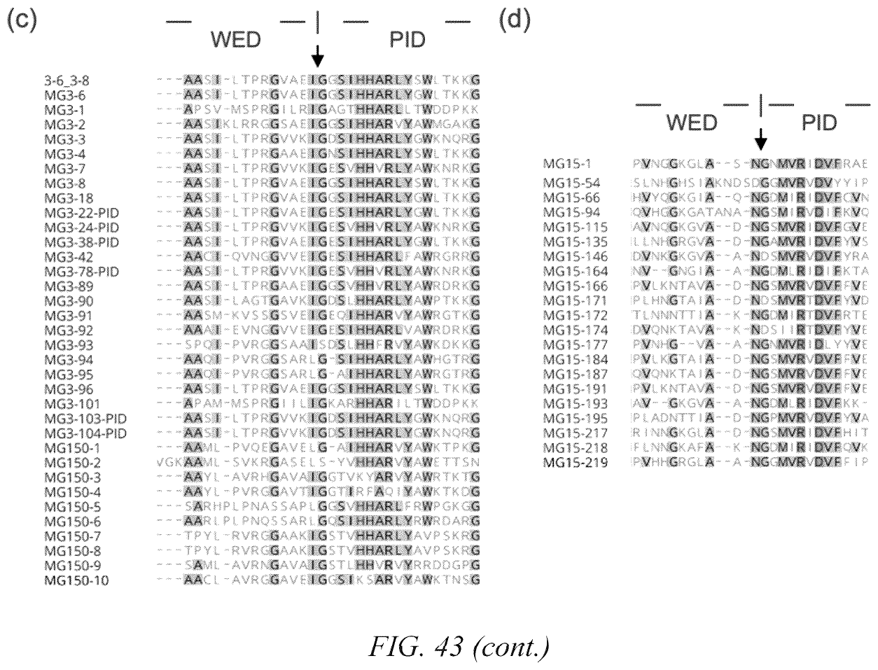

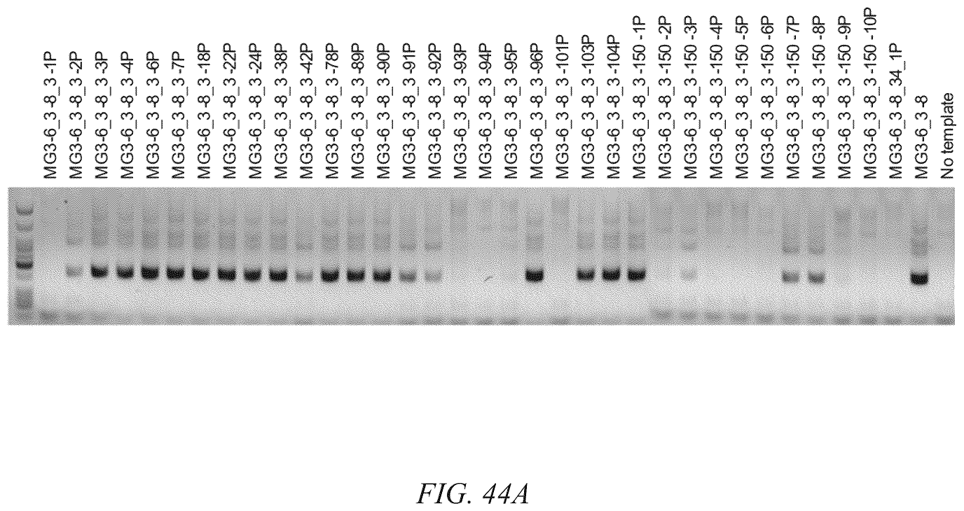

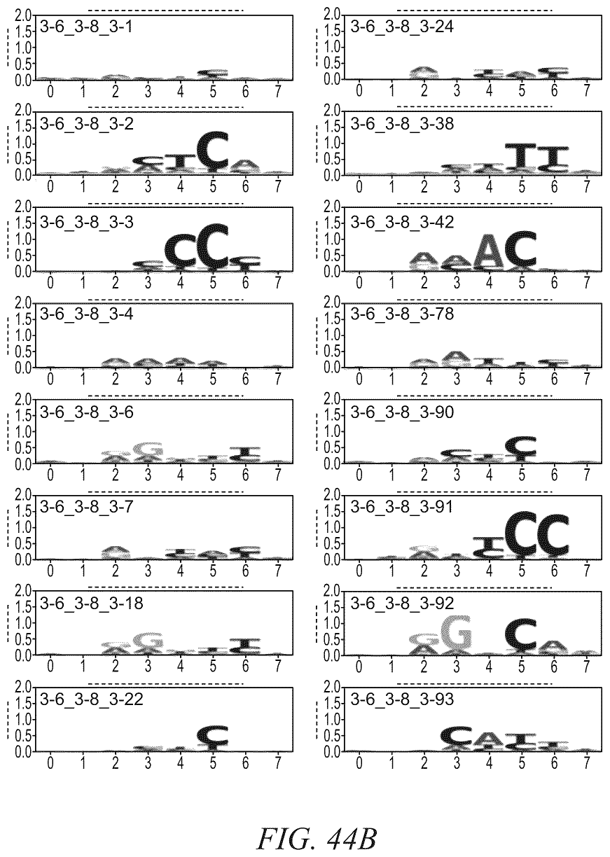

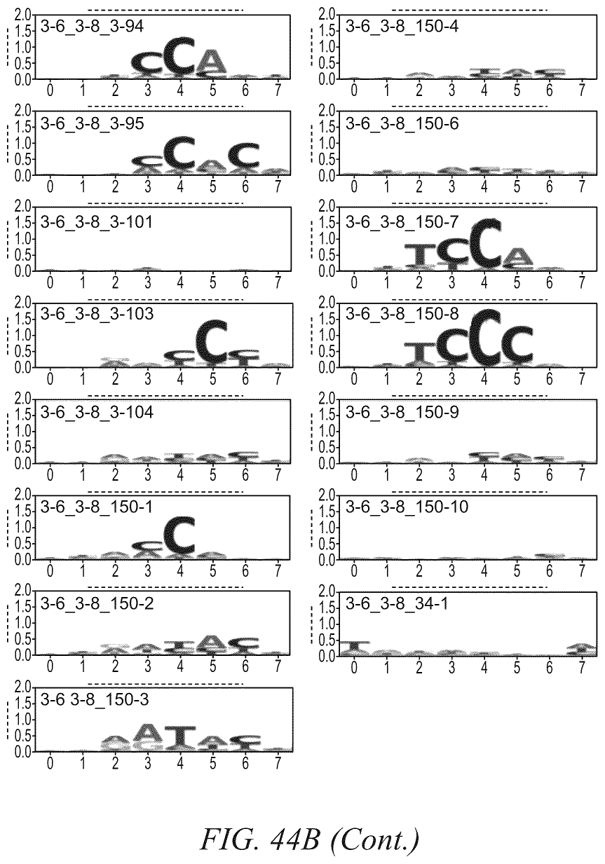

A domain swap of partial RuvC-III, WED, and PI of MG3-6 with MG3-8 altered its PAM from nnRGRYYn to nnRRnnYn, resulting in MG3-6_3-8 that was able to increase targeting density in the genome. To alter the PAM of MG3-6_3-8RWP without compromising its activity, N-terminal MG3-6 and partial RuvC-III plus WED domains from MG3-8 were maintained as the nuclease chassis for recombination. PID domains used for recombination were sourced from MG3, MG15, and MG150 members (see FIG. 43 panels (a) and (b)). The breakpoint of MG3-6_3-8 recombining MG3 and MG150 members was selected in the floppy loop among WED and PID (see FIG. 43 panel (c)) (SEQ ID NOs: 808-841). Similarly, the same region was chosen for recombination of MG3-6_3-8 recombining MG15 members (SEQ ID NOs: 842-862). Out of 33 chimeras tested, 22 showed distinct bands on the agarose gel, suggesting cleavage activities of these nucleases. Previously, MG3-6_3-2WP, MG3-6_42WP, and MG3-6_3-91WP did not display high activity as suggested agarose gel results. However, given a new breakpoint, it was observed that they may become more active using the chassis of MG3-6_3-8 (FIG. 44A). Subsequent NGS analysis revealed a panel of diversified PAMs for broader genome targeting (FIG. 44B).

### Example 27—Screening of Chimeras in Mammalian Cells

Nucleofection of mRNA and guide (500 ng mRNA, 150 pmol guide) was performed in K562 cells (200,000 cells per guide), Hep3B cells (100,000 cells per guide), or Hepa1-6 cells (100,000 cells per guide) using electroporation. Guides used for screening were chemically synthesized. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to determine gene editing.

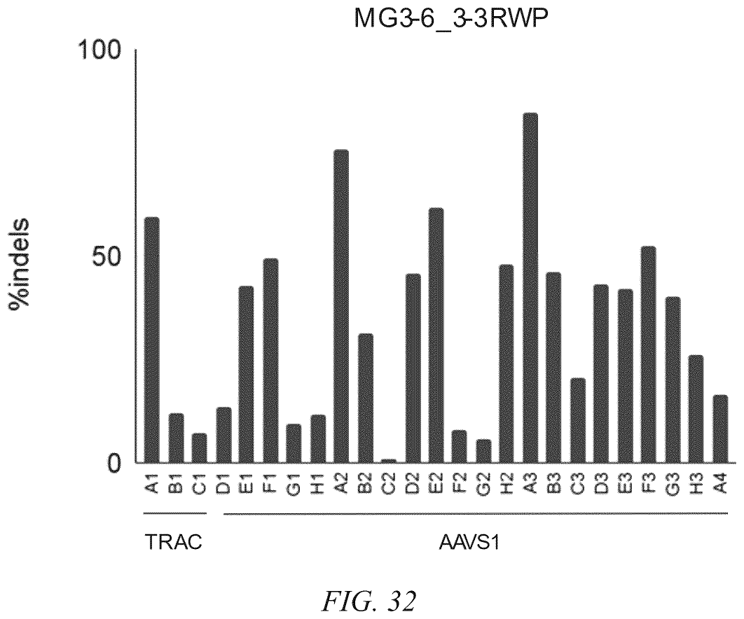

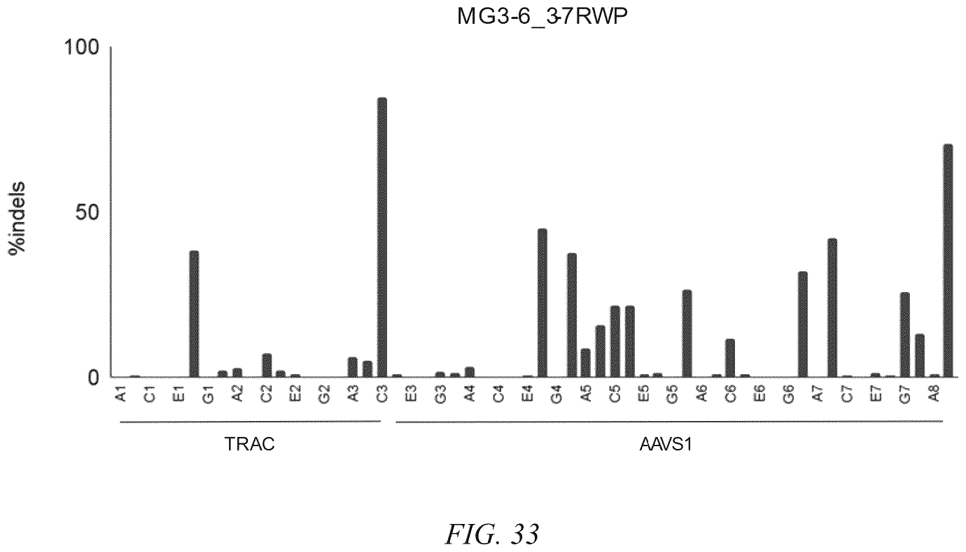

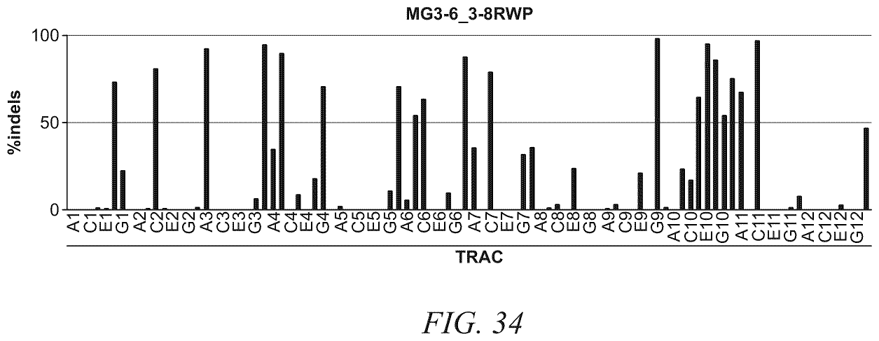

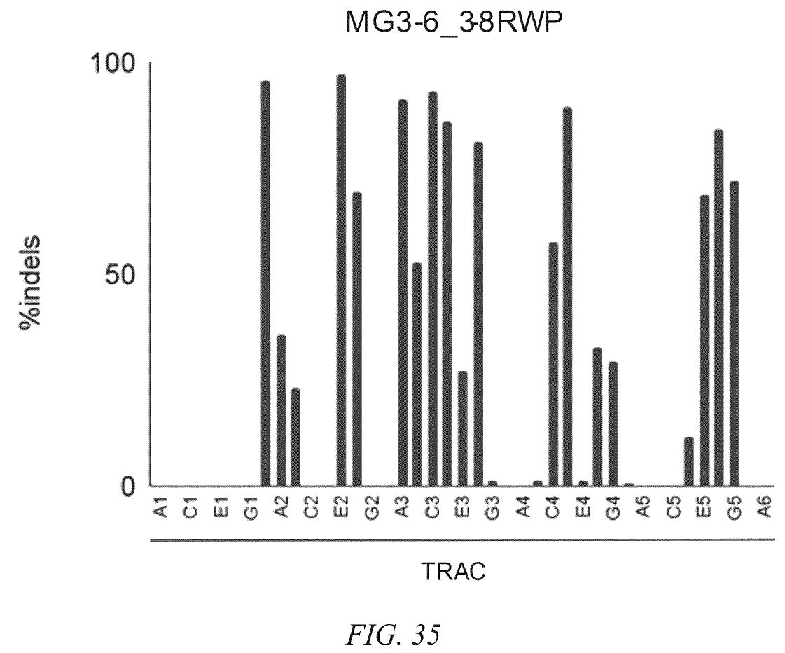

Gene editing activities of MG3-6_3-3RWP, MG3-6_3-7RWP, and MG3-6_3-8RWP were examined in K562 cells, where RWP stands for partial RuvC-III, WED, and PID (SEQ ID NOs: 771-773). PAMs of MG3-6_3-3RWP, MG3-6_3-7RWP, and MG3-6_3-8RWP are nnnCCCYR, nnRnYAYn, and nnRRnnYn, respectively. Screening of 25 guides for MG3-6_3-3RWP revealed that guide A1 on the TRAC locus and guides A2, E2, A3, and F3 on the AAVS1 locus showed more than 50% indels (FIG. 32) (SEQ ID NOs: 922-924 and 928-949). The same two loci were also used to screen 58 guides of MG3-6_3-7RWP, and the results showed that guide C3 of TRAC and guide B8 of AAVS1 exhibited more than 50% indels (FIG. 33) (SEQ ID NOs: 972-991 and 1012-1049). 237 guides were screened for MG3-6_3-8RWP on the TRAC locus, and the result demonstrated that 45 guides showed more than 50% editing. Noticeably, multiple sites showed more than 90% editing in the course of guide screens (FIGS. 34 and 35) (SEQ ID NOs: 1088-1183 and 1280-1320).

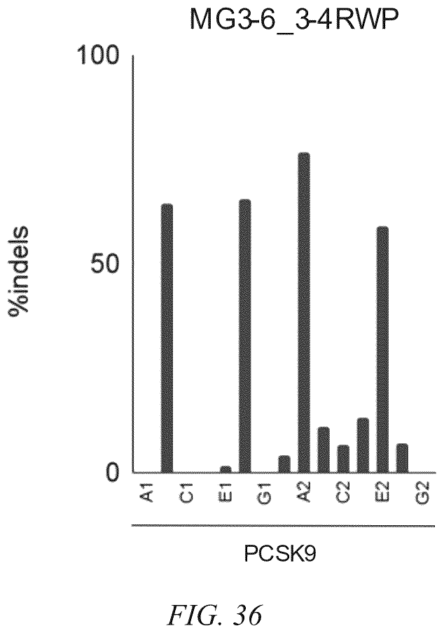

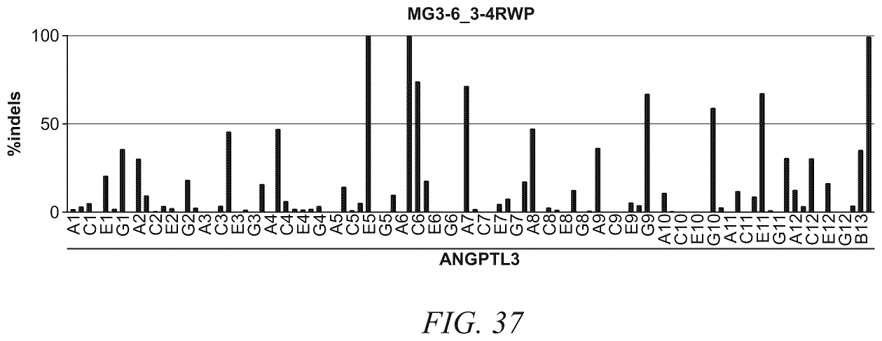

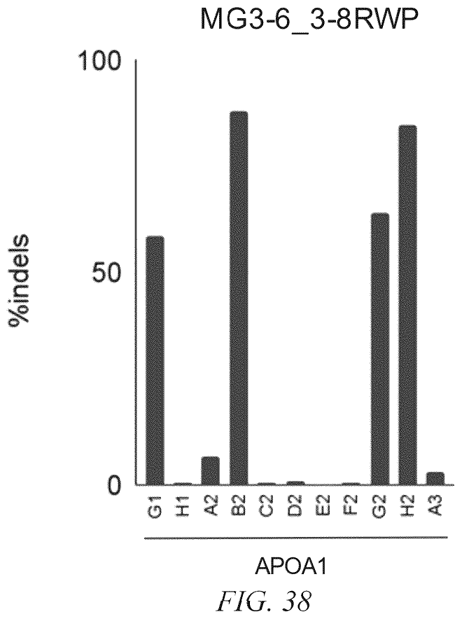

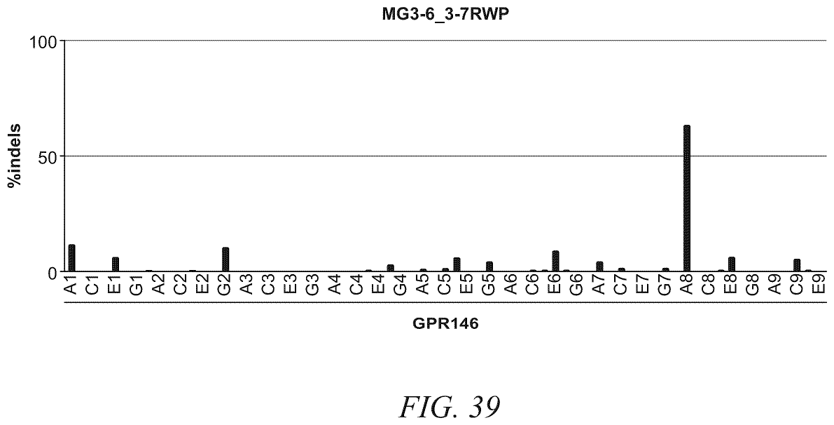

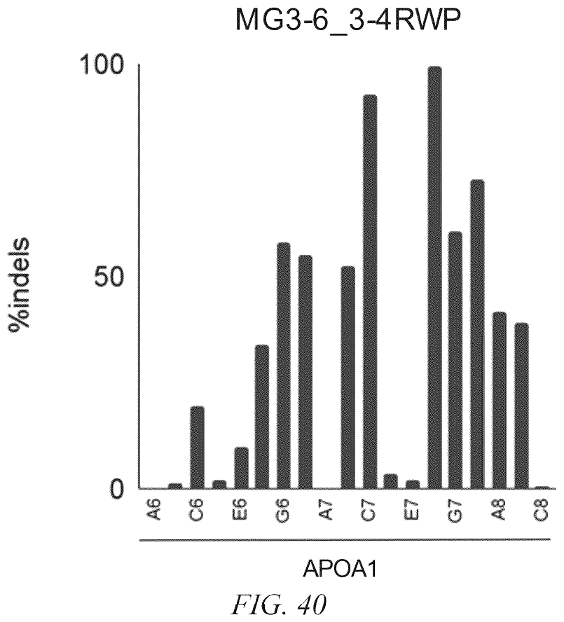

In addition to the TRAC and AAVS1 sites, guides were also designed for MG3-6_3-4RWP to target PCSK9 and ANGPTL3 in Hep3B cells, for MG3-6_3-8RWP to target APOA6 in Hep3B cells, and for MG3-6_3-7RWP to target GPR146 and APOA1 in Hepa1-6 cells. Screening results of MG3-6_3-4RWP showed that four guides on PCSK9 achieved indel rates over 50% and three guides on ANGPTL3 achieved indel rates close to 100% (FIGS. 36 and 37) (SEQ ID NOs: 1362-1376 and 1392-1489). Among the sites targeted for MG3-6_3-8RWP on APOA1, four guides showed more than 50% indel formation (FIG. 38) (SEQ ID NOs: 1764-1774). Screening of MG3-6_3-7RWP on GPR146 and APOA1 showed one and seven guides passing more than 50% indel rates (FIGS. 39 and 40) (SEQ ID NOs: 1588-1656 and 1726-1744).

### Example 28—In Vivo Editing Activity of MG3-6/3-4 Single Guide RNAs with Different Chemical Modifications

To evaluate the ability of the MG3-6/3-4 Type II nuclease to edit the genome in vivo in a living animal, lipid nanoparticles were used to deliver an mRNA encoding the MG3-6/-3-4 nuclease and one of five guide RNAs with the same nucleotide sequence but different RNA chemistries. The sequences of these guides are shown in Table 23.

TABLE 23
Sequences and chemical modifications of guide RNAs tested in vivo in mice

| | SEQ ID | |
|---|---|---|
| Guide RNA | NO: | Sequence (Spacer-Backbone) |
| mH364-7-46 | 1819 | mG*mA*mG*CUGGCCACUGUGCGAGGUA- |
| | | GUUGAGAAUCmGmAmAmAGAUUCUUAAUAAGGCAUCm |
| | | CmUmUmCmCGAUGCUGACUUCUCACCGUCCGUUUUCCm |
| | | AmAmUmAGGAGCGGGCGGUA*mU*mG*mU*mU*mU*mU |
| mH364-7-48 | 2960 | mG*mA*mG*fCfUfGfGfCfCfAfCfUfGfUfGfCfGAGGUA- |
| | | GUUGAGAAUCGAAAGAUUCUUAAUAAGGCAUCCUUCCG |
| | | AUGCUGACUUCUCACCGUCCGUUUUCCAAUAGGAGCGG |
| | | GCGGUA*mU*mG*mU*mU*mU*mU |
| mH364-7-50 | 2961 | mG*mA*mG*fCfUfGfGfCfCfAfCfUfGfUfGfCfGAGGUA- |
| | | GUUGAGAAUCmGmAmAmAGAUUCUUAAUAAGGCAUCm |
| | | CmUmUmCmCGAUGCUGACUUCUCACCGUCCGUUUUCCm |
| | | AmAmUmAGGAGCGGGCGGUA*mU*mG*mU*mU*mU*mU |
| mH364-7-52 | 2962 | mG*mA*mG*CfUGfGCfCAfCUfGUfGCGAGGUA- |
| | | GUUGAGAAUCmGmAmAmAGAUUCUUAAUAAGGCAUCm |
| | | CmUmUmCmCGAUGCUGACUUCUCACCGUCCGUUUUCCm |
| | | AmAmUmAGGAGCGGGCGGUA*mU*mG*mU*mU*mU*mU |
| mH364-7-53 | 2963 | mG*mA*mG*fCUfGGfCCfACfUGfUGfCGAGGUA- |
| | | GUUGAGAAUCmGmAmAmAGAUUCUUAAUAAGGCAUCm |
| | | CmUmUmCmCGAUGCUGACUUCUCACCGUCCGUUUUCCm |
| | | AmAmUmAGGAGCGGGCGGUA*mU*mG*mU*mU*mU*mU |

Notations for chemical modifications: m = 2′O-Methyl ribonucleotide (e.g., mC = cytosine ribonucleotide with 2′-O Methyl in place of 2′ hydroxyl); f = 2′Fluorine ribonucleotide (e.g., fC = cytosine ribonucleotide with 2′ fluorine in place of 2′ hydroxyl); * = phosphorothioate bond. All other bases are native ribonucleotides.

The spacer sequence in these guides targets the mouse HAO-1 gene. All the guide RNAs have the same end modifications consisting of three 2′ methyl groups with phosphorothioate linkages at the 5′ end and six 2′ methyl groups with phosphorothioate linkages at the 3′ end. Guide mH364-7-46, which had the highest editing activity in a previous mouse study, has additional 2′ methyl modifications in all three loops of the guide backbone sequence. Guide mH364-7-48 has 2′ fluorine modifications in the spacer sequence but no methyl modifications in the backbone loops. Guide mH364-7-50 combines the spacer 2′ fluorine and backbone loop 2′ methyl modifications of chemistries 46 and 48. Guide mH364-7-52 has fewer 2′ fluorines in the spacer than chemistry 50 but has the same backbone methyl modifications as chemistry 50. Guide mH364-7-53 has 2′ fluorines on different residues of the spacer than chemistry 52 and the same backbone methyl modifications as chemistries 50 and 52. Because the chemical modifications in the sgRNA impact sgRNA stability which is critical for in vivo potency it was important to evaluate the impact of these different modifications on in vivo editing.

Preparation of MG3-613-4 mRNA

The mRNA encoding MG3-6/3-4 was generated by in vitro transcription of a linearized plasmid template using T7 RNA polymerase. The protein coding sequence of the MG3-6/3-4 cassette comprises the following elements from 5′ to 3′: the nuclear localization signal from SV40, a five amino acid linker (GGGS (SEQ ID NO: 2918)), the protein coding sequence of the MG3-6/3-4 nuclease from which the initiating methionine codon was removed, a 3 amino acid linker (SGG), and the nuclear localization signal from nucleoplasmin. The DNA sequence of this cassette was codon optimized for human expression. An approximately 100 nucleotide polyA tail (SEQ ID NO: 2865) was encoded in the plasmid used for in vitro transcription and the mRNA was co-transcriptionally capped. Uridine in the mRNA was replaced with N1-methyl pseudouridine.

### Preparation of Lipid Nanoparticles

The lipid nanoparticle (LNP) formulation used to deliver the MG3-6/3-4 mRNA and the guide RNA is based on LNP formulations as described above. The four lipid components were dissolved in ethanol and mixed in an appropriate molar ratio to make the lipid working mix. The RNAs were diluted in 100 mM Sodium Acetate (pH 4.0) to make the RNA working stocks. The lipid working stock and the RNA working stocks were mixed in a microfluidics device at a flow rate ratio of 1:3, respectively, and a flow rate of 12 mL/min. The LNP were dialyzed against phosphate buffered saline (PBS) for 2 to 16 hours and then concentrated. The concentration of RNA in the LNP formulation was measured using the Ribogreen reagent. The diameter and polydispersity (PDI) of the LNP were determined by dynamic light scattering. Typical LNP had diameters ranging from 70 nm to 84 nm and PDI of 0.098 to 0.150.

### Mouse Dosing and Harvesting

LNPs for mRNA and sgRNA were mixed at 1:1 mass ratio and injected intravenously into 7 week-old C57B16 wild type mice via the tail vein (0.1 mL per mouse) at a total RNA dose of 0.5 mg RNA per kg body weight. Fourteen days after dosing, the mice were sacrificed, and the left lateral, medial, and right lateral lobes of the liver were collected for preparation of DNA, RNA, and protein, respectively. Blood was collected by exsanguination via cardiac puncture and collected onto BD microtainer (heparin coated). Samples were kept on wet ice for no longer than 30 minutes prior to centrifugation. Samples were centrifuged at 2,000 G for 10 minutes and the plasma transferred to 1.5 mL Cryotubes and stored at −80° C.

### Genomic DNA Preparation and Editing Analysis by Next-Generation Sequencing (NGS)

The left lateral lobe of the liver (100 mg) was homogenized in digestion buffer. Genomic DNA was purified from the resulting homogenate and quantified by measuring the absorbance at 260 nm. Genomic DNA purified from mice injected with PBS buffer alone was used as a control. The region of the HAO-1 gene targeted by each specific sgRNA was PCR amplified with Q5 high fidelity DNA polymerase and gene specific primers (Table 24) with adapters complementary to the barcoded primers used for next generation sequencing (NGS) for a total of 29 cycles. The product of this first PCR reaction was PCR amplified using the barcoded primers for NGS for a total of 10 cycles. The resulting product was subjected to NGS and the results were analyzed to generate the percentage of sequencing reads that contain insertions or deletions (INDELS) at the targeted site in the HAO-1 gene.

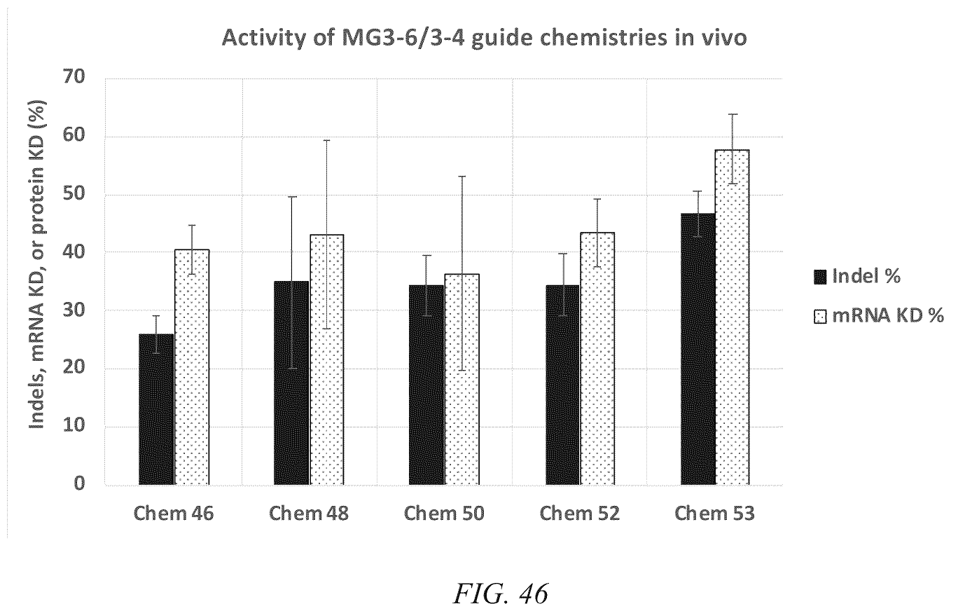

The NGS analysis of mouse liver genomic DNA showed that editing at the target site of the sgRNA in the HAO-1 gene of the groups dosed with LNP encapsulating MG3-6/3-4 mRNA and each of the five sgRNA ranged from 26% to 47% of total liver genomic DNA (FIG. 46). Chemistry 46 edited at the lowest efficiency. Guides with chemistry 48, 50, and 52 exhibited similar or somewhat higher levels of editing than the guide with chemistry 46. The guide with chemistry 53 had the highest editing efficiency at 47%, which was nearly twice the editing achieved with the guide with the same nucleotide sequence with chemistry 46.

TABLE 24
Primers used to amplify HAO1 guide target site and for NGS

| Guide | Primer | | |
|---|---|---|---|
| spacer | Set Name | Fwd with Miseq adapter | Rev with Miseq adapter |
| mH364-7 | mHAO1-NGS-P4 | GCTCTTCCGATCTNNNNNG | GCTCTTCCGATCTNNNNN |
| (spacer 7) | | TAAAGAAAAACAAGGAAT | ATCTGTCAACTTCTGTTTT |
| | | GTAAT (SEQ ID NO: 2910) | AGGAC (SEQ ID NO: 2911) |

### RNA Preparation and Analysis by RT-ddPCR

The medial lobe of the liver was stored in storage buffer to preserve the integrity of the RNA prior to homogenization. A maximum of 10 mg of tissue was transferred to a 2 mL tube containing 1.4 mm ceramic beads and homogenized. Homogenized tissue was then processed with an additional 45 minute on-column DNase I digestion treatment. The RNA products were quantified by measuring the absorbance at 260 nm. Isolated RNA samples then underwent an additional DNase treatment and reverse transcription. The cDNA product was then quantified by measuring the absorbance at 260 nm.

HAO1 mRNA levels were quantified using a custom ddPCR probe-based assay that was multiplexed with the housekeeping gene GAPDH. HAO1 was labeled with a HEX probe while GAPDH was labeled with a FAM probe. Both probes were flanked by primers specific to the respective genes creating amplicons of about 115 bp. The template material for the assay was 25 ng of cDNA product from the process above mixed with 900 nM of each primer and 250 nM of probe per gene assay, subject to ddPCR, and analyzed. Results were divided into four sections: negative droplets (no fluorescence), single positive droplets (HEX or FAM positive fluorescence), and double positive droplets (both HEX and FAM fluorescence). Copies per μL of HAO1 and GAPDH were calculated for each sample. The ratio of HAO1 to GAPDH was compared for mice treated with mH29-29 against mice treated with buffer only.

The knockdown of HAO1 mRNA in the livers of mice from the groups treated with LNP encapsulating the mH364-7 sgRNA and MG3-6/3-4 mRNA ranged from 36% to 58% lower than the untreated control group (FIG. 46). Editing of the HAO-1 gene that introduces out of frame (OOF) INDELS into the coding sequence may result in a reduction in the amount of HAO-1 mRNA as a result of nonsense mediated mRNA decay. The magnitude of reduction in HAO-1 mRNA by the guides with the 5 different chemical modifications correlated with the magnitude of genome editing efficiency, with guide chemistry 53 having the greatest knockdown, consistent with it having the highest indel %. Taken together, the genome editing and HAO-1 mRNA knockdown data indicate that guide chemistry 53 has improved potency in vivo.

TABLE 25
Primers and probes used in the amplification HAO1 and GAPDH ddPCR
assays

| Target | Probe | Probe | Fwd Primer | Rev primer |
|---|---|---|---|---|
| Gene | Fluorescence | Sequence | Sequence | sequence |
| HAO1 | HEX | AGTG+GGTG+C+C | GGGGAGAAAGGTG | CTCACCAATGTCT |
| | | A+GAAT+GTGAA | TTCAAGATGT (SEQ | TGTCGATGA (SEQ |
| | | (SEQ ID NO: 2912) | ID NO: 2914) | ID NO: 2916) |
| GAPDH | FAM | CATGACCA+CAGT | GCACCACCAACTGC | CCATCCACAGTCT |
| | | +C+CATGCCATC | TTAG (SEQ ID NO: | TCTGGG (SEQ ID |
| | | (SEQ ID NO: 2913) | 2915) | NO: 2917) |

Notations for chemical modifications: + = Locked Nucleic Acid (LNA) base modification.

### Example 29—Mammalian Cell Screening of MG3-6 Chimera (MG3-6/3-8) Across Multiple Gene Targets in Two Different Cell Types

It was previously demonstrated that a chimeric protein created by swapping the C-terminal end of the MG3-6 enzyme with its closely related homologs, specifically the WED and PAM-interacting domains, recognizes a new PAM sequence which corresponds to the PAM from the homolog Type II enzyme. MG3-6/3-8 (SEQ ID NO: 12), a chimera created by combining MG3-6 with the WED and PID domains from MG3-8, can successfully edit multiple target sites in human cell line. These results are expanded on herein by using the MG3-6/3-8 chimera at four additional loci in two new cell lines.

mRNA Production

Sequences for the MG3-6/3-8 chimera (SEQ ID NO: 12) mRNA were codon optimized for human expression, then synthesized and cloned into a high copy ampicillin plasmid. Synthesized constructs encoding a T7 promoter, UTRs, chimera ORF, and NLS sequences were digested from the backbone with HindII and BamHI, and ligated into a pUC19 plasmid backbone. The complete chimera mRNA plasmid consists of an origin of replication, ampicillin resistance cassette, the synthesized construct, and an encoded polyA tail. mRNA was synthesized via in vitro transcription (IVT) using the linearized chimera mRNA plasmid. This plasmid was linearized and purified with Phenol:Chloroform:Isoamyl Alcohol (25:24:1, v/v), precipitated in EtOH, and resuspended in nuclease free water at an adjusted concentration of 500 ng/μL. The IVT reaction to generate the mRNA was performed at 50° C. for 1 hour. After 1 hour, IVT was stopped, and plasmid DNA was digested with the addition of 250 U/mL DNaseI and incubated for 10 min at 37° C. Purification of mRNA was performed and the transcript concentration was determined by UV and further analyzed by capillary gel electrophoresis.

### Cell Culture, Transfections, Next Generation Sequencing, and Editing Analysis for Screens

Nucleofection of mRNA and guide (500 ng mRNA, 150 pmol guide) was performed in Hepa1-6 cells (100,000 cells per guide) and Hep3B cells (100,000 cells per guide) using an electroporator. Guides used for screening were chemically synthesized. Cells were harvested and genomic DNA prepared three days post-transfection. PCR primers appropriate for use in NGS-based DNA sequencing were generated, optimized, and used to amplify the individual target sequences for each guide RNA. The amplicons were sequenced and analyzed to measure gene editing.

### Results

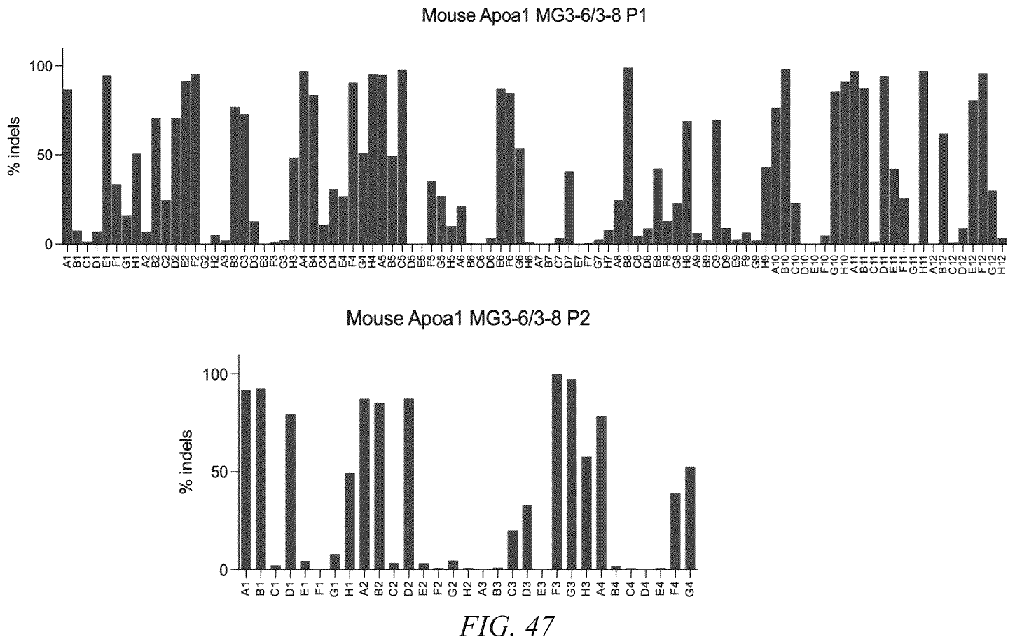

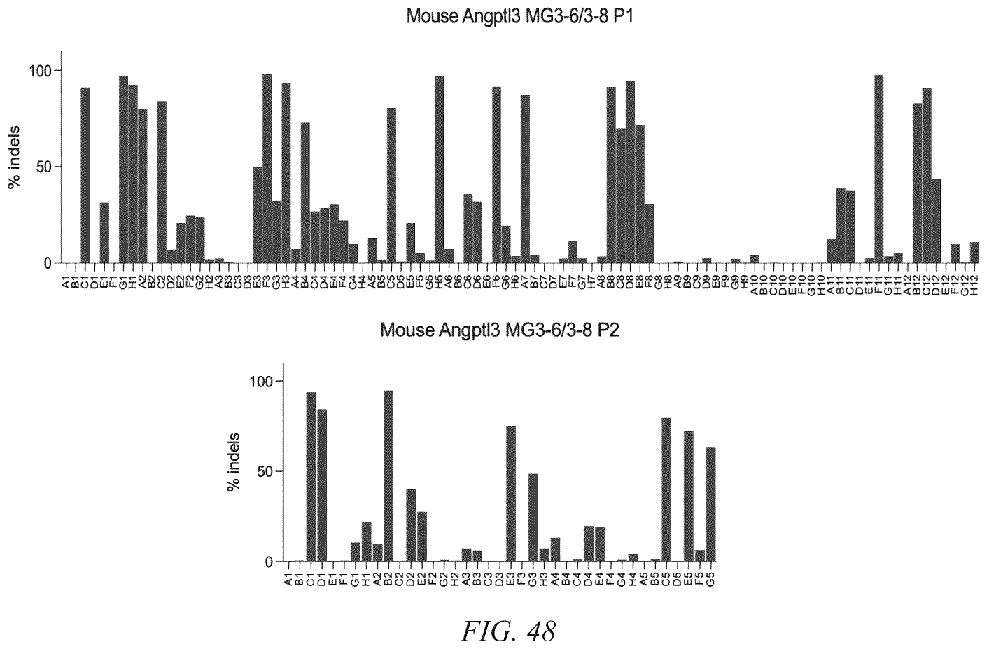

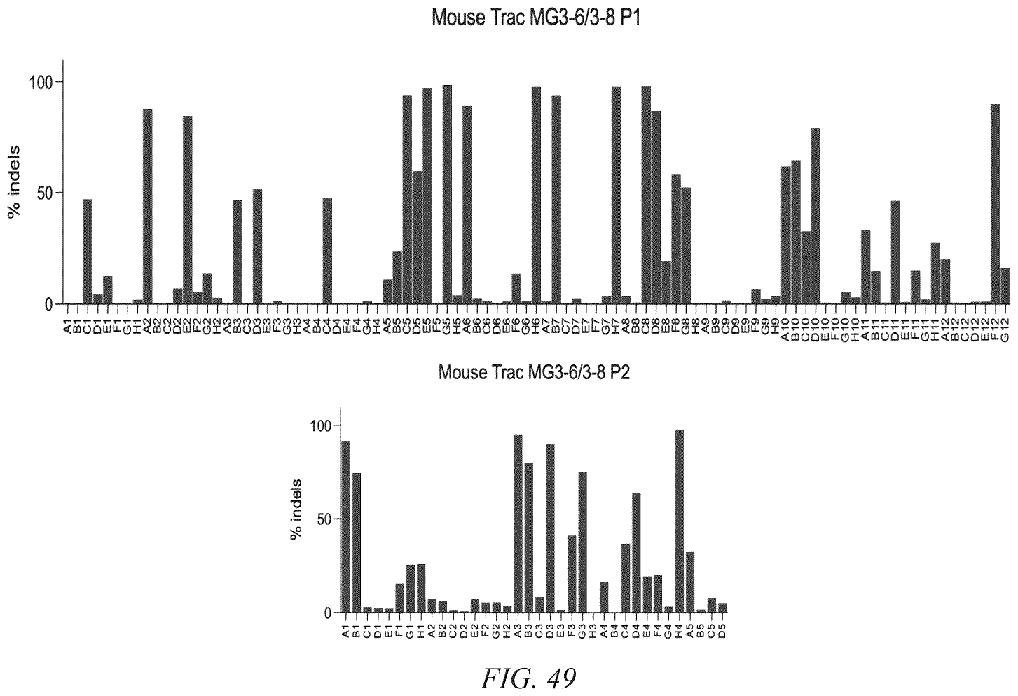

To test the gene editing activity of the engineered MG3-6/3-8 chimera (SEQ ID NO: 12), 394 guides targeting the APOA1 (SEQ ID NOs: 1866-1961 and 2058-2088), ANPTL3 (SEQ ID NOs: 2120-2215 and 2312-2350), and TRAC (SEQ ID NOs: 2390-2485 and 2582-2617) genes in the mouse genome with MG3-6/3-8 (PAM sequence: nnRRnnYn (SEQ ID NO: 2863) were designed and purchased. These guides were co-transfected with in vitro-synthesized mRNA in Hepa1-6 (a mouse immortalized mouse hepatocyte cell line) or Hep3B (a human liver cell line) via nucleofection and were assayed for indel formation three days post-nucleofection. Altogether, the average indel formation was 40.7% at APOA1 (across 127 guides), 27.4% at ANGPTL3 (across 135 guides), and 25.1% at TRAC site (across 132 guides). 179 guides demonstrated greater than 80% indel formation. In conclusion, MG3-6/3-8 chimera shows robust gene editing across four independent loci, in both mouse and human cell lines (FIGS. 47-49).

### Example 30—In Vivo Gene Editing in Liver of Mice by the Chimeric Nuclease MG3-6/3-8 Delivered by Systemic Administration of a Lipid Nanoparticle

To evaluate the ability of the MG3-6/3-8 chimeric Type II nuclease to edit the genome in vivo, a lipid nanoparticle was used to deliver an mRNA encoding the MG3-6/3-8 nuclease and single guide RNAs (sgRNAs) that target exonic regions of the mouse APOA1 and ANGPTL3 genes (Table 26).

TABLE 26
sgRNAs and targeting spacer sequences used in mouse study

| Target | | |
|---|---|---|
| Site | Spacer DNA | |
| Name | sequence | sgRNA sequence |
| Apoa1 | TCTCCTGGAAAA | mU*mC*mU*rCrCrUrGrGrArArArArCrUrGrGrGrArCrArCrU |
| A4 | CTGGGACACT | rGrUrUrGrArGrArArUrCrGrArArArGrArUrUrCrUrUrArArUr |
| | (SEQ ID NO: 1986) | ArArGrGrCrArUrCrCrUrUrCrCrGrArUrGrCrUrGrArCrUrUrC |
| | | rUrCrArCrCrGrUrCrCrGrUrUrUrUrCrCrArArUrArGrGrArGr |
| | | CrGrGrGrCrGrGrUrArUrGrU*mU*mU*mU (SEQ ID NO: |
| | | 1890) |
| Apoa1 | CTGGGATAACCT | mC*mU*mG*rGrGrArUrArArCrCrUrGrGrArGrArArArGrArA |
| A5 | GGAGAAAGAA | rGrUrUrGrArGrArArUrCrGrArArArGrArUrUrCrUrUrArArUr |
| | (SEQ ID NO: 1994) | ArArGrGrCrArUrCrCrUrUrCrCrGrArUrGrCrUrGrArCrUrUrC |
| | | rUrCrArCrCrGrUrCrCrGrUrUrUrUrCrCrArArUrArGrGrArGr |
| | | CrGrGrGrCrGrGrUrArUrGrU*mU*mU*mU (SEQ ID NO: |
| | | 1898) |
| Apoa1 | TTGGGTGAGACA | mU*mU*mG*rGrGrUrGrArGrArCrArGrGrArGrArUrGrArAr |
| C5 | GGAGATGAAC | CrGrUrUrGrArGrArArUrCrGrArArArGrArUrUrCrUrUrArArU |
| | (SEQ ID NO: 1996) | rArArGrGrCrArUrCrCrUrUrCrCrGrArUrGrCrUrGrArCrUrUr |
| | | CrUrCrArCrCrGrUrCrCrGrUrUrUrUrCrCrArArUrArGrGrArG |
| | | rCrGrGrGrCrGrGrUrArUrGrU*mU*mU*mU (SEQ ID NO: |
| | | 1900) |
| Apoa1 | AGGAACGGCTG | mA*mG*mG*rArArCrGrGrCrUrGrGrGrCrCrCrArUrUrGrArC |
| F4 | GGCCCATTGAC | rGrUrUrGrArGrArArUrCrGrArArArGrArUrUrCrUrUrArArUr |
| | (SEQ ID NO: 1991) | ArArGrGrCrArUrCrCrUrUrCrCrGrArUrGrCrUrGrArCrUrUrC |
| | | rUrCrArCrCrGrUrCrCrGrUrUrUrUrCrCrArArUrArGrGrArGr |
| | | CrGrGrGrCrGrGrUrArUrGrU*mU*mU*mU (SEQ ID NO: |
| | | 1895) |
| Apoa1 | GGAACGGCTGG | mG*mG*mA*rArCrGrGrCrUrGrGrGrCrCrCrArUrUrGrArCrU |
| G4 | GCCCATTGACT | rGrUrUrGrArGrArArUrCrGrArArArGrArUrUrCrUrUrArArUr |
| | (SEQ ID NO: 1992) | ArArGrGrCrArUrCrCrUrUrCrCrGrArUrGrCrUrGrArCrUrUrC |
| | | rUrCrArCrCrGrUrCrCrGrUrUrUrUrCrCrArArUrArGrGrArGr |
| | | CrGrGrGrCrGrGrUrArUrGrU*mU*mU*mU (SEQ ID NO: |
| | | 1896) |
| Angptl | ACTATTAAACCA | mA*mC*mU*rArUrUrArArArCrCrArArGrArArArCrUrCrCrC |
| 3 C12 | AGAAACTCCC | rGrUrUrGrArGrArArUrCrGrArArArGrArUrUrCrUrUrArArUr |
| | (SEQ ID NO: 1993) | ArArGrGrCrArUrCrCrUrUrCrCrGrArUrGrCrUrGrArCrUrUrC |
| | | rUrCrArCrCrGrUrCrCrGrUrUrUrUrCrCrArArUrArGrGrArGr |
| | | CrGrGrGrCrGrGrUrArUrGrU*mU*mU*mU (SEQ ID NO: |
| | | 2210) |

(r = native ribose base, m = 2′-O methyl modified base, * = phosphorothioate bond)

The screen of sgRNAs targeting the APOA1 and ANGPTL3 coding sequences in mouse hepatocytes (Example 29) was used to identify highly active guides which were used for the in vivo mouse study (FIGS. 47-49). Specifically, sgRNAs MG3-6/3-8 mApoa1P1-A4 (SEQ ID NO: 1890), MG3-6/3-8 mApoa1P1-A5 (SEQ ID NO: 1898), MG3-6/3-8 mApoa1P1-C5 (SEQ ID NO: 1900), MG3-6/3-8 mApoa1P1-F4 (SEQ ID NO: 1895), and MG3-6/3-8 mAngptl3P1-C12 (SEQ ID NO: 2210), demonstrated greater than 95% editing in Hepa1-6 when transfected with the mRNA encoding the MG3-6/3-8 nuclease, and were selected for testing in mice. Additionally, MG3-6/3-8 mApoa1P1-G4 (SEQ ID NO: 1896), which demonstrated comparatively less editing (53%), was also chosen for the mouse study to inform on sites with moderate activity. Moreover, a number of chemical modifications of the native RNA structure were incorporated into these sgRNAs. These chemical modifications were selected based on their ability to improve the stability of the sgRNA in vitro when incubated in extracts from mammalian cells without negatively impacting editing activity.

Preparation of MG3-613-8 mRNA

The mRNA encoding the MG3-6/3-8 nuclease was generated by in vitro transcription of a linearized plasmid template. The DNA sequence that was transcribed into RNA comprised the following elements in order from 5′ to 3′: the T7 RNA polymerase promoter, a 5′ untranslated region (5′ UTR), a nuclear localization signal, a short linker, the coding sequence for the MG3-6/3-8 nuclease, a short linker, a nuclear localization signal, a 3′ untranslated region, and an approximately 100 nucleotide polyA tail (SEQ ID NO: 2865).

The protein sequence encoded in the synthetic mRNA encoded in this MG3-6/3-8 cassette comprises the following elements from 5′ to 3′: the nuclear localization signal from SV40, a five amino acid linker (GGGGS (SEQ ID NO: 2864)), the protein coding sequence of the MG3-6/3-8 nuclease from which the initiating methionine codon was removed, a 3 amino acid linker (SGG) and the nuclear localization signal from nucleoplasmin. The DNA sequence of the protein coding region of this cassette was modified to reflect the codon usage in humans using a commercially available algorithm. An approximately 100-nucleotide polyA tail (SEQ ID NO: 2865) was encoded in the plasmid used for in vitro transcription and the mRNA was co-transcriptionally capped. Uridine in the mRNA was replaced with N1-methyl pseudouridine.

### Preparation of Lipid Nanoparticles

The lipid nanoparticle (LNP) formulation used to deliver the MG3-6/3-8 mRNA and the guide RNA is based on LNP formulations as described above. The four lipid components were dissolved in ethanol and mixed in an appropriate molar ratio to make the lipid working mix. The mRNA and the guide RNA were mixed prior to formulation at a 1:1 mass ratio. RNA was diluted in 100 mM Sodium Acetate (pH 4.0) to make the RNA working stock. The lipid working stock and the RNA working stock were mixed in a microfluidics device. The LNPs were dialyzed against phosphate buffered saline (PBS) for 2 to 16 hours and then concentrated. The concentration of RNA in the LNP formulation was measured using the Ribogreen reagent. The diameter and polydispersity (PDI) of the LNP were determined by dynamic light scattering. Representative LNP diameters ranged from 65 nm to 120 nm with PDI of 0.05 to 0.20.

### Mouse Dosing and Harvesting

LNP for mRNA and sgRNA were mixed at 1:1 mass ratio and injected intravenously into 7-week-old C57B16 wild type mice via the tail vein (0.1 mL per mouse) at a total RNA dose of 1 mg RNA per kg body weight. Seven days post-dosing, all four mice in each group were sacrificed. The left liver lobe was collected and flash frozen.

### Genomic DNA Preparation and Editing Analysis by Next-Generation Sequencing (NGS)

The left lateral lobe of the liver (100 mg) was homogenized in digestion buffer. Genomic DNA was purified from the resulting homogenate and quantified by measuring the absorbance at 260 nm. Genomic DNA purified from mice injected with PBS buffer alone was used as a control. The region of the APOA1 and ANGPTL3 genes targeted by each specific sgRNA was PCR amplified with Q5 high fidelity DNA polymerase and gene specific primers with adapters complementary to the barcoded primers used for next generation sequencing (NGS) for a total of 29 cycles. The product of this first PCR reaction was PCR amplified using the barcoded primers for NGS for a total of 10 cycles. The resulting product was subjected to NGS, and the results were analyzed for the percentage of sequencing reads that contain insertions or deletions (indels) at the targeted site in the APOA1 and ANGPTL3 genes.

### Results

The gene editing results are summarized in Table 27.

TABLE 27
Analysis of the frequency of edits that result in frame shifts
(insertions/deletions) in the liver of mice
treated with LNP encapsulating MG3-6/3-8
mRNA and sgRNAs that target the APOA-1 and ANGPTL3 genes

| | | Mean | Stdev of | Mean OOF | Stdev OOF |
|---|---|---|---|---|---|
| Treatment | Guide | INDELS | INDELS | % total | % total |
| PBS control | Apoa1 A4 | 0.03 | 0.02 | 0.03 | 0.02 |
| | Apoa1 A5 | 0.01 | 0.01 | 0.01 | 0.01 |
| | Apoa1 C5 | 0.01 | 0.01 | 0.01 | 0.01 |
| | Apoa1 F4 | 0.04 | 0.01 | 0.04 | 0.01 |
| | Apoa1 G4 | 0.04 | 0.01 | 0.03 | 0.01 |
| | Angpt13 C12 | 0.02 | 0.01 | 0.02 | 0.01 |
| MG3-6_3-8 | Apoa1 A4 | 79.30 | 6.23 | 75.40 | 5.87 |
| | Apoa1 A5 | 65.23 | 3.96 | 55.51 | 3.49 |
| | Apoa1 C5 | 77.82 | 1.88 | 73.90 | 1.55 |
| | Apoa1 F4 | 43.07 | 13.67 | 30.37 | 11.77 |
| | Apoa1 G4 | 34.76 | 9.83 | 38.97 | 8.64 |
| | Angpt13 C12 | 42.80 | 3.34 | 37.18 | 3.12 |

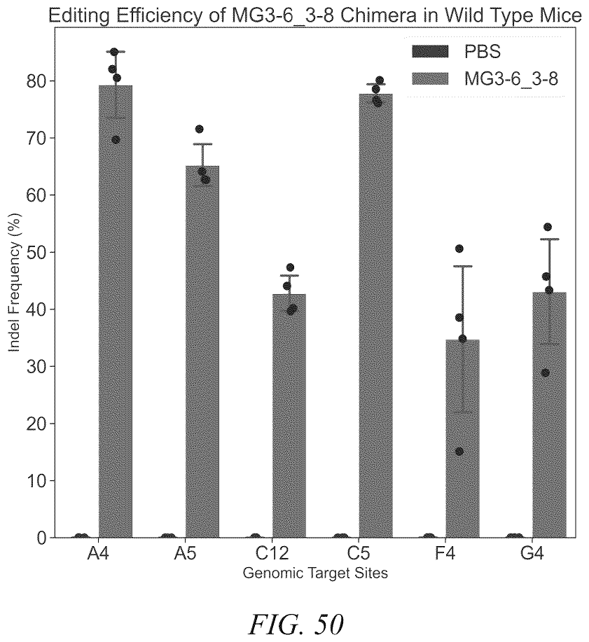

The NGS analysis of mouse liver genomic DNA showed that editing at the target site of the sgRNA in the APOA1 and ANGPTL3 genes of the groups dosed with LNP encapsulating MG3-6/3-8 mRNA and each of the six sgRNA ranged from 35% to 80% of total liver genomic DNA (FIG. 50). There were minor differences between most of the treated groups, indicating robust and reproducible editing outcomes. Overall, the genome editing at APOA1 and ANGPTL3 sites to knockdown gene to regulate the downstream protein product using the MG3-6/3-8 chimeric nuclease demonstrates its therapeutic potential through targeted gene editing.

### Example 31—In Vitro Cleavage Activity of Type V-A Chimeric Nucleases

### Bioinformatics Analyses

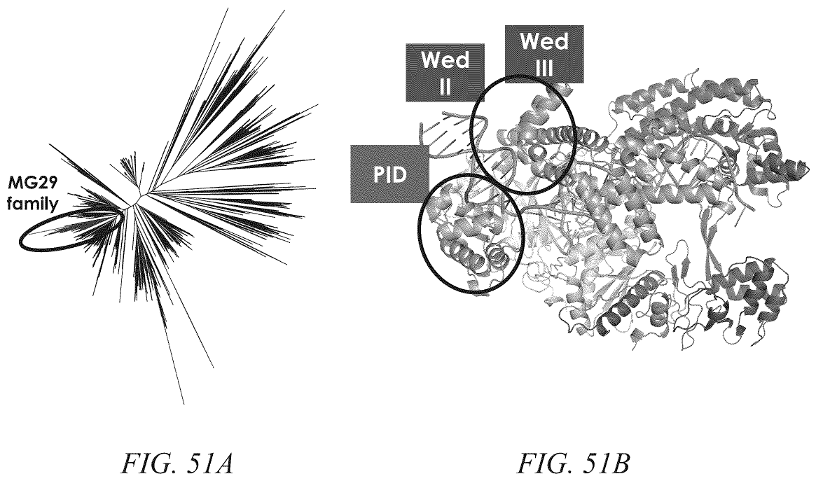

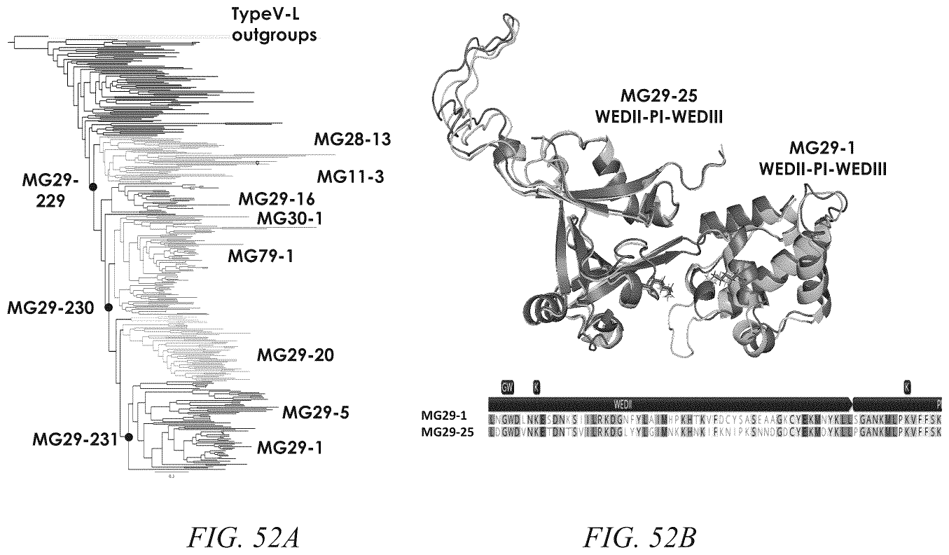

14 new protein variants of the Type V-A enzyme MG29-1 were engineered by concatenating domains from diverse homologs as well as from novel artificially designed nuclease sequences obtained through ancestral sequence reconstruction (ASR) (FIGS. 51-52). These novel Type V-A chimeras expand the PAM-targeting capabilities of MG29-1 enzyme beyond its canonical PAM sequence. Similar to previously engineered Type V-A chimeras MG29-1/29-5 (SEQ ID NO: 109) and MG29-1/57-1 (SEQ ID NO: 110), chimeric sequences were designed with novel MG29-1 homologs MG29-13 to MG29-21 (SEQ ID NOs: 2842-2849), as well as with more distant Type V-A nucleases, namely MG11-18 and MG79-1 (SEQ ID NOs: 2850-2851).

Additionally, in an effort to generate further diversity of Type V-A nucleases, ancestral sequence reconstruction (ASR) algorithms were used. ASR is a computational technique that uses existing protein sequences and the relationships inferred between them to reconstruct the sequences of ancient, now extinct, proteins. This technique was used to computationally reconstruct novel sequences of the Type V-A family to use for MG29-1 chimeragenesis. For this analysis, 330 Type V-A protein sequences were aligned and a phylogenetic tree was built (FIG. 52A). The trees were rooted using four Type V-L sequences as outgroups. Sequences were reconstructed and insertions and deletions were identified manually for each reconstructed node. Ultimately, three Type V-A ancestral sequences (MG29-229 to MG29-231) were reconstructed with high confidence and were chosen for chimeragenesis with MG29-1 (SEQ ID NOs: 2852-2854) (Table 28 and FIG. 52).

TABLE 28
Comparison of computationally-derived Type V-A ancestors
vs MG29-1, for Wed-II, PI, and Wed-III domains.

| Ancestral Parent | | | Swapped Domain |
|---|---|---|---|
| (WED II + | Mean | MG29-1 | Length |
| PID + WED-III ) | Support | % Identity | (amino acids) |
| MG29-229 | 0.94 | 65 | 295 |
| MG29-230 | 0.97 | 65 | 295 |
| MG29-231 | 0.96 | 64 | 294 |

Mean support values indicate the average probability for the reconstructed sequence, on a scale from 0 to 1. Support values >0.7 indicate high confidence in the reconstructed sequence.

Based on the predicted structure of MG29-1 and its alignment with the substrate-bound R-loop structure of FnCas12a (also called Cpf1), the boundaries of the WED-II, PID, and WED-III domains of MG29-1, its homologs, and the ancestral sequences, which are likely responsible the PAM sequence recognition and binding, were identified (FIGS. 51B and 52B).

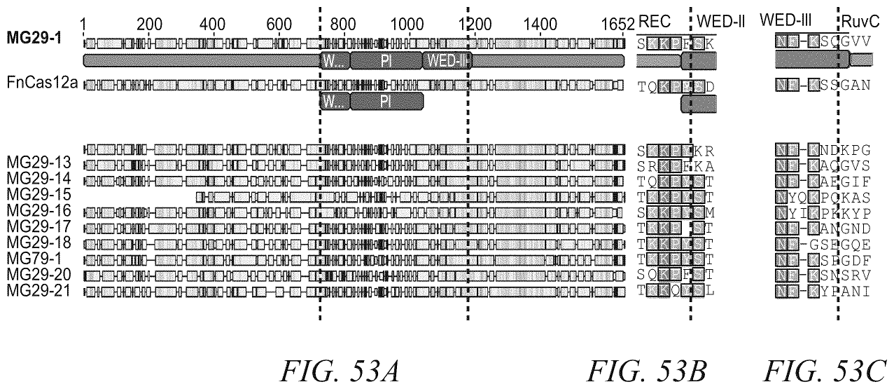

A multiple sequence alignment of the select wild-type and ASR-generated Type V-A endonucleases, along with MG29-1 and the reference Cpf1, was constructed (FIG. 53A). Based on this alignment, the various functional domains of these novel MG enzymes were defined and used to subsequently determine the appropriate REC-WED-II and WED-III-RuvC breakpoints for chimeragenesis (FIGS. 53B-53C). Details of chimeric compositions are shown in Table 29.

TABLE 29
Chimeric architecture for using MG29-1 as chassis

| Chimera Type | Chimera name | WED-I REC | WED-II + PI + WED-III | RuvC + Nuc |
|---|---|---|---|---|
| Combination of all | MG29-1_29-13 | MG29-1(1, 560) | MG29-13 (558, 859) | MG29-1(862, 1280) |
| natural sequences | MG29-1_29-14 | MG29-1(1, 560) | MG29-14 (544, 845) | MG29-1(862, 1280) |
| | MG29-1_29-15 | MG29-1(1, 560) | MG29-15 (344, 645) | MG29-1(862, 1280) |
| | MG29-1_29-16 | MG29-1(1, 560) | MG29-16 (524, 825) | MG29-1(862, 1280) |
| | MG29-1_29-17 | MG29-1(1, 560) | MG29-17 (565, 866) | MG29-1(862, 1280) |
| | MG29-1_29-18 | MG29-1(1, 560) | MG29-18 (537, 830) | MG29-1(862, 1280) |
| | MG29-1_29-20 | MG29-1(1, 560) | MG29-20 (556, 857) | MG29-1(862, 1280) |
| | MG29-1_29-21 | MG29-1(1, 560) | MG29-21 (514, 815) | MG29-1(862, 1280) |
| | MG29-1_11-18 | MG29-1(1, 560) | MG11-18 (527, 872) | MG29-1(862, 1280) |
| | MG29-1_79-1 | MG29-1(1, 560) | MG79-1 (564, 865) | MG29-1(862, 1280) |
| Combination of natural | MG29-1_29-229 | MG29-1(1, 560) | MG29-1_29-229 (561, 855) | MG29-1(862, 1280) |
| sequence and ASR | MG29-1_29-230 | MG29-1(1, 560) | MG29-1_29-230 (561, 855) | MG29-1(862, 1280) |
| generated sequences | MG29-1_29-231 | MG29-1(1, 560) | MG29-1_29-231 (561, 854) | MG29-1(862, 1280) |

### Results

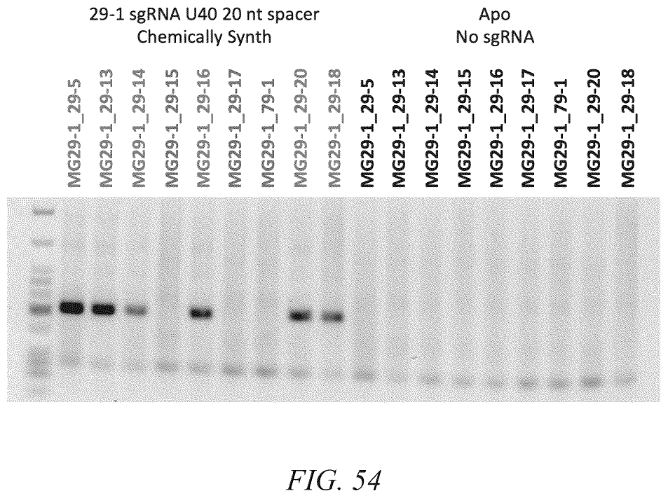

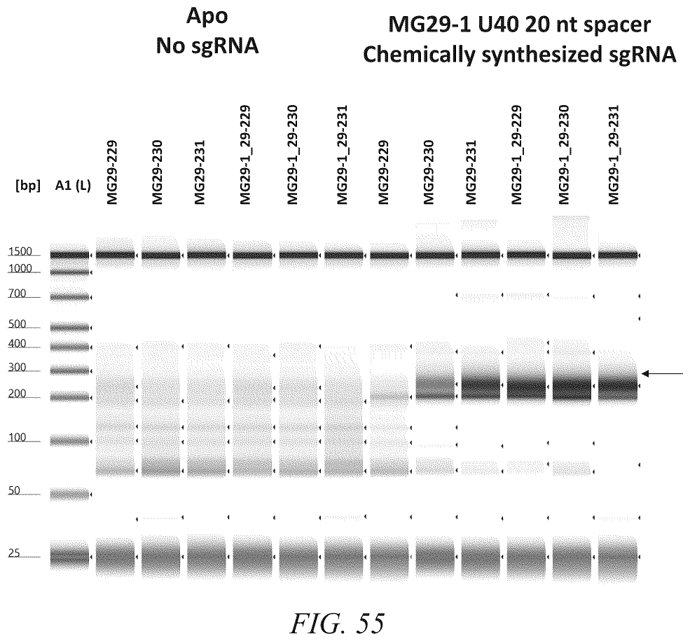

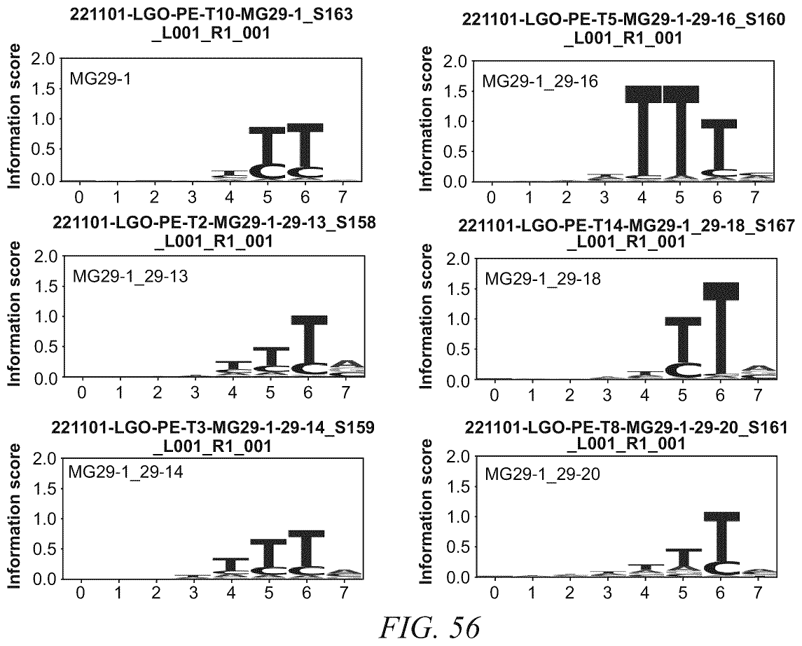

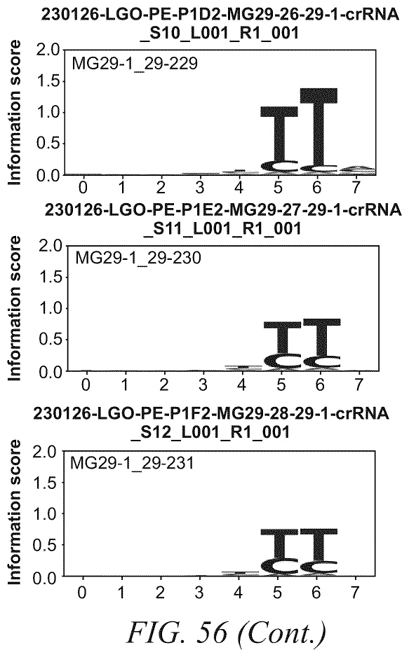

The PAM sequences of nucleases utilized herein were determined via expression in either an E. coli lysate-based expression system or reconstituted in in vitro translation. PAM enrichment assays with chimeric nucleases using the MG29-1 backbone and PID and WED domains from MG29 family homologs and ancestral sequences indicated that nine Type V-A chimeras were active when tested with the MG29-1 targeting guide RNA (FIGS. 54-55). The required PAM motifs are shown in FIG. 56 and summarized in Table 30.

TABLE 30
PAMs associated with the active chimeras derived from MG29-1.

| Chimera name | PAM sequence |
|---|---|
| MG29-1_29-13 | TTTn |
| MG29-1_29-14 | TYYn |
| MG29-1_29-15 | inactive |
| MG29-1_29-16 | TTTn |
| MG29-1_29-17 | inactive |
| MG29-1 29-18 | nYTn |
| MG29-1_29-20 | TTYn |
| MG29-1_29-21 | inactive |
| MG29-1_11-18 | inactive |
| MG29-1_79-1 | inactive |
| MG29-1_29-229 | nTTn |
| MG29-1_29-230 | nYYn |
| MG29-1_29-231 | nYYn |

### REFERENCES

Kauffman, K. J. et al. Optimization of Lipid Nanoparticle Formulations for mRNA Delivery in Vivo with Fractional Factorial and Definitive Screening Designs. Nano Lett 15, 7300-7306 (2015).
Harms, M. & Thornton J. W. Analyzing protein structure and function using ancestral gene reconstruction. Current Opinion in Structural Biology, 20:360-366 (2010).
Katoh K, Standley D M. MAFFT multiple sequence alignment software version 7: improvements in performance and usability. Mol Biol Evol. 2013; 30(4):772-780. doi:10.1093/molbev/mst010
Stamatakis, A. RAxML version 8: a tool for phylogenetic analysis and post-analysis of large phylogenies. Bioinformatics 30(9), 1312-1313 (2014).
Yang, Z. PAML 4: a program package for phylogenetic analysis by maximum likelihood. Molecular Biology and Evolution 24: 1586-1591 (2007).

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

What is claimed is:

1. An engineered endonuclease, comprising:
a) an N-terminal portion comprising a sequence having at least 80% sequence identity to SEQ ID NO: 696; and
b) a C-terminal portion comprising a sequence having at least 80% sequence identity to any one of SEQ ID NOs: 697-721.

2. The engineered endonuclease of claim 1, wherein the N-terminal portion and the C-terminal portion are fused directly to each other.

3. The engineered endonuclease of claim 1, wherein the N-terminal portion and the C-terminal portion are joined by a linker.

4. The engineered endonuclease of claim 3, wherein the linker is a glycine and/or serine-rich linker, a large protein domain, a long helix structure, or a short helix structure.

5. The engineered endonuclease of any one of claims 3-4, wherein the linker is (GGGGS)n, and wherein n is an integer from 1 to 20 SEQ ID NO: 2950).

6. The engineered endonuclease of any one of claims 3-4, wherein the linker is GGGGS (SEQ ID NO: 2864).

7. The engineered endonuclease of any one of claims 1-6, wherein the N-terminal portion comprises a sequence having at least 90% sequence identity to SEQ ID NO: 696.

8. The engineered endonuclease of any one of claims 1-6, wherein the N-terminal portion comprises a sequence having 100% sequence identity to SEQ ID NO: 696.

9. The engineered endonuclease of any one of claims 1-8, wherein the C-terminal portion comprises a sequence having at least 90% sequence identity to any one of SEQ ID NOs: 697-721.

10. The engineered endonuclease of any one of claims 1-8, wherein the C-terminal portion comprises a sequence having 100% sequence identity to any one of SEQ ID NOs: 697-721.

11. The engineered endonuclease of any one of claims 1-10, wherein the engineered endonuclease is configured to bind to a PAM that comprises any one of SEQ ID NOs: 60-66, 117, 865-919, and 2855-2863.

12. An engineered endonuclease comprising a sequence having at least 80% sequence identity to any one of SEQ ID NOs: 1-27 and 771-862.

13. The engineered endonuclease of claim 12, wherein the engineered endonuclease comprises a sequence having at least 90% sequence identity to any one of SEQ ID NOs: 1-27 and 771-862.

14. The engineered endonuclease of claim 12, wherein the engineered endonuclease comprises a sequence having 100% sequence identity to any one of SEQ ID NOs: 1-27 and 771-862.

15. An engineered endonuclease comprising a sequence having at least 80% sequence identity to SEQ ID NO: 10.

16. The engineered endonuclease of claim 15, wherein the engineered endonuclease comprises a sequence having at least 90% sequence identity to SEQ ID NO: 10.

17. The engineered endonuclease of claim 15, wherein the engineered endonuclease comprises a sequence having 100% sequence identity to SEQ ID NO: 10.

18. An engineered endonuclease comprising a sequence having at least 80% sequence identity to any one of SEQ ID NOs: 109-110 and 2842-2854.

19. The engineered endonuclease of claim 18, wherein the engineered endonuclease comprises a sequence having at least 90% sequence identity to any one of SEQ ID NOs: 109-110 and 2842-2854.

20. The engineered endonuclease of claim 18, wherein the engineered endonuclease comprises a sequence having 100% sequence identity to any one of SEQ ID NOs: 109-110 and 2842-2854.

21. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and to hybridize to a target nucleic acid sequence.

22. The engineered nuclease system of claim 21, wherein the engineered endonuclease comprises a sequence having at least 90% sequence identity to any one of SEQ ID NOs: 1-27 and 771-862.

23. The engineered nuclease system of claim 21, wherein the engineered endonuclease comprises a sequence having 100% sequence identity to any one of SEQ ID NOs: 1-27 and 771-862.

24. The engineered nuclease system of any one of claims 21-23, wherein the engineered guide polynucleotide is a single guide nucleic acid.

25. The engineered nuclease system of any one of claims 21-23, wherein the engineered guide polynucleotide is a dual guide nucleic acid.

26. The engineered nuclease system of any one of claims 21-23, wherein the engineered guide polynucleotide is RNA.

27. The engineered nuclease system of any one of claims 21-26, wherein the engineered endonuclease binds non-covalently to the engineered guide polynucleotide.

28. The engineered nuclease system of any one of claims 21-26, wherein the endonuclease is covalently linked to the engineered guide polynucleotide.

29. The engineered nuclease system of any one of claims 21-26, wherein the endonuclease is fused to the engineered guide polynucleotide.

30. The engineered nuclease system of any one of claims 21-29, wherein the engineered guide polynucleotide comprises a sequence having at least 90% sequence identity to any one of SEQ ID NOs: 28-45, 605-610, 646-695, 863, and 1789-1826.

31. The engineered nuclease system of any one of claims 21-29, wherein the engineered guide polynucleotide comprises a sequence having 100% sequence identity to any one of SEQ ID NOs: 28-45, 605-610, 646-695, 863, and 1789-1826.

32. The engineered nuclease system of any one of claims 21-31, wherein the engineered endonuclease is configured to bind to a PAM that comprises any one of SEQ ID NOs: 60-66, 865-919, and 2863.

33. An engineered nuclease system, comprising:
a) an engineered endonuclease comprising a sequence having at least 80% sequence identity to any one of SEQ ID NOs: 109-110 and 2842-2854; and
b) an engineered guide polynucleotide configured to form a complex with the endonuclease and to hybridize to a target nucleic acid sequence.

34. The engineered nuclease system of claim 33, wherein the engineered endonuclease comprises a sequence having at least 90% sequence identity to any one of SEQ ID NOs: 109-110 and 2842-2854.

35. The engineered nuclease system of claim 33, wherein the engineered endonuclease comprises a sequence having 100% sequence identity to any one of SEQ ID NOs: 109-110 and 2842-2854.

36. The engineered nuclease system of any one of claims 33-35, wherein the engineered guide polynucleotide is a single guide nucleic acid.

37. The engineered nuclease system of any one of claims 33-35, wherein the engineered guide polynucleotide is a dual guide nucleic acid.

38. The engineered nuclease system of any one of claims 33-35, wherein the engineered guide polynucleotide is RNA.

39. The engineered nuclease system of any one of claims 33-38, wherein the engineered endonuclease binds non-covalently to the engineered guide polynucleotide.

40. The engineered nuclease system of any one of claims 33-38, wherein the endonuclease is covalently linked to the engineered guide polynucleotide.

41. The engineered nuclease system of any one of claims 33-38, wherein the endonuclease is fused to the engineered guide polynucleotide.

42. The engineered nuclease system of any one of claims 33-41, wherein the engineered guide polynucleotide comprises a sequence having at least 90% sequence identity to any one of SEQ ID NOs: 111-113.

43. The engineered nuclease system of any one of claims 33-41, wherein the engineered guide polynucleotide comprises a sequence having 100% sequence identity to any one of SEQ ID NOs: 111-113.

44. The engineered nuclease system of any one of claims 33-43, wherein the engineered endonuclease is configured to bind to a PAM that comprises any one of SEQ ID NOs: 117 and 2855-2862.

45. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within an albumin gene or within an intron of an endogenous gene, the engineered guide polynucleotide comprising a sequence having at least 90% sequence identity to any one of SEQ ID NOs: 67-86.

46. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a TRAC gene or within an intron of an endogenous gene, the engineered guide polynucleotide comprising a sequence having at least 90% sequence identity to any one of SEQ ID NOs: 119-138, 922-924, 972-991, 1088-1183, 1280-1320, 2390-2485, and 2582-2617.

47. The engineered nuclease system of claim 46, wherein the target nucleic acid sequence comprises a sequence having at least 90% sequence identity to any one of SEQ ID NOs: 139-158,925-927,992-1011, 1184-1279, 1321-1361, 2486-2581, and 2618-2653.

48. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a B2M gene or within an intron of an endogenous gene, the engineered guide polynucleotide comprising a sequence having at least 90% sequence identity to any one of SEQ ID NOs: 159-184.

49. The engineered nuclease system of claim 48, wherein the target nucleic acid sequence comprises a sequence having at least 90% sequence identity to any one of SEQ ID NOs: 185-210.

50. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a TRBC1 gene or within an intron of an endogenous gene, the engineered guide polynucleotide comprising a sequence having at least 90% sequence identity to any one of SEQ ID NOs: 211-251.

51. The engineered nuclease system of claim 50, wherein the target nucleic acid sequence comprises a sequence having at least 90% sequence identity to any one of SEQ ID NOs: 252-292.

52. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a TRBC2 gene or within an intron of an endogenous gene, the engineered guide polynucleotide comprising a sequence having at least 90% sequence identity to any one of SEQ ID NOs: 293-337.

53. The engineered nuclease system of claim 52, wherein the target nucleic acid sequence comprises a sequence having at least 90% sequence identity to any one of SEQ ID NOs: 338-382.

54. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within an ANGPTL3 gene or within an intron of an endogenous gene, the engineered guide polynucleotide comprising a sequence having at least 90% sequence identity to any one of SEQ ID NOs: 383-477, 1392-1489, 2120-2215, and 2312-2350.

55. The engineered nuclease system of claim 54, wherein the target nucleic acid sequence comprises a sequence having at least 90% sequence identity to any one of SEQ ID NOs: 478-572, 1490-1587, 2216-2311, and 2351-2389.

56. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a PCSK9 gene or within an intron of an endogenous gene, the engineered guide polynucleotide comprising a sequence having at least 90% sequence identity to any one of SEQ ID NOs: 573-587 and 1362-1376.

57. The engineered nuclease system of claim 56, wherein the target nucleic acid sequence comprises a sequence having at least 90% sequence identity to any one of SEQ ID NOs: 588-602 and 1377-1391.

58. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a VCP gene or within an intron of an endogenous gene, the engineered guide polynucleotide comprising a sequence having at least 90% sequence identity to any one of SEQ ID NOs: 723-738 and 755-762.

59. The engineered nuclease system of claim 58, wherein the target nucleic acid sequence comprises a sequence having at least 90% sequence identity to any one of SEQ ID NOs: 739-754 and 763-770.

60. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within an AAVS1 locus or within an intron of an endogenous gene, the engineered guide polynucleotide comprising a sequence having at least 90% sequence identity to any one of SEQ ID NOs: 928-949 and 1012-1049.

61. The engineered nuclease system of claim 60, wherein the target nucleic acid sequence comprises a sequence having at least 90% sequence identity to any one of SEQ ID NOs: 950-971 and 1050-1087.

62. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a GPR146 gene or within an intron of an endogenous gene, the engineered guide polynucleotide comprising a sequence having at least 90% sequence identity to any one of SEQ ID NOs: 1588-1656.

63. The engineered nuclease system of claim 62, wherein the target nucleic acid sequence comprises a sequence having at least 90% sequence identity to any one of SEQ ID NOs: 1657-1725.

64. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within an APOA1 gene or within an intron of an endogenous gene, the engineered guide polynucleotide comprising a sequence having at least 90% sequence identity to any one of SEQ ID NOs: 1726-1744, 1764-1774, 1866-1961 and 2058-2088.

65. The engineered nuclease system of claim 64, wherein the target nucleic acid sequence comprises a sequence having at least 90% sequence identity to any one of SEQ ID NOs: 1745-1763, 1775-1785, 1962-2057, and 2089-2119.

66. An engineered nuclease system, comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least 90% sequence identity to any one of SEQ ID NOs: 611-633, 1789-1826, and 1827-1865.

67. A method for modifying a target nucleic acid sequence comprising contacting the target nucleic acid sequence using the endonuclease of any one of claims 1-32 or the engineered nuclease system of any one of claims 33-66.

68. The method of claim 67, wherein modifying the target nucleic acid sequence comprises binding, nicking, or cleaving, the target nucleic acid sequence.

69. The method of any one of claims 67-68, wherein the target nucleic acid sequence comprises genomic DNA, viral DNA, viral RNA, or bacterial DNA.

70. The method of any one of claims 67-69, wherein the modification is in vitro.

71. The method of any one of claims 67-69, wherein the modification is in vivo.

72. The method of any one of claims 67-69, wherein the modification is ex vivo.

73. The method of any one of claims 67-72, wherein the gRNA is encoded by a sequence having any one of SEQ ID NOs: 251-260, 271-274, and 279-290.

74. The method of any one of claims 67-72, wherein the target nucleic acid sequence comprises a sequence having any one of SEQ ID NOs: 261-270, 275-278, and 291-302.

75. A method of modifying a target nucleic acid sequence in a mammalian cell comprising contacting the mammalian cell using the endonuclease of any one of claims 1-32 or the engineered nuclease system of any one of claims 33-66.

76. The method of claim 75, further comprising selecting cells comprising the modification.

77. A method of modifying an albumin gene comprising contacting the albumin gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the albumin gene or within an intron of an endogenous gene, the engineered guide polynucleotide comprising a sequence having at least 90% sequence identity to any one of SEQ ID NOs: 67-86.

78. A method of modifying a TRAC gene comprising contacting the TRAC gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the TRAC gene or within an intron of an endogenous gene, the engineered guide polynucleotide comprising a sequence having at least 90% sequence identity to any one of SEQ ID NOs: 119-138, 922-924, 972-991, 1088-1183, 1280-1320, 2390-2485, and 2582-2617.

79. The method of claim 78, wherein the target nucleic acid sequence comprises a sequence having at least 90% sequence identity to any one of SEQ ID NOs: 139-158, 925-927, 992-1011, 1184-1279, 1321-1361, 2486-2581, and 2618-2653.

80. A method of modifying a B2M gene comprising contacting the B2M gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the B2M gene or within an intron of an endogenous gene, the engineered guide polynucleotide comprising a sequence having at least 90% sequence identity to any one of SEQ ID NOs: 159-184.

81. The method of claim 80, wherein the target nucleic acid sequence comprises a sequence having at least 90% sequence identity to any one of SEQ ID NOs: 185-210.

82. A method of modifying a TRBC1 gene comprising contacting the TRBC1 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the TRBC1 gene or within an intron of an endogenous gene, the engineered guide polynucleotide comprising a sequence having at least 90% sequence identity to any one of SEQ ID NOs: 211-251.

83. The method of claim 82, wherein the target nucleic acid sequence comprises a sequence having at least 90% sequence identity to any one of SEQ ID NOs: 252-292.

84. A method of modifying a TRBC2 gene comprising contacting the TRBC2 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the TRBC2 gene or within an intron of an endogenous gene, the engineered guide polynucleotide comprising a sequence having at least 90% sequence identity to any one of SEQ ID NOs: 293-337.

85. The method of claim 84, wherein the target nucleic acid sequence comprises a sequence having at least 90% sequence identity to any one of SEQ ID NOs: 338-382.

86. A method of modifying an ANGPTL3 gene comprising contacting the ANGPTL3 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the ANGPTL3 gene or within an intron of an endogenous gene, the engineered guide polynucleotide comprising a sequence having at least 90% sequence identity to any one of SEQ ID NOs: 383-477, 1392-1489, 2120-2215, and 2312-2350.

87. The method of claim 86, wherein the target nucleic acid sequence comprises a sequence having at least 90% sequence identity to any one of SEQ ID NOs: 478-572, 1490-1587, 2216-2311, and 2351-2389.

88. A method of modifying a PCSK9 gene comprising contacting the PCSK9 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the PCSK9 gene or within an intron of an endogenous gene, the engineered guide polynucleotide comprising a sequence having at least 90% sequence identity to any one of SEQ ID NOs: 573-587 and 1362-1376.

89. The method of claim 88, wherein the target nucleic acid sequence comprises a sequence having at least 90% sequence identity to any one of SEQ ID NOs: 588-602 and 1377-1391.

90. A method of modifying a VCP gene comprising contacting the VCP gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the VCP gene or within an intron of an endogenous gene, the engineered guide polynucleotide comprising a sequence having at least 90% sequence identity to any one of SEQ ID NOs: 723-738 and 755-762.

91. The method of claim 90, wherein the target nucleic acid sequence comprises a sequence having at least 90% sequence identity to any one of SEQ ID NOs: 739-754 and 763-770.

92. A method of modifying an AAVS1 locus comprising contacting the AAVS1 locus using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the AAVS1 locus or within an intron of an endogenous gene, the engineered guide polynucleotide comprising a sequence having at least 90% sequence identity to any one of SEQ ID NOs: 928-949 and 1012-1049.

93. The method of claim 92, wherein the target nucleic acid sequence comprises a sequence having at least 90% sequence identity to any one of SEQ ID NOs: 950-971 and 1050-1087.

94. A method of modifying a GPR146 gene comprising contacting the GPR146 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the GPR146 gene or within an intron of an endogenous gene, the engineered guide polynucleotide comprising a sequence having at least 90% sequence identity to any one of SEQ ID NOs: 1588-1656.

95. The method of claim 94, wherein the target nucleic acid sequence comprises a sequence having at least 90% sequence identity to any one of SEQ ID NOs: 1657-1725.

96. A method of modifying an APOA1 gene comprising contacting the APOA1 gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within the APOA1 gene or within an intron of an endogenous gene, the engineered guide polynucleotide comprising a sequence having at least 90% sequence identity to any one of SEQ ID NOs: 1726-1744, 1764-1774, 1866-1961 and 2058-2088.

97. The method of claim 96, wherein the target nucleic acid sequence comprises a sequence having at least 90% sequence identity to any one of SEQ ID NOs: 1745-1763, 1775-1785, 1962-2057, and 2089-2119.

98. A method of modifying a TRAC gene comprising contacting the TRAC gene using an engineered nuclease system comprising:
a) an engineered endonuclease comprising a sequence having at least 80% sequence identity to any one of SEQ ID NOs: 1-27 and 771-862; and
b) an engineered guide polynucleotide configured to form a complex with the endonuclease and comprising a spacer sequence configured to hybridize to at least a portion of a target nucleic acid sequence within a HAO1 gene or within an intron of an endogenous gene, the engineered guide polynucleotide comprising a sequence having at least 90% sequence identity to any one of SEQ ID NOs: 611-633, 1789-1826, and 1827-1865.

99. A cell comprising the endonuclease of any one of claims 1-32 or the engineered nuclease system of any one of claims 33-66.

100. The cell of claim 99, wherein the cell is a eukaryotic cell.

101. The cell of claim 99, wherein the cell is a mammalian cell.

102. The cell of claim 99, wherein the cell is an immortalized cell.

103. The cell of claim 99, wherein the cell is an insect cell.

104. The cell of claim 99, wherein the cell is a yeast cell.

105. The cell of claim 99, wherein the cell is a plant cell.

106. The cell of claim 99, wherein the cell is a fungal cell.

107. The cell of claim 99, wherein the cell is a prokaryotic cell.

108. The cell of claim 99, wherein the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof.

109. The cell of claim 99, wherein the cell is an engineered cell.

110. The cell of claim 99, wherein the cell is a stable cell.
